SEQUENCE LISTING (404 sequences)

SEQ ID NO: 1 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 2 - protein, 853 aa, Homo sapiens
MAVTSHHMIPVMVVLMSACLATAGPEPSTRCELSPINASHPVQALMESFTVLSGCASRGTTGLPREVHVLNLRSTDQGPGQRQREVTLHLNPIASVHTHHKPIVFLLNSPQPLVWHLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKCKKSVNWVIKSFDVKGNLKVIAPNSIGFGKESERSMTMTKLVRDDIPSTQENLMKWALDNGYRPVTSYTMAPVANRFHLRLENNEEMRDEEVHTIPPELRILLDPDHPPALDNPLFPGEGSPNGGLPFPFPDIPRRGWKEGEDRIPRPKQPIVPSVQLLPDHREPEEVQGGVDIALSVKCDHEKMVVAVDKDSFQTNGYSGMELTLLDPSCKAKMNGTHFVLESPLNGCGTRHRRSTPDGVVYYNSIVVQAPSPGDSSGWPDGYEDLESGDNGFPGDGDEGETAPLSRAGVVVFNCSLRQLRNPSGFQGQLDGNATFNMELYNTDLFLVPSPGVFSVAENEHVYVEVSVTKADQDLGFAIQTCFLSPYSNPDRMSDYTIIENICPKDDSVKFYSSKRVHFPIPHAEVDKKRFSFLFKSVFNTSLLFLHCELTLCSRKKGSLKLPRCVTPDDACTSLDATMIWTMMQNKKTFTKPLAVVLQVDYKENVPSTKDSSPIPPPPPQIFHGLDTLTVMGIAFAAFVIGALLTGALWYIYSHTGETARRQQVPTSPPASENSSAAHSIGSTQSTPCSSSSTA

SEQ ID NO: 3 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 4 - protein, 21 aa, Artificial Sequence
  Synthetic Construct
MKWVTFLLLLFISGSAFSAAA

SEQ ID NO: 5 - protein, 657 aa, Artificial Sequence
  Synthetic Construct
MKWVTFLLLLFISGSAFSAAANGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSGDDDDDDDDDD

SEQ ID NO: 6 - protein, 3 aa, Artificial Sequence
  Synthetic Construct
GGG

SEQ ID NO: 7 - protein, 5 aa, Artificial Sequence
  Synthetic Construct
GGGGS

SEQ ID NO: 8 - protein, 16 aa, Artificial Sequence
  Synthetic Construct
GGGGSGGGGSGGGGSG

SEQ ID NO: 9 - protein, 590 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 10 - protein, 118 aa, Homo sapiens
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 11 - protein, 112 aa, Homo sapiens
PQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 12 - protein, 360 aa, Artificial Sequence
  Synthetic Construct
GPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPD

SEQ ID NO: 13 - protein, 360 aa, Homo sapiens
GPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPG

SEQ ID NO: 14 - protein, 851 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 15 - protein, 841 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 863 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 17 - protein, 853 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 869 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 19 - protein, 859 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 873 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 21 - protein, 863 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 873 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 23 - protein, 863 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 875 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 25 - protein, 865 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 875 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 27 - protein, 865 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 884 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 29 - protein, 874 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 871 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 31 - protein, 861 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 884 aa, Artificial Sequence
  Synthetic Construct
DDDDDDDDDDGGGGSGGGGSGGGGSGGGGSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 33 - protein, 853 aa, Artificial Sequence
  Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 34 - protein, 884 aa, Artificial Sequence
  Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSGGGGSGDDDDDDDDDD

SEQ ID NO: 35 - protein, 853 aa, Artificial Sequence; identical to SEQ ID NO: 33
  Synthetic Construct

SEQ ID NO: 36 - protein, 4 aa, Artificial Sequence
  Synthetic Construct
TGGG

SEQ ID NO: 37 - protein, 22 aa, Artificial Sequence
  Synthetic Construct
LLLVIFQVTGISLLPPLGGGGS

SEQ ID NO: 38 - protein, 26 aa, Artificial Sequence
  Synthetic Construct
LLLVIFQVTGISLLPPLGVAISVIII

SEQ ID NO: 39 - protein, 17 aa, Artificial Sequence
  Synthetic Construct
LLLVIFQVTGISLLPPL

SEQ ID NO: 40 - protein, 17 aa, Artificial Sequence
  Synthetic Construct
GLGPVESSPGHGLDTAA

SEQ ID NO: 41 - protein, 21 aa, Artificial Sequence
  Synthetic Construct
GGGGSGGGGSGGGGSGGGGSG

SEQ ID NO: 42 - protein, 18 aa, Artificial Sequence
  Synthetic Construct
MMSFVSLLLVGILFHATQ

SEQ ID NO: 43 - protein, 118 aa, Homo sapiens; identical to SEQ ID NO: 10

SEQ ID NO: 44 - protein, 363 aa, Homo sapiens
AGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGAL

SEQ ID NO: 45 - protein, 112 aa, Homo sapiens; identical to SEQ ID NO: 11

SEQ ID NO: 46 - protein, 10 aa, Artificial Sequence
  Synthetic Construct
DDDDDDDDDD

SEQ ID NO: 47 - protein, 227 aa, Artificial Sequence
  Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 610 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 49 - protein, 610 aa, Artificial Sequence
  Synthetic Construct
NGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLAGPEPSTRCELSPINASHPVQALMESFTVLSGCASHGTTGLPREVHVLNLRSTDQGPGQRQREVTLHLNPIASVHTHHKPIVFLLNSPQPLVWRLKTERLAAGVPRLFLVSEGSVVQFPSGNFSLTAETEERNFPQENEHLLRWAQKEYGAVTSFTELKIARNIYIKVGEDQVFPPTCNIGKNFLSLNYLAEYLQPKAAEGCVLPSQPHEKEVHIIELITPSSNPYSAFQVDIIVDIRPAQEDPEVVKNLVLILKSKKSVNWVIKSFDVKGNLKVIAPNSIGFGKESERSMTMTKLVRDDIPSTQENLMKWALDAGYRPVTSYTMAPVANRFHLRLENNEEMRDEEVHTIPPELRILLDPDKLPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 50 - protein, 612 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 51 - protein, 612 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGLGPVESSPGHGLDTAAAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 52 - protein, 621 aa, Artificial Sequence
  Synthetic Construct
ADNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKSKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 53 - protein, 10 aa, Artificial Sequence
  Synthetic Construct
TGGGDGGGGS

SEQ ID NO: 54 - protein, 31 aa, Artificial Sequence
  Synthetic Construct
GGGGSLLLVIFQVTGISLLPPLGVAISVIII

SEQ ID NO: 55 - protein, 22 aa, Artificial Sequence
  Synthetic Construct
GGGGSLLLVIFQVTGISLLPPL

SEQ ID NO: 56 - protein, 27 aa, Artificial Sequence
  Synthetic Construct
GGGGSGGGGSLLLVIFQVTGISLLPPL

SEQ ID NO: 57 - protein, 22 aa, Artificial Sequence
  Synthetic Construct
GGGGSGLGPVESSPGHGLDTAA

SEQ ID NO: 58 - protein, 27 aa, Artificial Sequence
  Synthetic Construct
GGGGSGGGGSGLGPVESSPGHGLDTAA

SEQ ID NO: 59 - protein, 2 aa, Artificial Sequence
  Synthetic Construct
KL

SEQ ID NO: 60 - protein, 20 aa, Artificial Sequence
  Synthetic Construct
  VARIANT (5)..(20): Xaa′s at spots 6-8 match the status (present or absent) of Xaa at spot 5; Xaa′s at spots 10-12 match the status of Xaa at spot 9; Xaa′s at spots 14-16 match the status of Xaa at spot 13, and Xaa′s at spots 18-20 match the status of Xaa at spot 17;
  VARIANT (5)..(20): If Xaa at position 5 is present, then Xaa′s at positions 6-8 are present; If Xaa at position 5 is absent, then Xaa′s at positions 6-20 are also absent;
  VARIANT (5)..(20): If Xaa at position 9 is present, then Xaa′s at positions 5-8 and 10-12 are present; If Xaa at position 9 is absent, then Xaa′s at positions 10-20 are also absent;
  VARIANT (5)..(20): If Xaa at position 13 is present, then Xaa′s at positions 5-12 and 14-16 are present; If Xaa at position 13 is absent, then Xaa′s at positions 14-20 are also absent;
  VARIANT (5)..(20): If Xaa at position 17 is present, then Xaa′s at positions 5-16 and 18-20 are present; If Xaa at position 17 is absent, then Xaa′s at positions 18-20 are also absent;
  MISC_FEATURE (5)..(5): Xaa is Gly or absent
  MISC_FEATURE (6)..(6): Xaa is Gly or absent
  MISC_FEATURE (7)..(7): Xaa is Gly or absent
  MISC_FEATURE (8)..(8): Xaa is Ser or absent
  MISC_FEATURE (9)..(9): Xaa is Gly or absent
  MISC_FEATURE (10)..(10): Xaa is Gly or absent
  MISC_FEATURE (11)..(11): Xaa is Gly or absent
  MISC_FEATURE (12)..(12): Xaa is Ser or absent
  MISC_FEATURE (13)..(13): Xaa is Gly or absent
  MISC_FEATURE (14)..(14): Xaa is Gly or absent
  MISC_FEATURE (15)..(15): Xaa is Gly or absent
  MISC_FEATURE (16)..(16): Xaa is Ser or absent
  MISC_FEATURE (17)..(17): Xaa is Gly or absent
  MISC_FEATURE (18)..(18): Xaa is Gly or absent
  MISC_FEATURE (19)..(19): Xaa is Gly or absent
  MISC_FEATURE (20)..(20): Xaa is Ser or absent
GGGSXXXXXXXXXXXXXXXX

SEQ ID NO: 61 - protein, 25 aa, Artificial Sequence
  Synthetic Construct
  VARIANT (6)..(25): Xaa′s at spots 7-10 match the status (present or absent) of Xaa at spot 6; Xaa′s at spots 12-15 match the status of Xaa at spot 11; Xaa′s at spots 17-20 match the status of Xaa at spot 16, and Xaa′s at spots 22-25 match the status of Xaa at spot 21;
  VARIANT (6)..(25): If Xaa at position 6 is present, then Xaa′s at positions 7-10 are present; If Xaa at position 6 is absent, then Xaa′s at positions 7-25 are also absent;
  VARIANT (6)..(25): If Xaa at position 11 is present, then Xaa′s at positions 6-10 and 12-15 are present; If Xaa at position 11 is absent, then Xaa′s at positions 12-25 are also absent;
  VARIANT (6)..(25): If Xaa at position 16 is present, then Xaa′s at positions 6-15 and 17-20 are present; If Xaa at position 16 is absent, then Xaa′s at positions 17-25 are also absent;
  VARIANT (6)..(25): If Xaa at position 21 is present, then Xaa′s at positions 6-20 and 22-25 are present; If Xaa at position 21 is absent, then Xaa′s at positions 22-25 are also absent;
  MISC_FEATURE (6)..(6): Xaa is Gly or absent
  MISC_FEATURE (7)..(7): Xaa is Gly or absent
  MISC_FEATURE (8)..(8): Xaa is Gly or absent
  MISC_FEATURE (9)..(9): Xaa is Gly or absent
  MISC_FEATURE (10)..(10): Xaa is Ser or absent
  MISC_FEATURE (11)..(11): Xaa is Gly or absent
  MISC_FEATURE (12)..(12): Xaa is Gly or absent
  MISC_FEATURE (13)..(13): Xaa is Gly or absent
  MISC_FEATURE (14)..(14): Xaa is Gly or absent
  MISC_FEATURE (15)..(15): Xaa is Ser or absent
  MISC_FEATURE (16)..(16): Xaa is Gly or absent
  MISC_FEATURE (17)..(17): Xaa is Gly or absent
  MISC_FEATURE (18)..(18): Xaa is Gly or absent
  MISC_FEATURE (19)..(19): Xaa is Gly or absent
  MISC_FEATURE (20)..(20): Xaa is Ser or absent
  MISC_FEATURE (21)..(21): Xaa is Gly or absent
  MISC_FEATURE (22)..(22): Xaa is Gly or absent
  MISC_FEATURE (23)..(23): Xaa is Gly or absent
  MISC_FEATURE (24)..(24): Xaa is Gly or absent
  MISC_FEATURE (25)..(25): Xaa is Ser or absent
GGGGSXXXXXXXXXXXXXXXXXXXX

SEQ ID NO: 62 - protein, 457 aa, Artificial Sequence
  Synthetic Construct
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKDDDDDDDDDD

SEQ ID NO: 63 - protein, 215 aa, Artificial Sequence
  Synthetic Construct
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 5 aa, Artificial Sequence
  Synthetic Construct
SNVIS

SEQ ID NO: 65 - protein, 17 aa, Artificial Sequence
  Synthetic Construct
GVIPIVDIANYAQRFKG

SEQ ID NO: 66 - protein, 11 aa, Artificial Sequence
  Synthetic Construct
TLGLVLDAMDY

SEQ ID NO: 67 - protein, 12 aa, Artificial Sequence
  Synthetic Construct
RASQSLGSSYLA

SEQ ID NO: 68 - protein, 7 aa, Artificial Sequence
  Synthetic Construct
GASSRAP

SEQ ID NO: 69 - protein, 9 aa, Artificial Sequence
  Synthetic Construct
QQYADSPIT

SEQ ID NO: 70 - protein, 120 aa, Artificial Sequence
  Synthetic Construct
QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS

SEQ ID NO: 71 - protein, 108 aa, Artificial Sequence
  Synthetic Construct
ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK

SEQ ID NO: 72 - protein, 19 aa, Artificial Sequence
  Synthetic Construct
WIWLDTNMGYRIYQEFEVT

SEQ ID NO: 73 - protein, 1428 aa, Artificial Sequence
  Synthetic Construct
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGYRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPLAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 74 - protein, 19 aa, Artificial Sequence
  Synthetic Construct
WIWLDTNMGSRIYQEFEVT

SEQ ID NO: 75 - protein, 1428 aa, Artificial Sequence
  Synthetic Construct
MQGPPLLTAAHLLCVCTAALAVAPGPRFLVTAPGIIRPGGNVTIGVELLEHCPSQVTVKAELLKTASNLTVSVLEAEGVFEKGSFKTLTLPSLPLNSADEIYELRVTGRTQDEILFSNSTRLSFETKRISVFIQTDKALYKPKQEVKFRIVTLFSDFKPYKTSLNILIKDPKSNLIQQWLSQQSDLGVISKTFQLSSHPILGDWSIQVQVNDQTYYQSFQVSEYVLPKFEVTLQTPLYCSMNSKHLNGTITAKYTYGKPVKGDVTLTFLPLSFWGKKKNITKTFKINGSANFSFNDEEMKNVMDSSNGLSEYLDLSSPGPVEILTTVTESVTGISRNVSTNVFFKQHDYIIEFFDYTTVLKPSLNFTATVKVTRADGNQLTLEERRNNVVITVTQRNYTEYWSGSNSGNQKMEAVQKINYTVPQSGTFKIEFPILEDSSELQLKAYFLGSKSSMAVHSLFKSPSKTYIQLKTRDENIKVGSPFELVVSGNKRLKELSYMVVSRGQLVAVGKQNSTMFSLTPENSWTPKACVIVYYIEDDGEIISDVLKIPVQLVFKNKIKLYWSKVKAEPSEKVSLRISVTQPDSIVGIVAVDKSVNLMNASNDITMENVVHELELYNTGYYLGMFMNSFAVFQECGLWVLTDANLTKDYIDGVYDNAEYAERFMEENEGHIVDIHDFSLGSSPHVRKHFPETWIWLDTNMGSRIYQEFEVTVPDSITSWVATGFVISEDLGLGLTTTPVELQAFQPFFIFLNLPYSVIRGEEFALEITIFNYLKDATEVKVIIEKSDKFDILMTSNEINATGHQQTLLVPSEDGATVLFPIRPTHLGEIPITVTALSPTASDAVTQMILVKAEGIEKSYSQSILLDLTDNRLQSTLKTLSFSFPPNTVTGSERVQITAIGDVLGPSINGLASLIRMPYGCGEQNMINFAPNIYILDYLTKKKQLTDNLKEKALSFMRQGYQRELLYQREDGSFSAFGNYDPSGSTWLSAFVLRCFLEADPYIDIDQNVLHRTYTWLKGHQKSNGEFWDPGRVIHSELQGGNKSPVTLTAYIVTSLLGYRKYQPNIDVQESIHFLESEFSRGISDNYTLALITYALSSVGSPKAKEALNMLTWRAEQEGGMQFWVSSESKLSDSWQPRSLDIEVAAYALLSHFLQFQTSEGIPIMRWLSRQRNSLGGFASTQDTTVALKALSEFAALMNTERTNIQVTVTGPSSPSPLAVVQPTAVNISANGFGFAICQLNVVYNVKASGSSRRRRSIQNQEAFDLDVAVKENKDDLNHVDLNVCTSFSGPGRSGMALMEVNLLSGFMVPSEAISLSETVKKVEYDHGKLNLYLDSVNETQFCVNIPAVRNFKVSNTQDASVSIVDYYEPRRQAVRSYNSEVKLSSCDLCSDVQGCRPCEDGASGSHHHSSVIFIFCFKLLYFMELWL

SEQ ID NO: 76 - protein, 33 aa, Artificial Sequence
  Synthetic Construct
IDGVYDNAEYAERFMEENEGHIVDIHDFSLGSS

SEQ ID NO: 77 - protein, 161 aa, Artificial Sequence
  Synthetic Construct
TMENVVHELELYNTGYYLGMFMNSFAVFQECGLWVLTDANLTKDYIDGVYDNAEYAERFMEENEGHIVDIHDFSLGSSPHVRKHFPETWIWLDTNMGSRIYQEFEVTVPDSITSWVATGFVISEDLGLGLTTTPVELQAFQPFFIFLNLPYSVIRGEEFAL

SEQ ID NO: 78 - protein, 25 aa, Artificial Sequence
  Synthetic Construct
RKHFPETWIWLDTNMGYRIYQEFEV

SEQ ID NO: 79 - protein, 16 aa, Artificial Sequence
  Synthetic Construct
ANFCLGPCPYIWSLDT

SEQ ID NO: 80 - protein, 16 aa, Artificial Sequence
  Synthetic Construct
ANFCSGPCPYLRSADT

SEQ ID NO: 81 - protein, 10 aa, Artificial Sequence
  Synthetic Construct
PYIWSLDTQY

SEQ ID NO: 82 - protein, 10 aa, Artificial Sequence
  Synthetic Construct
PYLWSSDTQH

SEQ ID NO: 83 - protein, 10 aa, Artificial Sequence
  Synthetic Construct
PYLRSADTTH

SEQ ID NO: 84 - protein, 4 aa, Artificial Sequence
  Synthetic Construct
  MISC_FEATURE (3)..(3): Xaa is any naturally occurring amino acid
WSXD

SEQ ID NO: 85 - protein, 4 aa, Artificial Sequence
  Synthetic Construct
  MISC_FEATURE (3)..(3): Xaa is any naturally occurring amino acid
RSXD

SEQ ID NO: 86 - protein, 12 aa, Artificial Sequence
  Synthetic Construct
TSLDATMIWTMM

SEQ ID NO: 87 - protein, 15 aa, Artificial Sequence
  Synthetic Construct
SNPYSAFQVDIIVDI

SEQ ID NO: 88 - protein, 9 aa, Artificial Sequence
  Synthetic Construct
TSLMIWTMM

SEQ ID NO: 89 - protein, 14 aa, Artificial Sequence
  Synthetic Construct
TSLDASIIWAMMQN

SEQ ID NO: 90 - protein, 14 aa, Artificial Sequence
  Synthetic Construct
SNPYSAFQVDITID

SEQ ID NO: 91 - protein, 15 aa, Artificial Sequence
  Synthetic Construct
EAVLILQGPPYVSWL

SEQ ID NO: 92 - protein, 15 aa, Artificial Sequence
  Synthetic Construct
LDSLSFQLGLYLSPH

SEQ ID NO: 93 - protein, 15 aa, Artificial Sequence
  Synthetic Construct
HANFCLGPCPYIWSL

SEQ ID NO: 94 - protein, 14 aa, Artificial Sequence
  Synthetic Construct
FCLGPCPYIWSLDT

SEQ ID NO: 95 - protein, 16 aa, Artificial Sequence
  Synthetic Construct
HEPKGYHANFCLGPCP

SEQ ID NO: 96 - protein, 14 aa, Artificial Sequence
  Synthetic Construct
TSLDASIIWAMMQN

SEQ ID NO: 97 - protein, 15 aa, Artificial Sequence
  Synthetic Construct
KRIWFIPRSSWYERA

SEQ ID NO: 98 - protein, 11 aa, Artificial Sequence
  Synthetic Construct
KRIWFIPRSSW

SEQ ID NO: 99 - protein, 11 aa, Artificial Sequence
  Synthetic Construct
  MOD_RES (11)..(11): AMIDATION
KRIWFIPRSSW

SEQ ID NO: 100 - protein, 11 aa, Artificial Sequence
  Synthetic Construct
  MOD_RES (1)..(1): ACETYLATION
  MOD_RES (11)..(11): AMIDATION
KRIWFIPRSSW

SEQ ID NO: 101 - protein, 15 aa, Artificial Sequence
  Synthetic Construct
HANFCLGPCPYIWSL

SEQ ID NO: 102 - protein, 14 aa, Artificial Sequence
  Synthetic Construct
FCLGPCPYIWSLDT

SEQ ID NO: 103 - protein, 12 aa, Artificial Sequence
  Synthetic Construct
TSLDATMIWTMM

SEQ ID NO: 104 - protein, 15 aa, Artificial Sequence
  Synthetic Construct
SNPYSAFQVDIIVDI

SEQ ID NO: 105 - protein, 9 aa, Artificial Sequence
  Synthetic Construct
TSLMIWTMM

SEQ ID NO: 106 - protein, 14 aa, Artificial Sequence
  Synthetic Construct
TSLDASIIWAMMQN

SEQ ID NO: 107 - protein, 14 aa, Artificial Sequence
  Synthetic Construct
SNPYSAFQVDITID

SEQ ID NO: 108 - protein, 15 aa, Artificial Sequence
  Synthetic Construct
EAVLILQGPPYVSWL

SEQ ID NO: 109 - protein, 15 aa, Artificial Sequence
  Synthetic Construct
LDSLSFQLGLYLSPH

SEQ ID NO: 110 - protein, 23 aa, Artificial Sequence
  Synthetic Construct
HEPKGYHANFCLGPCPYIWSLDT

SEQ ID NO: 111 - protein, 15 aa, Artificial Sequence
  Synthetic Construct
WHKYFLRRPLSVRTR

SEQ ID NO: 112 - protein, 15 aa, Artificial Sequence
  Synthetic Construct
RFFTRFPWHYHASRL

SEQ ID NO: 113 - protein, 15 aa, Artificial Sequence
  Synthetic Construct
RKWFLQHRRMPVSVL

SEQ ID NO: 114 - protein, 15 aa, Artificial Sequence
  Synthetic Construct
SGRRHLHRHHIFSLP

SEQ ID NO: 115 - protein, 15 aa, Artificial Sequence
  Synthetic Construct
RLAHSHRHRSHVALT

SEQ ID NO: 116 - protein, 15 aa, Artificial Sequence
  Synthetic Construct
PPYHRFWRGHRHAVQ

SEQ ID NO: 117 - protein, 15 aa, Artificial Sequence
  Synthetic Construct
KRIWFIPRSSWYERA

SEQ ID NO: 118 - protein, 15 aa, Artificial Sequence
  Synthetic Construct
MPLSRYWWLFSHRPR

SEQ ID NO: 119 - protein, 14 aa, Artificial Sequence
  Synthetic Construct
KRIWFIPRSSWYER

SEQ ID NO: 120 - protein, 12 aa, Artificial Sequence
  Synthetic Construct
KRIWFIPRSSWY

SEQ ID NO: 121 - protein, 11 aa, Artificial Sequence
  Synthetic Construct
KRIWFIPRSSW

SEQ ID NO: 122 - protein, 11 aa, Artificial Sequence
  Synthetic Construct
  MOD_RES (11)..(11): AMIDATION
KRIWFIPRSSW

SEQ ID NO: 123 - protein, 11 aa, Artificial Sequence
  Synthetic Construct
  MOD_RES (1)..(1): ACETYLATION
  MOD_RES (11)..(11): AMIDATION
KRIWFIPRSSW

SEQ ID NO: 124 - protein, 662 aa, Artificial Sequence
  Synthetic Construct
MRPQILLLLALLTLGLAAQHQDKVPCKMVDKKVSCQVLGLLQVPSVLPPDTETLDLSGNQLRSILASPLGFYTALRHLDLSTNEISFLQPGAFQALTHLEHLSLAHNRLAMATALSAGGLGPLPRVTSLDLSGNSLYSGLLERLLGEAPSLHTLSLAENSLTRLTRHTFRDMPALEQLDLHSNVLMDIEDGAFEGLPRLTHLNLSRNSLTCISDFSLQQLRVLDLSCNSIEAFQTASQPQAEFQLTWLDLRENKLLHFPDLAALPRLIYLNLSNNLIRLPTGPPQDSKGIHAPSEGWSALPLSAPSGNASGRPLSQLLNLDLSYNEIELIPDSFLEHLTSLCFLNLSRNCLRTFEARRLGSLPCLMLLDLSHNALETLELGARALGSLRTLLLQGNALRDLPPYTFANLASLQRLNLQGNRVSPCGGPDEPGPSGCVAFSGITSLRSLSLVDNEIELLRAGAFLHTPLTELDLSSNPGLEVATGALGGLEASLEVLALQGNGLMVLQVDLPCFICLKRLNLAENRLSHLPAWTQAVSLEVLDLRNNSFSLLPGSAMGGLETSLRRLYLQGNPLSCCGNGWLAAQLHQGRVDVDATQDLICRFSSQEEVSLSHVRPEDCEKGGLKNINLIIILTFILVSAILLTTLAACCCVRRQKFNQQYKA

SEQ ID NO: 125 - protein, 121 aa, Artificial Sequence
  Synthetic Construct
PVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 126 - protein, 215 aa, Artificial Sequence
  Synthetic Construct
EIKDLSENKLPVIYMHVPKSGALNQKVVFYGKGTYDPDGSIAGYQWDFGDGSDFSSEQNPSHVYTKKGEYTVTLRVMDSSGQMSEKTMKIKITDPVYPIGTEKEPNNSKETASGPIVPGIPVSGTIENTSDQDYFYFDVITPGEVKIDINKLGYGGATWVVYDENNNAVSYATDDGQNLSGKFKADKPGRYYIHLYMFNGSYMPYRINIEGSVGR

SEQ ID NO: 127 - protein, 183 aa, Artificial Sequence
  Synthetic Construct
QSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVT

SEQ ID NO: 128 - protein, 28 aa, Artificial Sequence
  Synthetic Construct
NGVFKYRPRYFLYKHAYFYPPLKRFPVQ

SEQ ID NO: 129 - protein, 14 aa, Artificial Sequence
  Synthetic Construct
NGVFKYRPRYFLYK

SEQ ID NO: 130 - protein, 14 aa, Artificial Sequence
  Synthetic Construct
HAYFYPPLKRFPVQ

SEQ ID NO: 131 - protein, 7 aa, Artificial Sequence
  Synthetic Construct
RPHTITN

SEQ ID NO: 132 - protein, 7 aa, Artificial Sequence
  Synthetic Construct
QSSYNPI

SEQ ID NO: 133 - protein, 7 aa, Artificial Sequence
  Synthetic Construct
QTHARHQ

SEQ ID NO: 134 - protein, 7 aa, Artificial Sequence
  Synthetic Construct
ETRTQLL

SEQ ID NO: 135 - protein, 7 aa, Artificial Sequence
  Synthetic Construct
HHQRSPA

SEQ ID NO: 136 - protein, 7 aa, Artificial Sequence
  Synthetic Construct
LQKSPSL

SEQ ID NO: 137 - protein, 7 aa, Artificial Sequence
  Synthetic Construct
PPKDSRG

SEQ ID NO: 138 - protein, 7 aa, Artificial Sequence
  Synthetic Construct
SAKKVFS

SEQ ID NO: 139 - protein, 7 aa, Artificial Sequence
  Synthetic Construct
SQHSTQD

SEQ ID NO: 140 - protein, 7 aa, Artificial Sequence
  Synthetic Construct
TIHSKPA

SEQ ID NO: 141 - protein, 7 aa, Artificial Sequence
  Synthetic Construct
TKDWLPS

SEQ ID NO: 142 - protein, 7 aa, Artificial Sequence
  Synthetic Construct
ANPPLSL

SEQ ID NO: 143 - protein, 7 aa, Artificial Sequence
  Synthetic Construct
AKQTVPV

SEQ ID NO: 144 - protein, 7 aa, Artificial Sequence
  Synthetic Construct
ATFSPPL

SEQ ID NO: 145 - protein, 7 aa, Artificial Sequence
  Synthetic Construct
DQYWGLR

SEQ ID NO: 146 - protein, 7 aa, Artificial Sequence
  Synthetic Construct
EPNHTRF

SEQ ID NO: 147 - protein, 7 aa, Artificial Sequence
  Synthetic Construct
HMLAQTF

SEQ ID NO: 148 - protein, 7 aa, Artificial Sequence
  Synthetic Construct
IGYPVLP

SEQ ID NO: 149 - protein, 7 aa, Artificial Sequence
  Synthetic Construct
KLSAWSF

SEQ ID NO: 150 - protein, 7 aa, Artificial Sequence
  Synthetic Construct
MYPLPAP

SEQ ID NO: 151 - protein, 7 aa, Artificial Sequence
  Synthetic Construct
FTLPTIR

SEQ ID NO: 152 - protein, 7 aa, Artificial Sequence
  Synthetic Construct
SMAAKSS

SEQ ID NO: 153 - protein, 7 aa, Artificial Sequence
  Synthetic Construct
SMYDTHS

SEQ ID NO: 154 - protein, 7 aa, Artificial Sequence
  Synthetic Construct
STLASMR

SEQ ID NO: 155 - protein, 7 aa, Artificial Sequence
  Synthetic Construct
TLMTTPP

SEQ ID NO: 156 - protein, 7 aa, Artificial Sequence
  Synthetic Construct
WLPPRTQ

SEQ ID NO: 157 - protein, 12 aa, Artificial Sequence
  Synthetic Construct
RTPLQPLEDFRP

SEQ ID NO: 158 - protein, 12 aa, Artificial Sequence
  Synthetic Construct
NTTTDIPSPSQF

SEQ ID NO: 159 - protein, 12 aa, Artificial Sequence
  Synthetic Construct
TLDKYTRLLSRY

SEQ ID NO: 160 - protein, 12 aa, Artificial Sequence
  Synthetic Construct
YPIMSHTCCHGV

SEQ ID NO: 161 - protein, 7 aa, Artificial Sequence
  Synthetic Construct
YEPAAAE

SEQ ID NO: 162 - protein, 7 aa, Artificial Sequence
  Synthetic Construct
ANPYHRH

SEQ ID NO: 163 - protein, 7 aa, Artificial Sequence
  Synthetic Construct
ASGPTNV

SEQ ID NO: 164 - protein, 7 aa, Artificial Sequence
  Synthetic Construct
QNYLLPK

SEQ ID NO: 165 - protein, 7 aa, Artificial Sequence
  Synthetic Construct
GTQTPQP

SEQ ID NO: 166 - protein, 7 aa, Artificial Sequence
  Synthetic Construct
HSTGPTR

SEQ ID NO: 167 - protein, 7 aa, Artificial Sequence
  Synthetic Construct
LSKNPLL

SEQ ID NO: 168 - protein, 7 aa, Artificial Sequence
  Synthetic Construct
LSKNPLL

SEQ ID NO: 169 - protein, 7 aa, Artificial Sequence
  Synthetic Construct
KLHASLA

SEQ ID NO: 170 - protein, 7 aa, Artificial Sequence
  Synthetic Construct
PLTQPSH

SEQ ID NO: 171 - protein, 7 aa, Artificial Sequence
  Synthetic Construct
PHNPGKL

SEQ ID NO: 172 - protein, 7 aa, Artificial Sequence
  Synthetic Construct
PTTMTRW

SEQ ID NO: 173 - protein, 7 aa, Artificial Sequence
  Synthetic Construct
VHLTHGQ

SEQ ID NO: 174 - protein, 7 aa, Artificial Sequence
  Synthetic Construct
TLAPTFR

SEQ ID NO: 175 - protein, 7 aa, Artificial Sequence
  Synthetic Construct
VHPRPSL

SEQ ID NO: 176 - protein, 7 aa, Artificial Sequence
  Synthetic Construct
TLLRTQV

SEQ ID NO: 177 - protein, 7 aa, Artificial Sequence
  Synthetic Construct
SSPPRVY

SEQ ID NO: 178 - protein, 7 aa, Artificial Sequence
  Synthetic Construct
SSVPGRP

SEQ ID NO: 179 - protein, 7 aa, Artificial Sequence
  Synthetic Construct
LPFQPPI

SEQ ID NO: 180 - protein, 7 aa, Artificial Sequence
  Synthetic Construct
IQHQAKT

SEQ ID NO: 181 - protein, 12 aa, Artificial Sequence
  Synthetic Construct
LPRDLHATPQQI

SEQ ID NO: 182 - protein, 12 aa, Artificial Sequence
  Synthetic Construct
LTPTMFNMHGVL

SEQ ID NO: 183 - protein, 12 aa, Artificial Sequence
  Synthetic Construct
SIPKMIPTESLL

SEQ ID NO: 184 - protein, 12 aa, Artificial Sequence
  Synthetic Construct
SFQSMSLMTLVV

SEQ ID NO: 185 - protein, 12 aa, Artificial Sequence
  Synthetic Construct
TQTWPQSSSHGL

SEQ ID NO: 186 - protein, 6 aa, Artificial Sequence
  Synthetic Construct
YELQMP

SEQ ID NO: 187 - protein, 12 aa, Artificial Sequence
  Synthetic Construct
AMSQTMTAAIEK

SEQ ID NO: 188 - protein, 12 aa, Artificial Sequence
  Synthetic Construct
GSAGLKYPLYKS

SEQ ID NO: 189 - protein, 12 aa, Artificial Sequence
  Synthetic Construct
INFQFLKPSTTR

SEQ ID NO: 190 - protein, 7 aa, Artificial Sequence
  Synthetic Construct
RHTLPLH

SEQ ID NO: 191 - protein, 7 aa, Artificial Sequence
  Synthetic Construct
NFAMNLR

SEQ ID NO: 192 - protein, 7 aa, Artificial Sequence
  Synthetic Construct
NFAMNLR

SEQ ID NO: 193 - protein, 7 aa, Artificial Sequence
  Synthetic Construct
NPQMQRS

SEQ ID NO: 194 - protein, 7 aa, Artificial Sequence
  Synthetic Construct
NPQMQRS

SEQ ID NO: 195 - protein, 7 aa, Artificial Sequence
  Synthetic Construct
NPQMQRS

SEQ ID NO: 196 - protein, 7 aa, Artificial Sequence
  Synthetic Construct
NYPTLKS

SEQ ID NO: 197 - protein, 7 aa, Artificial Sequence
  Synthetic Construct
NYPTLKS

SEQ ID NO: 198 - protein, 7 aa, Artificial Sequence
  Synthetic Construct
QNPRQIY

SEQ ID NO: 199 - protein, 7 aa, Artificial Sequence
  Synthetic Construct
QNPRQIY

SEQ ID NO: 200 - protein, 7 aa, Artificial Sequence
  Synthetic Construct
QNPRQIY

SEQ ID NO: 201 - protein, 7 aa, Artificial Sequence
  Synthetic Construct
QNPRQIY

SEQ ID NO: 202 - protein, 7 aa, Artificial Sequence
  Synthetic Construct
ETYARPL

SEQ ID NO: 203 - protein, 7 aa, Artificial Sequence
  Synthetic Construct
ETVCASS

SEQ ID NO: 204 - protein, 7 aa, Artificial Sequence
  Synthetic Construct
KPMQFVH

SEQ ID NO: 205 - protein, 7 aa, Artificial Sequence
  Synthetic Construct
KPMQFVH

SEQ ID NO: 206 - protein, 7 aa, Artificial Sequence
  Synthetic Construct
PAKQKAH

SEQ ID NO: 207 - protein, 7 aa, Artificial Sequence
  Synthetic Construct
PTTWGHL

SEQ ID NO: 208 - protein, 7 aa, Artificial Sequence
  Synthetic Construct
PTTWGHL

SEQ ID NO: 209 - protein, 7 aa, Artificial Sequence
  Synthetic Construct
SASGTPS

SEQ ID NO: 210 - protein, 7 aa, Artificial Sequence
  Synthetic Construct
SSYEYHA

SEQ ID NO: 211 - protein, 7 aa, Artificial Sequence
  Synthetic Construct
SSYEYHA

SEQ ID NO: 212 - protein, 7 aa, Artificial Sequence
  Synthetic Construct
STQAHPW

SEQ ID NO: 213 - protein, 7 aa, Artificial Sequence
  Synthetic Construct
TVLGTFP

SEQ ID NO: 214 - protein, 7 aa, Artificial Sequence
  Synthetic Construct
WYPNHLA

SEQ ID NO: 215 - protein, 7 aa, Artificial Sequence
  Synthetic Construct
TTYNSPP

SEQ ID NO: 216 - protein, 7 aa, Artificial Sequence
  Synthetic Construct
MTSQTLR

SEQ ID NO: 217 - protein, 12 aa, Artificial Sequence
  Synthetic Construct
WPANKLSTKSMY

SEQ ID NO: 218 - protein, 12 aa, Artificial Sequence
  Synthetic Construct
WPANKLSTKSMY

SEQ ID NO: 219 - protein, 12 aa, Artificial Sequence
  Synthetic Construct
NPYHPTIPQSVH

SEQ ID NO: 220 - protein, 7 aa, Artificial Sequence
  Synthetic Construct
DKLHRLA

SEQ ID NO: 221 - protein, 7 aa, Artificial Sequence
  Synthetic Construct
QPGLWPS

SEQ ID NO: 222 - protein, 7 aa, Artificial Sequence
  Synthetic Construct
ESLKSIS

SEQ ID NO: 223 - protein, 7 aa, Artificial Sequence
  Synthetic Construct
GSCPPKK

SEQ ID NO: 224 - protein, 7 aa, Artificial Sequence
  Synthetic Construct
GSLFKAL

SEQ ID NO: 225 - protein, 7 aa, Artificial Sequence
  Synthetic Construct
HQWDHKY

SEQ ID NO: 226 - protein, 7 aa, Artificial Sequence
  Synthetic Construct
LSAPMEY

SEQ ID NO: 227 - protein, 7 aa, Artificial Sequence
  Synthetic Construct
MKVHERS

SEQ ID NO: 228 - protein, 7 aa, Artificial Sequence
  Synthetic Construct
FVNLLGQ

SEQ ID NO: 229 - protein, 7 aa, Artificial Sequence
  Synthetic Construct
PIDAFFD

SEQ ID NO: 230 - protein, 7 aa, Artificial Sequence
  Synthetic Construct
PPNMARA

SEQ ID NO: 231 - protein, 7 aa, Artificial Sequence
  Synthetic Construct
PTNKPHT

SEQ ID NO: 232 - protein, 7 aa, Artificial Sequence
  Synthetic Construct
SPNNTRE

SEQ ID NO: 233 - protein, 7 aa, Artificial Sequence
  Synthetic Construct
SPEMKPR

SEQ ID NO: 234 - protein, 7 aa, Artificial Sequence
  Synthetic Construct
SSSMAKM

SEQ ID NO: 235 - protein, 7 aa, Artificial Sequence
  Synthetic Construct
TDHPPKA

SEQ ID NO: 236 - protein, 7 aa, Artificial Sequence
  Synthetic Construct
TLAFQTA

SEQ ID NO: 237 - protein, 7 aa, Artificial Sequence
  Synthetic Construct
APLSLSL

SEQ ID NO: 238 - protein, 7 aa, Artificial Sequence
  Synthetic Construct
HYPTVNF

SEQ ID NO: 239 - protein, 12 aa, Artificial Sequence
  Synthetic Construct
QHNFRGASSSAP

SEQ ID NO: 240 - protein, 12 aa, Artificial Sequence
  Synthetic Construct
  misc_feature (5)..(5): Xaa can be any naturally occurring amino acid
HQFPXSNLVWKP

SEQ ID NO: 241 - protein, 12 aa, Artificial Sequence
  Synthetic Construct
LSLRASAATDFQ

SEQ ID NO: 242 - protein, 12 aa, Artificial Sequence
  Synthetic Construct
MQFTPAPSPSDH

SEQ ID NO: 243 - protein, 12 aa, Artificial Sequence
  Synthetic Construct
SVFLPTRHSPDL

SEQ ID NO: 244 - protein, 12 aa, Artificial Sequence
  Synthetic Construct
SVSVGMKPSPRP

SEQ ID NO: 245 - protein, 12 aa, Artificial Sequence
  Synthetic Construct
SVSVGMKPSPRP

SEQ ID NO: 246 - protein, 12 aa, Artificial Sequence
  Synthetic Construct
SVSVGMKPSPRP

SEQ ID NO: 247 - protein, 12 aa, Artificial Sequence
  Synthetic Construct
SVSVGMKPSPRP

SEQ ID NO: 248 - protein, 11 aa, Artificial Sequence
  Synthetic Construct
SVSVGMNAESA

SEQ ID NO: 249 - protein, 7 aa, Artificial Sequence
  Synthetic Construct
RHTLPLH

SEQ ID NO: 250 - protein, 7 aa, Artificial Sequence
  Synthetic Construct
NPQMQRS

SEQ ID NO: 251 - protein, 7 aa, Artificial Sequence
  Synthetic Construct
NYPTLKS

SEQ ID NO: 252 - protein, 7 aa, Artificial Sequence
  Synthetic Construct
NYPTLKS

SEQ ID NO: 253 - protein, 7 aa, Artificial Sequence
  Synthetic Construct
DMRQQRS

SEQ ID NO: 254 - protein, 7 aa, Artificial Sequence
  Synthetic Construct
QNPRQIY

SEQ ID NO: 255 - protein, 7 aa, Artificial Sequence
  Synthetic Construct
QNPRQIY

SEQ ID NO: 256 - protein, 7 aa, Artificial Sequence
  Synthetic Construct
QNPRQIY

SEQ ID NO: 257 - protein, 7 aa, Artificial Sequence
  Synthetic Construct
QNPRQIY

SEQ ID NO: 258 - protein, 7 aa, Artificial Sequence
  Synthetic Construct
QNPRQIY

SEQ ID NO: 259 - protein, 7 aa, Artificial Sequence
  Synthetic Construct
QNPRQIY

SEQ ID NO: 260 - protein, 7 aa, Artificial Sequence
  Synthetic Construct
QNPRQIY

SEQ ID NO: 261 - protein, 7 aa, Artificial Sequence
  Synthetic Construct
QNPRQIY

SEQ ID NO: 262 - protein, 7 aa, Artificial Sequence
  Synthetic Construct
QNPRQIY

SEQ ID NO: 263 - protein, 7 aa, Artificial Sequence
  Synthetic Construct
QTHSSLW

SEQ ID NO: 264 - protein, 7 aa, Artificial Sequence
  Synthetic Construct
ETYQQPL

SEQ ID NO: 265 - protein, 7 aa, Artificial Sequence
  Synthetic Construct
ETYARPL

SEQ ID NO: 266 - protein, 7 aa, Artificial Sequence
  Synthetic Construct
GTSRLFS

SEQ ID NO: 267 - protein, 7 aa, Artificial Sequence
  Synthetic Construct
LTQTLQY

SEQ ID NO: 268 - protein, 7 aa, Artificial Sequence
  Synthetic Construct
KAFDKHG

SEQ ID NO: 269 - protein, 7 aa, Artificial Sequence
  Synthetic Construct
RPMQFVH

SEQ ID NO: 270 - protein, 7 aa, Artificial Sequence
  Synthetic Construct
KPMQFVH

SEQ ID NO: 271 - protein, 7 aa, Artificial Sequence
  Synthetic Construct
KPMQFIH

SEQ ID NO: 272 - protein, 7 aa, Artificial Sequence
  Synthetic Construct
PAKQKAH

SEQ ID NO: 273 - protein, 7 aa, Artificial Sequence
  Synthetic Construct
SASGTPS

SEQ ID NO: 274 - protein, 7 aa, Artificial Sequence
  Synthetic Construct
SSHHHRH

SEQ ID NO: 275 - protein, 7 aa, Artificial Sequence
  Synthetic Construct
SSYEYHA

SEQ ID NO: 276 - protein, 7 aa, Artificial Sequence
  Synthetic Construct
TGPTSLS

SEQ ID NO: 277 - protein, 12 aa, Artificial Sequence
  Synthetic Construct
LRAFPSLPHTVT

SEQ ID NO: 278 - protein, 7 aa, Artificial Sequence
  Synthetic Construct
NPRSQAT

SEQ ID NO: 279 - protein, 7 aa, Artificial Sequence
  Synthetic Construct
HRLGHMS

SEQ ID NO: 280 - protein, 7 aa, Artificial Sequence
  Synthetic Construct
LLPLKFK

SEQ ID NO: 281 - protein, 7 aa, Artificial Sequence
  Synthetic Construct
LPSIHNL

SEQ ID NO: 282 - protein, 7 aa, Artificial Sequence
  Synthetic Construct
KATITGM

SEQ ID NO: 283 - protein, 7 aa, Artificial Sequence
  Synthetic Construct
PDIPLSR

SEQ ID NO: 284 - protein, 7 aa, Artificial Sequence
  Synthetic Construct
PSMKHWR

SEQ ID NO: 285 - protein, 7 aa, Artificial Sequence
  Synthetic Construct
SAKGRAD

SEQ ID NO: 286 - protein, 7 aa, Artificial Sequence
  Synthetic Construct
SRTGAHH

SEQ ID NO: 287 - protein, 7 aa, Artificial Sequence
  Synthetic Construct
SKTSSTS

SEQ ID NO: 288 - protein, 7 aa, Artificial Sequence
  Synthetic Construct
SPNNPRE

SEQ ID NO: 289 - protein, 7 aa, Artificial Sequence
  Synthetic Construct
TLQRMGQ

SEQ ID NO: 290 - protein, 7 aa, Artificial Sequence
  Synthetic Construct
TMTNMAK

SEQ ID NO: 291 - protein, 7 aa, Artificial Sequence
  Synthetic Construct
TTLSPRT

SEQ ID NO: 292 - protein, 7 aa, Artificial Sequence
  Synthetic Construct
TTKNFNK

SEQ ID NO: 293 - protein, 7 aa, Artificial Sequence
  Synthetic Construct
YPKALRN

SEQ ID NO: 294 - protein, 7 aa, Artificial Sequence
  Synthetic Construct
VVKSNGE

SEQ ID NO: 295 - protein, 5 aa, Artificial Sequence
  Synthetic Construct
ITGAY

SEQ ID NO: 296 - protein, 7 aa, Artificial Sequence
  Synthetic Construct
LPLTPLP

SEQ ID NO: 297 - protein, 12 aa, Artificial Sequence
  Synthetic Construct
HSMPHMGTYLLT

SEQ ID NO: 298 - protein, 12 aa, Artificial Sequence
  Synthetic Construct
MQFTPAPSPSDH

SEQ ID NO: 299 - protein, 12 aa, Artificial Sequence
  Synthetic Construct
MPQTLVLPRSLL

SEQ ID NO: 300 - protein, 12 aa, Artificial Sequence
  Synthetic Construct
SSTQVQHTLLQT

SEQ ID NO: 301 - protein, 12 aa, Artificial Sequence
  Synthetic Construct
SWPLYSRDSGLG

SEQ ID NO: 302 - protein, 12 aa, Artificial Sequence
  Synthetic Construct
  misc_feature (11)..(11): Xaa can be any naturally occurring amino acid
SVSVGTEAESXA

SEQ ID NO: 303 - protein, 12 aa, Artificial Sequence
  Synthetic Construct
SVSVGMKPSPRP

SEQ ID NO: 304 - protein, 12 aa, Artificial Sequence
  Synthetic Construct
SVSVGMKPSPRP

SEQ ID NO: 305 - protein, 12 aa, Artificial Sequence
  Synthetic Construct
SVSVGMKPSPRP

SEQ ID NO: 306 - protein, 12 aa, Artificial Sequence
  Synthetic Construct
SVSVGMNAESYG

SEQ ID NO: 307 - protein, 12 aa, Artificial Sequence
  Synthetic Construct
THPVVFEDERLF

SEQ ID NO: 308 - protein, 12 aa, Artificial Sequence
  Synthetic Construct
TLPSPLALLTVH

SEQ ID NO: 309 - protein, 12 aa, Artificial Sequence
  Synthetic Construct
WPTYLNPSSLKA

SEQ ID NO: 310 - protein, 7 aa, Artificial Sequence
  Synthetic Construct
ASHNPKL

SEQ ID NO: 311 - protein, 7 aa, Artificial Sequence
  Synthetic Construct
PAKQKAH

SEQ ID NO: 312 - protein, 7 aa, Artificial Sequence
  Synthetic Construct
PAKQKAH

SEQ ID NO: 313 - protein, 7 aa, Artificial Sequence
  Synthetic Construct
SASGTPS

SEQ ID NO: 314 - protein, 7 aa, Artificial Sequence
  Synthetic Construct
TRFYDSL

SEQ ID NO: 315 - protein, 7 aa, Artificial Sequence
  Synthetic Construct
QNPRQIY

SEQ ID NO: 316 - protein, 7 aa, Artificial Sequence
  Synthetic Construct
QNPRQIY

SEQ ID NO: 317 - protein, 7 aa, Artificial Sequence
  Synthetic Construct
QNPRQIY

SEQ ID NO: 318 - protein, 7 aa, Artificial Sequence
  Synthetic Construct
QNPRQIY

SEQ ID NO: 319 - protein, 7 aa, Artificial Sequence
  Synthetic Construct
TGPTSLS

SEQ ID NO: 320 - protein, 7 aa, Artificial Sequence
  Synthetic Construct
TGPTSLS

SEQ ID NO: 321 - protein, 7 aa, Artificial Sequence
  Synthetic Construct
NPQMQRS

SEQ ID NO: 322 - protein, 7 aa, Artificial Sequence
  Synthetic Construct
NPQMQRS

SEQ ID NO: 323 - protein, 7 aa, Artificial Sequence
  Synthetic Construct
NPQMQRS

SEQ ID NO: 324 - protein, 7 aa, Artificial Sequence
  Synthetic Construct
NPQMQRS

SEQ ID NO: 325 - protein, 7 aa, Artificial Sequence
  Synthetic Construct
NPQMQRS

SEQ ID NO: 326 - protein, 7 aa, Artificial Sequence
  Synthetic Construct
KPMQFVH

SEQ ID NO: 327 - protein, 7 aa, Artificial Sequence
  Synthetic Construct
SSYEYHA

SEQ ID NO: 328 - protein, 7 aa, Artificial Sequence
  Synthetic Construct
STQAHPW

SEQ ID NO: 329 - protein, 7 aa, Artificial Sequence
  Synthetic Construct
GTSRLFS

SEQ ID NO: 330 - protein, 7 aa, Artificial Sequence
  Synthetic Construct
NYPTLKS

SEQ ID NO: 331 - protein, 7 aa, Artificial Sequence
  Synthetic Construct
NYPTLKS

SEQ ID NO: 332 - protein, 7 aa, Artificial Sequence
  Synthetic Construct
NYPTLKS

SEQ ID NO: 333 - protein, 7 aa, Artificial Sequence
  Synthetic Construct
NYPTLKS

SEQ ID NO: 334 - protein, 7 aa, Artificial Sequence
  Synthetic Construct
NYPTLKS

SEQ ID NO: 335 - protein, 7 aa, Artificial Sequence
  Synthetic Construct
NYPTLKS

SEQ ID NO: 336 - protein, 7 aa, Artificial Sequence
  Synthetic Construct
NYPTLKS

SEQ ID NO: 337 - protein, 7 aa, Artificial Sequence
  Synthetic Construct
HAPVQPN

SEQ ID NO: 338 - protein, 12 aa, Artificial Sequence
  Synthetic Construct
NPYHPTIPQSVH

SEQ ID NO: 339 - protein, 12 aa, Artificial Sequence
  Synthetic Construct
NPYHPTIPQSVH

SEQ ID NO: 340 - protein, 12 aa, Artificial Sequence
  Synthetic Construct
NPYHPTIPQSVH

SEQ ID NO: 341 - protein, 12 aa, Artificial Sequence
  Synthetic Construct
NPYHPTIPQSVH

SEQ ID NO: 342 - protein, 12 aa, Artificial Sequence
  Synthetic Construct
HQFISPEPFLIS

SEQ ID NO: 343 - protein, 17 aa, Artificial Sequence
  Synthetic Construct
SPNFSWLPLGTTSPNFS

SEQ ID NO: 344 - protein, 7 aa, Artificial Sequence
  Synthetic Construct
WLPLGTT

SEQ ID NO: 345 - protein, 12 aa, Artificial Sequence
  Synthetic Construct
SVSVGMKPSPRP

SEQ ID NO: 346 - protein, 12 aa, Artificial Sequence
  Synthetic Construct
SVSVGMKPSPRP

SEQ ID NO: 347 - protein, 12 aa, Artificial Sequence
  Synthetic Construct
TPLTSPSLVRPQ

SEQ ID NO: 348 - protein, 12 aa, Artificial Sequence
  Synthetic Construct
TPLSYLKGLVTV

SEQ ID NO: 349 - protein, 7 aa, Artificial Sequence
  Synthetic Construct
NPMIMNQ

SEQ ID NO: 350 - protein, 7 aa, Artificial Sequence
  Synthetic Construct
NPMIMNQ

SEQ ID NO: 351 - protein, 7 aa, Artificial Sequence
  Synthetic Construct
NITQLGS

SEQ ID NO: 352 - protein, 7 aa, Artificial Sequence
  Synthetic Construct
HTLLSTT

SEQ ID NO: 353 - protein, 7 aa, Artificial Sequence
  Synthetic Construct
HTLLSTT

SEQ ID NO: 354 - protein, 7 aa, Artificial Sequence
  Synthetic Construct
HTLLSTT

SEQ ID NO: 355 - protein, 7 aa, Artificial Sequence
  Synthetic Construct
LGPGKAF

SEQ ID NO: 356 - protein, 7 aa, Artificial Sequence
  Synthetic Construct
LGPGKAF

SEQ ID NO: 357 - protein, 7 aa, Artificial Sequence
  Synthetic Construct
LGPGKAF

SEQ ID NO: 358 - protein, 7 aa, Artificial Sequence
  Synthetic Construct
LGPGKAF

SEQ ID NO: 359 - protein, 7 aa, Artificial Sequence
  Synthetic Construct
KTSSWAN

SEQ ID NO: 360 - protein, 7 aa, Artificial Sequence
  Synthetic Construct
KMNHMPN

SEQ ID NO: 361 - protein, 7 aa, Artificial Sequence
  Synthetic Construct
SLLTPWL

SEQ ID NO: 362 - protein, 7 aa, Artificial Sequence
  Synthetic Construct
TLGLPML

SEQ ID NO: 363 - protein, 6 aa, Artificial Sequence
  Synthetic Construct
TGLAKT

SEQ ID NO: 364 - protein, 5 aa, Artificial Sequence
  Synthetic Construct
IRLIS

SEQ ID NO: 365 - protein, 7 aa, Artificial Sequence
  Synthetic Construct
LGPGKAF

SEQ ID NO: 366 - protein, 7 aa, Artificial Sequence
  Synthetic Construct
LGPGKAF

SEQ ID NO: 367 - protein, 7 aa, Artificial Sequence
  Synthetic Construct
LGPGKAF

SEQ ID NO: 368 - protein, 12 aa, Artificial Sequence
  Synthetic Construct
DLNYFTLSSKRE

SEQ ID NO: 369 - protein, 12 aa, Artificial Sequence
  Synthetic Construct
DLNYFTLSSKRE

SEQ ID NO: 370 - protein, 12 aa, Artificial Sequence
  Synthetic Construct
TMGFTAPRFPHY

SEQ ID NO: 371 - protein, 12 aa, Artificial Sequence
  Synthetic Construct
TMGFTAPRFPHY

SEQ ID NO: 372 - protein, 12 aa, Artificial Sequence
  Synthetic Construct
TMGFTAPRFPHY

SEQ ID NO: 373 - protein, 12 aa, Artificial Sequence
  Synthetic Construct
TMGFTAPRFPHY

SEQ ID NO: 374 - protein, 7 aa, Artificial Sequence
  Synthetic Construct
HTLLSTT

SEQ ID NO: 375 - protein, 7 aa, Artificial Sequence
  Synthetic Construct
HTLLSTT

SEQ ID NO: 376 - protein, 7 aa, Artificial Sequence
  Synthetic Construct
LASTTHV

SEQ ID NO: 377 - protein, 7 aa, Artificial Sequence
  Synthetic Construct
LGPGKAF

SEQ ID NO: 378 - protein, 7 aa, Artificial Sequence
  Synthetic Construct
LGPGKAF

SEQ ID NO: 379 - protein, 7 aa, Artificial Sequence
  Synthetic Construct
LGPGKAF

SEQ ID NO: 380 - protein, 7 aa, Artificial Sequence
  Synthetic Construct
SLLTPWL

SEQ ID NO: 381 - protein, 7 aa, Artificial Sequence
  Synthetic Construct
NERQMEL

SEQ ID NO: 382 - protein, 7 aa, Artificial Sequence
  Synthetic Construct
NKPLSTL

SEQ ID NO: 383 - protein, 7 aa, Artificial Sequence
  Synthetic Construct
HTLLSTT

SEQ ID NO: 384 - protein, 7 aa, Artificial Sequence
  Synthetic Construct
LKPFSGA

SEQ ID NO: 385 - protein, 7 aa, Artificial Sequence
  Synthetic Construct
LGPGKAF

SEQ ID NO: 386 - protein, 7 aa, Artificial Sequence
  Synthetic Construct
LGPGKAF

SEQ ID NO: 387 - protein, 7 aa, Artificial Sequence
  Synthetic Construct
LGPGKAF

SEQ ID NO: 388 - protein, 7 aa, Artificial Sequence
  Synthetic Construct
LGPGKAF

SEQ ID NO: 389 - protein, 7 aa, Artificial Sequence
  Synthetic Construct
STSAKHW

SEQ ID NO: 390 - protein, 12 aa, Artificial Sequence
  Synthetic Construct
TMGFTAPRFPHY

SEQ ID NO: 391 - protein, 12 aa, Artificial Sequence
  Synthetic Construct
TMGFTAPRFPHY

SEQ ID NO: 392 - protein, 12 aa, Artificial Sequence
  Synthetic Construct
TMGFTAPRFPHY

SEQ ID NO: 393 - protein, 12 aa, Artificial Sequence
  Synthetic Construct
TMGFTAPRFPHY

SEQ ID NO: 394 - protein, 12 aa, Artificial Sequence
  Synthetic Construct
TMGFTAPRFPHY

SEQ ID NO: 395 - protein, 12 aa, Artificial Sequence
  Synthetic Construct
TMGFTAPRFPHY

SEQ ID NO: 396 - protein, 12 aa, Artificial Sequence
  Synthetic Construct
TMGFTAPRFPHY

SEQ ID NO: 397 - protein, 9 aa, Artificial Sequence
  Synthetic Construct
CNYPTLKSC

SEQ ID NO: 398 - DNA, 2712 bp, Artificial Sequence
  Synthetic Construct
tttaagcttgccgccaccatgatgtcctttgtctctctgctcctggttggcatcctattccatgccacccaggccgacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggagagactttcttcatgtgttcctgtagctctgatgagtgcaatgacaacatcatcttctcagaagaatataacaccagcaatcctgacttgttgctagtcatatttcaagtgacaggcatcagcctcctgccaccactggcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtctaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgaggagatgggagatgaggaagtccacactattcctcctgagctacggatcctgctggaccctggtgccctgccgcaactttgcaagttctgcgacgtgcgattctctacgtgcgataatcaaaagtcctgtatgtcaaactgcagtattacttctatttgtgagaagcctcaggaggtttgtgtcgcggtctggcggaaaaacgacgaaaatatcacattggaaacggtctgccacgaccccaaacttccctatcatgatttcatacttgaggatgcagcttcacctaagtgtattatgaaagagaagaagaaaccaggcgaaacgttctttatgtgcagttgctcctccgatgaatgcaatgataacatcattttctccgaggagtacaatacttcaaatccagacctccttctcgtcatttttcaagttacaggtatttcactgctcccccctctcggcgttgcgatatcagttatcatcatcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctcccgggaaagacgacgatgatgatgacgatgacgacgattgatgactcgagttt

SEQ ID NO: 399 - DNA, 2712 bp, Artificial Sequence
  Synthetic Construct
tttaagcttgccgccaccatgatgtcctttgtctctctgctcctggttggcatcctattccatgccacccaggccgacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggagagactttcttcatgtgttcctgtagctctgatgagtgcaatgacaacatcatcttctcagaagaatataacaccagcaatcctgacggtcttggccccgtcgagagtagccctgggcatggccttgataccgcagcggcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtctaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgaggagatgggagatgaggaagtccacactattcctcctgagctacggatcctgctggaccctggtgccctgccgcaactttgcaagttctgcgacgtgcgattctctacgtgcgataatcaaaagtcctgtatgtcaaactgcagtattacttctatttgtgagaagcctcaggaggtttgtgtcgcggtctggcggaaaaacgacgaaaatatcacattggaaacggtctgccacgaccccaaacttccctatcatgatttcatacttgaggatgcagcttcacctaagtgtattatgaaagagaagaagaaaccaggcgaaacgttctttatgtgcagttgctcctccgatgaatgcaatgataacatcattttctccgaggagtacaatacttcaaatccagacctccttctcgtcatttttcaagttacaggtatttcactgctcccccctctcggcgttgcgatatcagttatcatcatcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctcccgggaaagacgacgatgatgatgacgatgacgacgattgatgactcgagttt

SEQ ID NO: 400 - DNA, 2739 bp, Artificial Sequence
  Synthetic Construct
tttaagcttgccgccaccatgatgtcctttgtctctctgctcctggttggcatcctattccatgccacccaggccgacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggagagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcgcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtctaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgaggagatgggagatgaggaagtccacactattcctcctgagctacggatcctgctggaccctggtgccctgccgcaactttgcaagttctgcgacgtgcgattctctacgtgcgataatcaaaagtcctgtatgtcaaactgcagtattacttctatttgtgagaagcctcaggaggtttgtgtcgcggtctggcggaaaaacgacgaaaatatcacattggaaacggtctgccacgaccccaaacttccctatcatgatttcatacttgaggatgcagcttcacctaagtgtattatgaaagagaagaagaaaccaggcgaaacgttctttatgtgcagttgctcctccgatgaatgcaatgataacatcattttctccgaggagtacaatacttcaaatccagacctccttctcgtcatttttcaagttacaggtatttcactgctcccccctctcggcgttgcgatatcagttatcatcatcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctcccgggaaagacgacgatgatgatgacgatgacgacgattgatgactcgagttt

SEQ ID NO: 401 - DNA, 2700 bp, Artificial Sequence
  Synthetic Construct
tttaagcttgccgccaccatgatgtcctttgtctctctgctcctggttggcatcctattccatgccacccaggccgacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggagagactttcttcatgtgttcctgtagctctgatgagtgcaatgacaacatcatcttctcagaagaatataacaccagcaatcctgacggtcttggccccgtcgagagtagccctgggcatggccttgataccgcagcggcaggtccagagcctggtgcactgtgtgaactgtcacctgtcagtgcctcccatcctgtccaggccttgatggagagcttcactgttttgtcaggctgtgccagcagaggcacaactgggctgccacaggaggtgcatgtcctgaatctccgcactgcaggccaggggcctggccagctacagagagaggtcacacttcacctgaatcccatctcctcagtccacatccaccacaagtctgttgtgttcctgctcaactccccacaccccctggtgtggcatctgaagacagagagacttgccactggggtctccagactgtttttggtgtctgagggttctgtggtccagttttcatcagcaaacttctccttgacagcagaaacagaagaaaggaacttcccccatggaaatgaacatctgttaaattgggcccgaaaagagtatggagcagttacttcattcaccgaactcaagatagcaagaaacatttatattaaagtgggggaagatcaagtgttccctccaaagtgcaacatagggaagaattttctctcactcaattaccttgctgagtaccttcaacccaaagcagcagaagggtgtgtgatgtccagccagccccagaatgaggaagtacacatcatcgagctaatcacccccaactctaacccctacagtgctttccaggtggatataacaattgatataagaccttctcaagaggatcttgaagtggtcaaaaatctcatcctgatcttgaagtctaaaaagtctgtcaactgggtgatcaaatcttttgatgttaagggaagcctgaaaattattgctcctaacagtattggctttggaaaagagagtgaaagatctatgacaatgaccaaatcaataagagatgacattccttcaacccaagggaatctggtgaagtgggctttggacaatggctatagtccaataacttcatacacaatggctcctgtggctaatagatttcatcttcggcttgaaaataatgaggagatgggagatgaggaagtccacactattcctcctgagctacggatcctgctggaccctggtgccctgccgcaactttgcaagttctgcgacgtgcgattctctacgtgcgataatcaaaagtcctgtatgtcaaactgcagtattacttctatttgtgagaagcctcaggaggtttgtgtcgcggtctggcggaaaaacgacgaaaatatcacattggaaacggtctgccacgaccccaaacttccctatcatgatttcatacttgaggatgcagcttcacctaagtgtattatgaaagagaagaagaaaccaggcgaaacgttctttatgtgcagttgctcctccgatgaatgcaatgataacatcattttctccgaggagtacaatacttcaaatccagacctccttctcgtcatttttcaagttacaggtatttcactgctcccccctctcggcggaggcggttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctcccgggaaagacgacgatgatgatgacgatgacgacgattgatgactcgagttt

SEQ ID NO: 402 - protein, 10 aa, Artificial Sequence
  Synthetic Construct
GGGGSGGGGS

SEQ ID NO: 403 - protein, 29 aa, Artificial Sequence
  Synthetic Construct
MMSFVSLLLVGILFHATQADNGAVKFPQL

SEQ ID NO: 404 - protein, 10 aa, Artificial Sequence
  Synthetic Construct
CRKQYEDQLS

(19) United States
(12) Patent Application Publication
(10) Pub. No.: US 2019/0248881 A1
(43) Pub. Date: Aug. 15, 2019

(54) TGF-ß RECEPTOR FUSION PROTEINS AND OTHER TGF-ß ANTAGONISTS FOR REDUCING TGF-ß SIGNALING

(71) Applicant: PreciThera, Inc., Montreal (CA)

(72) Inventors: Philippe CRINE, Montreal (CA); Susan SCHIAVI, Hopkinton, MA (US)

(21) Appl. No.: 16/209,889

(22) Filed: Dec. 4, 2018

Related U.S. Application Data
(60) Provisional application No. 62/594,226, filed on Dec. 4, 2017.
(60) Provisional application No. 62/594,288, filed on Dec. 4, 2017.
(60) Provisional application No. 62/678,229, filed on May 30, 2018.
(60) Provisional application No. 62/753,481, filed on Oct. 31, 2018.
(60) Provisional application No. 62/753,487, filed on Oct. 31, 2018.

(51) Int. Cl. C07K 16/22; A61K 47/54; A61P 19/08; A61K 47/65; C07K 14/495

(52) U.S. Cl. CPC C07K 16/22; A61K 47/548; C07K 14/495; A61K 47/65; A61P 19/08

(57) ABSTRACT

The present invention provides TGF-β antagonists and conjugates thereof, as well as methods of using such compositions for attenuating TGF-β signaling. These novel compositions and methods may be useful for treating individuals suffering from devastating diseases associated with elevated TGF-β signaling, including skeletal disorders, such as osteogenesis imperfecta (OI), and muscular diseases, such as muscular dystrophies.

### RELATED APPLICATIONS

The present application claims priority to, and the benefit of, U.S. Provisional Patent Application No. 62/594,226, filed Dec. 4, 2017, U.S. Provisional Patent Application No. 62/594,288, filed Dec. 4, 2017, U.S. Provisional Patent Application No. 62/678,229, filed May 30, 2018, U.S. Provisional Patent Application No. 62/753,481, filed Oct. 31, 2018, and U.S. Provisional Patent Application No. 62/753,487, filed Oct. 31, 2018, each of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Apr. 16, 2019, is named 51159-006006_Sequence_Listing_4.16.19_ST25 and is 374,027 bytes in size.

### FIELD OF THE INVENTION

The present invention relates to the fields of peptide and protein therapy and provides therapeutic conjugates, compositions, and methods capable of attenuating TGF-β signaling for the treatment of diseases associated with elevated TGF-β signaling, such as skeletal and muscle disorders.

### BACKGROUND OF THE INVENTION

Transforming growth factor-β (TGF-β) is a multifunctional cytokine that performs many cellular functions. For example, TGF-β is an important regulator of bone homeostasis, and the activity of this protein promotes a balance between bone building and degradation. Elevations in active TGF-β and increased downstream signaling in the TGF-β pathway are associated with a variety of pathologies, including skeletal disorders, such as osteogenesis imperfecta (OI), as well as various muscle disorders, such as muscular dystrophies. There remains a need for the development of therapeutic compounds capable of attenuating TGF-β signal transduction for treating individuals suffering from disorders associated with elevated TGF-β signaling, particularly at the site of bone tissue.

### SUMMARY OF THE INVENTION

We have discovered that heterotrimeric fusion proteins comprising portions of transforming growth factor-β (TGF-β) receptor proteins can suppress TGF-β signaling in bone, and can restore and/or improve muscle function in patients suffering from a variety of skeletal disorders, such as osteogenesis imperfecta and other disorders associated with elevated bone turnover, as well as various muscle disorders, such as muscular dystrophies (e.g., Duchenne muscular dystrophy).

The invention provides therapeutic conjugates and compositions containing TGF-β antagonists, such as TGF-β receptor fusion proteins and TGF-β antibodies targeted to the bone, which localizes the antagonist to human bone tissue. In many cases, the TGF-β receptor fusion proteins of the invention contain one or more domains of TGF-β receptor II covalently bound to one or more domains of TGF-β receptor III, e.g., fusion proteins containing the ectodomain of TGF-β receptor II, or a portion or variant thereof, bound to the endoglin domain of TGF-β receptor III, or a portion or variant thereof. Particular constructs described include fusion proteins in which two TGF-β receptor II ectodomains, or fragments or variants thereof, are each independently bound to a single TGF-β receptor III endoglin domain, or a portion or variant thereof. Fusion proteins containing one or more TGF-β ectodomains, or fragments or variants thereof, bound to a TGF-β endoglin domain, or a portion or fragment thereof, are high-affinity inhibitors of TGF-β capable of sequestering this growth factor and attenuating TGF-β signal transduction. Compounds of the invention that may have particular efficacy in treating bone and muscle disorders include those TGF-β receptor fusion proteins that are fused to targeting moieties that specifically bind hydroxyapatite in bone tissue.

The TGF-β antagonists, such as the novel TGF-β receptor fusion proteins, including those that are fused to targeting moieties, e.g., bone-targeting moieties that specifically bind hydroxyapatite, described herein, can be used in methods of the invention to treat a variety of skeletal and muscle disorders associated with elevated TGF-β signaling, including in bone tissue and at the skeletal-muscular interface. It should be noted that the novel TGF-β antagonists, described herein, can also be used to treat other diseases that result from elevated TGF-β signaling and for improving muscle function in individuals suffering from diseases associated with elevated TGF-β signaling.

In a first aspect, the invention features a composition containing a TGF-β antagonist, wherein the TGF-β antagonist is a fusion protein that comprises a homodimer of a compound of the formula: I(a). (A-L¹-B-L²-Z), I(b). (Z-L²-B-L¹-A), or I(c). (B-L¹-A-L²-Z), where A is an RER heterotrimeric fusion polypeptide; L¹ is a linker; B is an Fc domain of an immunoglobulin or is absent; L² is a linker or is absent; Z is a bone-targeting moiety or is absent; and where A, the RER heterotrimeric fusion polypeptide, includes a polypeptide sequence of the formula: W-L³-X-L⁴-Y, where W is a TGF-β type II receptor ectodomain or a portion thereof; L³ is a linker or is absent; X is a TGF-β type III receptor endoglin domain or a portion thereof; L⁴ is a linker or is absent; Y is a TGF-β type II receptor ectodomain or a portion thereof, and where the amino acid sequence of A is not the amino acid sequence of SEQ ID NO: 48.

Certain embodiments of the above composition may vary in ways described below.

In some embodiments, the linker L¹ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some embodiments, B, the Fc domain of an immunoglobulin is present. In some embodiments, B, the Fc domain of an immunoglobulin is absent. In some embodiments, the Fc domain of an immunoglobulin includes the Fc domain of human IgG, human IgA, human IgM, human IgE, or human IgD; or a variant of said domain. In some embodiments, B, the Fc domain of human IgG is IgG1, IgG2, IgG3, or IgG4; or a variant thereof. In some embodiments, the Fc domain of human includes the amino acid sequence of SEQ ID NO: 47; or a variant of said amino acid sequence.

In some embodiments, the linker L² is present. In some embodiments, the linker L² is absent. In some embodiments, the linker L² includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some embodiments, Z, the bone-targeting moiety is present. In some embodiments, Z, the bone-targeting moiety, is absent. In some embodiments, Z, the bone-targeting moiety includes a polyanionic peptide, a bisphosphonate, or the amino acid sequence of SEQ ID NO: 46; or a variant of said amino acid sequence.

In some embodiments, the TGF-β type II receptor ectodomain W is at the N-terminus of the RER heterotrimeric fusion polypeptide and the TGF-β type II receptor ectodomain Y is at the C-terminus of the RER heterotrimeric fusion polypeptide. In some embodiments, the C-terminus of the TGF-β type II receptor ectodomain Y is covalently joined to the N-terminus of B, the Fc domain of an immunoglobulin, via the linker L¹ as in formula I(a). In some embodiments, the N-terminus of the TGF-β type II receptor ectodomain W is covalently joined to the C-terminus of B, the Fc domain of an immunoglobulin, via the linker L¹ as in formula I(b) or I(c).

In some embodiments, the amino acid sequence of the TGF-β type II receptor ectodomain W is identical to the amino acid sequence of the TGF-β type II receptor ectodomain Y. In some embodiments, the amino acid sequence of the TGF-β type II receptor ectodomain W is different than the amino acid sequence of the TGF-β type II receptor ectodomain Y. In some embodiments, the TGF-β type II receptor ectodomains W and/or Y includes an amino acid sequence extending from amino acid residues 22 to 139 of SEQ ID NO: 5, 520 to 631 of SEQ ID NO: 5, 1 to 118 of SEQ ID NO: 9, 479 to 590 of SEQ ID NO: 9, 1 to 118 of SEQ ID NO: 48, 499 to 610 of SEQ ID NO: 48, 1 to 118 of SEQ ID NO: 49, 499 to 610 of SEQ ID NO: 49, 1 to 120 of SEQ ID NO: 50, 501 to 612 of SEQ ID NO: 50, 1 to 120 of SEQ ID NO: 51, 501 to 612 of SEQ ID NO: 51, 1 to 120 of SEQ ID NO: 52, or 510 to 621 of SEQ ID NO: 52; or a variant of said amino acid sequences.

In some embodiments, the linker L³ is present. In some embodiments, the linker L³ is absent. In some embodiments, the linker L³ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some embodiments, the TGF-β type III receptor endoglin domain X includes an amino acid sequence extending from amino acid residues 157 to 517 of SEQ ID NO: 5, 119 to 478 of SEQ ID NO: 9, 136 to 496 of SEQ ID NO: 48, 136 to 496 of SEQ ID NO: 49, 138 to 500 of SEQ ID NO: 50, 138 to 500 of SEQ ID NO: 51, or 147 to 509 of SEQ ID NO: 52; or a variant of said amino acid sequences.

In some embodiments, the linker L⁴ is present. In some embodiments, the linker L⁴ is absent. In some embodiments, the linker L⁴ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some embodiments, the RER heterotrimeric fusion polypeptide includes an amino acid sequence selected from the group comprising SEQ ID NO: 9, SEQ ID NO: 49, SEQ ID NO: 50, SEQ ID NO: 51, and SEQ ID NO: 52; or a variant of said amino acid sequences. In some embodiments, the RER heterotrimeric fusion polypeptide includes the amino acid sequence of SEQ ID NO: 51; or a variant of said amino acid sequence. In some embodiments, the RER heterotrimeric fusion polypeptide includes the amino acid sequence of SEQ ID NO: 52; or a variant of said amino acid sequence.

In some embodiments, the homodimer includes an amino acid sequence selected from the group comprising SEQ ID NO: 22, SEQ ID NO: 24, SEQ ID NO: 26, SEQ ID NO: 28, and SEQ ID NO: 30; or a variant of said amino acid sequences. In some embodiments, the homodimer includes an amino acid sequence selected from the group comprising SEQ ID NO: 9, SEQ ID NO: 23, SEQ ID NO: 25, SEQ ID NO: 27, SEQ ID NO: 29, and SEQ ID NO: 31; or a variant of said amino acid sequences.

In a second aspect, the invention features a composition containing a TGF-β antagonist, wherein the TGF-β antagonist is a fusion protein that includes a homodimer of a compound of the formula: I(a). (A-L¹-B-L²-Z); where A is an RER heterotrimeric fusion polypeptide; L¹ is a linker; B is an Fc domain of an immunoglobulin; L² is a linker that is absent; Z is a bone-targeting moiety; and A, the RER heterotrimeric fusion polypeptide, includes a polypeptide sequence of the formula: W-L³-X-L⁴-Y, where W is a TGF-β type II receptor ectodomain or a portion thereof; L³ is a linker; X is a TGF-β type III receptor endoglin domain or a portion thereof; L⁴ is a linker that is absent; and Y is a TGF-β type II receptor ectodomain or a portion thereof; and the amino acid sequence of A is not the amino acid sequence of SEQ ID NO: 48.

In some embodiments, the homodimer is PCT-0025 having the amino acid sequence of SEQ ID NO: 28; or a variant of said amino acid sequence. In some embodiments, the homodimer is PCT-0026 having the amino acid sequence of SEQ ID NO: 30; or a variant of said amino acid sequence.

In another aspect, the invention features a composition containing a TGF-β antagonist, wherein the TGF-β antagonist is a fusion protein that includes a homodimer of a compound of the formula: II(a). (A-L¹-B-L²-Z), II(b). (Z-L²-B-L¹-A), or II(c). (B-L¹-A-L²-Z), where A is an RER heterotrimeric fusion polypeptide; L¹ is a linker; B is an Fc domain of an immunoglobulin or is absent; L² is a linker or is absent; Z is a bone-targeting moiety; A, the RER heterotrimeric fusion polypeptide, includes a polypeptide sequence of the formula: W-L³-X-L⁴-Y, where W is a TGF-β type II receptor ectodomain or a portion thereof; L³ is a linker or is absent; X is a TGF-β type III receptor endoglin domain or a portion thereof; L⁴ is a linker or is absent; Y is a TGF-β type II receptor ectodomain or a portion thereof, and where A includes the amino acid sequence of SEQ ID NO: 48.

Certain embodiments of the above composition may vary in ways described below.

In some embodiments, the linker L¹ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some embodiments, B, the Fc domain of an immunoglobulin is present. In some embodiments, B, the Fc domain of an immunoglobulin is absent. In some embodiments, B, the Fc domain of an immunoglobulin includes the Fc domain of human IgG, human IgA, human IgM, human IgE, or human IgD; or a variant of said domain. In some embodiments, the Fc domain of human IgG is IgG1, IgG2, IgG3, or IgG4; or a variant thereof. In some embodiments, the Fc domain of human includes the amino acid sequence of SEQ ID NO: 47; or a variant of said amino acid sequence.

In some embodiments, the linker L² is present. In some embodiments, the linker L² is absent. In some embodiments, the linker L² includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some embodiments, the bone-targeting moiety includes a polyanionic peptide, a bisphosphonate, or the amino acid sequence of SEQ ID NO: 46; or a variant of said amino acid sequence.

In some embodiments, the TGF-β type II receptor ectodomain W is at the N-terminus of the RER heterotrimeric fusion polypeptide and the TGF-β type II receptor ectodomain Y is at the C-terminus of the RER heterotrimeric fusion polypeptide. In some embodiments, the C-terminus of the TGF-β type II receptor ectodomain Y is covalently joined to the N-terminus of B, Fc domain of an immunoglobulin, via the linker L¹ as in formula I(a). In some embodiments, the N-terminus of the TGF-β type II receptor ectodomain W is covalently joined to the C-terminus of B via the linker L¹ as in formula I(b) or I(c).

In some embodiments, the amino acid sequence of the TGF-β type II receptor ectodomain W is identical to the amino acid sequence of the TGF-β type II receptor ectodomain Y. In some embodiments, the amino acid sequence of the TGF-β type II receptor ectodomain W is different than the amino acid sequence of the TGF-β type II receptor ectodomain Y. In some embodiments, the TGF-β type II receptor ectodomains W and/or Y includes an amino acid sequence extending from amino acid residues 22 to 139 of SEQ ID NO: 5, 520 to 631 of SEQ ID NO: 5, 1 to 118 of SEQ ID NO: 9, 479 to 590 of SEQ ID NO: 9, 1 to 118 of SEQ ID NO: 48, 499 to 610 of SEQ ID NO: 48, 1 to 118 of SEQ ID NO: 49, 499 to 610 of SEQ ID NO: 49, 501 to 612 of SEQ ID NO: 50, 501 to 612 of SEQ ID NO: 51, or 510 to 621 of SEQ ID NO: 52; or a variant of said amino acid sequences. In some embodiments, the TGF-β type II receptor ectodomains W and/or Y does not comprise an amino acid sequence extending from amino acid residues 22 to 139 of SEQ ID NO: 5, 520 to 631 of SEQ ID NO: 5, 1 to 118 of SEQ ID NO: 9, 479 to 590 of SEQ ID NO: 9, 1 to 118 of SEQ ID NO: 48, 499 to 610 of SEQ ID NO: 48, 1 to 118 of SEQ ID NO: 49, 499 to 610 of SEQ ID NO: 49, 501 to 612 of SEQ ID NO: 50, 501 to 612 of SEQ ID NO: 51, or 510 to 621 of SEQ ID NO: 52; or a variant of said amino acid sequences.

In some embodiments, the linker L³ is present. In some embodiments, the linker L³ is absent. In some embodiments, the linker L³ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some embodiments, the TGF-β type III receptor endoglin domain X includes an amino acid sequence extending from amino acid residues 157 to 517 of SEQ ID NO: 5, 136 to 496 of SEQ ID NO: 48, or 136 to 496 of SEQ ID NO: 49; or a variant of said amino acid sequences. In some embodiments, the TGF-β type III receptor endoglin domain X does not comprise an amino acid sequence extending from amino acid residues 157 to 517 of SEQ ID NO: 5, 136 to 496 of SEQ ID NO: 48, or 136 to 496 of SEQ ID NO: 49; or a variant of said amino acid sequences.

In some embodiments, the linker L⁴ is present. In some embodiments, the linker L⁴ is absent. In some embodiments, the linker L⁴ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some embodiments, the RER heterotrimeric fusion polypeptide includes the amino acid sequence of SEQ ID NO: 48; or a variant of said amino acid sequences.

In some embodiments, the homodimer includes an amino acid sequence selected from the group comprising SEQ ID NO: 14, SEQ ID NO: 16, SEQ ID NO: 18, SEQ ID NO: 32, and SEQ ID NO: 34; or a variant of said amino acid sequences.

In another aspect, the invention features a composition containing a TGF-β antagonist, wherein the TGF-β antagonist is a fusion protein that includes a homodimer of a compound of the formula: III(a). (A-L¹-B-L²-Z), III(b). (Z-L²-B-L¹-A), or III(c). (B-L¹-A-L²-Z), where A is an RER heterotrimeric fusion polypeptide; L¹ is a linker; B is an Fc domain of an immunoglobulin or is absent; L² is a linker or is absent; Z is a bone-targeting moiety or is absent; and where at least one of the following is present:
a. A, the RER heterotrimeric fusion polypeptide, includes an amino acid sequence selected from the group consisting of SEQ ID NO: 9, SEQ ID NO: 49, SEQ ID NO: 50, SEQ ID NO: 51, and SEQ ID NO: 52; or
b. the linker L¹ includes an amino acid sequence selected from the group consisting of SEQ ID NO: 8, SEQ ID NO: 36, SEQ ID NO: 37, and SEQ ID NO: 38; or
c. the linker L² is present and includes an amino acid sequence of SEQ ID NO: 8, or SEQ ID NO: 41; or
d. the linker L³ is present and includes the amino acid sequence of SEQ ID NO: 38 or SEQ ID NO: 39: or
e. X, the TGF-β type III receptor endoglin domain, includes the amino acid sequence of SEQ ID NO: 44.

In some embodiments, the novel TGF-β receptor fusion protein constructs or antagonists of the invention are those with the D10 bone-targeting moiety (SEQ ID NO: 46) and includes the amino acid sequence of SEQ ID NO: 5, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, or SEQ ID NO: 34, or a variant of said amino acid sequences. The TGF-β receptor fusion protein constructs or antagonists with the D10 bone-targeting moiety can be used to treat a variety of disorders associated with elevated TGF-β signaling in bone tissue.

In other embodiments, the novel TGF-β receptor fusion protein constructs or antagonists of the invention are those without the D10 bone-targeting moiety and includes the amino acid sequence of SEQ ID NO: 9, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, or SEQ ID NO: 35, or a variant of said amino acid sequences. The TGF-β receptor fusion protein constructs or antagonists without the D10 bone-targeting moiety can be used to treat a variety of disorders associated with elevated TGF-β signaling in both bone tissue and tissues other than bone.

Other bone-targeting moieties, as described herein, may be used in lieu of the D10 bone-targeting moiety, as appropriate.

The above TGF-β antagonist constructs and conjugates may be used appropriately and interchangeably with the TGF-β antagonist constructs and conjugates of any of the aspects or embodiments of the invention and the TGF-β antagonist constructs and conjugates described below.

In some embodiments, the TGF-β antagonist binds TGF-β. In some embodiments, the TGF-β antagonist binds and neutralizes TGF-β, for instance, thereby suppressing TGF-β signal transduction. In some embodiments, the TGF-β antagonist is a protein, peptide, antibody, or small molecule that binds TGF-β.

In general, the TGF-β antagonist of the invention include a protein that contains one or more soluble TGF-β receptors, or domains or fragments thereof. For instance, the TGF-β antagonist may be a fusion protein that contains one or more TGF-β receptors. Exemplary fusion protein TGF-β antagonists that may be used in conjunction with the compositions and methods described herein include fusion proteins that contains one or more domains of TGF-β receptor II each joined to one or more domains of TGF-β receptor III.

In some embodiments, the invention features a conjugate containing a TGF-β antagonist bound to a targeting moiety, wherein the TGF-β antagonist is a fusion protein that contains one or more domains of TGF-β receptor II each joined to one or more domains of TGF-β receptor III. In some embodiments, the TGF-β antagonist described herein is bound to a targeting moiety that specifically binds hydroxyapatite.

In some embodiments, the TGF-β antagonist contains a TGF-β receptor II ectodomain bound to a TGF-β receptor III endoglin domain.

In some embodiments, the C-terminal region of the TGF-β receptor II ectodomain is bound to the N-terminal region of the TGF-β receptor III endoglin domain. For instance, the C-terminal amino acid residue of the TGF-β receptor II ectodomain may be bound to the N-terminal amino acid residue of the TGF-β receptor III endoglin domain. In some embodiments, the C-terminal region of the TGF-β receptor II ectodomain is bound to the N-terminal region of the TGF-β receptor III endoglin domain by a linker. In some embodiment, the linker is a peptidic linker.

In some embodiments, the N-terminal region of the TGF-β receptor II ectodomain is bound to the C-terminal region of the TGF-β receptor III endoglin domain. For instance, the N-terminal amino acid residue of the TGF-β receptor II ectodomain may be bound to the C-terminal amino acid residue of the TGF-β receptor III endoglin domain. In some embodiments, the N-terminal region of the TGF-β receptor II ectodomain is bound to the C-terminal region of the TGF-β receptor III endoglin domain by a linker. In some embodiments, the linker is a peptidic linker.

In the above embodiments where a peptidic linker is present, the linker may include amino acid residues from the first 35 amino acid residues of the TGF-β receptor as appropriate (e.g., the first 2, 3, 4, 5, 6, 7, 8, 9, 10, 11, 12, 13, 14, 15, 16, 17, 18, 19, 20, 21, 22, 23, 24, 25, 26, 27, 28, 29, 30, or 35 amino acid residues of the TGF-β receptor II ectodomain or TGF-β receptor III endoglin domain). In some embodiments, the peptidic linker may include amino acid residues from the first 10 amino acid residues of the TGF-β receptor as appropriate, i.e., the TGF-β receptor II ectodomain or TGF-β receptor III endoglin domain.

In the above embodiments where a peptidic linker is present, the linker may include amino acid residues from the final 35 amino acid residues of the TGF-β receptor as appropriate (e.g., the final 2, 3, 4, 5, 6, 7, 8, 9, 10, 11, 12, 13, 14, 15, 16, 17, 18, 19, 20, 21, 22, 23, 24, 25, 26, 27, 28, 29, 30, or 35 amino acid residues of the TGF-β receptor II ectodomain or TGF-β receptor III endoglin domain). In some embodiments, the peptidic linker may include amino acid residues from the final 10 amino acid residues of the TGF-β receptor as appropriate, i.e., the TGF-β receptor II ectodomain or TGF-β receptor III endoglin domain.

In the above embodiments where a peptidic linker is present, the linker may include a naturally-occurring amino acid residue. In some embodiments, the naturally-occurring amino acid residue is selected from the group consisting of lysine, aspartic acid, glutamic acid, asparagine, glutamine, serine, threonine, and cysteine.

In the above embodiments where a peptidic linker is present, the linker may include a non-natural amino acid residue. In some embodiments, the non-natural amino acid residue contains a reactive substituent selected from the group consisting of amino, carboxy, acetyl, hydrazino, hydrazido, hydroxy, semicarbazido, mercapto, sulfanyl, azido, alkenyl, and alkynyl.

In some embodiments, the TGF-β receptor II ectodomain is from human TGF-β receptor II.

In some embodiments, the TGF-β receptor II ectodomain has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to the sequence of amino acid residues 24-160 of SEQ ID NO: 1, 42-159 of SEQ ID NO: 1, or 48-159 of SEQ ID NO: 1. In some embodiments, the TGF-β receptor II ectodomain has an amino acid sequence having at least 90% sequence identity to the sequence of amino acid residues 24-160 of SEQ ID NO: 1, 42-159 of SEQ ID NO: 1, or 48-159 of SEQ ID NO: 1. In some embodiments, the TGF-β receptor II ectodomain has an amino acid sequence having at least 95% sequence identity to the sequence of amino acid residues 24-160 of SEQ ID NO: 1, 42-159 of SEQ ID NO: 1, or 48-159 of SEQ ID NO: 1. In some embodiments, the TGF-β receptor II ectodomain has the amino acid sequence of amino acid residues 24-160 of SEQ ID NO: 1, 42-159 of SEQ ID NO: 1, or 48-159 of SEQ ID NO: 1. In some embodiments, the TGF-β receptor II ectodomain has an amino acid sequence that differs from the sequence of amino acid residues 24-160 of SEQ ID NO: 1, 42-159 of SEQ ID NO: 1, or 48-159 of SEQ ID NO: 1 by one or more conservative substitutions (e.g., by 1, 2, 3, 4, 5, 6, 7, 8, 9, 10, 15, 20, or more, conservative substitutions). In some embodiments, the TGF-β receptor II ectodomain has an amino acid sequence that differs from the sequence of amino acid residues 24-160 of SEQ ID NO: 1, 42-159 of SEQ ID NO: 1, or 48-159 of SEQ ID NO: 1 by fewer than 10 non-conservative substitutions (e.g., by 10, 9, 8, 7, 6, 5, 4, 3, 2, 1, or 0 non-conservative substitutions). In some embodiments, the TGF-β receptor II ectodomain has an amino acid sequence that differs from the sequence of amino acid residues 24-160 of SEQ ID NO: 1, 42-159 of SEQ ID NO: 1, or 48-159 of SEQ ID NO: 1 only by one or more conservative substitutions (e.g., only by 1, 2, 3, 4, 5, 6, 7, 8, 9, 10, 15, 20, or more, conservative substitutions). In some embodiments, the TGF-β receptor II ectodomain contains amino acid residues 50-53 of SEQ ID NO: 1 (i.e., has a sub-sequence that has 100% sequence identity to the sequence of amino acid residues 50-53 of SEQ ID NO: 1).

In some embodiments, the TGF-β receptor III endoglin domain is from rat TGF-β receptor III.

In some embodiments, the TGF-β receptor III endoglin domain has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to the sequence of amino acid residues 24-383 of SEQ ID NO: 2 or 24-409 of SEQ ID NO: 2. In some embodiments, the TGF-β receptor III endoglin domain has an amino acid sequence having at least 90% sequence identity to the sequence of amino acid residues 24-383 of SEQ ID NO: 2 or 24-409 of SEQ ID NO: 2. In some embodiments, the TGF-β receptor III endoglin domain has an amino acid sequence having at least 95% sequence identity to the sequence of amino acid residues 24-383 of SEQ ID NO: 2 or 24-409 of SEQ ID NO: 2. In some embodiments, the TGF-β receptor III endoglin domain has the amino acid sequence of amino acid residues 24-383 of SEQ ID NO: 2 or 24-409 of SEQ ID NO: 2. In some embodiments, the TGF-β receptor III endoglin domain has an amino acid sequence that differs from the sequence of amino acid residues 24-383 of SEQ ID NO: 2 or 24-409 of SEQ ID NO: 2 by one or more conservative substitutions (e.g., by 1, 2, 3, 4, 5, 6, 7, 8, 9, 10, 15, 20, or more, conservative substitutions). In some embodiments, the TGF-β receptor III endoglin domain has an amino acid sequence that differs from the sequence of amino acid residues 24-383 of SEQ ID NO: 2 or 24-409 of SEQ ID NO: 2 by fewer than 10 non-conservative substitutions (e.g., by 10, 9, 8, 7, 6, 5, 4, 3, 2, 1, or 0 non-conservative substitutions). In some embodiments, the TGF-β receptor III endoglin domain has an amino acid sequence that differs from the sequence of amino acid residues 24-383 of SEQ ID NO: 2 or 24-409 of SEQ ID NO: 2 only by one or more conservative substitutions (e.g., only by 1, 2, 3, 4, 5, 6, 7, 8, 9, 10, 15, 20, or more, conservative substitutions). In some embodiments, the TGF-β receptor III endoglin domain contains R58H, H116R, C278S, and N337A substitutions relative the sequence of amino acid residues 24-383 of SEQ ID NO: 2 or 24-409 of SEQ ID NO: 2.

In some embodiments, the TGF-β receptor III endoglin domain has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to the sequence of SEQ ID NO: 12. In some embodiments, the TGF-β receptor III endoglin domain has an amino acid sequence having at least 90% sequence identity to the sequence of SEQ ID NO: 12. In some embodiments, the TGF-β receptor III endoglin domain has an amino acid sequence having at least 95% sequence identity to the sequence of SEQ ID NO: 12. In some embodiments, the TGF-β receptor III endoglin domain has the amino acid sequence of SEQ ID NO: 12. In some embodiments, the TGF-β receptor III endoglin domain has an amino acid sequence that differs from the sequence of SEQ ID NO: 12 by one or more conservative substitutions (e.g., by 1, 2, 3, 4, 5, 6, 7, 8, 9, 10, 15, 20, or more, conservative substitutions). In some embodiments, the TGF-β receptor III endoglin domain has an amino acid sequence that differs from the sequence of SEQ ID NO: 12 by fewer than 10 non-conservative substitutions (e.g., by 10, 9, 8, 7, 6, 5, 4, 3, 2, 1, or 0 non-conservative substitutions). In some embodiments, the TGF-β receptor III endoglin domain has an amino acid sequence that differs from the sequence of SEQ ID NO: 12 only by one or more conservative substitutions (e.g., only by 1, 2, 3, 4, 5, 6, 7, 8, 9, 10, 15, 20, or more, conservative substitutions).

In some embodiments, the TGF-β receptor III endoglin domain is from human TGF-β receptor III.

In some embodiments, the TGF-β receptor III endoglin domain has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to the sequence of amino acid residues 21-380 of SEQ ID NO: 3 or 21-406 of SEQ ID NO: 3. In some embodiments, the TGF-β receptor III endoglin domain has an amino acid sequence having at least 90% sequence identity to the sequence of amino acid residues 21-380 of SEQ ID NO: 3 or 21-406 of SEQ ID NO: 3. In some embodiments, the TGF-β receptor III endoglin domain has an amino acid sequence having at least 95% sequence identity to the sequence of amino acid residues 21-380 of SEQ ID NO: 3 or 21-406 of SEQ ID NO: 3. In some embodiments, the TGF-β receptor III endoglin domain has the amino acid sequence of amino acid residues 21-380 of SEQ ID NO: 3 or 21-406 of SEQ ID NO: 3. In some embodiments, the TGF-β receptor III endoglin domain has an amino acid sequence that differs from the sequence of amino acid residues 21-380 of SEQ ID NO: 3 or 21-406 of SEQ ID NO: 3 by one or more conservative substitutions (e.g., by 1, 2, 3, 4, 5, 6, 7, 8, 9, 10, 15, 20, or more, conservative substitutions). In some embodiments, the TGF-β receptor III endoglin domain has an amino acid sequence that differs from the sequence of amino acid residues 21-380 of SEQ ID NO: 3 or 21-406 of SEQ ID NO: 3 by fewer than 10 non-conservative substitutions (e.g., by 10, 9, 8, 7, 6, 5, 4, 3, 2, 1, or 0 non-conservative substitutions). In some embodiments, the TGF-β receptor III endoglin domain has an amino acid sequence that differs from the sequence of amino acid residues 21-380 of SEQ ID NO: 3 or 21-406 of SEQ ID NO: 3 only by one or more conservative substitutions (e.g., only by 1, 2, 3, 4, 5, 6, 7, 8, 9, 10, 15, 20, or more, conservative substitutions). In some embodiments, the TGF-β receptor III endoglin domain contains one or more, or all, of the mutations R55H, H113R, C275S, and N334A substitutions relative the sequence of amino acid residues 21-380 of SEQ ID NO: 3 or 21-406 of SEQ ID NO: 3.

In some embodiments, the targeting moiety is bound to the TGF-β receptor II ectodomain of the TGF-β antagonist.

In some embodiments, the targeting moiety is bound to the N-terminal region of the TGF-β receptor II ectodomain. For instance, the targeting moiety may be bound to the N-terminal amino acid residue of the TGF-β receptor II ectodomain. In some embodiments, the targeting moiety may be bound to the N-terminal region of the TGF-β receptor II ectodomain by a peptidic linker.

In some embodiments, the targeting moiety is bound to the C-terminal region of the TGF-β receptor II ectodomain. For instance, the targeting moiety may be bound to the C-terminal amino acid residue of the TGF-β receptor II ectodomain. In some embodiments, the targeting moiety is bound to the C-terminal region of the TGF-β receptor II ectodomain by a linker. In some embodiments, the linker is a peptidic linker.

In some embodiments, the targeting moiety is bound to the TGF-β receptor III endoglin domain of the TGF-β antagonist.

In some embodiments, the targeting moiety is bound to the N-terminal region of the TGF-β receptor III endoglin domain. For instance, the targeting moiety may be bound to the N-terminal amino acid residue of the TGF-β receptor III endoglin domain. In some embodiments, the targeting moiety is bound to the N-terminal region of the TGF-β receptor II ectodomain by a linker. In some embodiments, the linker is a peptidic linker.

In some embodiments, the targeting moiety is bound to the C-terminal region of the TGF-β receptor III endoglin domain. For instance, the targeting moiety may be bound to the C-terminal amino acid residue of the TGF-β receptor III endoglin domain. In some embodiments, the targeting moiety may be bound the C-terminal region of the TGF-β receptor III endoglin domain by a linker. In some embodiments, the linker is a peptidic linker.

In the above embodiments where a peptidic linker is present, the linker may include amino acid residues from the first 35 amino acid residues of the TGF-β receptor as appropriate (e.g., the first 2, 3, 4, 5, 6, 7, 8, 9, 10, 11, 12, 13, 14, 15, 16, 17, 18, 19, 20, 21, 22, 23, 24, 25, 26, 27, 28, 29, 30, or 35 amino acid residues, of the TGF-β receptor II ectodomain or TGF-β receptor III endoglin domain). In some embodiments, the peptidic linker may include amino acid residues from the first 10 amino acid residues of the TGF-β receptor as appropriate, i.e., the TGF-β receptor II ectodomain or TGF-β receptor III endoglin domain.

In the above embodiments where a peptidic linker is present, the linker may include amino acid residues from the final 35 amino acid residues of the TGF-β receptor as appropriate (e.g., the final 2, 3, 4, 5, 6, 7, 8, 9, 10, 11, 12, 13, 14, 15, 16, 17, 18, 19, 20, 21, 22, 23, 24, 25, 26, 27, 28, 29, 30, or 35 amino acid residues, of the TGF-β receptor II ectodomain or TGF-β receptor III endoglin domain). In some embodiments, the linker may include amino acid residues from the final 10 amino acid residues of the TGF-β receptor as appropriate, i.e., the TGF-β receptor II ectodomain or TGF-β receptor III endoglin domain.

In the above embodiments, the peptidic linker may include a naturally-occurring amino acid residue. In some embodiments, the naturally-occurring amino acid residue is selected from the group consisting of lysine, aspartic acid, glutamic acid, asparagine, glutamine, serine, threonine, and cysteine.

In the above embodiments, the peptidic linker may include a non-natural amino acid residue. In some embodiments, the non-natural amino acid residue contains a reactive substituent selected from the group consisting of amino, carboxy, acetyl, hydrazino, hydrazido, hydroxy, semicarbazido, mercapto, sulfanyl, azido, alkenyl, and alkynyl.

In some embodiments, the TGF-β antagonist contains:
(a) a first TGF-β receptor II ectodomain or a portion thereof;
(b) a TGF-β receptor III endoglin domain or a portion thereof; and
(c) a second TGF-β receptor II ectodomain or a portion thereof.

In some embodiments, the first and second TGF-β receptor II ectodomains are each independently bound to the TGF-β receptor III endoglin domain.

In some embodiments, the TGF-β antagonist contains, from N-terminus to C-terminus:
(a) a first TGF-β receptor II ectodomain or a portion thereof;
(b) a TGF-β receptor III endoglin domain or a portion thereof; and
(c) a second TGF-β receptor II ectodomain or a portion thereof.

In some embodiments, the first and second TGF-β receptor II ectodomains are each independently bound to the TGF-β receptor III endoglin domain.

In some embodiments, the C-terminal region of the first TGF-β receptor II ectodomain is bound to the N-terminal region of the TGF-β receptor III endoglin domain. In some embodiments, the C-terminal region of the TGF-β receptor III endoglin domain is bound to the N-terminal region of the second TGF-β receptor II ectodomain.

In some embodiments, the C-terminal amino acid residue of the first TGF-β receptor II ectodomain is bound to the N-terminal amino acid residue of the TGF-β receptor III endoglin domain. In some embodiments, the C-terminal region of the first TGF-β receptor II ectodomain is bound to the N-terminal region of the TGF-β receptor III endoglin domain by a linker. In some embodiments, the linker is a peptidic linker.

In some embodiments, the N-terminal amino acid residue of the second TGF-β receptor II ectodomain is bound to the C-terminal amino acid residue of the TGF-β receptor III endoglin domain. In some embodiments, the N-terminal region of the second TGF-β receptor II ectodomain is bound to the C-terminal region of the TGF-β receptor III endoglin domain by a linker. In some embodiments, the linker is a peptidic linker.

In the above embodiments where a peptidic linker is present, the linker may include amino acid residues from the first 35 amino acid residues of the TGF-β receptor as appropriate (e.g., the first 2, 3, 4, 5, 6, 7, 8, 9, 10, 11, 12, 13, 14, 15, 16, 17, 18, 19, 20, 21, 22, 23, 24, 25, 26, 27, 28, 29, 30, or 35 amino acid residues, of the first TGF-β receptor II ectodomain, second TGF-β receptor II ectodomain, or the TGF-β receptor III endoglin domain). In some embodiments, the peptidic linker may include amino acid residues from the first 10 amino acid residues of the TGF-β receptor as appropriate, i.e., the first TGF-β receptor II ectodomain, second TGF-β receptor II ectodomain, or the TGF-β receptor III endoglin domain.

In the above embodiments where a peptidic linker is present, the linker may include amino acid residues from the final 35 amino acid residues of the TGF-β receptor as appropriate (e.g., the final 2, 3, 4, 5, 6, 7, 8, 9, 10, 11, 12, 13, 14, 15, 16, 17, 18, 19, 20, 21, 22, 23, 24, 25, 26, 27, 28, 29, 30, or 35 amino acid residues, of the first TGF-β receptor II ectodomain, second TGF-β receptor II ectodomain, or the TGF-β receptor III endoglin domain). In some embodiments, the peptidic linker may include amino acid residues from the final 10 amino acid residues of the TGF-β receptor as appropriate, i.e., the first TGF-β receptor II ectodomain, second TGF-β receptor II ectodomain, or the TGF-β receptor III endoglin domain.

In the above embodiments, the peptidic linker may include a naturally-occurring amino acid residue. In some embodiments, the naturally-occurring amino acid residue is selected from the group consisting of lysine, aspartic acid, glutamic acid, asparagine, glutamine, serine, threonine, and cysteine.

In the above embodiments, the peptidic linker may include a non-natural amino acid residue. In some embodiments, the non-natural amino acid residue contains a reactive substituent selected from the group consisting of amino, carboxy, acetyl, hydrazino, hydrazido, hydroxy, semicarbazido, mercapto, sulfanyl, azido, alkenyl, and alkynyl.

In some embodiment, the first TGF-β receptor II ectodomain is from human TGF-β receptor II.

In some embodiments, the first TGF-β receptor II ectodomain has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to the sequence of amino acid residues 24-160 of SEQ ID NO: 1, 42-159 of SEQ ID NO: 1, or 48-159 of SEQ ID NO: 1. In some embodiments, the first TGF-β receptor II ectodomain has an amino acid sequence having at least 90% sequence identity to the sequence of amino acid residues 24-160 of SEQ ID NO: 1, 42-159 of SEQ ID NO: 1, or 48-159 of SEQ ID NO: 1. In some embodiments, the first TGF-β receptor II ectodomain has an amino acid sequence having at least 95% sequence identity to the sequence of amino acid residues 42-159 of SEQ ID NO: 1. In some embodiments, the first TGF-β receptor II ectodomain has the amino acid sequence of amino acid residues 24-160 of SEQ ID NO: 1, 42-159 of SEQ ID NO: 1, or 48-159 of SEQ ID NO: 1. In some embodiments, the first TGF-β receptor II ectodomain has an amino acid sequence that differs from the sequence of amino acid residues 24-160 of SEQ ID NO: 1, 42-159 of SEQ ID NO: 1, or 48-159 of SEQ ID NO: 1 by one or more conservative substitutions (e.g., by 1, 2, 3, 4, 5, 6, 7, 8, 9, 10, 15, 20, or more, conservative substitutions). In some embodiments, the first TGF-β receptor II ectodomain has an amino acid sequence that differs from the sequence of amino acid residues 24-160 of SEQ ID NO: 1, 42-159 of SEQ ID NO: 1, or 48-159 of SEQ ID NO: 1 by fewer than 10 non-conservative substitutions (e.g., by 10, 9, 8, 7, 6, 5, 4, 3, 2, 1, or 0 non-conservative substitutions). In some embodiments, the first TGF-β receptor II ectodomain has an amino acid sequence that differs from the sequence of amino acid residues 24-160 of SEQ ID NO: 1, 42-159 of SEQ ID NO: 1, or 48-159 of SEQ ID NO: 1 only by one or more conservative substitutions (e.g., only by 1, 2, 3, 4, 5, 6, 7, 8, 9, 10, 15, 20, or more, conservative substitutions). In some embodiments, the first TGF-β receptor II ectodomain contains amino acid residues 50-53 of SEQ ID NO: 1 (i.e., has a sub-sequence that has 100% sequence identity to the sequence of amino acid residues 50-53 of SEQ ID NO: 1).

In some embodiment, the second TGF-β receptor II ectodomain is from human TGF-β receptor II.

In some embodiments, the second TGF-β receptor II ectodomain has an amino acid sequence having at least 75% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to the sequence of amino acid residues 24-160 of SEQ ID NO: 1, 42-159 of SEQ ID NO: 1, or 48-159 of SEQ ID NO: 1. In some embodiments, the second TGF-β receptor II ectodomain has an amino acid sequence having at least 90% sequence identity to the sequence of amino acid residues 24-160 of SEQ ID NO: 1, 42-159 of SEQ ID NO: 1, or 48-159 of SEQ ID NO: 1. In some embodiments, the second TGF-β receptor II ectodomain has an amino acid sequence having at least 95% sequence identity to the sequence of amino acid residues 24-160 of SEQ ID NO: 1, 42-159 of SEQ ID NO: 1, or 48-159 of SEQ ID NO: 1. In some embodiments, the second TGF-β receptor II ectodomain has the amino acid sequence of amino acid residues 24-160 of SEQ ID NO: 1, 42-159 of SEQ ID NO: 1, or 48-159 of SEQ ID NO: 1. In some embodiments, the second TGF-β receptor II ectodomain has an amino acid sequence that differs from the sequence of amino acid residues 24-160 of SEQ ID NO: 1, 42-159 of SEQ ID NO: 1, or 48-159 of SEQ ID NO: 1 by one or more conservative substitutions (e.g., by 1, 2, 3, 4, 5, 6, 7, 8, 9, 10, 15, 20, or more, conservative substitutions). In some embodiments, the second TGF-β receptor II ectodomain has an amino acid sequence that differs from the sequence of amino acid residues 24-160 of SEQ ID NO: 1, 42-159 of SEQ ID NO: 1, or 48-159 of SEQ ID NO: 1 by fewer than 10 non-conservative substitutions (e.g., by 10, 9, 8, 7, 6, 5, 4, 3, 2, 1, or 0 non-conservative substitutions). In some embodiments, the second TGF-β receptor II ectodomain has an amino acid sequence that differs from the sequence of amino acid residues 24-160 of SEQ ID NO: 1, 42-159 of SEQ ID NO: 1, or 48-159 of SEQ ID NO: 1 only by one or more conservative substitutions (e.g., only by 1, 2, 3, 4, 5, 6, 7, 8, 9, 10, 15, 20, or more, conservative substitutions). In some embodiments, the second TGF-β receptor II ectodomain contains amino acid residues 50-53 of SEQ ID NO: 1 (i.e., has a sub-sequence that has 100% sequence identity to the sequence of amino acid residues 50-53 of SEQ ID NO: 1).

In some embodiments, the TGF-β receptor III endoglin domain is from rat TGF-β receptor III.

In some embodiments, the TGF-β receptor III endoglin domain has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to the sequence of amino acid residues 24-383 of SEQ ID NO: 2 or 24-409 of SEQ ID NO: 2. In some embodiments, the TGF-β receptor III endoglin domain has an amino acid sequence having at least 90% sequence identity to the sequence of amino acid residues 24-383 of SEQ ID NO: 2 or 24-409 of SEQ ID NO: 2. In some embodiments, the TGF-β receptor III endoglin domain has an amino acid sequence having at least 95% sequence identity to the sequence of amino acid residues 24-383 of SEQ ID NO: 2 or 24-409 of SEQ ID NO: 2. In some embodiments, the TGF-β receptor III endoglin domain has the amino acid sequence of amino acid residues 24-383 of SEQ ID NO: 2 or 24-409 of SEQ ID NO: 2. In some embodiments, the TGF-β receptor III endoglin domain has an amino acid sequence that differs from the sequence of amino acid residues 24-383 of SEQ ID NO: 2 or 24-409 of SEQ ID NO: 2 by one or more conservative substitutions (e.g., by 1, 2, 3, 4, 5, 6, 7, 8, 9, 10, 15, 20, or more, conservative substitutions). In some embodiments, the TGF-β receptor III endoglin domain has an amino acid sequence that differs from the sequence of amino acid residues 24-383 of SEQ ID NO: 2 or 24-409 of SEQ ID NO: 2 by fewer than 10 non-conservative substitutions (e.g., by 10, 9, 8, 7, 6, 5, 4, 3, 2, 1, or 0 non-conservative substitutions). In some embodiments, the TGF-β receptor III endoglin domain has an amino acid sequence that differs from the sequence of amino acid residues 24-383 of SEQ ID NO: 2 or 24-409 of SEQ ID NO: 2 only by one or more conservative substitutions (e.g., only by 1, 2, 3, 4, 5, 6, 7, 8, 9, 10, 15, 20, or more, conservative substitutions). In some embodiments, the TGF-β receptor III endoglin domain contains R58H, H116R, C278S, and N337A substitutions relative the sequence of amino acid residues 24-383 of SEQ ID NO: 2 or 24-409 of SEQ ID NO: 2.

In some embodiments, the TGF-β receptor III endoglin domain has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to the sequence of SEQ ID NO: 12. In some embodiments, the TGF-β receptor III endoglin domain has an amino acid sequence having at least 90% sequence identity to the sequence of SEQ ID NO: 12. In some embodiments, the TGF-β receptor III endoglin domain has an amino acid sequence having at least 95% sequence identity to the sequence of SEQ ID NO: 12. In some embodiments, the TGF-β receptor III endoglin domain has the amino acid sequence of SEQ ID NO: 12. In some embodiments, the TGF-β receptor III endoglin domain has an amino acid sequence that differs from the sequence of SEQ ID NO: 12 by one or more conservative substitutions (e.g., by 1, 2, 3, 4, 5, 6, 7, 8, 9, 10, 15, 20, or more, conservative substitutions). In some embodiments, the TGF-β receptor III endoglin domain has an amino acid sequence that differs from the sequence of SEQ ID NO: 12 by fewer than 10 non-conservative substitutions (e.g., by 10, 9, 8, 7, 6, 5, 4, 3, 2, 1, or 0 non-conservative substitutions). In some embodiments, the TGF-β receptor III endoglin domain has an amino acid sequence that differs from the sequence of SEQ ID NO: 12 only by one or more conservative substitutions (e.g., only by 1, 2, 3, 4, 5, 6, 7, 8, 9, 10, 15, 20, or more, conservative substitutions).

In some embodiments, the TGF-β receptor III endoglin domain is from human TGF-β receptor III.

In some embodiments, the TGF-β receptor III endoglin domain has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to the sequence of amino acid residues 21-380 of SEQ ID NO: 3 or 21-406 of SEQ ID NO: 3. In some embodiments, the TGF-β receptor III endoglin domain has an amino acid sequence having at least 90% sequence identity to the sequence of amino acid residues 21-380 of SEQ ID NO: 3 or 21-406 of SEQ ID NO: 3. In some embodiments, the TGF-β receptor III endoglin domain has an amino acid sequence having at least 95% sequence identity to the sequence of amino acid residues 21-380 of SEQ ID NO: 3 or 21-406 of SEQ ID NO: 3. In some embodiments, the TGF-β receptor III endoglin domain has the amino acid sequence of amino acid residues 21-380 of SEQ ID NO: 3 or 21-406 of SEQ ID NO: 3. In some embodiments, the TGF-β receptor III endoglin domain has an amino acid sequence that differs from the sequence of amino acid residues 21-380 of SEQ ID NO: 3 or 21-406 of SEQ ID NO: 3 by one or more conservative substitutions (e.g., by 1, 2, 3, 4, 5, 6, 7, 8, 9, 10, 15, 20, or more, conservative substitutions). In some embodiments, the TGF-β receptor III endoglin domain has an amino acid sequence that differs from the sequence of amino acid residues 21-380 of SEQ ID NO: 3 or 21-406 of SEQ ID NO: 3 by fewer than 10 non-conservative substitutions (e.g., by 10, 9, 8, 7, 6, 5, 4, 3, 2, 1, or 0 non-conservative substitutions). In some embodiments, the TGF-β receptor III endoglin domain has an amino acid sequence that differs from the sequence of amino acid residues 21-380 of SEQ ID NO: 3 or 21-406 of SEQ ID NO: 3 only by one or more conservative substitutions (e.g., only by 1, 2, 3, 4, 5, 6, 7, 8, 9, 10, 15, 20, or more, conservative substitutions). In some embodiments, the TGF-β receptor III endoglin domain contains one or more, or all, of the mutations R55H, H113R, C275S, and N334A substitutions relative the sequence of amino acid residues 21-380 of SEQ ID NO: 3 or 21-406 of SEQ ID NO: 3.

In some embodiments, the targeting moiety is bound to the first TGF-β receptor II ectodomain of the TGF-β antagonist. In some embodiments, the targeting moiety is bound to the N-terminal region of the first TGF-β receptor II ectodomain. In some embodiments, the targeting moiety is bound to the N-terminal region of the first TGF-β receptor II ectodomain by a linker. In some embodiments, the linker is a peptidic linker.

In some embodiments, the targeting moiety is bound to the C-terminal region of the first TGF-β receptor II ectodomain. For instance, the targeting moiety may be bound to the C-terminal amino acid residue of the first TGF-β receptor II ectodomain. In some embodiments, the targeting moiety is bound to the C-terminal region of the first TGF-β receptor II ectodomain by a linker. In some embodiments, the linker is a peptidic linker.

In some embodiments, the targeting moiety is bound to the second TGF-β receptor II ectodomain of the TGF-β antagonist. In some embodiments, the targeting moiety is bound to the N-terminal region of the second TGF-β receptor II ectodomain. In some embodiments, the targeting moiety is bound to the N-terminal region of the second TGF-β receptor II ectodomain by a linker. In some embodiments, the linker is a peptidic linker.

In some embodiments, the targeting moiety is bound to the C-terminal region of the second TGF-β receptor II ectodomain. For instance, the targeting moiety may be bound to the C-terminal amino acid residue of the second TGF-β receptor II ectodomain. In some embodiments, the targeting moiety is bound to the C-terminal region of the second TGF-β receptor II ectodomain by a linker. In some embodiments, the linker is a peptidic linker.

In some embodiments, the targeting moiety is bound to the TGF-β receptor III endoglin domain of the TGF-β antagonist. In some embodiments, the targeting moiety is bound to the N-terminal region of the TGF-β receptor III endoglin domain. In some embodiments, the targeting moiety is bound to the N-terminal region of the TGF-β receptor III endoglin domain by a linker. In some embodiments, the linker is a peptidic linker.

In some embodiments, the targeting moiety is bound to the C-terminal region of the TGF-β receptor III endoglin domain. For instance, the targeting moiety may be bound to the C-terminal amino acid residue of the TGF-β receptor III endoglin domain. In some embodiments, the targeting moiety is bound to the C-terminal region of the TGF-β receptor III endoglin domain by a linker. In some embodiments, the linker is a peptidic linker.

In the above embodiments where a peptidic linker is present, the linker may include amino acid residues from the first 35 amino acid residues of the TGF-β receptor as appropriate (e.g., the first 2, 3, 4, 5, 6, 7, 8, 9, 10, 11, 12, 13, 14, 15, 16, 17, 18, 19, 20, 21, 22, 23, 24, 25, 26, 27, 28, 29, 30, or 35 amino acid residues, of the first TGF-β receptor II ectodomain, second TGF-β receptor II ectodomain, or the TGF-β receptor III endoglin domain). In the above embodiments, the peptidic linker may include amino acid residues from the first 10 amino acid residues of the TGF-β receptor as appropriate, i.e., the first TGF-β receptor II ectodomain, second TGF-β receptor II ectodomain, or the TGF-β receptor III endoglin domain.

In the above embodiments where a peptidic linker is present, the linker may include amino acid residues from the final 35 amino acid residues of the TGF-β receptor as appropriate (e.g., the final 2, 3, 4, 5, 6, 7, 8, 9, 10, 11, 12, 13, 14, 15, 16, 17, 18, 19, 20, 21, 22, 23, 24, 25, 26, 27, 28, 29, 30, or 35 amino acid residues, of the first TGF-β receptor II ectodomain, second TGF-β receptor II ectodomain, or the TGF-β receptor III endoglin domain). In the above embodiments, the peptidic linker may include amino acid residues from the final 10 amino acid residues of the TGF-β receptor as appropriate, i.e., the first TGF-β receptor II ectodomain, second TGF-β receptor II ectodomain, or the TGF-β receptor III endoglin domain.

In the above embodiments, the peptidic linker may include a naturally-occurring amino acid residue. In some embodiments, the naturally-occurring amino acid residue is selected from the group consisting of lysine, aspartic acid, glutamic acid, asparagine, glutamine, serine, threonine, and cysteine.

In the above embodiments, the peptidic linker may include a non-natural amino acid residue. In some embodiments, the non-natural amino acid residue contains a reactive substituent selected from the group consisting of amino, carboxy, acetyl, hydrazino, hydrazido, hydroxy, semicarbazido, mercapto, sulfanyl, azido, alkenyl, and alkynyl.

In some embodiments, the invention features a method of treating a human patient suffering from a disease associated with elevated TGF-β signaling by administering to the patient a therapeutically effective amount of a TGF-β antagonist that includes an antibody or an antigen-binding fragment thereof that binds TGF-β.

In some embodiments, the antibody or antigen-binding fragment thereof that binds TGF-β is conjugated to a targeting moiety that binds a protein or mineral present in human bone tissue.

In some embodiments, the invention features a method of treating a human patient suffering from a bone disease associated with elevated TGF-β signaling by administering to the patient a therapeutically effective amount of a TGF-β antagonist that includes a TGF-β-binding antibody or an antigen-binding fragment thereof that is conjugated to a targeting moiety that binds a protein or mineral present in human bone tissue.

In some embodiments, the invention features a method of treating a human patient suffering from a bone disease associated with elevated TGF-β signaling by administering to the patient a therapeutically effective amount of a TGF-β antagonist that includes a TGF-β-binding antibody or an antigen-binding fragment thereof that is not conjugated to a targeting moiety that binds a protein or mineral present in human bone tissue.

In some embodiments, the invention features a method of improving muscle function in a human patient suffering from a disease associated with elevated TGF-β signaling by administering to the patient a therapeutically effective amount of a TGF-β antagonist that includes a TGF-β-binding antibody or an antigen-binding fragment thereof that is conjugated to a targeting moiety that binds a protein or mineral present in human bone tissue.

In some embodiments, the invention features a method of improving muscle function in a human patient suffering from a disease associated with elevated TGF-β signaling by administering to the patient a therapeutically effective amount of a TGF-β antagonist that includes a TGF-β-binding antibody or an antigen-binding fragment thereof that is not conjugated to a targeting moiety that binds a protein or mineral present in human bone tissue.

In some embodiments of the above methods of the invention, the disease is a disease associated with elevated bone turnover. In some embodiments of the above methods of the invention, the disease is a bone disease. In other embodiments of the above methods of the invention, the disease is a muscle disease.

In some embodiments of the above methods of the invention, the invention features a method of improving muscle function in a human patient suffering from a disease associated with elevated bone turnover, by administering to the patient a therapeutically effective amount of a conjugate or pharmaceutical composition of any of the above aspects or embodiments of the invention.

In some embodiments of the above methods of the invention, the disease is selected from the group consisting of renal osteodystrophy, hyperparathyroid induced bone disease, diabetic bone disease, osteoarthritis, steroid induced bone disease, disuse osteoporosis, and Cerebral Palsy.

In some embodiments of the above methods of the invention, the disease is selected from the group consisting of osteogenesis imperfecta, McCune-Albright Syndrome, Gaucher Disease, Hyperoxaluria, Paget Disease of bone, and Juvenile Paget Disease.

In some embodiments of the above methods of the invention, the disease is osteogenesis imperfecta, such as Type I osteogenesis imperfecta, Type II osteogenesis imperfecta, Type III osteogenesis imperfecta, Type IV osteogenesis imperfecta, Type V osteogenesis imperfecta, Type VI osteogenesis imperfecta, Type VII osteogenesis imperfecta, Type VIII osteogenesis imperfecta, Type IX osteogenesis imperfecta, Type X osteogenesis imperfecta, or Type XI osteogenesis imperfecta.

In some embodiments of the above methods of the invention, the disease is metastatic bone cancer. In some embodiments, the patient is suffering from breast cancer or prostate cancer.

In some embodiments of the above methods of the invention, the disease is selected from the group consisting of osteoporosis, fibrous dysplasia, Calmurati-Engleman Disease, Marfan's Syndrome, osteoglophonic dysplasia, autosomal dominant osteopetrosis, osteoporosis, osteoporosis-pseudoglioma syndrome, juvenile, gerodermia osteodysplastica, Duchenne muscular dystrophy, osteosarcoma, osteogenesis imperfecta congenita, microcephaly, and cataracts.

In some embodiment of the above methods of the invention, the disease is selected from the group consisting of pseudohypoparathyroidism, Cleidocranial Dysplasia, Dyskeratosis Congenita, Exudative Vitreoretinopathy 1, Schimmelpenning-Feuerstein-Mims Syndrome, Prader-Willi Syndrome, Achondrogenesis, Antley-Bixler Syndrome, Aspartylglucosaminuria, Celiac Disease, Cerebrooculofacioskeletal Syndrome 1, Lysinuric Protein Intolerance, neuropathy, dyskeratosis congenita, Ehlers-Danlos Syndrome, epiphyseal dysplasia, hyaline fibromatosis syndrome, Perrault Syndrome 1, hemochromatosis, homocystinuria (e.g., due to cystathionine beta-synthase deficiency), hypophosphatemic rickets with hypercalciuria, desbuquois dysplasia, multiple pterygium syndrome, lethal congenital contracture syndrome 1, mitochondrial DNA depletion Ssndrome 6 (hepatocerebral Type), Niemann-Pick Disease, osteopetrosis, porphyria, Rothmund-Thomson Syndrome, Wilson Disease, Dent Disease 1, occipital horn syndrome, hyperglycerolemia, hypophosphatemic rickets, Lowe Oculocerebrorenal Syndrome, renal tubulopathy, diabetes mellitus, cerebellar ataxia, vitamin D hydroxylation-deficient rickets, Warburg micro syndrome 1, Stuve-Wiedemann Syndrome, Blue Rubber Bleb Nevus syndrome, Singleton-Merten Syndrome, microcephalic osteodysplastic primordial dwarfism, dysosteosclerosis, Hallermann-Streiff Syndrome, Bruck Syndrome 1, multiple pterygium syndrome (e.g., X-Linked), spondylometaphyseal dysplasia with dentinogenesis imperfecta, Hall-Riggs Mental Retardation Syndrome, infantile multisystem neurologic disease with osseous fragility, acrocephalopolysyndactyly Type III, acroosteolysis, ACTH-independent macronodular adrenal hyperplasia, amino aciduria with mental deficiency, arthropathy, bone fragility (e.g., with craniosynostosis, ocular proptosis, hydrocephalus, and distinctive facial features), brittle cornea syndrome, cerebrotendinous xanthomatosis, Cri-Du-Chat Syndrome, dysplasia epiphysealis hemimelica, autosomal dominant Ehlers-Danlos Syndrome, familial osteodysplasia, Flynn-Aird Syndrome, gerodermia osteodysplastica, Duchenne muscular dystrophy, osteosarcoma, glycogen storage disease Ia, Hutchinson-Gilford Progeria Syndrome, Infantile Systemic Hyalinosis, hypertrichotic osteochondrodysplasia, hyperzincemia with functional zinc depletion, hypophosphatasia, autosomal dominant hypophosphatemic rickets, X-linked recessive hypophosphatemic rickets, Lichtenstein Syndrome, macroepiphyseal dysplasia (e.g., with osteoporosis wrinkled skin, and aged appearance), Menkes Disease, Mental Retardation (e.g., X-Linked, Snyder-Robinson type), Jansen type metaphyseal chondrodysplasia, microspherophakia-metaphyseal dysplasia, morquio syndrome a, Morquio Syndrome B, ossified ear cartilages (e.g., with mental deficiency, muscle wasting, and osteocraniostenosis), osteoporosis and oculocutaneous hypopigmentation syndrome, osteoporosis-pseudoglioma syndrome, juvenile osteoporosis, osteosclerosis with ichthyosis and fractures, ovarian dysgenesis 1, ovarian dysgenesis 2, ovarian dysgenesis 3, ovarian dysgenesis 4, pituitary adenoma, polycystic lipomembranous osteodysplasia with sclerosing leukoencephalopathy, Prader-Willi Habitus, osteopenia, Okamoto type premature aging syndrome, Prieto X-linked mental retardation syndrome, pycnodysostosis, Pyle Disease, Reifenstein Syndrome, autosomal dominant distal renal tubular acidosis, Type 1 Schwartz-Jampel Syndrome, Type 2 Schwartz-Jampel Syndrome, Type 3 Schwartz-Jampel Syndrome, Type 4 Schwartz-Jampel Syndrome, X-linked spondyloepiphyseal dysplasia tarda, and Torg-Winchester Syndrome.

In another aspect, the invention features a method of improving muscle function in a human patient suffering from a disease associated with a pathological increase in TGF-β activity in a human patient by administering to the human patient a pharmaceutical formulation of any of the aspects or embodiments of the invention described herein. In some embodiments of the above methods of the invention, the disease associated with a pathological increase in TGF-β activity is fibrosis, liver fibrosis, non-alcoholic steatohepatitis, a pathological skin fibrotic condition, a wound, delayed wound healing, scarring, hypertrophic scarring, keloid scarring, an internal wound, an internal wound caused by a surgical procedure, a burn, epidermal burn, superficial dermal burn, mid-dermal burn, deep dermal burn, a full thickness burn, a pulmonary disease, asthma, chronic obstructive pulmonary disease, and fibroproliferative lung disease, a renal disease, or diabetic nephropathy.

In some embodiments of the above methods of the invention, the disease is an autoimmune disease, such as psoriasis or scleroderma.

In some embodiments of the above methods of the invention, the disease is cancer. In some embodiments of the above methods of the invention, the cancer is carcinoma, pancreatic cancer, glioblastoma, myeloid leukemia, head and neck cancer, melanoma, breast cancer, or colorectal cancer. In some embodiments, the carcinoma is selected from the group consisting of squamous cell carcinoma, epidermoid carcinoma, urothelial carcinoma, adenocarcinoma, adrenocortical carcinoma, basal cell carcinoma, ductal carcinoma in situ (DCIS), invasive ductal carcinoma, Merkel cell carcinoma, midline tract carcinoma, thymic carcinoma, and renal cell carcinoma. In some embodiments of the above methods of the invention, the carcinoma is squamous cell carcinoma. In other embodiments, the squamous cell carcinoma is vulvar squamous cell carcinoma, epidermal squamous cell carcinoma, oral squamous cell carcinoma, pulmonary squamous cell carcinoma, or head and neck squamous cell carcinoma.

In some aspects, the method of administering to the patient a therapeutically effective amount of a TGF-β antagonist, such as a TGF-β-binding antibody or an antigen-binding fragment thereof, of any of the above aspects or embodiments of the invention results in the patient exhibiting an increase in muscle mass, muscle strength, and/or muscle quality.

In some aspects, the method of administering to the patient a therapeutically effective amount of a TGF-β antagonist, such as a TGF-β-binding antibody or an antigen-binding fragment thereof that is conjugated to a targeting moiety that binds a protein or mineral present in human bone tissue, results in the patient exhibiting an increase in muscle mass, muscle strength, and/or muscle quality. In another aspect, the method of administering to the patient a therapeutically effective amount of a TGF-β antagonist, such as a TGF-β-binding antibody or an antigen-binding fragment thereof that is not conjugated to a targeting moiety that binds a protein or mineral present in human bone tissue, results in the patient exhibiting an increase in muscle mass, muscle strength, and/or muscle quality.

In some embodiments of the invention discussed above, the TGF-β antagonist is an antibody or an antigen-binding fragment thereof that binds TGF-β, such as an isoform of TGF-β (e.g., TGFβ1, TGF-β2, and/or TGF-β3). In some embodiments, the antibody or antigen-binding fragment thereof contains one or more, or all, of the following complementarity determining regions (CDRs):
(a) a CDR-H1 having the amino acid sequence SNVIS (SEQ ID NO: 64);
(b) a CDR-H2 having the amino acid sequence GVIPIVDIANYAQRFKG (SEQ ID NO: 65);
(c) a CDR-H3 having the amino acid sequence TLGLVLDAMDY (SEQ ID NO: 66);
(d) a CDR-L1 having the amino acid sequence RASQSLGSSYLA (SEQ ID NO: 67);
(e) a CDR-L2 having the amino acid sequence GASSRAP (SEQ ID NO: 68); and
(f) a CDR-L3 having the amino acid sequence QQYADSPIT (SEQ ID NO: 69).

In some embodiments of the invention discussed above, the antibody or antigen-binding fragment thereof contains one or more CDRs that have at least 50% sequence identity (e.g., at least 50%, 55%, 60%, 65%, 70%, 75%, 80%, 85%, 90%, 95%, 97%, 98%, 99%, or more, sequence identity) to the corresponding CDRs of SEQ ID NOs: 64-69. In some embodiments, the antibody or antigen-binding fragment thereof contains a set of six CDRs that each have at least 50% sequence identity (e.g., at least 50%, 55%, 60%, 65%, 70%, 75%, 80%, 85%, 90%, 95%, 97%, 98%, 99%, or more, sequence identity) to the foregoing CDRs.

In some embodiments of the invention discussed above, the antibody contains a heavy chain variable region having the amino acid sequence of QVQLVQSGAEVKKPGSSVKVSCKASGYTFSSNVISWVRQAPGQGLEWMGGVIPIVDIANYAQRFKGRVTITADESTSTTYMELSSLRSEDTAVYYCASTLGLVLDAMDYWGQGTLVTVSS (SEQ ID NO: 70), or a heavy chain variable region having an amino acid sequence that has at least 85% sequence identity (e.g., at least 85%, 90%, 95%, 97%, 98%, 99%, or more, sequence identity) to SEQ ID NO: 70. In some embodiments of the invention discussed above, the antibody or antigen-binding fragment thereof has a light chain variable region having the amino acid sequence of ETVLTQSPGTLSLSPGERATLSCRASQSLGSSYLAWYQQKPGQAPRLLIYGASSRAPGIPDRFSGSGSGTDFTLTISRLEPEDFAVYYCQQYADSPITFGQGTRLEIK (SEQ ID NO: 71), or a light chain variable region having an amino acid sequence that has at least 85% sequence identity (e.g., at least 85%, 90%, 95%, 97%, 98%, 99%, or more, sequence identity) to SEQ ID NO: 71. Antibodies containing the foregoing CDRs, as well as the above heavy chain variable region and light chain variable regions, are described, e.g., in U.S. Pat. No. 9,598,486, the disclosure of which is incorporated herein by reference in its entirety.

In some embodiments of the invention discussed above, the antibody or antigen-binding fragment thereof is a humanized antibody or antigen-binding fragment thereof, such as a humanized antibody or antigen-binding fragment thereof of the 1D11 antibody (PCT-001), described herein. In some embodiments, the humanized antibody or antigen-binding fragment thereof of the 1D11 antibody is Genzyme's monoclonal antibody GC1008 (Fresolimumab).

In some embodiment of the invention discussed above s, the humanized antibody or antigen-binding fragment thereof further includes the D10 bone-targeting moiety (10 aspartate repeat). In some embodiments, the humanized antibody or antigen-binding fragment thereof including the D10 bone-targeting moiety is PCT-0011, which is the humanized monoclonal antibody GC1008 (the humanized version of the mouse monoclonal antibody 1D11) with the D10 bone-targeting moiety.

In some embodiments of the invention discussed above, the bone-targeting antibody PCT-0011 contains a heavy chain, which includes the D10 bone-targeting moiety, having the amino acid sequence of SEQ ID NO: 62, or a heavy chain having an amino acid sequence that has at least 85% sequence identity (e.g., at least 85%, 90%, 95%, 97%, 98%, 99%, or more, sequence identity) to SEQ ID NO: 62. In some embodiments, the antibody or antigen-binding fragment thereof has a light chain having the amino acid sequence of SEQ ID NO: 63, or a light chain having an amino acid sequence that has at least 85% sequence identity (e.g., at least 85%, 90%, 95%, 97%, 98%, 99%, or more, sequence identity) to SEQ ID NO: 63.

In some embodiments of the invention discussed above, the humanized antibody or antigen-binding fragment thereof is Eli Lilly's monoclonal antibody TβM1 (LY2382770). The TβM1 (LY2382770) antibody sequences are described in detail in, e.g., WO 2005/010049, the disclosure of which is incorporated herein by reference in its entirety.

In some embodiments of the invention discussed above, the antibody or antigen-binding fragment thereof is an optimized antibody or antigen-binding fragment thereof, such as an optimized variant of the 1D11, GC1008, PCT-0011, and/or TβM1 (LY2382770) antibodies, described herein.

In some embodiments of the invention discussed above, the optimized antibody or antigen-binding fragment thereof is an affinity-matured antibody or antigen-binding fragment thereof, such as an affinity-matured variant of the 1D11, GC1008, PCT-0011, and/or TβM1 (LY2382770) antibodies, described herein.

In some embodiments of the invention discussed above, the antibody or antigen-binding fragment thereof, described herein, is a monoclonal antibody or antigen-binding fragment thereof, a polyclonal antibody or antigen-binding fragment thereof, a humanized antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)₂ molecule, or a tandem di-scFV.

In some embodiments, the antibody is a single-chain molecule, such as a scFv, a diabody, or a triabody, among others described herein.

In some embodiments, the antibody is a scFv.

In some embodiments of the invention discussed above, the antibody or antigen-binding fragment thereof has an isotype selected from the group consisting of IgG, IgA, IgM, IgD, and IgE.

In some embodiments of the invention discussed above, the antibody or antigen-binding fragment thereof is conjugated to a targeting moiety that is an agent that binds a protein or mineral present in human bone tissue.

In some embodiments of the invention discussed above, the targeting moiety is an agent that binds a protein present in human bone tissue. In some embodiments, the protein present in human bone tissue is collagen.

In some embodiments of the invention discussed above, the targeting moiety is an agent capable of binding a mineral present in human bone tissue. In some embodiments, the mineral present in human bone tissue is hydroxyapatite.

In some embodiments, the targeting moiety is a polyanionic peptide such as a polyanionic peptide that includes one or more amino acids bearing a side-chain substituent selected from the group consisting of carboxylate, sulfonate, phosphonate, and phosphate.

In some embodiments, the polyanionic peptide includes 1 to 25 glutamate residues. In some embodiments, the polyanionic peptide comprises 10 glutamate residues.

In some embodiments, the polyanionic peptide includes 1 to 25 aspartate residues. In some embodiments, the polyanionic peptide comprises 10 aspartate residues.

In some embodiments, the glutamate or aspartate residues are consecutive. In other embodiments, the glutamate or aspartate residues are discontinuous.

In some embodiments, the polyanionic peptide has the amino acid sequence of SEQ ID NO: 46.

In some embodiments of the above methods of the invention, the antibody or antigen-binding fragment thereof includes a heavy chain having the amino acid sequence of SEQ ID NO: 62, or an amino acid sequence that is at least 85% identical thereto.

In some embodiments of the above methods of the invention, the antibody or antigen-binding fragment thereof includes a light chain having the amino acid sequence of SEQ ID NO: 63, or an amino acid sequence that is at least 85% identical thereto.

In some embodiments of the above methods of the invention, the antibody or antigen-binding fragment thereof includes a heavy chain having the amino acid sequence of SEQ ID NO: 62, or an amino acid sequence that is at least 85% identical thereto, and a light chain having the amino acid sequence of SEQ ID NO: 63, or an amino acid sequence that is at least 85% identical thereto.

In some embodiments, the TGF-β antagonist is a peptide. For instance, the peptide may be derived from (e.g., a domain, fragment, or variant of) a TGF-β co-receptor, e.g., CD109. In some embodiments, the peptide is a fragment of CD109 that contains the amino acid sequence IDGVYDNAEYAERFMEENEGHIVDIHDFSLGSS (SEQ ID NO: 76), or a sequence having at least 85% sequence identity (e.g., at least 85%, 90%, 95%, 97%, 99%, or greater) thereto and/or having one or more conservative amino acid substitutions with respect to this sequence. In some embodiments, the peptide is a fragment of CD109 that contains the amino acid sequence WIWLDTNMGYRIYQEFEVT (SEQ ID NO: 72), or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains a fragment having the amino acid sequence of one or more portions of SEQ ID NO: 73, which corresponds to the amino acid sequence of an active form of CD109 that contains a tyrosine residue at amino acid position 703. For instance, the peptide may contain the amino acid sequence of residues 21-1404 of SEQ ID NO: 73, or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of residues 21-1428 of SEQ ID NO: 73, or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of SEQ ID NO: 73, or a sequence having at least 85% sequence identity (e.g., at least 85%, 90%, 95%, 97%, 99%, or greater) thereto and/or having one or more conservative amino acid substitutions with respect to this sequence.

In some embodiments, the peptide contains the amino acid sequence WIWLDTNMGSRIYQEFEVT (SEQ ID NO: 74), or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains a fragment having the amino acid sequence of one or more portions of SEQ ID NO: 75, which corresponds to the amino acid sequence of an active form of CD109 that contains a serine residue at amino acid position 703. In some embodiments, the peptide contains the amino acid sequence of residues 21-1404 of SEQ ID NO: 75, or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of residues 21-1428 of SEQ ID NO: 75, or a sequence having at least 85% sequence identity (e.g., at least 85%, 90%, 95%, 97%, 99%, or greater) thereto and/or having one or more conservative amino acid substitutions with respect to this sequence. In some embodiments, the peptide contains the amino acid sequence of SEQ ID NO: 75, or a sequence having at least 85% sequence identity (e.g., at least 85%, 90%, 95%, 97%, 99%, or greater) thereto and/or having one or more conservative amino acid substitutions with respect to this sequence.

In some embodiments, the peptide is a fragment of CD109 that contains the amino acid sequence of SEQ ID NO: 77, or a sequence having at least 85% sequence identity (e.g., at least 85%, 90%, 95%, 97%, 99%, or greater) thereto and/or having one or more conservative amino acid substitutions with respect to this sequence.

In some embodiments, the peptide is a fragment of CD109 that contains the amino acid sequence of RKHFPETWIWLDTNMGYRIYQEFEV (SEQ ID NO: 78), or a sequence having at least 50% sequence identity (e.g., at least 50%, 55%, 60%, 65%, 70%, 75%, 80%, 85%, 90%, 95%, 97%, 99%, or greater) thereto and/or having one or more conservative amino acid substitutions with respect to this sequence.

In some embodiments, the peptide contains an amino acid sequence selected from the group consisting of ANFCLGPCPYIWSLDT (SEQ ID NO: 79), ANFCSGPCPYLRSADT (SEQ ID NO: 80), PYIWSLDTQY (SEQ ID NO: 81), PYLWSSDTQH (SEQ ID NO: 82), PYLRSADTTH (SEQ ID NO: 83), WSXD (SEQ ID NO: 84), and RSXD (SEQ ID NO: 85), wherein X represents any naturally occurring amino acid. In some embodiments, the peptide contains an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences.

In some embodiments, the peptide contains an amino acid sequence selected from the group consisting of TSLDATMIWTMM (SEQ ID NO: 86), SNPYSAFQVDIIVDI (SEQ ID NO: 87), TSLMIWTMM (SEQ ID NO: 88), TSLDASIIWAMMQN (SEQ ID NO: 89), SNPYSAFQVDITID (SEQ ID NO: 90), EAVLILQGPPYVSWL (SEQ ID NO: 91), and LDSLSFQLGLYLSPH (SEQ ID NO: 92). In some embodiments, the peptide contains an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences.

In some embodiments, the peptide contains an amino acid sequence selected from the group consisting of TSLDASIIWAMMQN (SEQ ID NO: 96), KRIWFIPRSSWYERA (SEQ ID NO: 97), KRIWFIPRSSW (SEQ ID NO: 98), KRIWFIPRSSW (SEQ ID NO: 99), and KRIWFIPRSSW (SEQ ID NO: 100). In some embodiments, the peptide contains an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences.

In some embodiments, the peptide contains the amino acid sequence of any one of SEQ ID NOs: 101-123. In some embodiments, the peptide contains an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences.

In some embodiments, the peptide contains the amino acid sequence of glycoprotein-A repetitions predominant protein (GARP) (SEQ ID NO: 124). In some embodiments, the peptide contains an amino acid sequence having at least 85% sequence identity (e.g., at least 85%, 90%, 95%, 97%, 99%, or greater) to this sequences and/or having one or more conservative amino acid substitutions with respect to this sequence.

In some embodiments, the peptide contains an amino acid sequence selected from the group consisting of HANFCLGPCPYIWSL (SEQ ID NO: 93), FCLGPCPYIWSLDT (SEQ ID NO: 94), and HEPKGYHANFCLGPCP (SEQ ID NO: 95). In some embodiments, the peptide contains an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences.

In some embodiments, the TGF-β antagonist is conjugated to a targeting moiety that localizes to bone tissue. The targeting moiety may be, for instance, an agent that binds a protein (e.g., collagen) or mineral (e.g., hydroxyapatite) in bone tissue.

In some embodiments, the targeting moiety contains a peptide, such as a peptide that binds a protein present in human bone tissue. In some embodiments, the targeting moiety is a peptide, such as a peptide that binds a protein present in human bone tissue. In some embodiments, the protein present in human bone tissue is collagen. For instance, the peptide that binds the protein may contain the amino acid sequence of any one of SEQ ID NOs: 125-127. In some embodiments, the peptide that binds the protein contains the amino acid sequence of any one of SEQ ID NOs: 128-130. In some embodiments, the peptide that binds the protein contains the amino acid sequence of SEQ ID NO: 127. In some embodiments, the peptide that binds the protein contains an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences.

In some embodiments, the targeting moiety contains a peptide capable of binding a mineral present in human bone tissue, such as hydroxyapatite. In some embodiments, the peptide that binds the mineral contains the amino acid sequence of any one of SEQ ID NOs: 131-397. In some embodiments, the peptide that binds the mineral contains an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences.

In some embodiments, the targeting moiety, which may be capable of binding hydroxyapatite, is a polyanionic peptide. The polyanionicpeptide may contain, for instance, one or more amino acids bearing a side-chain substituent selected from the group consisting of carboxylate, sulfonate, phosphonate, and phosphate.

In some embodiments, the polyanionic peptide contains (e.g., consists of) one or more glutamate residues (e.g., 1-25 glutamate residues, or more, such as 1, 2, 3, 4, 5, 6, 7, 8, 9, 10, 11, 12, 13, 14, 15, 16, 17, 18, 19, 20, 21, 22, 23, 24, or 25, or more, glutamate residues). In some embodiments, the polyanionic peptide contains (e.g., consists of) from 3 to 20 glutamate residues (e.g., 3, 4, 5, 6, 7, 8, 9, 10, 11, 12, 13, 14, 15, 16, 17, 18, 19, or 20 glutamate residues). In some embodiments, the polyanionic peptide contains (e.g., consists of) from 5 to 15 glutamate residues (e.g., 5, 6, 7, 8, 9, 10, 11, 12, 13, 14, or 15 glutamate residues). In some embodiments, the polyanionic peptide contains (e.g., consists of) from 8 to 12 glutamate residues (e.g., 8, 9, 10, 11, or 12 glutamate residues). In some embodiments, the polyanionic peptide contains (e.g., consists of) 5 glutamate residues. In some embodiments, the polyanionic peptide contains (e.g., consists of) 6 glutamate residues. In some embodiments, the polyanionic peptide contains (e.g., consists of) 7 glutamate residues. In some embodiments, the polyanionic peptide contains (e.g., consists of) 8 glutamate residues. In some embodiments, the polyanionic peptide contains (e.g., consists of) 9 glutamate residues. In some embodiments, the polyanionic peptide contains (e.g., consists of) 10 glutamate residues. In some embodiments, the polyanionic peptide contains (e.g., consists of) 11 glutamate residues. In some embodiments, the polyanionic peptide contains (e.g., consists of) 12 glutamate residues. In some embodiments, the polyanionic peptide contains (e.g., consists of) 13 glutamate residues. In some embodiments, the polyanionic peptide contains (e.g., consists of) 14 glutamate residues. In some embodiments, the polyanionic peptide contains (e.g., consists of) 15 glutamate residues.

In some embodiments, the polyanionic peptide is a peptide of the formula Eₙ, wherein E designates a glutamate residue and n is an integer from 1 to 25. For instance, the polyanionic peptide may be of the formula E₁, E₂, E₃, E₄, E₅, E₆, E₇, E₈, E₉, E₁₀, E₁₁, E₁₂, E₁₃, E₁₄, E₁₅, E₁₆, E₁₇, E₁₈, E₁₉, E₂₀, E₂₁, E₂₂, E₂₃, E₂₄, or E₂₅. In some embodiments, the peptide is a peptide of the formula XₙEₘXₒEₚ, wherein E designates a glutamate residue, each X independently designates any naturally-occurring amino acid, m represents an integer from 1 to 25, and n and o each independently represent integers from 0 to 5, and p represents an integer from 1 to 10.

For instance, in some embodiments, the polyanionic peptide is a peptide of the formula E₂. In some embodiments, the polyanionic peptide is a peptide of the formula E₃. In some embodiments, the polyanionic peptide is a peptide of the formula E₄. In some embodiments, the polyanionic peptide is a peptide of the formula E₅. In some embodiments, the polyanionic peptide is a peptide of the formula E₆. In some embodiments, the polyanionic peptide is a peptide of the formula E₇. In some embodiments, the polyanionic peptide is a peptide of the formula E₈. In some embodiments, the polyanionic peptide is a peptide of the formula E₉. In some embodiments, the polyanionic peptide is a peptide of the formula E₁₀. In some embodiments, the polyanionic peptide is a peptide of the formula E₁₁. In some embodiments, the polyanionic peptide is a peptide of the formula E₁₂. In some embodiments, the polyanionic peptide is a peptide of the formula E₁₃. In some embodiments, the polyanionic peptide is a peptide of the formula E₁₄. In some embodiments, the polyanionic peptide is a peptide of the formula E₁₅. In some embodiments, the polyanionic peptide is a peptide of the formula E₁₆. In some embodiments, the polyanionic peptide is a peptide of the formula E₁₇. In some embodiments, the polyanionic peptide is a peptide of the formula E₁₈. In some embodiments, the polyanionic peptide is a peptide of the formula E₁₉. In some embodiments, the polyanionic peptide is a peptide of the formula E₂₀. In some embodiments, the polyanionic peptide is a peptide of the formula E₂₁. In some embodiments, the polyanionic peptide is a peptide of the formula E₂₂. In some embodiments, the polyanionic peptide is a peptide of the formula E₂₃. In some embodiments, the polyanionic peptide is a peptide of the formula E₂₄. In some embodiments, the polyanionic peptide is a peptide of the formula E₂₅.

In some embodiments, the polyanionic peptide is a peptide of the formula E₁₀.

In some embodiments, the glutamate residues are consecutive. In some embodiments, the glutamate residues are discontinuous.

In some embodiments, the polyanionic peptide contains (e.g., consists of) one or more aspartate residues (e.g., 1-25 aspartate residues, or more, such as 1, 2, 3, 4, 5, 6, 7, 8, 9, 10, 11, 12, 13, 14, 15, 16, 17, 18, 19, 20, 21, 22, 23, 24, or 25, or more, aspartate residues). In some embodiments, the polyanionic peptide contains (e.g., consists of) from 3 to 20 aspartate residues (e.g., 3, 4, 5, 6, 7, 8, 9, 10, 11, 12, 13, 14, 15, 16, 17, 18, 19, or 20 aspartate residues). In some embodiments, the polyanionic peptide contains (e.g., consists of) from 5 to 15 aspartate residues (e.g., 5, 6, 7, 8, 9, 10, 11, 12, 13, 14, or 15 aspartate residues). In some embodiments, the polyanionic peptide contains (e.g., consists of) from 8 to 12 aspartate residues (e.g., 8, 9, 10, 11, or 12 aspartate residues). In some embodiments, the polyanionic peptide contains (e.g., consists of) 5 aspartate residues. In some embodiments, the polyanionic peptide contains (e.g., consists of) 6 aspartate residues. In some embodiments, the polyanionic peptide contains (e.g., consists of) 7 aspartate residues. In some embodiments, the polyanionic peptide contains (e.g., consists of) 8 aspartate residues. In some embodiments, the polyanionic peptide contains (e.g., consists of) 9 aspartate residues. In some embodiments, the polyanionic peptide contains (e.g., consists of) 10 aspartate residues. In some embodiments, the polyanionic peptide contains (e.g., consists of) 11 aspartate residues. In some embodiments, the polyanionic peptide contains (e.g., consists of) 12 aspartate residues. In some embodiments, the polyanionic peptide contains (e.g., consists of) 13 aspartate residues. In some embodiments, the polyanionic peptide contains (e.g., consists of) 14 aspartate residues. In some embodiments, the polyanionic peptide contains (e.g., consists of) 15 aspartate residues.

In some embodiments, the polyanionic peptide is a peptide of the formula Dₙ, wherein D designates an aspartate residue and n is an integer from 1 to 25. For instance, the polyanionic peptide may be of the formula D₁, D₂, D₃, D₄, D₅, D₆, D₇, D₈, D₉, D₁₀, D₁₁, D₁₂, D₁₃, D₁₄, D₁₅, D₁₆, D₁₇, D₁₈, D₁₉, D₂₀, D₂₁, D₂₂, D₂₃, D₂₄, or D₂₅. In some embodiments, the peptide is a peptide of the formula wherein D designates an aspartate residue, each X independently designates any naturally-occurring amino acid, m represents an integer from 1 to 25, and n and o each independently represent integers from 0 to 5, and p represents an integer from 1 to 10.

For instance, in some embodiments, the polyanionic peptide is a peptide of the formula D₂. In some embodiments, the polyanionic peptide is a peptide of the formula D₃. In some embodiments, the polyanionic peptide is a peptide of the formula D₄. In some embodiments, the polyanionic peptide is a peptide of the formula D₅. In some embodiments, the polyanionic peptide is a peptide of the formula D₆. In some embodiments, the polyanionic peptide is a peptide of the formula D₇. In some embodiments, the polyanionic peptide is a peptide of the formula D₈. In some embodiments, the polyanionic peptide is a peptide of the formula D₉. In some embodiments, the polyanionic peptide is a peptide of the formula D₁₀. In some embodiments, the polyanionic peptide is a peptide of the formula D₁₁. In some embodiments, the polyanionic peptide is a peptide of the formula D₁₂. In some embodiments, the polyanionic peptide is a peptide of the formula D₁₃. In some embodiments, the polyanionic peptide is a peptide of the formula D₁₄. In some embodiments, the polyanionic peptide is a peptide of the formula D₁₅. In some embodiments, the polyanionic peptide is a peptide of the formula D₁₆. In some embodiments, the polyanionic peptide is a peptide of the formula D₁₇. In some embodiments, the polyanionic peptide is a peptide of the formula D₁₈. In some embodiments, the polyanionic peptide is a peptide of the formula D₁₉. In some embodiments, the polyanionic peptide is a peptide of the formula D₂₀. In some embodiments, the polyanionic peptide is a peptide of the formula D₂₁. In some embodiments, the polyanionic peptide is a peptide of the formula D₂₂. In some embodiments, the polyanionic peptide is a peptide of the formula D₂₃. In some embodiments, the polyanionic peptide is a peptide of the formula D₂₄. In some embodiments, the polyanionic peptide is a peptide of the formula D₂₅.

In some embodiments, the polyanionic peptide is a peptide of the formula D₁₀.

In some embodiments, the aspartate residues are consecutive. In some embodiments, the aspartate residues are discontinuous.

In some embodiments, the ratio of amino acids bearing a side-chain that is negatively-charged at physiological pH to the total quantity of amino acids in the polyanionic peptide is from about 0.5 to about 2.0.

In some embodiments, the targeting moiety is a bisphosphonate. The bisphosphonate may be, for instance, etidronate, clodronate, tiludronate, pamidronate, neridronate, olpadronate, alendronate, ibandronate, risedronate, or zoledronate, or a pharmaceutically acceptable salt thereof.

In some embodiments, the TGF-β antagonist is bound to the targeting moiety directly, e.g., by a covalent bond, such as an amide bond, disulfide bridge, thioether bond, or carbon-carbon bond, among others. In some embodiments, the TGF-β antagonist is bound to the targeting moiety by way of a linker, such as a peptidic linker or a synthetic linker described herein.

In some embodiments, the TGF-β antagonist is bound to the N-terminus of a peptidic targeting moiety. For instance, in some embodiments, the C-terminus of a peptidic TGF-β antagonist is bound to the N-terminus of a peptidic moiety. In some embodiments, the TGF-β antagonist is bound to the C-terminus of the targeting moiety. For instance, in some embodiments, the N-terminus of a peptidic TGF-β antagonist is bound to the C-terminus of a peptidic moiety.

In some embodiments, the TGF-β antagonist is bound to the targeting moiety by way of an immunoglobulin Fc domain.

In some embodiments, the TGF-β antagonist is bound to the N-terminus of the immunoglobulin Fc domain and the targeting moiety is bound to the C-terminus of the immunoglobulin Fc domain. In some embodiments, the TGF-β antagonist is bound to the C-terminus of the immunoglobulin Fc domain and the targeting moiety is bound to the N-terminus of the immunoglobulin Fc domain. In some embodiments, the immunoglobulin is selected from the group consisting of human IgG, human IgA, human IgM, human IgE, and human IgD, or is a modified immunoglobulin derived therefrom. In some embodiments, the IgG immunoglobulin domain is selected from IgG1, IgG2, IgG3, or IgG4 domains, or is a modified IgG domain as described in U.S. Pat. No. 5,925,734. In some embodiments, the immunoglobulin domain exhibits effector functions, particularly effector functions selected from ADCC and/or CDC. In some embodiments, however, modified immunoglobulin domains having modified, e.g. at least partially deleted, effector functions, may be used.

In some embodiments, the TGF-β antagonist, such as a TGF-β receptor fusion protein, is bound to a signal peptide that directs excretion of the TGF-β antagonist from a mammalian cell. Specific signal peptides, such as those described herein, can improve manufacturing of the TGF-β antagonists of the invention, or can be useful for administration of the TGF-β antagonists via nucleic acids encoding the TGF-β antagonists of the invention. Cleavage or other removal of the signal peptide from the TGF-β antagonist results in the mature form of the TGF-β antagonists of the invention. In some embodiments, the signal peptide is bound to a side-chain present within the N-terminal region of the TGF-β antagonist. In some embodiments, the side-chain present within the N-terminal region of the TGF-β antagonist is located within the first 25 amino acid residues of the TGF-β antagonist (e.g., within the first 2, 3, 4, 5, 6, 7, 8, 9, 10, 11, 12, 13, 14, 15, 16, 17, 18, 19, 20, 21, 22, 23, 24, or 25 amino acid residues of the TGF-β antagonist). In some embodiments, the side-chain present within the N-terminal region of the TGF-β antagonist is located within the first 10 amino acid residues of the TGF-β antagonist. In some embodiments, the side-chain present within the N-terminal region of the TGF-β antagonist is present within a naturally-occurring amino acid residue. In some embodiments, the side-chain present within the N-terminal region of the TGF-β antagonist is present within a non-natural amino acid residue.

In some embodiments, the naturally-occurring amino acid residue is selected from the group consisting of lysine, aspartic acid, glutamic acid, asparagine, glutamine, serine, threonine, and cysteine. In some embodiments, the non-natural amino acid residue contains a reactive substituent selected from the group consisting of amino, carboxy, acetyl, hydrazino, hydrazido, hydroxy, semicarbazido, mercapto, sulfanyl, azido, alkenyl, and alkynyl.

In some embodiments, the signal peptide is an albumin signal peptide MKWVTFLLLLFISGSAFSAAA (SEQ ID NO: 4). In other embodiments, the signal peptide is an alpha-lactalbumin peptide MMSFVSLLLVGILFHATQ (SEQ ID NO: 42).

In some embodiments, the signal peptide is an albumin signal peptide. In some embodiments, the albumin signal peptide has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity) to SEQ ID NO: 5. In some embodiments, the albumin signal peptide has an amino acid sequence having at least 90% sequence identity to SEQ ID NO: 5. In some embodiments, the albumin signal peptide has an amino acid sequence having at least 95% sequence identity to SEQ ID NO: 5. In some embodiments, the albumin signal peptide has an amino acid sequence that differs from the sequence of SEQ ID NO: 5 by one or more conservative substitutions (e.g., by 1, 2, 3, 4, 5, or more, conservative substitutions). In some embodiments, the albumin signal peptide has an amino acid sequence that differs from the sequence of SEQ ID NO: 5 by fewer than 5 non-conservative substitutions (e.g., by 5, 4, 3, 2, 1, or 0 non-conservative substitutions). In some embodiments, the albumin signal peptide has an amino acid sequence that differs from the sequence of SEQ ID NO: 5 only by one or more conservative substitutions (e.g., only by 1, 2, 3, 4, 5, more, conservative substitutions).

In some embodiments, the TGF-β antagonist is bound to the targeting moiety by way of a linker.

In some embodiments, the linker contains an immunoglobulin Fc domain. For instance, in some embodiments, the linker is an immunoglobulin Fc domain. In some embodiments, the TGF-β antagonist is bound to the N-terminus of the immunoglobulin Fc domain and the targeting moiety is bound to the C-terminus of the immunoglobulin Fc domain. In some embodiments, the TGF-β antagonist is bound to the C-terminus of the immunoglobulin Fc domain and the targeting moiety is bound to the N-terminus of the immunoglobulin Fc domain. In some embodiments, the immunoglobulin is selected from the group consisting of human IgG, human IgA, human IgM, human IgE, and human IgD, or is a modified immunoglobulin derived therefrom. In some embodiments, the IgG immunoglobulin domain is selected from IgG1, IgG2, IgG3, or IgG4 domains, or is a modified IgG domain as described in U.S. Pat. No. 5,925,734. In some embodiments, the immunoglobulin domain exhibits effector functions, particularly effector functions selected from ADCC and/or CDC. In some embodiments, however, modified immunoglobulin domains having modified, e.g. at least partially deleted, effector functions, may be used.

In some embodiments, the linker contains a coupling moiety set forth in Table 14 herein. In some embodiments, the linker contains a polypeptide, e.g., having only natural or non-natural amino acids covalently joined to one another by amide bonds. In some embodiments, the polypeptide contains one or more residues selected from the group consisting of glycine, serine, and threonine. In some embodiments, polypeptide linker include one or more glycines, e.g., 1, 2, 3, 4, 5, 6, 7, 8, 9, 10, 11, 12, 15, 18, or more glycines. In some embodiment, the linker contain the formula (GGG)n, where n=1, 2, 3, 4, 5, 6, 7, etc., such as GGG (SEQ ID NO: 6). In some embodiments, the polypeptide contains a repeating amino acid sequence of the formula (GGGS)n, where n=1, 2, 3, 4, 5, etc. (SEQ ID NO: 60) or the sequence of (GGGGS)n, where n=1, 2, 3, 4, 5, etc. (SEQ ID NO: 61). In some embodiments, the polypeptide has the amino acid sequence GGGGS (SEQ ID NO: 7). In some embodiments, the polypeptide has the sequence GGGGSGGGGSGGGGSG (SEQ ID NO: 8), or an amino acid sequence that differs from SEQ ID NO: 8 by less than 5 conservative substitutions. In some embodiments, the polypeptide has the amino acid sequence of SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, and SEQ ID NO: 59.

In some embodiments, the TGF-β antagonist is a protein that has an amino acid sequence having at least 70% sequence identity (e.g., at least 75%, 80%, 85%, 86%, 87%, 88%, 89%, 90%, 91%, 92%, 93%, 94%, 95%, 96%, 97%, 98%, 99%, or more, sequence identity to SEQ ID NO: 9). In some embodiments, the TGF-β antagonist is a protein that has an amino acid sequence having at least 90% sequence identity to SEQ ID NO: 5. In some embodiments, the TGF-β antagonist is a protein that has an amino acid sequence having at least 95% sequence identity to SEQ ID NO: 9. In some embodiments, the TGF-β antagonist has an amino acid sequence that differs from the sequence of SEQ ID NO: 9 by one or more conservative substitutions (e.g., by 1, 2, 3, 4, 5, 6, 7, 8, 9, 10, 15, 20, or more, conservative substitutions). In some embodiments, the TGF-β antagonist has an amino acid sequence that differs from the sequence of SEQ ID NO: 9 by fewer than 10 non-conservative substitutions (e.g., by 10, 9, 8, 7, 6, 5, 4, 3, 2, 1, or 0 non-conservative substitutions). In some embodiments, the TGF-β antagonist has an amino acid sequence that differs from the sequence of SEQ ID NO: 5 only by one or more conservative substitutions (e.g., only by 1, 2, 3, 4, 5, 6, 7, 8, 9, 10, 15, 20, or more, conservative substitutions).

In some embodiments, the TGF-β antagonist is a protein that has an amino acid sequence having at least 70% sequence identity to SEQ ID NO: 5 (e.g., at least 75%, 80%, 85%, 86%, 87%, 88%, 89%, 90%, 91%, 92%, 93%, 94%, 95%, 96%, 97%, 98%, 99%, or more, sequence identity to SEQ ID NO: 5). In some embodiments, the TGF-β antagonist is a protein that has an amino acid sequence having at least 90% sequence identity to SEQ ID NO: 5. In some embodiments, the TGF-β antagonist is a protein that has an amino acid sequence having at least 95% sequence identity to SEQ ID NO: 5. In some embodiments, the TGF-β antagonist has an amino acid sequence that differs from the sequence of SEQ ID NO: 9 by one or more conservative substitutions (e.g., by 1, 2, 3, 4, 5, 6, 7, 8, 9, 10, 15, 20, or more, conservative substitutions). In some embodiments, the TGF-β antagonist has an amino acid sequence that differs from the sequence of SEQ ID NO: 5 by fewer than 10 non-conservative substitutions (e.g., by 10, 9, 8, 7, 6, 5, 4, 3, 2, 1, or 0 non-conservative substitutions). In some embodiments, the TGF-β antagonist has an amino acid sequence that differs from the sequence of SEQ ID NO: 5 only by one or more conservative substitutions (e.g., only by 1, 2, 3, 4, 5, 6, 7, 8, 9, 10, 15, 20, or more, conservative substitutions).

The invention provides for variants of the above compounds, having, for example, at least 75%, 80%, 85%, 90%, 95%, 96%, 97%, 98%, 99%, or more, sequence identity to the amino acid sequences described therein.

In another aspect, the invention features a pharmaceutical composition containing the conjugate of any of the above aspects and embodiments of the invention and a pharmaceutically acceptable excipient. In some embodiments, the conjugate is formulated for subcutaneous, intradermal, intramuscular, intraperitoneal, intravenous, intranasal, epidural, or oral administration. For instance, the conjugate may be formulated for intramuscular administration. In some embodiments, the conjugate is formulated for intravenous administration.

In one aspect, the invention features a method of treating a human patient suffering from a bone disease associated with elevated TGF-β signaling by administering to the patient a therapeutically effective amount of a composition comprising a TGF-β receptor fusion protein antagonist bound to a bone-targeting moiety of any of the above aspects or embodiments of the invention. In one aspect, the disease is a disease associated with elevated bone turnover. In another aspect, the disease is selected from the group consisting of osteogenesis imperfecta, McCune-Albright syndrome, Gaucher disease, hyperoxaluria, Paget disease of bone, and juvenile Paget disease. In one aspect, the disease is osteogenesis imperfecta, such as Type I osteogenesis imperfecta, Type II osteogenesis imperfecta, Type III osteogenesis imperfecta, Type IV osteogenesis imperfecta, Type V osteogenesis imperfecta, Type VI osteogenesis imperfecta, Type VII osteogenesis imperfecta, Type VIII osteogenesis imperfecta, Type IX osteogenesis imperfecta, Type X osteogenesis imperfecta, or Type XI osteogenesis imperfecta.

In one aspect, the invention features a method of treating a human patient suffering from a bone disease associated with elevated TGF-β signaling by administering to the patient a therapeutically effective amount of a composition comprising a TGF-β receptor fusion protein antagonist bound to a bone-targeting moiety of any of the above aspects or embodiments of the invention. In some embodiments, the composition includes a homodimer of a compound that has the amino acid sequence of SEQ ID NO: 28, or a variant of the amino acid sequence. In some embodiments, the composition includes a homodimer of a compound that has the amino acid sequence of SEQ ID NO: 30, or a variant of the amino acid sequence.

In an additional aspect, the invention features a method of treating a human patient suffering from a disease associated with elevated TGF-β signaling by administering to the patient a therapeutically effective amount of the conjugate or pharmaceutical composition of any of the above aspects or embodiments of the invention.

In another aspect, the invention features a method of treating a human patient suffering from a bone disease associated with elevated TGF-β signaling by administering to the patient a therapeutically effective amount of a composition comprising a TGF-β receptor fusion protein antagonist of any of the above aspects or embodiments of the invention.

In one aspect, the above methods of the invention feature administering to the patient a therapeutically effective amount of a composition that includes a homodimer of an amino acid sequence selected from SEQ ID NO: 15, SEQ ID NO: 17, SEQ ID NO: 19, SEQ ID NO: 21, SEQ ID NO: 33, or SEQ ID NO: 35; or a variant of the amino acid sequences.

In another aspect, the above methods of the invention feature administering to the patient a therapeutically effective amount of a composition that includes a homodimer of an amino acid sequence selected from SEQ ID NO: 5, SEQ ID NO: 14, SEQ ID NO: 16, SEQ ID NO: 18, SEQ ID NO: 20, SEQ ID NO: 32, or SEQ ID NO: 34; or a variant of the amino acid sequences.

In another aspect, the above methods of the invention feature administering to the patient a therapeutically effective amount of a composition that includes a homodimer of an amino acid sequence selected from SEQ ID NO: 9, SEQ ID NO: 23, SEQ ID NO: 25, SEQ ID NO: 27, SEQ ID NO: 29, or SEQ ID NO: 31; or a variant of the amino acid sequences.

In another aspect, the above methods of the invention feature administering to the patient a therapeutically effective amount of a composition that includes a homodimer of an amino acid sequence selected from SEQ ID NO: 22, SEQ ID NO: 24, SEQ ID NO: 26, SEQ ID NO: 28, or SEQ ID NO: 30; or a variant of the amino acid sequences.

In another aspect, the above methods of the invention feature administering to the patient a therapeutically effective amount of a composition that includes a homodimer of the amino acid sequence of SEQ ID NO: 29; or a variant of the amino acid sequence.

In another aspect, the above methods of the invention feature administering to the patient a therapeutically effective amount of a composition that includes a homodimer of the amino acid sequence of SEQ ID NO: 28; or a variant of the amino acid sequence.

In another aspect, the above methods of the invention feature administering to the patient a therapeutically effective amount of a composition that includes a homodimer of the amino acid sequence of SEQ ID NO: 31; or a variant of the amino acid sequence.

In yet another aspect, the above methods of the invention feature administering to the patient a therapeutically effective amount of a composition that includes a homodimer of the amino acid sequence of SEQ ID NO: 30; or a variant of the amino acid sequence.

In an additional aspect, the invention features a method of improving muscle function in a human patient suffering from a disease associated with elevated TGF-β signaling by administering to the patient a therapeutically effective amount of the conjugate or pharmaceutical composition of any of the above aspects or embodiments of the invention.

In another aspect, the invention features a method for improving muscle function in a human patient suffering from pathologies associated with elevated TGF-β signaling by administering to the patient a therapeutically effective amount of the conjugate or pharmaceutical composition of any of the above aspects or embodiments of the invention.

In another aspect, the invention features a method of treating a human patient suffering from a disease associated with elevated bone turnover by administering to the patient a therapeutically effective amount of the conjugate or pharmaceutical composition of any of the above aspects or embodiments of the invention.

Using a method for assessing muscle function (e.g., muscle mass, muscle strength, or muscle quality) described herein or known in the art, a physician may determine that the patient exhibits a level of muscle function that is less than that of a muscle function reference level, such as the level of muscle function of a healthy patient (e.g., a healthy patient of the same gender, age, and/or body mass, among other characteristics, as the patient) or the level of muscle function exhibited by the patient as assessed before the patient was diagnosed as having the disease. A finding that the patient exhibits, for instance, a level of muscle function that is less than that of the muscle function reference level may indicate that the patient is likely to respond to treatment with a TGF-β antagonist, such as a TGF-β antagonist described herein. Further, one or more of the compositions and methods described herein may be used to monitor changes (e.g., improvements or lack of improvement) in muscle function over time, for instance, to evaluate therapeutic efficacy.

In one aspect, the invention features a method of improving muscle function in a human patient suffering from a disease associated with elevated TGF-β signaling, the method including:
(a) assessing a level of muscle function exhibited by the patient;
(b) comparing the level of muscle function exhibited by the patient to a muscle function reference level; and
(c) administering a therapeutically effective amount of a TGF-β antagonist to the patient if the level of muscle function exhibited by the patient is less than the muscle function reference level.

In some instances, the level of muscle function exhibited by the patient has previously been assessed. For instance, in an aspect, the invention features a method of improving muscle function in a human patient suffering from a disease associated with elevated TGF-β signaling, where a level of muscle function exhibited by the patient has been assessed, the method including:
(a) comparing the level of muscle function exhibited by the patient to a muscle function reference level; and
(b) administering a therapeutically effective amount of a TGF-β antagonist to the patient if the level of muscle function exhibited by the patient is less than the muscle function reference level.

In some instances, the patient is identified as exhibiting a level of muscle function that is less than a muscle function reference level, and, thus, is determined to be likely to benefit from treatment with a TGF-β antagonist. For instance, in an aspect, the invention features a method of improving muscle function in a human patient suffering from a disease associated with elevated TGF-β signaling, the method including:
(a) assessing a level of muscle function exhibited by the patient;
(b) determining that the level of muscle function exhibited by the patient is less than a muscle function reference level;
(c) identifying the patient as likely to benefit from treatment with a TGF-β antagonist; and
(d) administering a therapeutically effective amount of the TGF-β antagonist to the patient.

In some instances, the level of muscle function exhibited by the patient has previously been assessed. For instance, in an aspect, the invention features a method of improving muscle function in a human patient suffering from a disease associated with elevated TGF-β signaling, wherein a level of muscle function exhibited by the patient has been assessed, the method including:
(a) determining that the level of muscle function exhibited by the patient is less than a muscle function reference level;
(b) identifying the patient as likely to benefit from treatment with a TGF-β antagonist; and
(c) administering a therapeutically effective amount of the TGF-β antagonist to the patient.

In yet another aspect, the invention features a method of identifying whether a human patient suffering from a disease associated with elevated TGF-β signaling is likely to benefit from treatment with a TGF-β antagonist, the method including:
(a) assessing a level of muscle function exhibited by the patient;
(b) comparing the level of muscle function exhibited by the patient to a muscle function reference level; and
(c) identifying the patient as likely to benefit from treatment with a TGF-β antagonist if the level of muscle function exhibited by the patient is less than the muscle function reference level.

In some instances, the level of muscle function exhibited by the patient has previously been assessed. For instance, in an aspect, the invention features a method of identifying whether a human patient suffering from a disease associated with elevated bone turnover is likely to benefit from treatment with a TGF-β antagonist, wherein a level of muscle function exhibited by the patient has been assessed, the method including:
(a) comparing the level of muscle function exhibited by the patient to a muscle function reference level; and
(b) identifying the patient as likely to benefit from treatment with a TGF-β antagonist if the level of muscle function exhibited by the patient is less than the muscle function reference level.

In some embodiments, the method further includes administering a therapeutically effective amount of the TGF-β antagonist to the patient.

In some embodiments of the above methods of the invention, the muscle function reference level is a level of muscle function of a subject (e.g., a human subject), optionally of the same gender, age, and/or body mass as the patient, that does not have the disease. In some embodiments of the above methods of the invention, the muscle function reference level is a prior level of muscle function exhibited by the patient before the patient was diagnosed as having the disease.

In some embodiments of the above methods of the invention, the muscle function in the patient refers to any one of muscle mass, muscle strength, and/or muscle quality.

In some embodiments of the above methods of the invention, the disease is a disease associated with elevated bone turnover. In some embodiments of the above methods of the invention, the disease is a bone disease. In other embodiments of the above methods of the invention, the disease is a muscle disease.

In some embodiments of the above methods of the invention, the invention features a method of improving muscle function in a human patient suffering from a disease associated with elevated bone turnover, by administering to the patient a therapeutically effective amount of a conjugate or pharmaceutical composition of any of the above aspects or embodiments of the invention.

In some embodiments of the above methods of the invention, the disease is selected from the group consisting of renal osteodystrophy, hyperparathyroid induced bone disease, diabetic bone disease, osteoarthritis, steroid induced bone disease, disuse osteoporosis, and Cerebral Palsy.

In some embodiments of the above methods of the invention, the disease is selected from the group consisting of osteogenesis imperfecta, McCune-Albright Syndrome, Gaucher Disease, Hyperoxaluria, Paget Disease of bone, and Juvenile Paget Disease.

In some embodiments of the above methods of the invention, the disease is osteogenesis imperfecta, such as Type I osteogenesis imperfecta, Type II osteogenesis imperfecta, Type III osteogenesis imperfecta, Type IV osteogenesis imperfecta, Type V osteogenesis imperfecta, Type VI osteogenesis imperfecta, Type VII osteogenesis imperfecta, Type VIII osteogenesis imperfecta, Type IX osteogenesis imperfecta, Type X osteogenesis imperfecta, or Type XI osteogenesis imperfecta.

In some embodiments of the above methods of the invention, the disease is metastatic bone cancer. In some embodiments, the patient is suffering from breast cancer or prostate cancer.

In some embodiments of the above methods of the invention, the disease is selected from the group consisting of osteoporosis, fibrous dysplasia, Calmurati-Engleman Disease, Marfan's Syndrome, osteoglophonic dysplasia, autosomal dominant osteopetrosis, osteoporosis, osteoporosis-pseudoglioma syndrome, juvenile, gerodermia osteodysplastica, Duchenne muscular dystrophy, osteosarcoma, osteogenesis imperfecta congenita, microcephaly, and cataracts.

In some embodiment of the above methods of the invention, the disease is selected from the group consisting of pseudohypoparathyroidism, Cleidocranial Dysplasia, Dyskeratosis Congenita, Exudative Vitreoretinopathy 1, Schimmelpenning-Feuerstein-Mims Syndrome, Prader-Willi Syndrome, Achondrogenesis, Antley-Bixler Syndrome, Aspartylglucosaminuria, Celiac Disease, Cerebrooculofacioskeletal Syndrome 1, Lysinuric Protein Intolerance, neuropathy, dyskeratosis congenita, Ehlers-Danlos Syndrome, epiphyseal dysplasia, hyaline fibromatosis syndrome, Perrault Syndrome 1, hemochromatosis, homocystinuria (e.g., due to cystathionine beta-synthase deficiency), hypophosphatemic rickets with hypercalciuria, desbuquois dysplasia, multiple pterygium syndrome, lethal congenital contracture syndrome 1, mitochondrial DNA depletion Ssndrome 6 (hepatocerebral Type), Niemann-Pick Disease, osteopetrosis, porphyria, Rothmund-Thomson Syndrome, Wilson Disease, Dent Disease 1, occipital horn syndrome, hyperglycerolemia, hypophosphatemic rickets, Lowe Oculocerebrorenal Syndrome, renal tubulopathy, diabetes mellitus, cerebellar ataxia, vitamin D hydroxylation-deficient rickets, Warburg micro syndrome 1, Stuve-Wiedemann Syndrome, Blue Rubber Bleb Nevus syndrome, Singleton-Merten Syndrome, microcephalic osteodysplastic primordial dwarfism, dysosteosclerosis, Hallermann-Streiff Syndrome, Bruck Syndrome 1, multiple pterygium syndrome (e.g., X-Linked), spondylometaphyseal dysplasia with dentinogenesis imperfecta, Hall-Riggs Mental Retardation Syndrome, infantile multisystem neurologic disease with osseous fragility, acrocephalopolysyndactyly Type III, acroosteolysis, ACTH-independent macronodular adrenal hyperplasia, amino aciduria with mental deficiency, arthropathy, bone fragility (e.g., with craniosynostosis, ocular proptosis, hydrocephalus, and distinctive facial features), brittle cornea syndrome, cerebrotendinous xanthomatosis, Cri-Du-Chat Syndrome, dysplasia epiphysealis hemimelica, autosomal dominant Ehlers-Danlos Syndrome, familial osteodysplasia, Flynn-Aird Syndrome, gerodermia osteodysplastica, Duchenne muscular dystrophy, osteosarcoma, glycogen storage disease Ia, Hutchinson-Gilford Progeria Syndrome, Infantile Systemic Hyalinosis, hypertrichotic osteochondrodysplasia, hyperzincemia with functional zinc depletion, hypophosphatasia, autosomal dominant hypophosphatemic rickets, X-linked recessive hypophosphatemic rickets, Lichtenstein Syndrome, macroepiphyseal dysplasia (e.g., with osteoporosis wrinkled skin, and aged appearance), Menkes Disease, Mental Retardation (e.g., X-Linked, Snyder-Robinson type), Jansen type metaphyseal chondrodysplasia, microspherophakia-metaphyseal dysplasia, morquio syndrome a, Morquio Syndrome B, ossified ear cartilages (e.g., with mental deficiency, muscle wasting, and osteocraniostenosis), osteoporosis and oculocutaneous hypopigmentation syndrome, osteoporosis-pseudoglioma syndrome, juvenile osteoporosis, osteosclerosis with ichthyosis and fractures, ovarian dysgenesis 1, ovarian dysgenesis 2, ovarian dysgenesis 3, ovarian dysgenesis 4, pituitary adenoma, polycystic lipomembranous osteodysplasia with sclerosing leukoencephalopathy, Prader-Willi Habitus, osteopenia, Okamoto type premature aging syndrome, Prieto X-linked mental retardation syndrome, pycnodysostosis, Pyle Disease, Reifenstein Syndrome, autosomal dominant distal renal tubular acidosis, Type 1 Schwartz-Jampel Syndrome, Type 2 Schwartz-Jampel Syndrome, Type 3 Schwartz-Jampel Syndrome, Type 4 Schwartz-Jampel Syndrome, X-linked spondyloepiphyseal dysplasia tarda, and Torg-Winchester Syndrome.

In another aspect, the invention features a method of improving muscle function in a human patient suffering from a disease associated with a pathological increase in TGF-β activity in a human patient by administering to the human patient a pharmaceutical formulation of any of the aspects or embodiments of the invention described herein. In some embodiments of the above methods of the invention, the disease associated with a pathological increase in TGF-β activity is fibrosis, liver fibrosis, non-alcoholic steatohepatitis, a pathological skin fibrotic condition, a wound, delayed wound healing, scarring, hypertrophic scarring, keloid scarring, an internal wound, an internal wound caused by a surgical procedure, a burn, epidermal burn, superficial dermal burn, mid-dermal burn, deep dermal burn, a full thickness burn, a pulmonary disease, asthma, chronic obstructive pulmonary disease, and fibroproliferative lung disease, a renal disease, or diabetic nephropathy.

In some embodiments of the above methods of the invention, the disease is an autoimmune disease, such as psoriasis or scleroderma.

In some embodiments of the above methods of the invention, the disease is cancer. In some embodiments of the above methods of the invention, the cancer is carcinoma, pancreatic cancer, glioblastoma, myeloid leukemia, head and neck cancer, melanoma, breast cancer, or colorectal cancer. In some embodiments, the carcinoma is selected from the group consisting of squamous cell carcinoma, epidermoid carcinoma, urothelial carcinoma, adenocarcinoma, adrenocortical carcinoma, basal cell carcinoma, ductal carcinoma in situ (DCIS), invasive ductal carcinoma, Merkel cell carcinoma, midline tract carcinoma, thymic carcinoma, and renal cell carcinoma. In some embodiments of the above methods of the invention, the carcinoma is squamous cell carcinoma. In other embodiments, the squamous cell carcinoma is vulvar squamous cell carcinoma, epidermal squamous cell carcinoma, oral squamous cell carcinoma, pulmonary squamous cell carcinoma, or head and neck squamous cell carcinoma.

In another aspect, the invention features a method of improving muscle function in a human patient suffering from a disease associated with a pathological increase in TGF-β activity in a human patient by administering to the patient a pharmaceutical formulation of a composition of any of the above aspects or embodiments of the invention. In some embodiments of the above methods of the invention, the composition includes a homodimer of a compound that has an amino acid sequence selected from the group consisting of SEQ ID NO: 9, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, and SEQ ID NO: 35. In some embodiments of the above methods of the invention, the disease is selected from the group comprising fibrosis, liver fibrosis, non-alcoholic steatohepatitis, a pathological skin fibrotic condition, a wound, delayed wound healing, scarring, hypertrophic scarring, keloid scarring, an internal wound, an internal wound caused by a surgical procedure, a burn, epidermal burn, superficial dermal burn, mid-dermal burn, deep dermal burn, a full thickness burn, a pulmonary disease, asthma, chronic obstructive pulmonary disease, and fibroproliferative lung disease, a renal disease, diabetic nephropathy, an autoimmune disease (e.g., psoriasis, or scleroderma), and cancer (e.g., carcinoma, pancreatic cancer, glioblastoma, myeloid leukemia, head and neck cancer, melanoma, breast cancer, colorectal cancer, prostate cancer).

In some embodiments of the above methods of the invention, the carcinoma is selected from the group comprising squamous cell carcinoma (e.g., vulvar squamous cell carcinoma, epidermal squamous cell carcinoma, oral squamous cell carcinoma, pulmonary squamous cell carcinoma, or head and neck squamous cell carcinoma), epidermoid carcinoma, urothelial carcinoma, adenocarcinoma, adrenocortical carcinoma, basal cell carcinoma, ductal carcinoma in situ (DCIS), invasive ductal carcinoma, Merkel cell carcinoma, midline tract carcinoma, thymic carcinoma, and renal cell carcinoma.

In some aspects, the method of administering to the patient a therapeutically effective amount of a conjugate or pharmaceutical composition, such as a TGF-β antagonist, of any of the above aspects or embodiments of the invention results in the patient exhibiting an increase in muscle mass, muscle strength, and/or muscle quality.

In some embodiment, the invention features a method of treating a human patient suffering from a muscle disease associated with elevated TGF-β signaling by administering to the patient a therapeutically effective amount of the conjugate or pharmaceutical composition of any of the above aspects or embodiments of the invention. In some embodiments, the muscle disease is a muscular dystrophy. The muscular dystrophy may be an inherited muscular dystrophy, such as laminin-α2-deficient congenital muscular dystrophy or muscular dystrophy associated with one or more mutations in the gene encoding caveolin-3. In some embodiments, the muscular dystrophy is Duchenne muscular dystrophy. In some embodiments, the muscle disease is an acquired muscle disease, such as sarcopenia.

In some embodiments, the method includes administering the conjugate or pharmaceutical composition of any of the above aspects or embodiments of the invention to the patient subcutaneously, intradermally, intramuscularly, intraperitoneally, intravenously, or orally, or by nasal or epidural administration. For instance, the method may include administering the conjugate or pharmaceutical composition to the patient intramuscularly. In some embodiments, the method includes administering the conjugate or pharmaceutical composition to the patient intravenously.

In an additional aspect, the invention features a kit containing the conjugate or pharmaceutical composition of any of the above aspects or embodiments of the invention and a package insert. The package insert may instruct a user of the kit to treat a human patient suffering from a disease associated with elevated TGF-β signaling, such as disease associated with elevated TGF-β signaling described herein, by administering to the patient a therapeutically effective amount of the conjugate or the pharmaceutical composition of any of the above aspects or embodiments of the invention.

In an additional aspect, the invention features a kit containing the conjugate or pharmaceutical composition of any of the above aspects or embodiments of the invention and a package insert. The package insert may instruct a user of the kit to treat a human patient suffering from a disease associated with elevated TGF-β signaling, such as disease associated with elevated bone turnover or a muscular dystrophy described herein, by administering to the patient a therapeutically effective amount of the conjugate or the pharmaceutical composition of any of the above aspects or embodiments of the invention.

In an additional aspect, the invention features a kit containing the conjugate or pharmaceutical composition of any of the above aspects or embodiments of the invention and a package insert. The package insert may indicate that the kit is for improving muscle function in a human patient suffering from a disease associated with elevated TGF-β signaling, such as a disease associated with elevated TGF-β signaling described herein, by administering to the patient a therapeutically effective amount of the conjugate or the pharmaceutical composition of any of the above aspects or embodiments of the invention.

In an additional aspect, the invention features a kit containing the conjugate or pharmaceutical composition of any of the above aspects or embodiments of the invention and a package insert. The package insert may indicate that the kit is for improving muscle function in a human patient suffering from a disease associated with elevated TGF-β signaling, such as a skeletal disorder (e.g., a disease associated with elevated bone turnover) and a muscle disease (e.g., muscular dystrophy) described herein, by administering to the patient a therapeutically effective amount of the conjugate or the pharmaceutical composition of any of the above aspects or embodiments of the invention. In some aspects, the disease is fibrosis, an autoimmune disease, or cancer.

The invention also includes a cell containing a nucleic acid sequence encoding any of the above peptide components of the compounds or compounds. Such a nucleic acid may further include a signal sequence.

A method of manufacturing the compositions of the invention, may include the steps of culturing the cell aforementioned cell in a suitable growth medium and isolating the mature form of the polypeptide encoded by said nucleic acid.

### Definitions

As used herein, the term “about” refers to a value that is within 10% above or below the value being described. For instance, the phrase “about 50 nM” refers to a value between and including 45 nM and 55 nM.

As used herein, the term “affinity” refers to the strength of a binding interaction between two molecules, such as a ligand (such as an isoform of TGF-β) and a receptor. The term “K_{d}”, as used herein, is intended to refer to the dissociation constant, which can be obtained, for example, from the ratio of the rate constant for the dissociation of the two molecules (k_{d}) to the rate constant for the association of the two molecules (kₐ) and is expressed as a molar concentration (M). The range may be from 100 to 0.001 nM. K_{d} values for peptide-protein or protein-protein interactions can be determined, e.g., using methods established in the art. Methods that can be used to determine the K_{d} of a peptide-protein or protein-protein interaction include surface plasmon resonance, e.g., through the use of a biosensor system such as a BIACORE® system, as well as fluorescence anisotropy and polarization methods and calorimetry techniques known in the art, such as isothermal titration calorimetry (ITC).

As used herein, the term “antibody” refers to an immunoglobulin molecule that specifically binds to, or is immunologically reactive with, a particular antigen, and includes polyclonal, monoclonal, genetically engineered, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen binding fragments of antibodies, including, for example, Fab′, F(ab′)₂, Fab, Fv, rlgG, and scFv fragments. Unless otherwise indicated, the term “monoclonal antibody” (mAb) is meant to include both intact molecules, as well as antibody fragments (including, for example, Fab and F(ab′)₂ fragments) that are capable of specifically binding to a target protein. As used herein, the Fab and F(ab′)₂ fragments refer to antibody fragments that lack the Fc fragment of an intact antibody. Examples of these antibody fragments are described herein.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, for example, a Fab, F(ab′)₂, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)₂fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment that consists of a V_{H} domain (see, e.g., Ward et al., Nature 341:544-546, 1989); (vii) a dAb which consists of a V_{H} or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “anti-TGF-β antibody” refers to a protein or peptide-containing molecule that includes at least a portion of an immunoglobulin molecule, such as but not limited to at least one complementarity determining region (CDR) of a heavy or light chain or a ligand binding portion thereof, a heavy chain or light chain variable region, a heavy chain or light chain constant region, a framework region, or any portion thereof, that is capable of specifically binding to TGF-β. Anti-TGF-β antibodies also include antibody-like protein scaffolds, such as the tenth fibronectin type III domain (¹⁰Fn3), which contains BC, DE, and FG structural loops similar in structure and solvent accessibility to antibody CDRs. The tertiary structure of the ¹⁰Fn3 domain resembles that of the variable region of the IgG heavy chain, and one of skill in the art can graft, for example, the CDRs of an anti-TGF-β monoclonal antibody onto the fibronectin scaffold by replacing residues of the BC, DE, and FG loops of ¹⁰Fn3 with residues from the CDRH-1, CDRH-2, or CDRH-3 regions of an anti-TGF-β monoclonal antibody.

As used herein, the term “benefit’ in the context of a patient, such as a human patient suffering from a disease associated with elevated TGF-β signaling, such as osteogenesis imperfecta and muscular dystrophy, refers to any clinical improvement in the patient's condition, including, for example, a reduced progression of the disease or an attenuated severity of one or more symptoms associated with the disease, such as the propensity of the patient to suffer from recurring bone fractures or a decline in muscle function. Exemplary benefits in this context, such as in the context of a patient treated with a TGF-β antagonist, include, without limitation, an improvement of muscle function. A patient can be determined to benefit, for instance, from TGF-β antagonist treatment as described herein by observing an improvement in muscle function (e.g., muscle mass, muscle strength, and/or muscle quality) in the patient, as assessed, for instance, using a methodology known in the art or described herein.

Additionally or alternatively, a patient can be determined to benefit from TGF-β antagonist treatment as described herein by observing an increase in the integrity of one or more bones in the patient, a decrease in the rate or extent of resorption at one or more bones in the patient, and/or a restoration of homeostasis of bone turnover in the patient (e.g., a patient suffering from osteogenesis imperfecta). These benefits can be assessed, for instance, using methods for measuring and characterizing the structure, density, and/or quality of bone. Examples of such methods are known in the art and include, for instance, histology and histomorphometry, atomic force microscopy, confocal Raman microscopy, nanoindentation, three-point bending test, X-ray imaging, and micro computed tomography (μ-CT).

As used herein, the terms “bone targeting moiety”, “bone-targeting moiety”, and “bone anchor” refer to a polypeptide that utilize special affinities to target the mineral or protein components in bone tissue.

As used herein, the term “bone turnover” refers to the dual processes of resorption (e.g., by osteoclasts) and redeposition (e.g., by osteoblasts) of bone proteins, such as collagen and non-collagenous proteins, as well calcium and other minerals that comprise bone tissue (hereafter called one material). In healthy individuals, the net effect of these processes is the maintenance of a constant bone balance. In normal growing bones, the bone deposition is in equilibrium with the bone resorption, whereas in certain pathological conditions, bone resorption exceeds bone deposition. As used herein, the term “elevated bone turnover” in the context of a patient suffering from a pathological disease or condition refers to an increase in the rate of bone resorption and redeposition relative to a reference level, such as the rate of bone resorption and redeposition in a healthy subject not suffering from the disease or condition or the rate of resorption and redeposition in the subject of interest as measured prior to the subject being diagnosed with the disease or condition. Methods for assessing bone turnover include, for instance, measuring the concentration of one or more biomarkers of bone turnover in a subject, such as serum and bone alkaline phosphatase, serum osteocalcin (sOC), serum type I collagen C-telopeptide breakdown products (sCTX), urinary free-deoxypyridinoline (ufDPD), and urinary cross-linked N-telopeptides of type I collagen (uNTX) and comparing the concentration of the one or more biomarkers to that of a healthy subject, as described, for instance, in Braga et al. Bone 34:1013-1016 (2004), the disclosure of which is incorporated herein by reference as it pertains to biomarkers for assessing bone turnover.

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each comprise four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). As used herein, numbering of immunoglobulin amino acid residues is performed according to the immunoglobulin amino acid residue numbering system of Kabat et al., unless otherwise indicated.

As used herein in the context of conjugates, such as fusion proteins, the term “bound to” refers to the covalent joining of one molecule, such as a protein, polypeptide, or domain thereof, to another molecule, such as another protein, polypeptide, or domain thereof. Two molecules that are “bound to” one another as described herein may be directly bound to one another, for instance, without an intervening linker. Alternatively, two molecules that are “bound to” one another may be bound by way of a linker. Exemplary linkers include synthetic linkers containing coupling moieties listed in Table 14, herein, as well as peptidic linkers, such as those that contain one or more glycine, serine, and/or threonine residues. Additional examples of linkers that may be used in conjunction with the compositions and methods described herein include immunoglobulin Fc domains, as well as fragments thereof.

As used herein, the term “conjugate” refers to a compound formed by the chemical bonding of a reactive functional group of one molecule, such as protein, polypeptide, or domain thereof, with an appropriately reactive functional group of another molecule, such as another protein, polypeptide, or domain thereof. Optionally the molecule may be biologically or pharmacologically active or inactive. Conjugates include fusion proteins in which one or more polypeptides are joined covalently to one another by way of covalent bonds, such as by way of amide bonds between the N- and C-termini of the component fragments of the fusion protein. Such conjugates may be generated, for instance, by recombinant expression from a cell (e.g., a prokaryotic cell, such as a bacterial cell, or a eukaryotic cell, such as a mammalian cell). Conjugates may include a linker between the two molecules covalently bound to one another. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012).

As used herein, the terms “conservative mutation,” “conservative substitution,” or “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally-occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein in the context of conjugates, such as fusion proteins, the term “covalent bond” refers to the covalent joining of one molecule, such as a protein, polypeptide, or domain thereof, to another molecule, such as another protein, polypeptide, or domain thereof. Two molecules that are “covalently bound to” one another as described herein may be directly bound to one another, for instance, without an intervening linker. Alternatively, two molecules that are “covalently bound to” one another may be bound by way of a linker. Exemplary linkers include synthetic linkers containing coupling moieties listed in Table 14, herein, as well as peptidic linkers, such as those that contain one or more glycine, serine, and/or threonine residues. Additional examples of linkers that may be used in conjunction with the compositions and methods described herein include immunoglobulin Fc domains, as well as fragments thereof.

As used herein, the terms “decreasing,” “reducing,” “neutralizing,” attenuating,” “inhibiting,” “downregulating,” and “interfering,” are used interchangeably and refer to lowering the biological activity of TGF-β, e.g., TGF-β signaling. For example, a TGF-β antagonist may, for example, decrease or reduce TGF-β expression levels; bind to and neutralize the activity of TGF-β; attenuate TGF-β signaling, inhibit excess TGF-β signaling; downregulate the activity TGF-β; affect the stability or conversion of the precursor molecule to the active, mature form; interfere with the binding of TGF-β to one or more receptors, or it may interfere with intracellular signaling of a TGF-β receptor. The term “direct TGF-β antagonist” generally refers to any compound that directly downregulates the biological activity of TGF-β. A molecule “directly downregulates” the biological activity of TGF-β if it downregulates the activity by interacting with a TGF-β gene, a TGF-β transcript, a TGF-β ligand, or a TGF-β receptor.

As used herein, the term “dimer” refers to a multimeric form of a peptide conjugate. For instance, in the context of a TGF-β antagonist, such as a TGF-β receptor fusion protein conjugate (e.g., TGF-β RER trap) described herein, the conjugate may be present as homodimers with the two monomers linked by covalent bonds. Dimeric TGF-β traps may contain two copies of an Fc domain of an immunoglobulin linked to an RER peptide conjugate, and the two copies may be bound to one another by disulfide bridges between cysteine residues within the peptides or by way of a linker.

As used herein, the term “ectodomain” describes the domain of a membrane protein that extends into the extracellular space when the peptide sequence is present in the form of the full-length protein.

As used herein, the term “elevated TGF-β activity” in the context of a patient suffering from a pathological disease or condition refers to an increase in TGF-β signaling relative to a reference level, such as TGF-β signaling in a healthy subject not suffering from the disease or condition or TGF-β signaling in the subject of interest as measured prior to the subject being diagnosed with the disease or condition. Methods for assessing TGF-β signaling are known in the art and include, for instance, measuring the extent of transcription of a gene of interest under the control of a promoter regulated by a transcription factor (e.g., a Smad protein) that is activated by the TGF-β signal transduction cascade, as well as measuring the concentration or relative level of one or more phosphorylated Smad transcription factors.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials, such as recombinant fusion protein conjugates, include those that are provided from an external source to an organism or to cultured matter extracted therefrom.

As used herein, “endoglin” describes a type I membrane glycoprotein that is part of the TGF-β receptor complex that interacts with high affinity to TGF-β type III receptors.

As used herein, the term “elevated TGF-β activity” in the context of a patient suffering from a pathological disease or condition refers to an increase in TGF-β signaling relative to a reference level, such as TGF-β signaling in a healthy subject not suffering from the disease or condition or TGF-β signaling in the subject of interest as measured prior to the subject being diagnosed with the disease or condition. Methods for assessing TGF-β signaling are known in the art and include, for instance, measuring the extent of transcription of a gene of interest under the control of a promoter regulated by a transcription factor (e.g., a Smad protein) that is activated by the TGF-β signal transduction cascade, as well as measuring the concentration or relative level of one or more phosphorylated Smad transcription factors.

As used herein, an “Fc domain” of an immunoglobulin describes a polypeptide comprising the constant region of an antibody, excluding the hinge ligand. Thus, Fc may refer to the constant region immunoglobulin domains of IgA, IgD, IgG, IgE, and IgM.

As used herein, “Formula a” refers to a trimeric TGF-β receptor fusion protein conjugate in which the C-terminal of RER is bound via a hinge linker to an N-terminal of a Fc domain, and the C-terminal of the Fc domain is bound to a targeting linker (FIG. 33A). It should be noted that Formula a may also refer to a fusion protein conjugate in which there is no targeting moiety.

As used herein, “Formula b” refers to a trimeric TGF-β receptor fusion protein conjugate in which an N-terminal of RER is bound via a hinge linker to a C-terminal of a Fc domain, and the N-terminal of the Fc domain is bound to a targeting linker (FIG. 33B). It should be noted that Formula b may also refer to a fusion protein conjugate in which there is no targeting moiety.

As used herein, “Formula c” refers to a trimeric TGF-β receptor fusion protein conjugate in which an N-terminal of RER is bound via a hinge linker to a C-terminal of a Fc domain, and the C-terminal of RER is bound to a targeting linker (FIG. 33C). It should be noted that Formula c may also refer to a fusion protein conjugate in which there is no targeting moiety; when there is no targeting moiety, Formula b and c are identical.

The term “functional status” as used herein, unless otherwise specified, refers to the ability of a subject to perform activities associated with normal daily living (ADLs) including, but not limited to, getting out of bed, getting off of the toilet, personal hygiene, self-feeding, performing housework, maintaining sufficient walking gait speed for safe and effective transfers, facilitating automobile-dependent transportation, and shopping for groceries. Functional status may be measured, for example, by the Katz Index of Independence in Activities of Daily Living, the Tinetti Gait and Balance Scale, the Palliative Performance Scale, the Short Physical Performance Battery, and so forth.

As used herein, the terms “fusion protein” or “TGF-β receptor fusion protein” refer to a conjugate that contains one polypeptide bound to another polypeptide, for instance, by way of a linker or by direct covalent bonding of the two polypeptides without an intervening linking moiety.

As used herein, the term “framework region” or “FW region” includes amino acid residues that are adjacent to the CDRs of an antibody or antigen-binding fragment thereof. FW region residues may be present in, for example, human antibodies, humanized antibodies, monoclonal antibodies, antibody fragments, Fab fragments, single chain antibody fragments, scFv fragments, antibody domains, and bispecific antibodies, among others.

As used herein, a “hinge” or “hinge linker” or “immunoglobulin hinge region” a polypeptide comprising the amino acids between the Fc region and the RER domains.

As used herein, the term “human antibody” refers to an antibody in which substantially every part of the protein (for example, all CDRs, framework regions, C_{L}, C_{H} domains (e.g., C_{H}1, C_{H}2, C_{H}3), hinge, and V_{L} and V_{H} domains) is substantially non-immunogenic in humans, with only minor sequence changes or variations. A human antibody can be produced in a human cell (for example, by recombinant expression) or by a non-human animal or a prokaryotic or eukaryotic cell that is capable of expressing functionally rearranged human immunoglobulin (such as heavy chain and/or light chain) genes. When a human antibody is a single chain antibody, it can include a linker peptide that is not found in native human antibodies. For example, an Fv can contain a linker peptide, such as two to about eight glycine or other amino acid residues, which connects the variable region of the heavy chain and the variable region of the light chain. Such linker peptides are considered to be of human origin. Human antibodies can be made by a variety of methods known in the art including phage display methods using antibody libraries derived from human immunoglobulin sequences. Human antibodies can also be produced using transgenic mice that are incapable of expressing functional endogenous immunoglobulins, but which can express human immunoglobulin genes (see, for example, PCT Publication Nos. WO 1998/24893; WO 1992/01047; WO 1996/34096; WO 1996/33735; U.S. Pat. Nos. 5,413,923; 5,625,126; 5,633,425; 5,569,825; 5,661,016; 5,545,806; 5,814,318; 5,885,793; 5,916,771; and 5,939,598).

As used herein, the term “humanized” antibody refers to a non-human antibody that contains minimal sequences derived from non-human immunoglobulin. In general, a humanized antibody contains substantially all of at least one, and typically two, variable domains, in which all or substantially all of the CDR regions correspond to those of a non-human immunoglobulin. All or substantially all of the FW regions may also be those of a human immunoglobulin sequence. The humanized antibody can also comprise at least a portion of an immunoglobulin constant region (Fc), typically that of a human immunoglobulin consensus sequence. Methods of antibody humanization are known in the art and have been described, for example, in Riechmann et al., Nature 332:323-7, 1988; U.S. Pat. Nos: 5,530,101; 5,585,089; 5,693,761; 5,693,762; and 6,180,370.

As used herein, the term “linker” refers to a polypeptide comprising the amino acids between receptor proteins in RER, between RER and an antibody Fc domain, and between an antibody Fc domain and a targeting moiety. For example, linkers that may be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012). In another example, linkers that may be used to connect two monomers into a dimer include thioether or amide bonds between a cysteine or lysine residue within each copy of the peptide and a bivalent linking moiety. Exemplary linking moieties include, for instance, succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, for instance, Liu et al., 18:690-697, 1979, the disclosure of which is incorporated herein by reference as it pertains to linkers for chemical conjugation. Additional linkers include the non-cleavable maleimidocaproyl linkers, which are particularly useful for the conjugation of microtubule-disrupting agents such as auristatins, are described by Doronina et al., Bioconjugate Chem. 17:14-24, 2006, the disclosure of which is incorporated herein by reference as it pertains to linkers for chemical conjugation.

As used herein, the term “Linker 1” or “L¹” refers to the linker between the “RER heterotrimeric fusion polypeptide” (“A”) and the Fc domain of an immunoglobulin (“B”), described below and as shown in FIGS. 33A-C.

As used herein, the term “Linker 2” or “L²” refers to the linker between B, the Fc domain of an immunoglobulin, and the bone-targeting moiety (“Z”) (FIGS. 33A and 33B), or the linker between A, the RER heterotrimeric fusion polypeptide, and Z, the bone-targeting moiety (FIG. 33C).

As used herein, the terms “Linker 3” or “L³” and “Linker 4” or “L⁴” refer to the linkers between TGF-β receptor II ectodomain and TGF-β receptor III endoglin domain as shown in FIGS. 33A-C.

As used herein, the term “low-molecular weight” in the context of a peptide refers to a peptide that has a molecular weight of less than 10 kDa, such as a peptide that has a molecular weight of 9,000 Da, 8,000 Da, 7,000 Da, 6,000 Da, 5,000 Da, 4,000 Da, 3,000 Da, or less.

As used herein, the term “mineral” in the context of a bone-targeting moiety refers to an inorganic ion, complex, or compound, comprised of inorganic elements, that is present in bone. Exemplary minerals include, without limitation, Ca²⁺, PO₄³⁻, OH⁻, and other trace inorganic elements. The mineral can include, for instance, such compounds as crystalline, nanocrystalline or amorphous hydroxyapatite (Ca₁₀(PO₄)₆(OH)₂), calcium carbonate, and calcium phosphates with solubility behavior, under acidic and basic conditions, similar to that of hydroxyapatite, including, but not limited to, dicalcium phosphate, tricalcium phosphate, octacalcium phosphate or calcium phosphates.

The term “muscle disease” as used herein, refers to any muscle disease, including those that are associated with elevated TGF-β signaling. Muscular dystrophies, for example, are muscle diseases associated with elevated TGF-β signaling. Muscular dystrophies may be inherited, e.g., laminin-α2-deficient congenital muscular dystrophy, muscular dystrophy associated with one or more mutations in the gene encoding caveolin-3, or Duchenne muscular dystrophy. The muscle disease may also refer to an acquired muscle disease, such as sarcopenia. The terms “muscle disease”, “muscle disorder”, “muscular disease” and “muscular disorder” are used interchangeably.

The term “muscle function” as used herein, unless otherwise specified, refers to at least one of muscle mass, muscle strength, or muscle quality.

The term “muscle mass” as used herein, unless otherwise specified, refers to the amount or size of muscle or muscle groups, as expressed by muscle weight, mass, area, or volume. Muscle mass may also be expressed as total lean body mass, lean body mass of a body compartment such as the leg, or cross-sectional area of a leg or arm compartment. The volume or mass of the muscle can be determined using any known or otherwise effective technique that provides muscle area, volume, or mass, such as dual-energy X-ray absorptiometry (DEXA), or using visual or imaging techniques such as MRI or CT scans.

The term “muscle quality” as used herein, unless otherwise specified, refers to the amount of muscle strength (e.g., in units of force of angular velocity) per unit volume, cross-sectional area, or mass of the corresponding muscle, muscle groups, or arm or leg compartment, i.e., the term “muscle quality” refers to muscle strength per corresponding muscle volume, muscle strength per corresponding muscle cross-sectional area, or muscle strength per corresponding muscle mass. For example, leg muscle quality refers to leg muscle strength/leg muscle volume or leg muscle strength/leg muscle mass.

The term “muscle strength” as used herein, unless otherwise specified, refers to the amount of force a muscle, or muscle groups in sum, can exert. Muscle strength may be evaluated by a variety of methods such as grip strength, open and mobility field tests, one repetition maximum strength test, time-dependent tests of muscle endurance, time-dependent tests of muscle fatigue, or time-dependent tests of muscle endurance and fatigue, and so forth.

The term “muscle weakness” as used herein, unless otherwise specified, refers to a reduction in muscle function (e.g., muscle strength, muscle mass, or muscle quantity), or a lack of muscle function (e.g., muscle strength, muscle mass, or muscle quantity). Muscle weakness may be determined based on a quantitative assessment of muscle function (e.g., a reduction in muscle function relative to a reference value) or a qualitative assessment of muscle function (e.g., performance score or functional status assessment).

As used herein, the term “percent (%) sequence identity” refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using publicly available computer software, such as BLAST, ALIGN, or Megalign (DNASTAR) software. Those skilled in the art can determine appropriate parameters for measuring alignment, including any algorithms needed to achieve maximal alignment over the full length of the sequences being compared. For example, a reference sequence aligned for comparison with a candidate sequence may show that the candidate sequence exhibits from 50% to 100% sequence identity across the full length of the candidate sequence or a selected portion of contiguous amino acid (or nucleic acid) residues of the candidate sequence. The length of the candidate sequence aligned for comparison purposes may be, for example, at least 30%, (e.g., 30%, 40, 50%, 60%, 70%, 80%, 90%, or 100%) of the length of the reference sequence. When a position in the candidate sequence is occupied by the same amino acid residue as the corresponding position in the reference sequence, then the molecules are identical at that position.

As used herein, the term “pharmaceutical composition” or “pharmaceutical formulation” refers to a composition or formulation (e.g., a mixture) containing a therapeutic compound, such as a conjugate described herein, to be administered to a subject, such as a mammal, e.g., a human, in order to halt the progression, improve, restore, prevent, treat or control a particular disease or condition (such as a disease or condition associated with elevated TGF-β activity described herein) affecting or that may affect the mammal. For example, the pharmaceutical compositions or pharmaceutical formulations, referred herein, can be administered to attenuate TGF-β signaling for the treatment of diseases associated with elevated TGF-β signaling, such as skeletal and muscle disorders. Additionally or alternatively, the pharmaceutical compositions or pharmaceutical formulations referred herein can be administered for improving muscle function (e.g., muscle mass, muscle strength, and/or muscle quality) in a subject, such as a mammal, e.g., a human, suffering from pathologies associated with elevated TGF-β signaling, such as skeletal and muscle disorders.

As used herein, the term “pharmaceutically acceptable” refers to the suitability of a carrier or vehicle for use in mammals, including humans, without undue toxicity, incompatibility, instability, irritation, allergic response, and the like, commensurate with a reasonable benefit/risk ratio.

As used herein, the terms “polypeptide,” “protein,” and “polypeptide” are used interchangeably and generally have their art-recognized meaning of a polymer of at least three amino acids. The term “polypeptide” can also be used to refer to specific functional classes of polypeptides, such as, for example, “an RER heterotrimeric fusion polypeptide” as described herein. The term “polypeptide” can refer to polypeptides in their neutral (uncharged) forms or as salts, and either unmodified or modified, e.g., by glycosylation, side chain oxidation, or phosphorylation.

As used herein, the term “portion” or “portion thereof” when used in reference to a polypeptide, refers to a portion of a polypeptide that retains activity and shares at least about 30-40% overall sequence identity with the polypeptide. In some embodiments, a portion of a polypeptide shares at least 50%, at least 60%, at least 70%, at least 80%, at least 85%, at least 90%, or at least 95% amino acid sequence identity with the polypeptide. In some embodiments, a portion of a polypeptide includes at least one region of much higher identity (e.g., greater than 90% or even 95%, 96%, 97%, 98%, or 99%) than the overall amino sequence identity with the polypeptide. In some embodiments, the region of much higher identity, if present, includes one or more highly conserved regions, usually encompassing at least 3-4 and often up to 20 or more amino acids.

As used herein, the term “receptor linker” refers to a polypeptide that binds covalently to the amino or the carboxy ends of TGF-β receptors.

As used herein, the term “RIIₐ” refers to the TGF-β type II receptor in a TGF-β fusion protein conjugate that is further from the hinge linker as shown in “Formula a” of FIG. 33A, while “RII_{b}” refers to the TGF-β type II receptor in a TGF-β fusion protein conjugate that is closer to the hinge linker as shown in “Formula a” of FIG. 33A. As used herein, the term “RIIₐ” also refers to the TGF-β type II receptor in a TGF-β fusion protein conjugate that is closer to the hinge linker as shown in “Formula b” of FIG. 33B and “Formula c” of FIG. 33C, while “RII_{b}” refers to the TGF-β type II receptor in a TGF-β fusion protein conjugate that is further form the hinge linker as shown in “Formula b” of FIG. 33B and “Formula c” of FIG. 33C.

As used herein, “recombinant variant” or “recombinant variant amino acid sequence” is meant a protein that differs from that of a parent amino acid sequence by virtue of at least one amino acid modification. “Variant amino acid sequence” may refer to a protein, a composition comprising a protein, or an amino sequence that encodes it. Preferably, the variant has at least one amino acid modification compared to the parent protein, e.g. from about one to about seventy amino acid modifications, and preferably from about one to about five amino acid modifications compared to the parent.

As used herein in the context of muscle function (e.g., muscle mass, muscle strength, and/or muscle quality), the term “reference level” refers to a threshold of muscle function exhibited by a patient (e.g., a human patient suffering from a skeletal disorders, such as a disease associated with elevated bone turnover, or a human patient suffering from a muscle disorder, such as muscular dystrophy) that, below which, indicates that the patient is likely to benefit from TGF-β antagonist treatment. Muscle function reference levels, as described herein, may refer, for instance, to a muscle mass threshold, muscle strength threshold, or muscle quality threshold that, below which, indicates that the patient is likely to benefit from TGF-β antagonist treatment. In some embodiments, the muscle function reference level is the level of muscle function (e.g., muscle mass, muscle strength, and/or muscle quality) of a human subject that is not suffering from a disease associated with elevated bone turnover (such as osteogenesis imperfecta). For instance, exemplary muscle function reference levels include the level of muscle function of a healthy human subject. For the purposes of making relevant comparisons between a muscle function reference level and a the level of muscle function exhibited by a patient, such as a patient suffering from a muscle disorder (e.g., a muscular dystrophy) or a disease associated with elevated bone turnover (e.g., osteogenesis imperfecta), the muscle function reference level may be the level of muscle function (e.g., muscle mass, muscle strength, and/or muscle quality) exhibited by a healthy human subject of the same gender, age, and/or body mass as the patient or the level of muscle function exhibited by the patient as assessed before the patient was diagnosed as having the disease.

As used herein, the term “region” in the context of a polypeptide refers to a segment of the polypeptide containing up to 50 consecutive amino acid residues. For instance, the term “N-terminal region” of a polypeptide refers to a segment containing the first 50 consecutive amino acid residues of the polypeptide, starting from the N-terminal amino acid residue. Similarly, the term “C-terminal region” of a polypeptide refers to a segment containing the final 50 consecutive amino acids of the polypeptide, ending at the C-terminal amino acid residue.

As used herein, the phrase “RER heterotrimeric fusion polypeptide” (“A”) refers to a heterotrimeric fusion in which the ectodomains of TGF-β type II receptors are coupled to amino and carboxy ends of an endoglin-domain of a TGF-β type III receptors. In the first instance, the phrase RER heterotrimeric fusion polypeptide to refers to a polypeptide sequence of general formula: W-L³-X-L⁴-Y, wherein

W is a TGF-β type II receptor ectodomain or a portion thereof;

L³ is a linker or is absent;

X is a TGF-β type III receptor endoglin domain or a portion thereof;

L⁴ is a linker or is absent; and

Y is a TGF-β type II receptor ectodomain or a portion thereof.

In some embodiments, W is at the N-terminus of the RER heterotrimeric fusion polypeptide and Y is at the C-terminus of the RER heterotrimeric fusion polypeptide.

In some embodiments of an RER heterotrimeric fusion polypeptide sequence, the N-terminus of W is covalently joined to the C-terminus of another element directly or indirectly (e.g., via a covalent linker). For example, in formula I(b), II(b), III(b), I(c), II(c), or III(c), the N-terminus of W is covalently joined to the C-terminus of B directly or via a linker L¹.

In some embodiments of the RER heterotrimeric fusion polypeptide sequence, the C-terminus of Y is covalently joined to the N-terminus of another element directly or indirectly (e.g., via a covalent linker). For example, in formula I(a), II(a), or III(a), the C-terminus of Y is covalently joined to the N-terminus of B directly or via a linker L¹.

In some embodiments, the amino acid sequence of W is identical to the amino acid sequence of Y. In some embodiments, the amino acid sequence of W is different than the amino acid sequence of Y.

### Exemplary W and Y Sequences

In some embodiments of an RER heterotrimeric fusion polypeptide of formula I, W and/or Y comprises any of the amino acid sequence extending from residues 22 to 139 of SEQ ID NO: 5, 520 to 631 of SEQ ID NO: 5, 1 to 118 of SEQ ID NO: 9, 479 to 590 of SEQ ID NO: 9, 1 to 118 of SEQ ID NO: 48, 499 to 610 of SEQ ID NO: 48, 1 to 118 of SEQ ID NO: 49, 499 to 610 of SEQ ID NO: 49, 1 to 120 of SEQ ID NO: 50, 501 to 612 of SEQ ID NO: 50, 1 to 120 of SEQ ID NO: 51, 501 to 612 of SEQ ID NO: 51, 1 to 120 of SEQ ID NO: 52, or 510 to 621 of SEQ ID NO: 52.

In some embodiments of an RER heterotrimeric fusion polypeptide of formula II, W and/or Y comprises the amino acid sequence extending from residues 22 to 139 of SEQ ID NO: 5, 520 to 631 of SEQ ID NO: 5, 1 to 118 of SEQ ID NO: 9, 479 to 590 of SEQ ID NO: 9, 1 to 118 of SEQ ID NO: 48, 499 to 610 of SEQ ID NO: 48, 1 to 118 of SEQ ID NO: 49, 499 to 610 of SEQ ID NO: 49, 501 to 612 of SEQ ID NO: 50, 501 to 612 of SEQ ID NO: 51, or 510 to 621 of SEQ ID NO: 52.

In some embodiments of an RER heterotrimeric fusion polypeptide of formula II, W and/or of Y does not comprise any of the sequences extending from residues 22 to 139 of SEQ ID NO: 5, 520 to 631 of SEQ ID NO: 5, 1 to 118 of SEQ ID NO: 9, 479 to 590 of SEQ ID NO: 9, 1 to 118 of SEQ ID NO: 48, 499 to 610 of SEQ ID NO: 48, 1 to 118 of SEQ ID NO: 49, 499 to 610 of SEQ ID NO: 49, 501 to 612 of SEQ ID NO: 50, 501 to 612 of SEQ ID NO: 51, or 510 to 621 of SEQ ID NO: 52.

In some embodiments of an RER heterotrimeric fusion polypeptide of formula III, W and/or Y comprises the amino acid sequence extending from residues 1 to 120 of SEQ ID NO: 50, 1 to 120 of SEQ ID NO: 51, or 1 to 120 of SEQ ID NO: 52.

### Exemplary X Sequences

In some embodiments of an RER heterotrimeric fusion polypeptide of formula I, X comprises any of the amino acid sequences extending from residues 157 to 517 of SEQ ID NO: 5, 119 to 478 of SEQ ID NO: 9, 136 to 496 of SEQ ID NO: 48, 136 to 496 of SEQ ID NO: 49, 138 to 500 of SEQ ID NO: 50, 138 to 500 of SEQ ID NO: 51, or 147 to 509 of SEQ ID NO: 52.

In some embodiments of an RER heterotrimeric fusion polypeptide of formula II, X comprises the amino acid sequence extending from residues 157 to 517 of SEQ ID NO: 5, 136 to 496 of SEQ ID NO: 48, or 136 to 496 of SEQ ID NO: 49.

In some embodiments of an RER heterotrimeric fusion polypeptide of formula II, X does not comprise any of the sequences extending from residues 157 to 517 of SEQ ID NO: 5, 136 to 496 of SEQ ID NO: 48, or 136 to 496 of SEQ ID NO: 49.

In some embodiments of an RER heterotrimeric fusion polypeptide of formula III, X comprises the amino acid sequence extending from residues 119 to 478 of SEQ ID NO: 9, 138 to 500 of SEQ ID NO: 50, 138 to 500 of SEQ ID NO: 51, or 147 to 509 of SEQ ID NO: 52.

Exemplary L³ and L⁴ Linker Sequences

In some embodiments, L³ comprises an amino acid sequence of SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, or SEQ ID NO: 61.

In some embodiments, L⁴ comprises an amino acid sequence of SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, or SEQ ID NO: 61.

### Exemplary RER Heterotrimeric Fusion Polypeptide Sequences

In some embodiments, the RER heterotrimeric fusion polypeptide has an amino acid sequence comprising the amino acid sequence of SEQ ID NO: 9, SEQ ID NO: 48, SEQ ID NO: 49, SEQ ID NO: 50, SEQ ID NO: 51, or SEQ ID NO: 52.

In some embodiments, the RER heterotrimeric fusion polypeptide has an amino acid sequence comprising the amino acid sequence of SEQ ID NO: 9, SEQ ID NO: 49, SEQ ID NO: 50, SEQ ID NO: 51, or SEQ ID NO: 52.

In some embodiments, the RER heterotrimeric fusion polypeptide has an amino acid sequence that does not comprise any of the amino acid sequence of SEQ ID NO: 48.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject (e.g., a human subject, such as a human subject suffering from a disease or condition associated with elevated TGF-β activity, such as a decrease in muscle function, a skeletal disorder with elevated bone turnover (e.g., osteogenesis imperfecta), or a muscle disorder (e.g., a muscular dystrophy), as described herein.

As used herein, the term “scFv” refers to a single chain Fv antibody in which the variable domains of the heavy chain and the light chain from an antibody have been joined to form one chain. scFv fragments contain a single polypeptide chain that includes the variable region of an antibody light chain (V_{L}) (e.g., CDR-L1, CDR-L2, and/or CDR-L3) and the variable region of an antibody heavy chain (V_{H}) (e.g., CDR-H1, CDR-H2, and/or CDR-H3) separated by a linker. The linker that joins the V_{L} and V_{H} regions of a scFv fragment can be a peptide linker composed of proteinogenic amino acids. Alternative linkers can be used to so as to increase the resistance of the scFv fragment to proteolytic degradation (for example, linkers containing D-amino acids), in order to enhance the solubility of the scFv fragment (for example, hydrophilic linkers such as polyethylene glycol-containing linkers or polypeptides containing repeating glycine and serine residues), to improve the biophysical stability of the molecule (for example, a linker containing cysteine residues that form intramolecular or intermolecular disulfide bonds), or to attenuate the immunogenicity of the scFv fragment (for example, linkers containing glycosylation sites). It will also be understood by one of ordinary skill in the art that the variable regions of the scFv molecules described herein can be modified such that they vary in amino acid sequence from the antibody molecule from which they were derived. For example, nucleotide or amino acid substitutions leading to conservative substitutions or changes at amino acid residues can be made (e.g., in CDR and/or framework residues) so as to preserve or enhance the ability of the scFv to bind to the antigen recognized by the corresponding antibody.

As used herein, the phrases “specifically binds” and “binds” refer to a binding reaction which is determinative of the presence of a particular protein, mineral, or other particular compound in a heterogeneous population of proteins and other biological molecules that is recognized, e.g., by a ligand with particularity. A ligand (e.g., a protein, peptide, or small molecule) that specifically binds to a protein or mineral will bind to the protein or mineral, e.g., with a K_{D} of less than 100 μM. For example, a peptide (e.g., a TGF-β trapping peptide, a TGF-β-binding peptide, a collagen-binding peptide, or a hydroxyapatite-binding peptide) that specifically binds to a protein (e.g., TGF-β) or mineral (e.g., hydroxyapatite) may bind to the protein or mineral with a K_{D} of up to 1 μM (e.g., between 1 pM and 1 μM). A variety of assay formats may be used to determine the affinity of a ligand (e.g., a peptide, such as a TGF-β trapping peptide, a TGF-β-binding peptide, a collagen-binding peptide, or hydroxyapatite-binding peptide) for a specific protein (e.g., TGF-β or collagen) or mineral (e.g., hydroxyapatite). For example, solid-phase ELISA assays are routinely used to identify ligands that specifically bind a particular protein. See, e.g., Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of assay formats and conditions that can be used to determine specific protein binding.

As used herein, the terms “subject” and “patient” are interchangeable and refer to an organism that receives treatment for a particular disease or condition as described herein. Examples of subjects and patients include mammals, such as humans, receiving treatment for diseases or conditions, such as conditions associated with elevated TGF-β activity, such a skeletal disorder with elevated bone turnover (e.g. osteogenesis imperfecta) or a muscle disorder (e.g., a muscular dystrophy).

As used herein, the term “targeting linker” refers to a polypeptide that is covalently bound to a bone-targeting moiety.

As used herein, the term “targeting moiety” refers to a compound, such as a peptide, that specifically binds an endogenous component that is expressed in a particular tissue type. For instance, bone-targeting moieties described herein contain a compound, such as a peptide, that specifically binds to an endogenous component of osseous tissue. In the context of bone-targeting moieties, the endogenous component of osseous tissue may be, for example, a protein, such as collagen, or a mineral, such as hydroxyapatite. Thus, in the context of bone-targeting moieties, the moiety may be a collagen-binding domain or peptide or a bone-targeting hydroxyapatite-binding domain or peptide. Additionally, in the context of bone-targeting moieties, moieties described herein may be a polyanionic peptide, a bisphosphonate, or the amino acid sequence of SEQ ID NO: 46, or a variant of said amino acid sequence. Examples of bone-targeting moieties are provided throughout the specification, for example, in the section labeled “Bone-targeting Moieties.” Due to their specific binding affinity, targeting moieties can be capable of localizing a compound of interest, such as a TGF-β antagonist, to a particular tissue of interest, such as bone.

As used herein, the term “TGF-β antagonist” refers to a compound (e.g., a peptide) capable of inhibiting TGF-β signaling. A TGF-β antagonist may contain a peptide and, optionally, one or more non-peptidic molecules. A TGF-β antagonist may contain, consist of, or consist essentially of a TGF-β-binding peptide, which refers to a peptide capable of binding TGF-β. TGF-β antagonists useful in conjunction with the compositions and methods described herein include TGF-β receptors and fusion proteins thereof, such as those that contain one or more domains of TGF-β receptor II (e.g., one or more TGF-β receptor II ectodomain peptides) bound to one or more domains of TGF-β receptor III (e.g., a TGF-β receptor III ectodomain peptide). As used herein, the terms “TGF-β receptor fusion protein,” “RER fusion protein” and “TGF-β RER fusion conjugate” all refer to TGF-β receptors and fusion proteins thereof, as described herein. A TGF-β antagonist may contain a composition capable of inhibiting TGF-β signaling. A TGF-β antagonist may be a pan-TGF-β antagonist, such as 1D11 or its humanized version, Fresolimumab, or it may contain, consist of, or consist essentially of a TGF-β RER fusion conjugate capable of trapping TGF-β, i.e., a TGF-β trap. Trapping of TGF-β can be assessed, for instance, using a protein binding assay known in the art, such as ELISA, fluorescence anisotropy or fluorescence polarization, and calorimetry, such as isothermal titration calorimetry (ITC). Trapping of TGF-β can also be assessed by observing a decrease in TGF-β signaling. Binding of a peptide to TGF-β can be determined, for example, by observing peptide-mediated inhibition of TGF-β induced, Smad3-driven transcription. This can be measured, for example, using an in vitro reporter expression assay, such as an in vitro luciferase expression assay described herein. Binding of a peptide to TGF-β can be determined by measuring, for example, peptide-mediated inhibition of TGF-β induced, Smad3-driven expression of the reporter gene (e.g., luciferase) by from about 10% to about 75%, or more, such as from about 15% to about 70%, 20% to about 65%, 25% to about 60%, 30% to about 55%, or 35% to about 50%, e.g., relative to an untreated sample, for instance, as assessed by measuring the decrease in activity of a protein encoded by the reporter gene (e.g., luciferase activity in a luciferase reporter assay as known in the art or described herein). Trapping of TGF-β can be determined, for example, by observing antagonist-mediated inhibition of TGF-β-induced expression of a protein that is normally expressed as a result of TGF-β signal transduction, such as fibronectin, α-smooth muscle actin (α-SMA), Snail, and/or Slug. This can be measured, for example, using a cell-based immunoblot assay (e.g., as measured in squamous cell carcinoma A431 cells, for instance, as described herein). Trapping of TGF-β can also be determined by measuring, for example, antagonist-mediated inhibition of TGF-β-induced expression of fibronectin, α-SMA, Snail, and/or Slug by about 25% to about 75%, or more, e.g., relative to an untreated sample, such as about 25%, 30%, 35%, 40%, 45%, 50%, 55%, 60%, 65%, 70%, 75%, or more, for instance, as measured by densitometry analysis of a developed immunoblot as known in the art or described herein. Trapping of TGF-β can also be determined, for example, by observing antagonist-mediated inhibition of TGF-β-induced cancer cell invasion and metastasis (e.g., TGF-β-induced invasion of carcinoma cells, such as human squamous cell carcinoma cells), for instance, as assessed by a cancer cell invasion assay described herein. For instance, trapping of TGF-β can be measured by observing peptide-mediated reduction in cancer cell proliferation, for instance, as assessed by analysis of tumorigenicity and stem cell marker expression using techniques known in the art or described herein, and/or cancer cell migration (e.g., squamous cell carcinoma A431 cell migration, for instance, as measured using an in vitro wound closure assay). Trapping of TGF-β can be determined by measuring, for example, peptide-mediated attenuation of cancer cell migration (e.g., squamous cell carcinoma A431 cell migration) such that from about 20% to about 40%, or less, of a wound inflicted upon the cultured cancer cells has closed, for instance, after about 24 hours of co-incubation of the cancer cells in the presence of TGF-β and the TGF-β trap. For instance, binding of a TGF-β trap to TGF-β isoforms can be observed by detecting a reduction in TGF-β-induced cancer cell migration (e.g., squamous cell carcinoma A431 cell migration) such that about 40%, 45%, 40%, 35%, 30%, 25%, 20%, 15%, 10%, 5%, 4%, 3%, 2%, 1%, or less, of a wound inflicted upon the cultured cancer cells has closed, for instance, after about 24 hours of co-incubation of the cancer cells in the presence of TGF-β and the TGF-β antagonist. Trapping of TGF-β can also be observed, for instance, by detecting an antagonist-mediated decrease in the expression of fibronectin, plasminogen activator inhibitor-1 (PAI-1), and/or connective tissue growth factor (CTGF) in a cell-based immunoblot assay. For example, Trapping of TGF-β can be observed by detecting peptide-mediated inhibition of the expression (e.g., the TGF-β-induced expression) of one or more proteins involved in the epithelial-mesenchymal transition (EMT), such as E-cadherin, Twist, Snail, Slug, and α-smooth muscle actin (SMA). For instance, Trapping of TGF-β can be observed by detecting peptide-mediated inhibition of TGF-β-induced fibrosis and/or EMT such that expression of fibronectin, PAI-1, and/or GTGF is reduced by from about 15% to about 50%, or more, e.g., relative to an untreated sample, such as by about 15%, 20%, 25%, 30%, 35%, 40%, 45%, 50%, or more, for example, as measured by densitometry analysis of a developed immunoblot as known in the art or described herein. In addition, changes in the levels of all the aforementioned proteins which are indicative of a change in TGF-β activity may be ascertained using RT-PCR or any other method of measuring levels of transcription or translation.

As used herein, the term “TGF-β antagonist binding affinity” refers to the binding affinity of a TGF-β antagonist of the invention, such as a TGF-β receptor fusion protein, to any of the TGF-β isoforms. The binding of the TGF-β antagonists to the TGF-β isoforms can be measured in vitro by K_{D}, EC₅₀, or IC₅₀ values, for example, using an assay known in the art, e.g., by measurements in IL-11 release assay for TGF-β neutralization by the TGF-β antagonist described herein.

As used herein, the term “TGF-β isoforms” describes homodimeric polypeptides β1, β2, and β3 that bind to specific types of TGF-β receptors.

As used herein, the term “TGF-β receptor fusion protein” refers to a conjugate containing a TGF-β receptor, or a portion, domain, or variant thereof, bound to another TGF-β receptor, or a portion, domain, or variant thereof. This may also be referred to as “RER heterotrimeric fusion polypeptide” or simply “RER” as described above. Exemplary TGF-β receptor fusion proteins useful in conjunction with the compositions and methods described herein include conjugates containing TGF-β receptor II, or a portion, domain, or variant thereof, bound to TGF-β receptor III, or a portion, domain, or variant thereof. For instance, TGF-β receptor fusion proteins described herein include conjugates that contain a TGF-β receptor II ectodomain, or a portion, domain, or variant thereof, bound to a TGF-β receptor III endoglin domain, or a portion, domain, or variant thereof. TGF-β receptor fusion proteins include conjugates in which a plurality of TGF-β receptors, or fragments, domains, or variants thereof, are each bound to a single TGF-β receptor, or portion, domain, or fragment thereof, such as conjugates that contain two TGF-β receptor II ectodomains independently bound to different sites on a TGF-β receptor III endoglin domain. FIGS. 33A-C illustrate the three different formulas of the TGF-β receptor fusion protein or RER heterotrimeric fusion polypeptide.

As used herein, the term “TGF-β receptor Type II” describes a receptor that, on binding with a ligand in the TGF-β superfamily, forms a receptor complex consisting of two type II and two type I transmembrane serine/threonine kinases. Type-2 receptors phosphorylate and activate type I receptors which autophosphorylate, then bind and activate SMAD transcriptional regulators. The term “TGF-β receptor Type II” is used interchangeably with “TGF-β receptor II.”

As used herein, the term “TGF-β receptor Type III” or betaglycan describes a receptor that has two TGF-β binding sites in its extracellular domain, which are called the E and U domains, and has 200 to 300-fold greater affinity for binding TGF-β isoform 2 than does TGF-β receptor Type II. The term “TGF-β receptor Type III” is used interchangeably with “TGF-β receptor III.”

As used herein, the term “TGF-β signaling” refers to the endogenous signal transduction cascade by which TGF-β potentiates the intracellular activity of the TGF-β receptor so as to effect one or more biological responses. TGF-β signaling encompasses the TGF-β-mediated stimulation of a TGF-β receptor and concomitant phosphorylation and activation of receptor-associated Smad proteins. TGF-β signaling includes the translocation of one or more Smad transcription factors to the nucleus, for example, by way of an interaction between a Smad protein and nucleoporins. TGF-β signaling encompasses the release of one or more Smad protein from Smad Anchor for Receptor Activation (SARA), which sequesters Smad proteins in the cytoplasm and prevents their translocation into the nucleus. Methods for assessing TGF-β signaling are known in the art and is described under the definition of the term “elevated TGF-β activity.”

As used herein, the term “therapeutically effective amount” of a therapeutic agent, such as a conjugate described herein, refers to an amount that is sufficient, when administered to a subject suffering from or susceptible to a disease, disorder, and/or condition, (e.g., a disease, disorder, and/or condition associated with elevated TGF-β signaling or activity, such as a muscle disorder, and/or a skeletal disorder associated with bone turnover as described herein, such as osteogenesis imperfecta, to improve, treat, prevent, stop the progression of, and/or delay the onset of one or more symptom(s) of the disease, disorder, and/or condition. For instance, exemplary therapeutically effective amount of a therapeutic agent is an amount that is sufficient to restore, improve, treat, prevent, stop the progression of, and/or delay the onset of a decrease in muscle function in a subject suffering from a disease, disorder, and/or condition associated with elevated TGF-β activity, such as a muscle disorder (such as a muscular dystrophy) and/or a skeletal disorder (such as osteogenesis imperfecta), as described herein.

As used herein, the terms “treat” or “treatment” in the context of a subject at risk for or suffering from a disease or condition associated with elevated TGF-β activity, such as a skeletal disorder, a muscle disorder, a musculoskeletal disease, and/or bone turnover, refer to treatment, for instance, by contacting with or administering to a patient a conjugate containing a TGF-β antagonist and optionally, a bone-targeting moiety as described herein, with the intention of alleviating a phenotype associated with the disease or condition (e.g., a decrease in muscle function). For instance, exemplary forms of treatment include administration of a conjugate, such as a conjugate described herein, to a subject suffering from a skeletal disorder associated with elevated TGF-β signaling, such as osteogenesis imperfecta (e.g., osteogenesis imperfecta of Types I-XI), or a muscle disorder, such as a muscular dystrophy, so as to reduce the progression of the disease or attenuate the severity of one or more symptoms associated with the disease, such as the propensity of the subject to have decreased muscle function or to suffer from recurring bone fractures in the case of bone diseases. Treatment may also include improvement of muscle weakness in a patient suffering from a symptom of weakened muscle that results, at least in part, from excessive TGF-β signaling, including at the site of bone. A patients suffering from such disorders may be considered treated if the patient exhibits, for instance, a reduced progression of the disease or an attenuated severity of one or more symptoms associated with the disease, such as the propensity of the patient to have decreased muscle function or to suffer from recurring bone fractures (e.g., within one or more days, weeks, months, or years of administration of the conjugate to the patient). Among patients suffering from a muscle disease, such as a muscular dystrophy (e.g., Duchenne muscular dystrophy), a patient may be considered to be treated if the patient exhibits an improvement in muscle strength, muscle quality, muscle mass, and/or general functional status following administration of the conjugate to the patient (e.g., within one or more days, weeks, months, or years of administration of the conjugate to the patient).

As used herein, a “variant” of a polypeptide contains one or more amino acid substitutions, deletions, and/or additions as compared to the parent polypeptide. Exemplary variants of the polypeptides described herein have an amino acid sequence that is at least 70% identical (e.g., at least 75%, 80%, 85%, 90%, 95%, 97%, 98%, 99%, or 100%) identical to the amino acid sequence of the parent polypeptide. Exemplary variants of the polypeptides described herein may have preserved or improved properties as compared to the parent polypeptide. For instance, certain changes to the amino acid sequence of a parent peptide may not significantly alter the structure and/or activity of the parent polypeptide. Conservative amino acid substitutions represent one example of a type of change in the amino acid sequence of a parent polypeptide that may not alter the overall tertiary structure and/or activity of the polypeptide. As shown in Table 1, above, conservative amino acid substitutions involve changing one amino acid to another that has a side-chain that exhibits similar physicochemical properties. Additional examples of variants described herein include those that have small deletions, typically of from 1 to about 30 amino acids, relative to the amino acid sequence of a parent polypeptide, as well as variants that feature small amino- or carboxy-terminal extensions, such as an amino-terminal methionine residue, a small linker peptide of up to about 25 residues, or a small extension that facilitates purification, such as an affinity tag. Exemplary affinity tags include, for instance, a poly-histidine tract, protein A, glutathione S-transferase, and various other domains, such as those described in Ford et al., Protein Expression and Purification 1991; 2:95-107, the disclosure of which is incorporated herein by reference as it pertains to affinity tags for protein purification.

As used herein, the term “vector” includes nucleic acid vectors, such as a plasmid, a DNA vector, a plasmid, a RNA vector, virus, and other suitable replicons. Expression vectors described herein may contain a polynucleotide sequence as well as, for example, additional sequence elements used for the expression of proteins and/or the integration of these polynucleotide sequences into the genome of a cell. Certain vectors that can be used for the expression of fusion proteins include plasmids that contain regulatory sequences, such as promoter and enhancer regions, which direct gene transcription. Other useful vectors for expression of fusion proteins contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector.

### BRIEF DESCRIPTION OF THE DRAWINGS

FIG. 1: Sequence of Albumin Signal Peptide (SEQ ID NO: 4) and PCT-0015 (SEQ ID NO: 14)

FIG. 2: Expression vector pD2539dg RER-Fc

FIG. 3: Purification of PCT-0015 (SEQ ID NO: 14) on Protein A Sepharose

FIG. 4: Coomassie gel stain of PCT-0015 (SEQ ID NO: 14) purification from 150 ml culture (Pool 3). From Left to Right: Load, Flow Through, Wash, E1, E2, E3, Markers MW indicated in kDa

FIG. 5A: Size exclusion chromatography (SEC) of PCT-0015 (SEQ ID NO: 14) material purified by affinity chromatography on Superose 6 column

FIG. 5B: SEC-HPLC analysis of PCT-0015 (SEQ ID NO: 14)

FIG. 6: SDS-PAGE analysis of PCT-0015 (SEQ ID NO: 14) fractions from the size exclusion chromatography column (non-reducing conditions)

FIG. 7: SDS-PAGE SDS-PAGE analysis of PCT-0015 (SEQ ID NO: 14) fractions from the size exclusion chromatography column (reducing conditions)

FIG. 8: SPR analysis of controls: binding of PCT-0015 (SEQ ID NO: 14) fractions from SEC-HPLC to TGF-β1 surface

FIG. 9: SPR analysis of controls: binding of PCT-0015 fractions from SEC-HPLC to TGF-β3 surface

FIG. 10: SPR analysis of controls: binding of PCT-0015 fractions from SEC-HPLC to TGF-β2 surface

FIG. 11: TGF-β1 neutralization assay of selected SEC fractions of PCT-0015 (SEQ ID NO: 14)

FIG. 12: TGF-β3 neutralization assay of selected SEC fractions of PCT-0015 (SEQ ID NO: 14)

FIG. 13: TGF-β2 neutralization assay of selected SEC fractions of PCT-0015 (SEQ ID NO: 14)

FIG. 14: Sec-HPLC of PCT-0016NT (SEQ ID NO: 33)

FIG. 15: TGF-β1 neutralization assay of selected SEC fractions of PCT-0016NT (SEQ ID NO: 33)

FIG. 16: TGF-β3 neutralization assay of selected SEC fractions of PCT-0016NT (SEQ ID NO: 33)

FIG. 17: TGF-β2 neutralization assay of selected SEC fractions of PCT-0016NT (SEQ ID NO: 33)

FIG. 18: SEC-HPLC of PCT-0017 (SEQ ID NO: 32)

FIG. 19: SEC-HPLC of PCT-0018 (SEQ ID NO: 34)

FIG. 20: SEC-HPLC of PCT-0019 (SEQ ID NO: 16)

FIG. 21: SEC-HPLC of PCT-0020 (SEQ ID NO: 18)

FIG. 22: SEC-HPLC of PCT-0021 (SEQ ID NO: 20)

FIG. 23: Purification of PCT-0021 (SEQ ID NO: 20) by SEC chromatography on Superose 6.

FIG. 24: SDS-PAGE analysis of PCT-0021 (SEQ ID NO: 20) selected fractions from the SEC column under reducing and non-reducing conditions

FIG. 25: SEC-HPLC of PCT-0022 (SEQ ID NO: 22)

FIG. 26: Preparative SEC chromatography of PCT-0022 (SEQ ID NO: 22)

FIG. 27: SDS-PAGE analysis of PCT-0022 (SEQ ID NO: 22) selected fractions from the SEC column under reducing and non-reducing conditions

FIG. 28A: Neutralization data for PCT-0015 (SEQ ID NO: 14) and PCT-0016NT (SEQ ID NO: 33) for TGF-β1

FIG. 28B: Neutralization data for PCT-0015 (SEQ ID NO: 14) and PCT-0016NT (SEQ ID NO: 33) for TGF-β1

FIG. 29A: PCT-0020 (SEQ ID NO: 18) compared to PCT-0016NT (SEQ ID NO: 33) in neutralization of TGF-β1

FIG. 29B: PCT-0020 (SEQ ID NO: 18) compared to PCT-0016NT (SEQ ID NO: 33) in neutralization of TGF-β3

FIG. 29C: PCT-0020 (SEQ ID NO: 18) compared to PCT-0016NT (SEQ ID NO: 33) in neutralization of TGF-β2

FIG. 30A: PCT-0021 (SEQ ID NO: 20) compared to PCT-0022 (SEQ ID NO: 22) in neutralization of TGF-β1

FIG. 30B: PCT-0021 (SEQ ID NO: 20) compared to PCT-0022 (SEQ ID NO: 22) in neutralization of TGF-β2

FIG. 30C: PCT-0021 (SEQ ID NO: 20) compared to PCT-0022 (SEQ ID NO: 22) in neutralization of TGF-β3

FIG. 31: Illustration of ELISA capture method for assessment of TGF-β induced IL11 release

FIG. 32: Prediction sequence for signal peptide cleavage site

FIG. 33A: Formula a (Option 1) corresponding to SEQ ID NO: 14, 16, 18, 20, 22, 24, 26, 28, 30

FIG. 33B: Formula b (Option 2, version 1) corresponding to SEQ ID NO: 17

FIG. 33C: Formula c (Option 2, version 2) corresponding to SEQ ID NO: 18

FIG. 34: Neutralization of TGF-β1, TGF-β2, and TGF-β3 by PCT-0026 (SEQ ID NO: 30) compared to PCT-0020 (SEQ ID NO: 18) and 1D11 antibody

FIG. 35: Whole body positron emission tomography (PET) imaging of mice injected intraperitoneally with radiolabeled PCT-0026 (SEQ ID NO: 30) across 7-day experiment with analysis focused on long bone (femur)

FIG. 36A: Accumulation of Zn89-labeled PCT-0026 (SEQ ID NO: 30) in serum within the first 48 hours of study

FIG. 36B: Accumulation of Zn89-labeled PCT-0026 (SEQ ID NO: 30) in isolated femur (bone) within the first 48 hours of study

FIG. 37: RT-PCR of representative TGF-β responsive genes in OIM and WT bones

FIG. 38: The forelimb grip strength test is used to assess muscle strength in mice

FIG. 39: Grip strength in OIM mice and wild-type mice at 4 weeks and 16 weeks

FIG. 40: Immunostaining confirms presence of PCT-0011 in tibial bone of mouse treated with PCT-0011

FIG. 41: Details of treatment schedule of WT and OIM mice to assess effect of TGF-β neutralization on mobility and muscle strength

FIG. 42: Open field test with digital image processor used to measure mouse mobility

FIGS. 43A, 43B, and 43C: Mobility assessments of OIM mice treated with non-targeted TGF-β antagonist. Individual mice were assessed in an open field test apparatus over a 20-minute period. FIG. 43A. Distance traveled, FIG. 43B. Total activity, FIG. 43C. Mean Speed

FIGS. 44A, 44B, and 44C: Mobility assessments of OIM mice treated with bone-targeted TGF-β antagonist. Individual mice were assessed in an open field test apparatus over a 20-minute period. FIG. 44A. Distance traveled, FIG. 44B. Total activity, FIG. 44C. Mean Speed.

FIG. 45: Forelimb Grip Strength in mice treated with non-targeted TGF-β antagonist

FIG. 46: Forelimb Grip Strength in mice treated with bone-targeted TGF-β antagonist

### DETAILED DESCRIPTION

The invention features therapeutic conjugates, such as those that contain transforming growth factor-β (TGF-β) antagonists, including those bound to a bone-targeting moiety that localizes the antagonist to human bone tissue. Also included are TGF-β antagonists that may be used in the absence of a bone-targeting moiety to treat other conditions where lowered TGF-β biological activity is desired. TGF-β antagonists that may be used in conjunction with the compositions and methods described herein include TGF-β receptors, as well as domains and variants thereof. Additionally, TGF-β antagonists useful in the context of the compositions and methods described herein include TGF-β receptor fusion proteins, such as those that contain one or more TGF-β receptor II domains, fragments, or variants thereof bound to one or more TGF-β receptor III domains, fragments, or variants thereof. For instance, fusion proteins that may be used in conjunction with the compositions and methods described herein include those that contain one or more ectodomains of TGF-β receptor II, such as human or rat TGF-β receptor II, bound to one or more endoglin domains of TGF-β receptor III, such as human or rat TGF-β receptor III. In some embodiments, the TGF-β antagonist is a TGF-β receptor fusion protein that contains a TGF-β receptor II ectodomain bound to a TGF-β receptor III endoglin domain, such as a fusion protein in which two TGF-β receptor II ectodomain molecules are each independently bound to a single TGF-β receptor III endoglin domain molecule. As described herein, the component TGF-β receptors or domains, fragments, or variants thereof of a TGF-β receptor fusion protein may be bound to one another directly, for instance, by way of an amide bond between each component polypeptide, or indirectly by way of a linker. Similarly, the fusion proteins may be bound to a targeting moiety directly, for instance, by way of an amide bond, or indirectly by way of an Fc domain of an immunoglobulin.

In addition to a TGF-β antagonist, conjugates useful in conjunction with the compositions and methods described herein may contain a targeting moiety bound to the TGF-β antagonist, such as a polyanionic peptide capable of binding a mineral present in bone tissue, such as hydroxyapatite. In this way, the TGF-β antagonist can be administered to a patient, such as a human patient suffering from a disease associated with elevated osseous TGF-β signaling or heightened bone turnover, and may subsequently localize to bone tissue. The invention is based in part on the discovery that this site-selective localization of TGF-β antagonists, such as TGF-β receptor fusion proteins, to bone tissue promotes the attenuation of TGF-β signaling specifically at the site of damaged bone, while preserving TGF-β activity in healthy tissues. Administration of the conjugates described herein represents a useful therapeutic strategy for treating, for instance, disorders associated with heightened TGF-β-mediated osteoclast activity relative to osteoblast activity, such as osteogenesis imperfecta, which is characterized by elevated bone resorption due to the activity of osteoclasts induced by overactive TGF-β signal transduction. Additionally, the conjugates described herein can be used to treat muscular dystrophies associated with elevated TGF-β signaling. This beneficial activity is due, at least in part, to the ability of the conjugates to suppress TGF-β activity selectively at the skeletal-muscular interface, thus restoring muscle function and preserving TGF-β activity in healthy tissues.

The following sections describe, in further detail, various TGF-β antagonists, targeting moieties, and linkers that can be used to prepare exemplary conjugates, as well as methods of producing such agents and methods of using the same for the treatment of disorders characterized by elevated TGF-β signaling in osseous tissue.

### TGF-β Antagonists

### TGF-β Receptors and TGF-β Receptor Fusion Proteins

TGF-β antagonists that can be used in conjunction with the compositions and methods described herein include TGF-β receptors, as well as domains, fragments, and variants thereof. TGF-β receptors, such as TGF-β receptors I, II, and III, are capable of binding TGF-β isoforms with varying selectivity profiles. By binding TGF-β, exogenous receptors administered to a patient, such as a human patient suffering from a skeletal or muscular disease described herein, can sequester TGF-β and prevent it from engaging its endogenous TGF-β receptor target. In this way, soluble TGF-β receptors, and fusion proteins containing these molecules, can inhibit the activation of the TGF-β signal transduction pathway. This inhibition of TGF-β activity can have important therapeutic phenotypes, particularly at the site of osseous tissue in patients suffering from a disorder characterized by elevated TGF-β-mediated bone turnover, such as osteogenesis imperfecta, and at the skeletal-muscular interface in patients suffering from muscular dystrophies.

### TGF-β Isoforms and Endogenous Receptors

TGF-β isoforms (β1, β2, and β3) are homodimeric polypeptides of about 25 kDa. These isoforms are secreted in a latent form and only a small percentage of total secreted TGF-β isoforms are activated under physiological conditions. TGF-β binds to three different cell surface receptors called type I (RI, also referred to herein as TGF-β receptor I) type II (RII, also referred to herein as TGF-β receptor II), and type III (RIII, also referred to herein as TGF-β receptor III).

RI and RII are serine/threonine kinase receptors. RIII has two TGF-β binding sites in its extracellular domain, referred to as the endoglin and uromodulin domains of TGF-β receptor III. TGF-β1 and TGF-β3 bind RII with an affinity that is 200-300 fold higher than TGF-β2 (Baardsnes et al., Biochemistry, 48, 2146-55, 2009); accordingly, cells deficient in RIII are 200- to 300-fold less responsive to equivalent concentrations of TGF-β2 compared to TGF-β1 and TGF-β-3 (Chiefetz, et al (1990) J. Bio. Chem., 265, 20533-20538). However, in the presence of RIII, cells respond roughly equally to all three TGF-β isoforms, consistent with reports that show that RIII can sequester and present the ligand to RII to augment TGF-β activity when it is membrane-bound (Chen et al., J. Biol. Chem. 272, 12862-12867, 1997; Lopez-Casillas et al., Cell 73, 1435-1444, 1993; Wang et al., Cell 67, 797-805, 1991; Fukushima et al., J. Biol. Chem. 268, 22710-22715, 1993; Lopez-Casillas et al., J. Cell Biol. 124, 557-568, 1994). Binding of TGF-β to RII recruits and activates RI through phosphorylation (Wrana et al., Nature 370, 341-347, 1994). The activated RI phosphorylates intracellular Smad2 and Smad3, which then interact with Smad4 to regulate gene expression in the nucleus (Piek et al., FASEB J. 13, 2105-2124, 1999; Massague and Chen, Genes & Development 14, 627-644, 2000). Through its regulation of gene expression, TGF-β has been shown to influence many cellular functions, including bone turnover and osteoclast-mediated bone resorption.

### TGF-β Receptors as Inhibitors of TGF-β Signaling

Due in part to their ability to bind TGF-β and sequester this ligand from its endogenous receptor, exogenous TGF-β receptors and domains, fragments, and variants thereof can be used to inhibit TGF-β signaling, such as at the site of osseous tissue and at the skeletal-muscular interface. Exemplary TGF-β receptor domains that are useful in conjunction with the compositions and methods described herein include TGF-β receptor II and III domains, such as the TGF-β receptor II ectodomain and TGF-β receptor III endoglin domain. The TGF-β receptor II ectodomain binds TGF-β in a 1:1 stoichiometric ratio, while two molecules of TGF-β are bound by a single molecule of the TGF-β receptor III ectodomain. The amino acid sequences of various human and rat TGF-β receptors are shown in Table 2, below.

TABLE 2
Amino acid sequences of various human
and rat TGF-β receptors

| SEQ | | |
|---|---|---|
| ID | TGF-β | |
| NO. | Receptor | Amino Acid Sequence |
| 1 | Full-length | MGRGLLRGLWPLHIVLWTRIASTIPPHVQKSVN |
| | human | NDMIVTDNNGAVKFPQLCKFCDVRFSTCDNQK |
| | TGF-β | SCMSNCSITSICEKPQEVCVAVWRKNDENITLE |
| | receptor II | TVCHDPKLPYHDFILEDAASPKCIMKEKKKPGE |
| | | TFFMCSCSSDECNDNIIFSEEYNTSNPDLLLVIF |
| | | QVTGISLLPPLGVAISVIIIFYCYRVNRQQKLSST |
| | | WETGKTRKLMEFSEHCAIILEDDRSDISSTCAN |
| | | NINHNTELLPIELDTLVGKGRFAEVYKAKLKQNT |
| | | SEQFETVAVKIFPYEEYASWKTEKDIFSDINLK |
| | | HENILQFLTAEERKTELGKQYWLITAFHAKGNL |
| | | QEYLTRHVISWEDLRKLGSSLARGIAHLHSDHT |
| | | PCGRPKMPIVHRDLKSSNILVKNDLTCCLCDFG |
| | | LSLRLDPTLSVDDLANSGQVGTARYMAPEVLE |
| | | SRMNLENVESFKQTDVYSMALVLWEMTSRCN |
| | | AVGEVKDYEPPFGSKVREHPCVESMKDNVLR |
| | | DRGRPEIPSFWLNHQGIQMVCETLTECWDHD |
| | | PEARLTAQCVAERFSELEHLDRLSGRSCSEEKI |
| | | PEDGSLNTTK |
| 2 | Full-length | MAVTSHHMIPVMVVLMSACLATAGPEPSTRCE |
| | rat | LSPINASHPVQALMESFTVLSGCASRGTTGLPR |
| | TGF-β | EVHVLNLRSTDQGPGQRQREVTLHLNPIASVH |
| | receptor | THHKPIVFLLNSPQPLVWHLKTERLAAGVPRLF |
| | III | LVSEGSVVQFPSGNFSLTAETEERNFPQENEH |
| | | LLRWAQKEYGAVTSFTELKIARNIYIKVGEDQV |
| | | FPPTCNIGKNFLSLNYLAEYLQPKAAEGCVLPS |
| | | QPHEKEVHIIELITPSSNPYSAFQVDIIVDIRPAQ |
| | | EDPEVVKNLVLILKCKKSVNWVIKSFDVKGNLK |
| | | VIAPNSIGFGKESERSMTMTKLVRDDIPSTQEN |
| | | LMKWALDNGYRPVTSYTMAPVANRFHLRLEN |
| | | NEEMRDEEVHTIPPELRILLDPDHPPALDNPLF |
| | | PGEGSPNGGLPFPFPDIPRRGWKEGEDRIPRP |
| | | KQPIVPSVQLLPDHREPEEVQGGVDIALSVKCD |
| | | HEKMVVAVDKDSFQTNGYSGMELTLLDPSCKA |
| | | KMNGTHFVLESPLNGCGTRHRRSTPDGVVYY |
| | | NSIVVQAPSPGDSSGWPDGYEDLESGDNGFP |
| | | GDGDEGETAPLSRAGVVVFNCSLRQLRNPSG |
| | | FQGQLDGNATFNMELYNTDLFLVPSPGVFSVA |
| | | ENEHVYVEVSVTKADQDLGFAIQTCFLSPYSNP |
| | | DRMSDYTIIENICPKDDSVKFYSSKRVHFPIPHA |
| | | EVDKKRFSFLFKSVFNTSLLFLHCELTLCSRKK |
| | | GSLKLPRCVTPDDACTSLDATMIWTMMQNKKT |
| | | FTKPLAVVLQVDYKENVPSTKDSSPIPPPPPQI |
| | | FHGLDTLTVMGIAFAAFVIGALLTGALWYIYSHT |
| | | GETARRQQVPTSPPASENSSAAHSIGSTQSTP |
| | | CSSSSTA |
| 3 | Full-length | MTSHYVIAIFALMSSCLATAGPEPGALCELSPV |
| | human | SASHPVQALMESFTVLSGCASRGTTGLPQEVH |
| | TGF-β | VLNLRTAGQGPGQLQREVTLHLNPISSVHIHHK |
| | receptor | SVVFLLNSPHPLVWHLKTERLATGVSRLFLVSE |
| | III | GSVVQFSSANFSLTAETEERNFPHGNEHLLNW |
| | | ARKEYGAVTSFTELKIARNIYIKVGEDQVFPPKC |
| | | NIGKNFLSLNYLAEYLQPKAAEGCVMSSQPQN |
| | | EEVHIIELITPNSNPYSAFQVDITIDIRPSQEDLE |
| | | VVKNLILILKCKKSVNWVIKSFDVKGSLKIIAPNSI |
| | | GFGKESERSMTMTKSIRDDIPSTQGNLVKWAL |
| | | DNGYSPITSYTMAPVANRFHLRLENNEEMGDE |
| | | EVHTIPPELRILLDPGALPALQNPPIRGGEGQN |
| | | GGLPFPFPDISRRVWNEEGEDGLPRPKDPVIP |
| | | SIQLFPGLREPEEVQGSVDIALSVKCDNEKMIV |
| | | AVEKDSFQASGYSGMDVTLLDPTCKAKMNGT |
| | | HFVLESPLNGCGTRPRWSALDGVVYYNSIVIQ |
| | | VPALGDSSGWPDGYEDLESGDNGFPGDMDE |
| | | GDASLFTRPEIVVFNCSLQQVRNPSSFQEQPH |
| | | GNITFNMELYNTDLFLVPSQGVFSVPENGHVY |
| | | VEVSVTKAEQELGFAIQTCFISPYSNPDRMSHY |
| | | TIIENICPKDESVKFYSPKRVHFPIPQADMDKKR |
| | | FSFVFKPVFNTSLLFLQCELTLCTKMEKHPQKL |
| | | PKCVPPDEACTSLDASIIWAMMQNKKTFTKPLA |
| | | VIHHEAESKEKGPSMKEPNPISPPIFHGLDTLTV |
| | | MGIAFAAFVIGALLTGALWYIYSHTGETAGRQQ |
| | | VPTSPPASENSSAAHSIGSTQSTPCSSSSTA |

The ectodomain of human TGF-β receptor II corresponds to residues 24-160 of SEQ ID NO: 1. Human TGF-β receptor II ectodomains useful in conjunction with the compositions and methods described herein include those that contain, e.g., from residue 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 of SEQ ID NO: 1 to residue 145, 146, 147, 148, 149, 150, 151, 152, 153, 154, 155, 156, 157, 158, 159, 160, 161, 162, 163, 164, 165, 166, 167, 168, 169, or 170 of SEQ ID NO: 1. For instance, human TGF-β receptor II ectodomains that may be used in conjunction with the compositions and methods described herein include those that contain residues 24-160 of SEQ ID NO: 1, residues 42-159 of SEQ ID NO: 1, as well as those that contain residues 48-159 of SEQ ID NO: 1. Additional examples of TGF-β receptor II ectodomains that may be used in conjunction with the compositions and methods described herein include those ectodomains from rat TGF-β receptor II, among other mammals.

The endoglin domain of rat TGF-β receptor III corresponds to residues 24-409 of SEQ ID NO: 2. Rat TGF-β receptor III endoglin domains useful in conjunction with the compositions and methods described herein include those that contain, e.g., from residue 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 2 to residue 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, or 409 of SEQ ID NO: 2. TGF-β receptor III endoglin domains useful in conjunction with the compositions and methods described herein may also contain one or more, or all, of the mutations R58H, H116R, C278S, and N337A relative to SEQ ID NO: 2. For instance, rat TGF-β receptor III endoglin domains that may be used in conjunction with the compositions and methods described herein include those that contain residues 24-409 of SEQ ID NO: 2, as well as those that contain residues 24-383 of SEQ ID NO: 2. Additional rat TGF-β receptor III endoglin domains that may be used in conjunction with the compositions and methods described herein include those that have amino acid sequences that differ from residues 24-409 of SEQ ID NO: 2 by virtue of one or more, or all, of the mutations R58H, H116R, C278S, and N337A, as well as those that have amino acid sequences that differ from residues 24-383 of SEQ ID NO: 2 by virtue of one or more, or all, of the mutations R58H, H116R, C278S, and N337A.

The endoglin domain of human TGF-β receptor III corresponds to residues 21-406 of SEQ ID NO: 3. Human TGF-β receptor III endoglin domains useful in conjunction with the compositions and methods described herein include those that contain, e.g., from residue 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 3 to residue 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, or 409 of SEQ ID NO: 3. TGF-β receptor III endoglin domains useful in conjunction with the compositions and methods described herein may also contain one or more, or all, of the mutations R55H, H113R, C275S, and N334A relative to SEQ ID NO: 3. For instance, human TGF-β receptor III endoglin domains that may be used in conjunction with the compositions and methods described herein include those that contain residues 21-406 of SEQ ID NO: 3, as well as those that contain residues 21-380 of SEQ ID NO: 3. Additional human TGF-β receptor III endoglin domains that may be used in conjunction with the compositions and methods described herein include those that have amino acid sequences that differ from residues 21-406 of SEQ ID NO: 3 by virtue of one or more, or all, of the mutations R55H, H113R, C275S, and N334A, as well as those that have amino acid sequences that differ from residues 21-380 of SEQ ID NO: 3 by virtue of one or more, or all, of the mutations R55H, H113R, C275S, and N334A.

The amino acid sequences of various TGF-β receptor II and III domains are shown in Table 3, below.

TABLE 3
Amino acid sequences of various domains of human and
rat TGF-β receptors

| SEQ ID | | |
|---|---|---|
| NO. | TGF-β Receptor Domain | Amino Acid Sequence |
| 10 | Human | NGAVKFPQLCKFCDVRFSTCDNQKSCMSNCSITSI |
| | TGF-β receptor II | CEKPQEVCVAVWRKNDENITLETVCHDPKLPYHDF |
| | ectodomain | ILEDAASPKCIMKEKKKPGETFFMCSCSSDECNDNI |
| | (Residues 42-159 of human | IFSEEYNTSNPD |
| | TGF-β receptor II) | |
| 11 | Human | PQLCKFCDVRFSTCDNQKSCMSNCSITSICEKPQE |
| | TGF-β receptor II | VCVAVWRKNDENITLETVCHDPKLPYHDFILEDAAS |
| | ectodomain | PKCIMKEKKKPGETFFMCSCSSDECNDNIIFSEEYN |
| | (Residues 48-159 of human | TSNPD |
| | TGF-β receptor II) | |
| 12 | Rat | GPEPSTRCELSPINASHPVQALMESFTVLSGCASH |
| | TGF-β receptor III | GTTGLPREVHVLNLRSTDQGPGQRQREVTLHLNPI |
| | endoglin domain containing | ASVHTHHKPIVFLLNSPQPLVWRLKTERLAAGVPR |
| | R58H, H116R, C278S, and | LFLVSEGSVVQFPSGNFSLTAETEERNFPQENEHL |
| | N337A mutations | LRWAQKEYGAVTSFTELKIARNIYIKVGEDQVFPPT |
| | (Residues 24-383 of rat | CNIGKNFLSLNYLAEYLQPKAAEGCVLPSQPHEKE |
| | TGF-β receptor III containing | VHIIELITPSSNPYSAFQVDIIVDIRPAQEDPEVVKNL |
| | the foregoing mutations) | VLILKSKKSVNWVIKSFDVKGNLKVIAPNSIGFGKES |
| | | ERSMTMTKLVRDDIPSTQENLMKWALDAGYRPVT |
| | | SYTMAPVANRFHLRLENNEEMRDEEVHTIPPELRIL |
| | | LDPD |
| 13 | Human | GPEPGALCELSPVSASHPVQALMESFTVLSGCASR |
| | TGF-β receptor III | GTTGLPQEVHVLNLRTAGQGPGQLQREVTLHLNPI |
| | endoglin domain containing | SSVHIHHKSVVFLLNSPHPLVWHLKTERLATGVSRL |
| | C275S mutation | FLVSEGSVVQFSSANFSLTAETEERNFPHGNEHLL |
| | (Residues 21-380 of human | NWARKEYGAVTSFTELKIARNIYIKVGEDQVFPPKC |
| | TGF-β receptor III containing | NIGKNFLSLNYLAEYLQPKAAEGCVMSSQPQNEEV |
| | the foregoing mutation) | HIIELITPNSNPYSAFQVDITIDIRPSQEDLEVVKNLILI |
| | | LKSKKSVNWVIKSFDVKGSLKIIAPNSIGFGKESERS |
| | | MTMTKSIRDDIPSTQGNLVKWALDNGYSPITSYTM |
| | | APVANRFHLRLENNEEMGDEEVHTIPPELRILLDPG |

### TGF-β Receptor Fusion Proteins

TGF-β receptor fusion proteins useful in conjunction with the compositions and methods described herein include those that contain one or more TGF-β receptors, or a domain, fragment, or variant thereof, bound to another TGF-β receptor, or a domain, fragment, or variant thereof. Exemplary TGF-β receptor fusion proteins include those in which two TGF-β receptor II ectodomains, such as two human TGF-β receptor II ectodomains, are bound to a single TGF-β receptor III endoglin domain, such as a rat or human TGF-β receptor III endoglin domain. It has been discovered that the endoglin domain of TGF-β receptor III binds two TGF-β molecules, while the ectodomain of TGF-β receptor II binds a single TGF-β molecule. Additionally, it has been found that the binding of the TGF-β receptor II ectodomain to TGF-β occurs at a site that is sterically distal from the site bound by the TGF-β receptor III endoglin domain. The binding of TGF-β receptor II ectodomain to TGF-β thus occurs independently from the binding of TGF-β receptor III endoglin domain to TGF-β. A multimeric fusion protein containing one or more TGF-β receptor II ectodomains bound to one or more TGF-β receptor III ectodomains has the capacity to bind TGF-β with high affinity by virtue of engaging this ligand at multiple distinct and independent sites. For instance, a trimeric fusion protein containing a TGF-β receptor II ectodomain bound to a TGF-β receptor III ectodomain, which is in turn bound to another TGF-β receptor II ectodomain has the capacity to bind two TGF-β molecules per a single fusion protein. Due in part to the binding of the fusion protein to a total of four sites across the ensemble of bound TGF-β molecules, the affinity of this interaction is high, as fusion proteins of this structure exhibit low-nanomolar to sub-nanomolar affinity for TGF-β. Exemplary TGF-β fusion proteins useful in conjunction with the compositions and methods of the invention are described, for instance, in U.S. Pat. No. 9,611,306, the disclosure of which is incorporated herein by reference in its entirety.

Exemplary TGF-β receptor fusion proteins for use in conjunction with the compositions and methods described herein include those having the amino acid sequence of SEQ ID NO: 9, as well as those having at least 70% sequence identity thereto (e.g., at least 75%, 80%, 85%, 90%, 95%, 97%, 98%, 99%, or more, sequence identity thereto). The amino acid sequence of SEQ ID NO: 9 is composed of an N-terminal human TGF-β receptor II ectodomain (SEQ ID NO: 10) bound to a central rat TGF-β receptor III endoglin domain (SEQ ID NO: 12), which is in turn bound to a C-terminal human TGF-β receptor II ectodomain (SEQ ID NO: 11).

### SEQ ID NO: 9, Exemplary TGF-β Receptor Fusion Protein of the Structure:

### RII Ectodomain-RIII Endoglin Domain-RII Ectodomain (“RER”)

NGAVKFPQLCKFCDVRFSTCDNQKSCMSNCSITSICEKPQEVCVAVWRKN
DENITLETVCHDPKLPYHDFILEDAASPKCIMKEKKKPGETFFMCSCSSD
ECNDNIIFSEEYNTSNPDGPEPSTRCELSPINASHPVQALMESFTVLSGC
ASHGTTGLPREVHVLNLRSTDQGPGQRQREVTLHLNPIASVHTHHKPIVF
LLNSPQPLVWRLKTERLAAGVPRLFLVSEGSVVQFPSGNFSLTAETEERN
FPQENEHLLRWAQKEYGAVTSFTELKIARNIYIKVGEDQVFPPTCNIGKN
FLSLNYLAEYLQPKAAEGCVLPSQPHEKEVHIIELITPSSNPYSAFQVDI
IVDIRPAQEDPEVVKNLVLILKSKKSVNWVIKSFDVKGNLKVIAPNSIGF
GKESERSMTMTKLVRDDIPSTQENLMKWALDAGYRPVTSYTMAPVANRFH
LRLENNEEMRDEEVHTIPPELRILLDPDPQLCKFCDVRFSTCDNQKSCMS
NCSITSICEKPQEVCVAVWRKNDENITLETVCHDPKLPYHDFILEDAASP
KCIMKEKKKPGETFFMCSCSSDECNDNIIFSEEYNTSNPD

Additional exemplary TGF-β antagonists or conjugates are described below. These TGF-β antagonists or conjugates can be used appropriately or interchangeably with the TGF-β antagonist constructs and conjugates discussed above or with any of the aspects or embodiments of the invention discussed herein.

In some instances, the invention features a composition containing a TGF-β antagonist, wherein the TGF-β antagonist is a fusion protein that comprises a homodimer of a compound of the formula: I(a). (A-L¹-B-L²-Z), I(b). (Z-L²-B-L¹-A), or I(c). (B-L¹-A-L²-Z), where A is an RER heterotrimeric fusion polypeptide; L¹ is a linker; B is an Fc domain of an immunoglobulin or is absent; L² is a linker or is absent; Z is a bone-targeting moiety or is absent; and where A, the RER heterotrimeric fusion polypeptide, includes a polypeptide sequence of the formula: W-L³-X-L⁴-Y, where W is a TGF-β type II receptor ectodomain or a portion thereof; L³ is a linker or is absent; X is a TGF-β type III receptor endoglin domain or a portion thereof; L⁴ is a linker or is absent; Y is a TGF-β type II receptor ectodomain or a portion thereof, and where the amino acid sequence of A is not the amino acid sequence of SEQ ID NO: 48.

Certain aspects of the above composition may vary in ways described below.

In some instances, the linker L¹ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some instances, B, the Fc domain of an immunoglobulin is present. In some instances, B, the Fc domain of an immunoglobulin is absent. In some instances, B, the Fc domain of an immunoglobulin includes the Fc domain of human IgG, human IgA, human IgM, human IgE, or human IgD; or a variant of said domain. In some instances, the Fc domain of human IgG is IgG1, IgG2, IgG3, or IgG4; or a variant thereof. In some instances, the Fc domain of human includes the amino acid sequence of SEQ ID NO: 47; or a variant of said amino acid sequence.

In some instances, the linker L² is present. In some instances, the linker L² is absent. In some instances, the linker L² includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some instances, the bone-targeting moiety, is present. In some instances, the bone-targeting moiety, is absent. In some instances, the bone-targeting moiety includes a polyanionic peptide, a bisphosphonate, or the amino acid sequence of SEQ ID NO: 46; or a variant of said amino acid sequence.

In some instances, the TGF-β type II receptor ectodomain W is at the N-terminus of the RER heterotrimeric fusion polypeptide and the TGF-β type II receptor ectodomain Y is at the C-terminus of the RER heterotrimeric fusion polypeptide. In some instances, the C-terminus of the TGF-β type II receptor ectodomain Y is covalently joined to the N-terminus of B, Fc domain of an immunoglobulin, via the linker L¹ as in formula I(a). In some instances, the N-terminus of the TGF-β type II receptor ectodomain W is covalently joined to the C-terminus of B via the linker L¹ as in formula I(b) or I(c).

In some instances, the amino acid sequence of the TGF-β type II receptor ectodomain W is identical to the amino acid sequence of the TGF-β type II receptor ectodomain Y. In some instances, the amino acid sequence of the TGF-β type II receptor ectodomain W is different than the amino acid sequence of the TGF-β type II receptor ectodomain Y. In some instances, the TGF-β type II receptor ectodomains W and/or Y includes an amino acid sequence extending from amino acid residues 22 to 139 of SEQ ID NO: 5, 520 to 631 of SEQ ID NO: 5, 1 to 118 of SEQ ID NO: 9, 479 to 590 of SEQ ID NO: 9, 1 to 118 of SEQ ID NO: 48, 499 to 610 of SEQ ID NO: 48, 1 to 118 of SEQ ID NO: 49, 499 to 610 of SEQ ID NO: 49, 1 to 120 of SEQ ID NO: 50, 501 to 612 of SEQ ID NO: 50, 1 to 120 of SEQ ID NO: 51, 501 to 612 of SEQ ID NO: 51, 1 to 120 of SEQ ID NO: 52, or 510 to 621 of SEQ ID NO: 52; or a variant of said amino acid sequences.

In some instances, the linker L³ is present. In some instances, the linker L³ is absent. In some instances, the linker L³ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some instances, where the TGF-β type III receptor endoglin domain X includes an amino acid sequence extending from amino acid residues 157 to 517 of SEQ ID NO: 5, 119 to 478 of SEQ ID NO: 9, 136 to 496 of SEQ ID NO: 48, 136 to 496 of SEQ ID NO: 49, 138 to 500 of SEQ ID NO: 50, 138 to 500 of SEQ ID NO: 51, or 147 to 509 of SEQ ID NO: 52; or a variant of said amino acid sequences. In some instances, the linker L⁴ is present. In some instances, where the linker L⁴ is absent. In some instances, the linker L⁴ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some instances, the RER heterotrimeric fusion polypeptide includes an amino acid sequence selected from the group comprising SEQ ID NO: 9, SEQ ID NO: 49, SEQ ID NO: 50, SEQ ID NO: 51, and SEQ ID NO: 52; or a variant of said amino acid sequences. In some instances, the RER heterotrimeric fusion polypeptide includes the amino acid sequence of SEQ ID NO: 51; or a variant of said amino acid sequence. In some instances, the RER heterotrimeric fusion polypeptide includes the amino acid sequence of SEQ ID NO: 52; or a variant of said amino acid sequence.

In some instances, the homodimer includes an amino acid sequence selected from the group comprising SEQ ID NO: 22, SEQ ID NO: 24, SEQ ID NO: 26, SEQ ID NO: 28, and SEQ ID NO: 30; or a variant of said amino acid sequences. In some instances, the homodimer includes an amino acid sequence selected from the group comprising SEQ ID NO: 9, SEQ ID NO: 23, SEQ ID NO: 25, SEQ ID NO: 27, SEQ ID NO: 29, and SEQ ID NO: 31; or a variant of said amino acid sequences.

In a second aspect, In a first aspect, the invention features a composition containing a TGF-β antagonist, wherein the TGF-β antagonist is a fusion protein that includes a homodimer of a compound of the formula: I(a). (A-L¹-B-L²-Z); where A is an RER heterotrimeric fusion polypeptide; L¹ is a linker; B is an Fc domain of an immunoglobulin; L² is a linker that is absent; Z is a bone-targeting moiety; and A, the RER heterotrimeric fusion polypeptide, includes a polypeptide sequence of the formula: W-L³-X-L⁴-Y, where W is a TGF-β type II receptor ectodomain or a portion thereof; L³ is a linker; X is a TGF-β type III receptor endoglin domain or a portion thereof; L⁴ is a linker that is absent; and Y is a TGF-β type II receptor ectodomain or a portion thereof; and the amino acid sequence of A is not the amino acid sequence of SEQ ID NO: 48.

In some instances, the homodimer is PCT-0025 having the amino acid sequence of SEQ ID NO: 28; or a variant of said amino acid sequence. In some instances, the homodimer is PCT-0026 having the amino acid sequence of SEQ ID NO: 30; or a variant of said amino acid sequence.

In another aspect, the invention features a composition containing a TGF-β antagonist, wherein the TGF-β antagonist is a fusion protein that includes a homodimer of a compound of the formula: II(a). (A-L¹-B-L²-Z), II(b). (Z-L²-B-L¹-A), or II(c). (B-L¹-A-L²-Z), where A is an RER heterotrimeric fusion polypeptide; L¹ is a linker; B is an Fc domain of an immunoglobulin or is absent; L² is a linker or is absent; Z is a bone-targeting moiety; A, the RER heterotrimeric fusion polypeptide, includes a polypeptide sequence of the formula: W-L³-X-L⁴-Y, where W is a TGF-β type II receptor ectodomain or a portion thereof; L³ is a linker or is absent; X is a TGF-β type III receptor endoglin domain or a portion thereof; L⁴ is a linker or is absent; Y is a TGF-β type II receptor ectodomain or a portion thereof, and where A includes the amino acid sequence of SEQ ID NO: 48.

Certain aspects of the above composition may vary in ways described below.

In some instances, the linker L¹ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some instances, the Fc domain of an immunoglobulin is present. In some instances, the Fc domain of an immunoglobulin is absent. In some instances, the Fc domain of an immunoglobulin includes the Fc domain of human IgG, human IgA, human IgM, human IgE, or human IgD; or a variant of said domain. In some instances, the Fc domain of human IgG is IgG1, IgG2, IgG3, or IgG4; or a variant thereof. In some instances, the Fc domain of human includes the amino acid sequence of SEQ ID NO: 47; or a variant of said amino acid sequence.

In some instances, the linker L² is present. In some instances, the linker L² is absent. In some instances, the linker L² includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some instances, the bone-targeting moiety includes a polyanionic peptide, a bisphosphonate, or the amino acid sequence of SEQ ID NO: 46; or a variant of said amino acid sequence.

In some instances, the TGF-β type II receptor ectodomain W is at the N-terminus of the RER heterotrimeric fusion polypeptide and the TGF-β type II receptor ectodomain Y is at the C-terminus of the RER heterotrimeric fusion polypeptide. In some instances, the C-terminus of the TGF-β type II receptor ectodomain Y is covalently joined to the N-terminus of B, Fc domain of an immunoglobulin, via the linker L¹ as in formula I(a). In some instances, the N-terminus of the TGF-β type II receptor ectodomain W is covalently joined to the C-terminus of B via the linker L¹ as in formula I(b) or I(c).

In some instances, the amino acid sequence of the TGF-β type II receptor ectodomain W is identical to the amino acid sequence of the TGF-β type II receptor ectodomain Y. In some instances, the amino acid sequence of the TGF-β type II receptor ectodomain W is different than the amino acid sequence of the TGF-β type II receptor ectodomain Y. In some instances, the TGF-β type II receptor ectodomains W and/or Y includes an amino acid sequence extending from amino acid residues 22 to 139 of SEQ ID NO: 5, 520 to 631 of SEQ ID NO: 5, 1 to 118 of SEQ ID NO: 9, 479 to 590 of SEQ ID NO: 9, 1 to 118 of SEQ ID NO: 48, 499 to 610 of SEQ ID NO: 48, 1 to 118 of SEQ ID NO: 49, 499 to 610 of SEQ ID NO: 49, 501 to 612 of SEQ ID NO: 50, 501 to 612 of SEQ ID NO: 51, or 510 to 621 of SEQ ID NO: 52; or a variant of said amino acid sequences. In some instances, the TGF-β type II receptor ectodomains W and/or Y does not comprise an amino acid sequence extending from amino acid residues 22 to 139 of SEQ ID NO: 5, 520 to 631 of SEQ ID NO: 5, 1 to 118 of SEQ ID NO: 9, 479 to 590 of SEQ ID NO: 9, 1 to 118 of SEQ ID NO: 48, 499 to 610 of SEQ ID NO: 48, 1 to 118 of SEQ ID NO: 49, 499 to 610 of SEQ ID NO: 49, 501 to 612 of SEQ ID NO: 50, 501 to 612 of SEQ ID NO: 51, or 510 to 621 of SEQ ID NO: 52; or a variant of said amino acid sequences.

In some instances, the linker L³ is present. In some instances, the linker L³ is absent. In some instances, the linker L³ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some instances, the TGF-β type III receptor endoglin domain X includes an amino acid sequence extending from amino acid residues 157 to 517 of SEQ ID NO: 5, 136 to 496 of SEQ ID NO: 48, or 136 to 496 of SEQ ID NO: 49; or a variant of said amino acid sequences. In some instances, the TGF-β type III receptor endoglin domain X does not comprise an amino acid sequence extending from amino acid residues 157 to 517 of SEQ ID NO: 5, 136 to 496 of SEQ ID NO: 48, or 136 to 496 of SEQ ID NO: 49; or a variant of said amino acid sequences.

In some instances, the linker L⁴ is present. In some instances, the linker L⁴ is absent. In some instances, the linker L⁴ includes a natural peptidic linker, a synthetic linker, or an amino acid sequence selected from the group comprising SEQ ID NO: 6, SEQ ID NO: 7, SEQ ID NO: 8, SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, and SEQ ID NO: 61; or a variant of said amino acid sequences.

In some instances, the RER heterotrimeric fusion polypeptide includes the amino acid sequence of SEQ ID NO: 48; or a variant of said amino acid sequences.

In some instances, the homodimer includes an amino acid sequence selected from the group comprising SEQ ID NO: 14, SEQ ID NO: 16, SEQ ID NO: 18, SEQ ID NO: 32, and SEQ ID NO: 34; or a variant of said amino acid sequences.

In another aspect, the invention features a composition containing a TGF-β antagonist, wherein the TGF-β antagonist is a fusion protein that includes a homodimer of a compound of the formula: III(a). (A-L¹-B-L²-Z), III(b). (Z-L²-B-L¹-A), or III(c). (B-L¹-A-L²-Z), where A is an RER heterotrimeric fusion polypeptide; L¹ is a linker; B is an Fc domain of an immunoglobulin or is absent; L² is a linker or is absent; Z is a bone-targeting moiety or is absent; and where at least one of the following is present:
a. A, the RER heterotrimeric fusion polypeptide, includes an amino acid sequence selected from the group consisting of SEQ ID NO: 9, SEQ ID NO: 49, SEQ ID NO: 50, SEQ ID NO: 51, and SEQ ID NO: 52; or
b. the linker L¹ includes an amino acid sequence selected from the group consisting of SEQ ID NO: 8, SEQ ID NO: 36, SEQ ID NO: 37, and SEQ ID NO: 38; or
c. the linker L² is present and includes an amino acid sequence of SEQ ID NO: 8, or SEQ ID NO: 41; or
d. the linker L³ is present and includes the amino acid sequence of SEQ ID NO: 38 or SEQ ID NO: 39: or
e. X, the TGF-β type III receptor endoglin domain, includes the amino acid sequence of SEQ ID NO: 44.

Specific TGF-β receptor fusion protein constructs or antagonists of the invention with the D10 bone-targeting moiety are summarized in Table 4, below.

TABLE 4
TGF-β antagonists with the D10 bone-targeting moiety

| TGF-β | |
|---|---|
| Antagonist | SEQ ID NO. |
| PCT-0015 | 14 |
| PCT-0019 | 16 |
| PCT-0020 | 18 |
| PCT-0021 | 20 |
| PCT-0022 | 22 |
| PCT-0023 | 24 |
| PCT-0024 | 26 |
| PCT-0025 | 28 |
| PCT-0026 | 30 |
| PCT-0017 | 32 |
| PCT-0018 | 34 |

Specific TGF-β receptor fusion protein constructs or antagonists of the invention without the D10 bone-targeting moiety (“NT”) are summarized in Table 5, below.

TABLE 5
TGF-β antagonists without the D10 bone-targeting moiety

| TGF-β | |
|---|---|
| Antagonist | SEQ ID NO. |
| PCT-0015NT | 15 |
| PCT-0019NT | 17 |
| PCT-0020NT | 19 |
| PCT-0021NT | 21 |
| PCT-0022NT | 23 |
| PCT-0023NT | 25 |
| PCT-0024NT | 27 |
| PCT-0025NT | 29 |
| PCT-0026NT | 31 |
| PCT-0016NT | 33 |
| PCT-0018NT | 35 |

In some instances, the novel TGF-β receptor fusion protein constructs or antagonists of the invention are those with the D10 bone-targeting moiety (SEQ ID NO: 46) and includes the amino acid sequence of SEQ ID NO: 5, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, or SEQ ID NO: 34, or a variant of said amino acid sequences. The TGF-β receptor fusion protein constructs or antagonists with the D10 bone-targeting moiety can be used to treat a variety of disorders associated with elevated TGF-β signaling in bone tissue.

In other instances, the novel TGF-β receptor fusion protein constructs or antagonists of the invention are those without the D10 bone-targeting moiety and includes the amino acid sequence of SEQ ID NO: 9, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, or SEQ ID NO: 35, or a variant of said amino acid sequences. The TGF-β receptor fusion protein constructs or antagonists without the D10 bone-targeting moiety can be used to treat a variety of disorders associated with elevated TGF-β signaling in both bone tissue and tissues other than bone.

Other bone-targeting moieties, as described herein, may be used in lieu of the D10 bone-targeting moiety, as appropriate.

The TGF-β antagonist constructs described above may be used appropriately or interchangeably with the compositions and methods of any of the aspects or embodiments of the invention described herein.

Antibodies and Antigen-Binding Fragments Thereof that Bind TGF-β

Examples of TGF-β antagonists useful in conjunction with the compositions and methods described herein include antibodies and antigen-binding fragments thereof directed against one or more isoforms of TGF-β (such as those described in U.S. Pat. No. 5,571,714, as well as International Patent Application Publication No. WO 1997/013844, the disclosures of each of which are incorporated herein by reference), and antibodies directed against TGF-β receptors (such as those described in U.S. Pat. Nos. 5,693,607, 6,008,011, 6,001,969, and 6,010,872, as well as WO 92/00330, WO 93/09228, WO 95/10610, and WO 98/48024, the disclosures of which are incorporated herein by reference).

Particular TGF-β antagonists useful in conjunction with the compositions and methods described herein include anti-TGF-β antibody 1D11, as well as antigen-binding fragments thereof and human, humanized, and chimeric variants thereof. Anti-TGF-β antibody GC1008, a humanized variant of 1D11, is described in U.S. Pat. No. 9,958,486, the disclosure of which is incorporated herein by reference in its entirety. Anti-TGF-β antibody GC1008 contains the following complementarity determining regions (CDRs):
(a) a CDR-H1 having the amino acid sequence SNVIS (SEQ ID NO: 64);
(b) a CDR-H2 having the amino acid sequence GVIPIVDIANYAQRFKG (SEQ ID NO: 65);
(c) a CDR-H3 having the amino acid sequence TLGLVLDAMDY (SEQ ID NO: 66);
(d) a CDR-L1 having the amino acid sequence RASQSLGSSYLA (SEQ ID NO: 67);
(e) a CDR-L2 having the amino acid sequence GASSRAP (SEQ ID NO: 68); and
(f) a CDR-L3 having the amino acid sequence QQYADSPIT (SEQ ID NO: 69).

Anti-TGF-β antibody GC1008 contains a heavy chain variable region having the sequence of SEQ ID NO: 70, and a light chain variable region having the amino acid sequence of SEQ ID NO: 71, shown below:

GC1008 Heavy chain variable region amino acid
sequence

(SEQ ID NO: 70)

QVQLVQSGAEVKKPGSSVKVSCKASGYTFSSNVISWVRQAPGQGLEWMGG
VIPIVDIANYAQRFKGRVTITADESTSTTYMELSSLRSEDTAVYYCASTL
GLVLDAMDYWGQGTLVTVSS
GC1008 Light chain variable region amino acid
sequence

(SEQ ID NO: 71)

ETVLTQSPGTLSLSPGERATLSCRASQSLGSSYLAWYQQKPGQAPRLLIY
GASSRAPGIPDRFSGSGSGTDFTLTISRLEPEDFAVYYCQQYADSPITFG
QGTRLEIK

Anti-TGF-β antagonists useful in conjunction with the compositions and methods described herein include antibodies and antigen-binding fragments thereof containing one or more, or all, of the CDRs of GC1008, as well as those containing a set of CDRs that each have at least 50% sequence identity (e.g., at least 50%, 55%, 60%, 65%, 70%, 75%, 80%, 85%, 90%, 95%, 97%, 98%, 99%, or more, sequence identity) to the CDRs of GC1008, shown above.

Exemplary anti-TGF-β antagonists useful in conjunction with the compositions and methods described herein include monoclonal antibodies and antigen-binding fragments thereof, polyclonal antibodies and antigen-binding fragments thereof, humanized antibodies and antigen-binding fragments thereof, bispecific antibodies and antigen-binding fragments thereof, optimized antibodies and antigen-binding fragments thereof (e.g., affinity-matured antibodies and antigen-binding fragments thereof), dual-variable immunoglobulin domains, single-chain Fv molecules (scFvs), diabodies, triabodies, nanobodies, antibody-like protein scaffolds, Fv fragments, Fab fragments, F(ab′)₂ molecules, and tandem di-scFVs, among others, such as those that have one or more, or all, of the CDRs of GC1008, as well as those containing a set of CDRs that each have at least 50% sequence identity (e.g., at least 50%, 55%, 60%, 65%, 70%, 75%, 80%, 85%, 90%, 95%, 97%, 98%, 99%, or more, sequence identity) to the CDRs of GC1008, shown above.

Additionally, antibodies and antigen-binding fragments thereof that may be used in conjunction with the compositions and methods described herein include those that bind the same epitope on TGF-β as murine antibody 1D11, its humanized counterpart, GC1008, and antibodies or antigen-binding fragments thereof that have the same set of CDRs as 1D11 and GC1008. Exemplary methods that can be used to determine whether an antibody or antigen-binding fragment thereof binds the same epitope on TGF-β as a reference antibody, such as 1D11 or GC1008, include competitive binding experiments, such as competitive ELISA experiments or other competitive binding assays known in the art. An antibody or antigen-binding fragment thereof is considered to bind the same epitope on TGF-β as a reference antibody, such as 1D11 or GC1008, if the antibody or antigen-binding fragment thereof competitively inhibits the binding of TGF-β to the reference antibody. Competitive binding experiments that can be used to determine whether an antibody or antigen-binding fragment thereof binds to the same epitope on TGF-β as a reference antibody or antigen-binding fragment thereof are described, for instance, in Nagata et al., Journal of Immunological Methods 292:141-155 (2004), the disclosure of which is incorporated herein by reference in its entirety.

Thus, antibodies and antigen-binding fragments thereof useful in conjunction with the compositions and methods described herein include those that competitively inhibit the binding of TGF-β to an antibody or antigen-binding fragment thereof that contains the following CDRs:
(a) a CDR-H1 having the amino acid sequence SNVIS (SEQ ID NO: 64);
(b) a CDR-H2 having the amino acid sequence GVIPIVDIANYAQRFKG (SEQ ID NO: 65);
(c) a CDR-H3 having the amino acid sequence TLGLVLDAMDY (SEQ ID NO: 66);
(d) a CDR-L1 having the amino acid sequence RASQSLGSSYLA (SEQ ID NO: 67);
(e) a CDR-L2 having the amino acid sequence GASSRAP (SEQ ID NO: 68); and
(f) a CDR-L3 having the amino acid sequence QQYADSPIT (SEQ ID NO: 69).

Antibodies and antigen-binding fragments thereof that may be used with the compositions and methods described herein include those that competitively inhibit the binding of TGF-β to an antibody or antigen-binding fragment thereof having the heavy chain variable region set forth in SEQ ID NO: 70 and/or the light chain variable region set forth in SEQ ID NO: 71.

Additional TGF-β antagonists useful in conjunction with the compositions and methods described herein include anti-TGF-β antibody PCT-0011 (with the bone-targeting moiety D10), as well as antigen-binding fragments thereof. Antibodies and antigen-binding fragments thereof that may be used with the compositions and methods described herein include an antibody or antigen-binding fragment thereof having the heavy chain set forth in SEQ ID NO: 62, or a heavy chain having an amino acid sequence that has at least 85% sequence identity (e.g., at least 85%, 90%, 95%, 97%, 98%, 99%, or more, sequence identity) to SEQ ID NO: 62, and/or the light chain set forth in SEQ ID NO: 63, or a light chain having an amino acid sequence that has at least 85% sequence identity (e.g., at least 85%, 90%, 95%, 97%, 98%, 99%, or more, sequence identity) to SEQ ID NO: 63, that competitively inhibit the binding of TGF-β to an anti-TGF-β antibody or an antigen-binding fragment thereof, such as 1D11 or GC1008 antibody, or an an antigen-binding fragment thereof.

Additional TGF-β antagonists useful in conjunction with the compositions and methods described herein include anti-TGF-β antibody TβM1 (LY2382770). The TβM1 (LY2382770) antibody sequences are described in detail in, e.g., WO 2005/010049, the disclosure of which is incorporated herein by reference in its entirety.

TGF-β Antagonists from TGF-β Co-Receptors

Additional exemplary TGF-β antagonists that bind TGF-β and inhibit TGF-β signaling include peptides from TGF-β co-receptors, such as the TGF-β co-receptor, CD109. This peptide is described in detail, for instance, in U.S. Pat. No. 7,173,002 and in US 2012/0079614, the disclosures of each of which are incorporated herein by reference in their entirety. This 1428-residue peptide, as well as fragments thereof, have been shown to inhibit TGF-β signaling in mammalian cells. Active forms of this peptide may contain a tyrosine (SEQ ID NO: 73) or serine (SEQ ID NO: 75) residue at position 703 within the CD109 sequence. Additionally, fragments of CD109, such as those containing the amino acid sequence of residues 21-1404 or 21-1428, may be used as TGF-β antagonist peptides in the context of the conjugates, compositions, and methods described herein. Other fragments of CD109, such as those containing the amino acid sequence WIWLDTNMGYRIYQEFEVT (SEQ ID NO: 72) or WIWLDTNMGSRIYQEFEVT (SEQ ID NO: 74), which correspond to positions 694-712 of SEQ ID NO: 73 and SEQ ID NO: 75, respectively, may be used as TGF-β antagonists in the conjugates, compositions, and methods described herein, as these sequences may contain a putative TGF-β binding site. Additional fragment of the CD109 peptide that can be used as a TGF-β antagonist peptide in the conjugates, compositions, and methods described herein contain the amino acid sequence IDGVYDNAEYAERFMEENEGHIVDIHDFSLGSS (SEQ ID NO: 76), which corresponds to residues 651-683 of SEQ ID NO: 73, which may also contain a putative TGF-β binding site.

Additional fragments of CD109 that can be used in the conjugates, compositions, and methods described herein include a 161-residue portion of this protein that has the amino acid sequence TMENVVHELELYNTGYYLGMFMNSFAVFQECGLWVLTDANLTKDYIDGVYDNAEYAERFMEENEGHIVDIHDFSLGSSPHVRKHFPETWIWLDTNMGSRIYQEFEVTVPDSITSWVATGFVISEDLGLGLTTTPVELQAFQPFFIFLNLPYSVIRGEEFAL (SEQ ID NO: 77). Additional peptidic fragments of CD109 that can be used in the conjugates, compositions, and methods described herein may comprise at least 10, 15, 25, 50, 75, 100, 250, 500, 750, 1000, 1250, 1400 or more contiguous amino acids of SEQ ID NO: 73. Exemplary CD109 fragments that may be used in conjunction with the conjugates, compositions, and methods described herein include those that contain a putative TGF-β binding site, such as peptides containing the amino acid sequence RKHFPETWIWLDTNMGYRIYQEFEV (SEQ ID NO: 78), which corresponds to residues 687-711 of SEQ ID NO: 73.

In addition to the above, peptide antagonists of TGF-β useful in conjunction with the conjugates, compositions, and methods described herein include those containing an amino acid sequence having at least 85% sequence identity (e.g., at least 85%, 90%, 95%, 97%, 99%, or greater) to one of the foregoing sequences and/or having one or more conservative amino acid substitutions with respect to one of the foregoing sequences.

The foregoing antagonistic TGF-β peptides are summarized in Table 6, below.

TABLE 6
Exemplary TGF-β antagonist peptide sequences based on CD109

| SEQ ID | |
|---|---|
| NO. | Amino acid sequence |
| 72 | WIWLDTNMGYRIYQEFEVT |
| 73 | MQGPPLLTAAHLLCVCTAALAVAPGPRFLVTAPGIIRPGGNVTIGVELLEHCPSQVT |
| | VKAELLKTASNLTVSVLEAEGVFEKGSFKTLTLPSLPLNSADEIYELRVTGRTQDEIL |
| | FSNSTRLSFETKRISVFIQTDKALYKPKQEVKFRIVTLFSDFKPYKTSLNILIKDPKSNL |
| | IQQWLSQQSDLGVISKTFQLSSHPILGDWSIQVQVNDQTYYQSFQVSEYVLPKFEV |
| | TLQTPLYCSMNSKHLNGTITAKYTYGKPVKGDVTLTFLPLSFWGKKKNITKTFKING |
| | SANFSFNDEEMKNVMDSSNGLSEYLDLSSPGPVEILTTVTESVTGISRNVSTNVFFK |
| | QHDYIIEFFDYTTVLKPSLNFTATVKVTRADGNQLTLEERRNNVVITVTQRNYTEYW |
| | SGSNSGNQKMEAVQKINYTVPQSGTFKIEFPILEDSSELQLKAYFLGSKSSMAVHSL |
| | FKSPSKTYIQLKTRDENIKVGSPFELVVSGNKRLKELSYMVVSRGQLVAVGKQNST |
| | MFSLTPENSWTPKACVIVYYIEDDGEIISDVLKIPVQLVFKNKIKLYWSKVKAEPSEK |
| | VSLRISVTQPDSIVGIVAVDKSVNLMNASNDITMENVVHELELYNTGYYLGMFMNSF |
| | AVFQECGLWVLTDANLTKDYIDGVYDNAEYAERFMEENEGHIVDIHDFSLGSSPHV |
| | RKHFPETWIWLDTNMGYRIYQEFEVTVPDSITSWVATGFVISEDLGLGLTTTPVELQ |
| | AFQPFFIFLNLPYSVIRGEEFALEITIFNYLKDATEVKVIIEKSDKFDILMTSNEINATGH |
| | QQTLLVPSEDGATVLFPIRPTHLGEIPITVTALSPTASDAVTQMILVKAEGIEKSYSQS |
| | ILLDLTDNRLQSTLKTLSFSFPPNTVTGSERVQITAIGDVLGPSINGLASLIRMPYGCG |
| | EQNMINFAPNIYILDYLTKKKQLTDNLKEKALSFMRQGYQRELLYQREDGSFSAFGN |
| | YDPSGSTWLSAFVLRCFLEADPYIDIDQNVLHRTYTWLKGHQKSNGEFWDPGRVIH |
| | SELQGGNKSPVTLTAYIVTSLLGYRKYQPNIDVQESIHFLESEFSRGISDNYTLALITY |
| | ALSSVGSPKAKEALNMLTWRAEQEGGMQFWVSSESKLSDSWQPRSLDIEVAAYA |
| | LLSHFLQFQTSEGIPIMRWLSRQRNSLGGFASTQDTTVALKALSEFAALMNTERTNI |
| | QVTVTGPSSPSPLAVVQPTAVNISANGFGFAICQLNVVYNVKASGSSRRRRSIQN |
| | QEAFDLDVAVKENKDDLNHVDLNVCTSFSGPGRSGMALMEVNLLSGFMVPSEAIS |
| | LSETVKKVEYDHGKLNLYLDSVNETQFCVNIPAVRNFKVSNTQDASVSIVDYYEPR |
| | RQAVRSYNSEVKLSSCDLCSDVQGCRPCEDGASGSHHHSSVIFIFCFKLLYFMEL |
| | WL |
| 74 | WIWLDTNMGSRIYQEFEVT |
| 75 | MQGPPLLTAAHLLCVCTAALAVAPGPRFLVTAPGIIRPGGNVTIGVELLEHCPSQVT |
| | VKAELLKTASNLTVSVLEAEGVFEKGSFKTLTLPSLPLNSADEIYELRVTGRTQDEIL |
| | FSNSTRLSFETKRISVFIQTDKALYKPKQEVKFRIVTLFSDFKPYKTSLNILIKDPKSNL |
| | IQQWLSQQSDLGVISKTFQLSSHPILGDWSIQVQVNDQTYYQSFQVSEYVLPKFEV |
| | TLQTPLYCSMNSKHLNGTITAKYTYGKPVKGDVTLTFLPLSFWGKKKNITKTFKING |
| | SANFSFNDEEMKNVMDSSNGLSEYLDLSSPGPVEILTTVTESVTGISRNVSTNVFFK |
| | QHDYIIEFFDYTTVLKPSLNFTATVKVTRADGNQLTLEERRNNVVITVTQRNYTEYW |
| | SGSNSGNQKMEAVQKINYTVPQSGTFKIEFPILEDSSELQLKAYFLGSKSSMAVHSL |
| | FKSPSKTYIQLKTRDENIKVGSPFELVVSGNKRLKELSYMVVSRGQLVAVGKQNST |
| | MFSLTPENSWTPKACVIVYYIEDDGEIISDVLKIPVQLVFKNKIKLYWSKVKAEPSEK |
| | VSLRISVTQPDSIVGIVAVDKSVNLMNASNDITMENVVHELELYNTGYYLGMFMNSF |
| | AVFQECGLWVLTDANLTKDYIDGVYDNAEYAERFMEENEGHIVDIHDFSLGSSPHV |
| | RKHFPETWIWLDTNMGSRIYQEFEVTVPDSITSWVATGFVISEDLGLGLTTTPVELQ |
| | AFQPFFIFLNLPYSVIRGEEFALEITIFNYLKDATEVKVIIEKSDKFDILMTSNEINATGH |
| | QQTLLVPSEDGATVLFPIRPTHLGEIPITVTALSPTASDAVTQMILVKAEGIEKSYSQS |
| | ILLDLTDNRLQSTLKTLSFSFPPNTVTGSERVQITAIGDVLGPSINGLASLIRMPYGCG |
| | EQNMINFAPNIYILDYLTKKKQLTDNLKEKALSFMRQGYQRELLYQREDGSFSAFGN |
| | YDPSGSTWLSAFVLRCFLEADPYIDIDQNVLHRTYTWLKGHQKSNGEFWDPGRVIH |
| | SELQGGNKSPVTLTAYIVTSLLGYRKYQPNIDVQESIHFLESEFSRGISDNYTLALITY |
| | ALSSVGSPKAKEALNMLTWRAEQEGGMQFWVSSESKLSDSWQPRSLDIEVAAYA |
| | LLSHFLQFQTSEGIPIMRWLSRQRNSLGGFASTQDTTVALKALSEFAALMNTERTNI |
| | QVTVTGPSSPSPLAVVQPTAVNISANGFGFAICQLNVVYNVKASGSSRRRRSIQNQ |
| | EAFDLDVAVKENKDDLNHVDLNVCTSFSGPGRSGMALMEVNLLSGFMVPSEAISLS |
| | ETVKKVEYDHGKLNLYLDSVNETQFCVNIPAVRNFKVSNTQDASVSIVDYYEPRRQ |
| | AVRSYNSEVKLSSCDLCSDVQGCRPCEDGASGSHHHSSVIFIFCFKLLYFMELWL |
| 76 | IDGVYDNAEYAERFMEENEGHIVDIHDFSLGSS |
| 77 | TMENVVHELELYNTGYYLGMFMNSFAVFQECGLWVLTDANLTKDYIDGVYDNAEY |
| | AERFMEENEGHIVDIHDFSLGSSPHVRKHFPETWIWLDTNMGSRIYQEFEVTVPDSI |
| | TSWVATGFVISEDLGLGLTTTPVELQAFQPFFIFLNLPYSVIRGEEFAL |
| 78 | RKHFPETWIWLDTNMGYRIYQEFEV |

### Additional Peptidic and Proteinaceous TGF-β Antagonists

In addition to the above, peptide antagonists capable of binding TGF-β for use with the conjugates, compositions, and methods described herein include those described in U.S. Pat. No. 7,723,473, the disclosure of which is incorporated herein by reference in its entirety, as well as peptide antagonists of TGF-β containing an amino acid sequence having at least 85% sequence identity (e.g., at least 85%, 90%, 95%, 97%, 99%, or greater) to one of these sequences and/or having one or more conservative amino acid substitutions with respect to one of these sequences. These TGF-β antagonists specifically bind to TGF-β receptors, which include type I, type II, type III and type V receptors. It has been shown that these peptides, some of which correspond in sequence to amino acid numbers 41-65 of TGF-β₁, TGF-β₂, and TGF-β₃, inhibit the binding of TGF-β₁, TGF-β₂, and TGF-β₃, to TGF-β receptors. These peptides have been shown to attenuate TGF-β-induced growth inhibition and TGF-β-induced expression of PAI-1. It has also been shown that the W/RXXD motif found within these peptide sequences determines the specificity of activity of the antagonist peptide. These TGF-β antagonist peptides are summarized in Table 7, below.

TABLE 7
Exemplary TGF-β antagonist peptides

| SEQ ID NO. | Amino acid sequence |
|---|---|
| 79 | ANFCLGPCPYIWSLDT |
| 80 | ANFCSGPCPYLRSADT |
| 81 | PYIWSLDTQY |
| 82 | PYLWSSDTQH |
| 83 | PYLRSADTTH |
| 84 | WSXD x = any AA |
| 85 | RSXD x = any AA |

Additional peptidic antagonists of TGF-β that can be used in conjunction with the conjugates, compositions, and methods described herein include peptide antagonists described in U.S. Pat. No. 7,057,013, US 2009/0263410, and US 2011/0294734, the disclosures of which are incorporated herein by reference in its entirety, as well as peptide antagonists of TGF-β containing an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences. These TGF-β antagonist peptides are based on the structure of TGF-β or a TGF-β receptor, and were designed so as to disrupt the binding of endogenous TGF-β to a TGF-β receptor for the purposes of attenuating TGF-β signaling. These synthetic peptides are summarized in Tables 8 and 9, below.

TABLE 8
Exemplary TGF-β antagonist peptides
that bind TGF-β

| SEQ ID NO. | Amino acid sequence |
|---|---|
| 86 | TSLDATMIWTMM |
| 87 | SNPYSAFQVDIIVDI |
| 88 | TSLMIWTMM |
| 89 | TSLDASIIWAMMQN |
| 90 | SNPYSAFQVDITID |
| 91 | EAVLILQGPPYVSWL |
| 92 | LDSLSFQLGLYLSPH |

TABLE 9
Exemplary TGF-β antagonist peptides that bind
a TGF-β receptor

| SEQ ID NO. | Amino acid sequence |
|---|---|
| 93 | HANFCLGPCPYIWSL |
| 94 | FCLGPCPYIWSLDT |
| 95 | HEPKGYHANFCLGPCP |

Additional peptidic antagonists of TGF-β that can be used in conjunction with the conjugates, compositions, and methods described herein include peptide antagonists described in US 2009/0263410, the disclosure of which is incorporated herein by reference in its entirety, as well as peptide antagonists of TGF-β containing an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences. These peptides are summarized in Table 10, below.

TABLE 10
Exemplary TGF-β antagonist peptides
that bind TGF-β

| SEQ ID NO. | Amino acid sequence |
|---|---|
| 96 | TSLDASIIWAMMQN |
| 97 | KRIWFIPRSSWYERA |
| 98 | KRIWFIPRSSW |
| 99 | KRIWFIPRSSW (Amidated at C-terminus) |
| 100 | KRIWFIPRSSW (Acetylated at N-terminal |
| | K and amidated at C-terminus) |

Additional peptidic antagonists of TGF-β that can be used in conjunction with the conjugates, compositions, and methods described herein include peptide antagonists described in US 2011/0294734, the disclosure of which is incorporated herein by reference in its entirety, as well as peptide antagonists of TGF-β containing an amino acid sequence having at least 50% sequence identity (e.g., at least 50%, 55%, 60%, 65%, 70%, 75%, 80%, 85%, 90%, 95%, 97%, 99%, or greater) to one of these sequences and/or having one or more conservative amino acid substitutions with respect to one of these sequences. These peptides are summarized in Table 11, below.

TABLE 11
Exemplary TGF-β antagonist peptides

| SEQ ID NO. | Amino acid sequence |
|---|---|
| 101 | HANFCLGPCPYIWSL |
| 102 | FCLGPCPYIWSLDT |
| 103 | TSLDATMIWTMM |
| 104 | SNPYSAFQVDIIVDI |
| 105 | TSLMIWTMM |
| 106 | TSLDASIIWAMMQN |
| 107 | SNPYSAFQVDITID |
| 108 | EAVLILQGPPYVSWL |
| 109 | LDSLSFQLGLYLSPH |
| 110 | HEPKGYHANFCLGPCPYIWSLDT |
| 111 | WHKYFLRRPLSVRTR |
| 112 | RFFTRFPWHYHASRL |
| 113 | RKWFLQHRRMPVSVL |
| 114 | SGRRHLHRHHIFSLP |
| 115 | RLAHSHRHRSHVALT |
| 116 | PPYHRFWRGHRHAVQ |
| 117 | KRIWFIPRSSWYERA |
| 118 | MPLSRYWWLFSHRPR |
| 119 | KRIWFIPRSSWYER |
| 120 | KRIWFIPRSSWY |
| 121 | KRIWFIPRSSW |
| 122 | KRIWFIPRSSW (amidated at C-terminus) |
| 123 | KRIWFIPRSSW (acetylated at N-terminal K |
| | and amidated at C-terminus) |

Additional TGF-β antagonists useful in conjunction with the conjugates, compositions, and methods described herein include glycoprotein-A repetitions predominant protein (GARP), as well as well as peptide antagonists of TGF-β containing an amino acid sequence having at least 85% sequence identity (e.g., at least 85%, 90%, 95%, 97%, 99%, or greater) to this protein and/or having one or more conservative amino acid substitutions with respect to this protein. The antagonistic activity of this protein is described in detail, for example, in Wang et al., Molecular Biology of the Cell 23:1129-1139 (2012), the disclosure of which is incorporated herein by reference in its entirety. The amino acid sequence of GARP is shown below.

Glycoprotein-A repetitions predominant protein
(GARP):

(SEQ ID NO: 124)

MRPQILLLLALLTLGLAAQHQDKVPCKMVDKKVSCQVLGLLQVPSVLPPD
TETLDLSGNQLRSILASPLGFYTALRHLDLSTNEISFLQPGAFQALTHLE
HLSLAHNRLAMATALSAGGLGPLPRVTSLDLSGNSLYSGLLERLLGEAPS
LHTLSLAENSLTRLTRHTFRDMPALEQLDLHSNVLMDIEDGAFEGLPRLT
HLNLSRNSLTCISDFSLQQLRVLDLSCNSIEAFQTASQPQAEFQLTWLDL
RENKLLHFPDLAALPRLIYLNLSNNLIRLPTGPPQDSKGIHAPSEGWSAL
PLSAPSGNASGRPLSQLLNLDLSYNEIELIPDSFLEHLTSLCFLNLSRNC
LRTFEARRLGSLPCLMLLDLSHNALETLELGARALGSLRTLLLQGNALRD
LPPYTFANLASLQRLNLQGNRVSPCGGPDEPGPSGCVAFSGITSLRSLSL
VDNEIELLRAGAFLHTPLTELDLSSNPGLEVATGALGGLEASLEVLALQG
NGLMVLQVDLPCFICLKRLNLAENRLSHLPAWTQAVSLEVLDLRNNSFSL
LPGSAMGGLETSLRRLYLQGNPLSCCGNGWLAAQLHQGRVDVDATQDLIC
RFSSQEEVSLSHVRPEDCEKGGLKNINLIIILTFILVSAILLTTLAACCC
VRRQKFNQQYKA

Examples of additional TGF-β antagonists useful in conjunction with the conjugates, compositions, and methods described herein include latency associated peptide (see, e.g., WO 91/08291), large latent TGF-β (see, e.g., WO 94/09812), fetuin (see, e.g., U.S. Pat. No. 5,821,227), decorin and other proteoglycans such as biglycan, fibromodulin, lumican and endoglin (see, e.g., U.S. Pat. Nos. 5,583,103, 5,654,270, 5,705,609, 5,726,149, 5,824,655 5,830,847, 6,015,693, as well as WO 91/04748, WO 91/10727, WO 93/09800, and WO 94/10187).

Further examples of TGF-β antagonists that may be used in conjunction with the compositions and methods described herein include somatostatin (see, e.g., WO 98/08529), mannose-6-phosphate or mannose-1-phosphate (see, e.g., U.S. Pat. No. 5,520,926), prolactin (see, e.g., WO 97/40848), insulin-like growth factor II (see, e.g., WO 98/17304), IP-10 (see, e.g., WO97/00691), arg-gly-asp containing peptides (see, e.g., U.S. Pat. No. 5,958,411 and WO 93/10808), extracts of plants, fungi and bacteria (see, e.g., EP 813875, JP 8119984, and U.S. Pat. No. 5,693,610), antisense oligonucleotides (see, e.g., U.S. Pat. Nos. 5,683,988, 5,772,995, 5,821,234 and 5,869,462, as well as WO 94/25588), and a host of other proteins involved in TGF-β signaling, including SMADs and MADs (see, e.g., EP 874046, WO 97/31020, WO 97/38729, WO 98/03663, WO 98/07735, WO 98/07849, WO 98/45467, WO 98/53068, WO 98/55512, WO 98/56913, WO 98/53830, and WO 99/50296, as well as U.S. Pat. Nos. 5,834,248, 5,807,708, and 5,948,639), in addition to Ski and Sno (see, e.g., G. Vogel, Science, 286:665 (1999) and Stroschein et al., Science, 286:771-74 (1999)) and fragments and derivatives of any of the above molecules that retain the ability to inhibit the activity of TGF-β.

### Small Molecule TGF-β Antagonists

Additional examples of TGF-β antagonists include small molecules that inhibit TGF-β signal transduction. These agents can be classified on the basis of the core molecular scaffolds of these molecules. For example, TGF-β signaling inhibitors may contain a dihydropyrrlipyrazole, imidazole, pyrazolopyridine, pyrazole, imidazopyridine, triazole, pyridopyrimidine, pyrrolopyrazole, isothiazole, or oxazole functionality as the core structural fragment of the molecule. Some non-limiting examples of small molecule inhibitors of TGF-β signaling include ALK5 inhibitor II (also referred to as E-616452), LY364947 (also referred to as ALK5 Inhibitor I, TbR-I Inhibitor, Transforming Growth Factor-b Type I Receptor Kinase Inhibitor), A83-01, and DMH1, known in the art. Other examples of small molecule TGF-β antagonists that can be used in conjunction with the compositions and methods described herein include SB431542 (4-(5-Benzol[1,3]dioxol-5-yl-4-pyridin-2-yl-1H-imidazol-2-yl)-benzamide hydrate, 4-[4-(1,3-Benzodioxol-5-yl)-5-(2-pyridinyl)-1H-imidazol-2-yl]-benzamide hydrate, 4-[4-(3,4-Methylenedioxyphenyl)-5-(2-pyridyl)-1H-imidazol-2-yl]-benzamide hydrate, an Alk5 inhibitor), Galunisertib (LY2157299, an Alk5 inhibitor), LY2109761 (4-[2-[4-(2-pyridin-2-yl-5,6-dihydro-4H-pyrrolo[1,2-b]pyrazol-3-yl)quinolin-7-yl]oxyethyl]morpholine, an Alk5/TGFβRII inhibitor), SB525334 (6-[2-tert-butyl-5-(6-methylpyridin-2-yl)-1H-imidazol-4-yl]quinoxaline, an Alk5 inhibitor), GW788388 (N-(oxan-4-yl)-4-[4-(5-pyridin-2-yl-1H-pyrazol-4-yl)pyridin-2-yl]benzamide, an Alk5 inhibitor), K02288 (3-[6-amino-5-(3,4,5-trimethoxyphenyl)pyridin-3-yl]phenol, an Alk4/Alk5 inhibitor), SD-208 (2-(5-chloro-2-fluorophenyl)-N-pyridin-4-ylpteridin-4-amine, an Alk5 inhibitor), EW-7197 (N-((4-([1,2,4]triazolo[1,5-a]pyridin-6-yl)-5-(6-methylpyridin-2-yl)-1H-imidazol-2-yl)methyl)-2-fluoroaniline, an Alk4/Alk5 inhibitor), and LDN-212854(5-[6-[4-(1-Piperazinyl)phenyl]pyrazolo[1,5-a]pyrimidin-3-yl]-quinoline, an Alk4/Alk5 inhibitor).

Additional examples of small molecule TGF-β antagonists include those that bind TGF-β receptors, such as 2-(3-(6-Methylpyridin-2-yl)-1H-pyrazol-4-yl)-1,5 napththyridine, [3-(Pyridin-2-yl)-4-(4-quinoyl)]-1H-pyrazole, and 3-(6-Methylpyridin-2-yl)-4-(4-quinolyl)-1-phenylthiocarbamoyl-1H-pyrazole. Other small molecule inhibitors include, but are not limited to, SB-431542, (4-[4-(1,3-Benzodioxol-5-yl)-5-(2-pyridinyl)-1H-imidazol-2-yl]-benzamide, described in Halder et al., Neoplasia 7(5):509-521 (2005)), SM16, a small molecule inhibitor of TGFβ receptor ALK5, the structure of which is shown below (Fu, K et al., Arteriosclerosis, Thrombosis and Vascular Biology 28(4):665 (2008)), SB-505124 (an Alk4/Alk5 inhibitor, structure shown below, described in Dacosta Byfield, S., et al., Molecular Pharmacology 65:744-752 (2004)), and 6-bromo-indirubin-3′-oxime (described in U.S. Pat. No. 8,298,825), the disclosures of each of which are incorporated herein by reference.

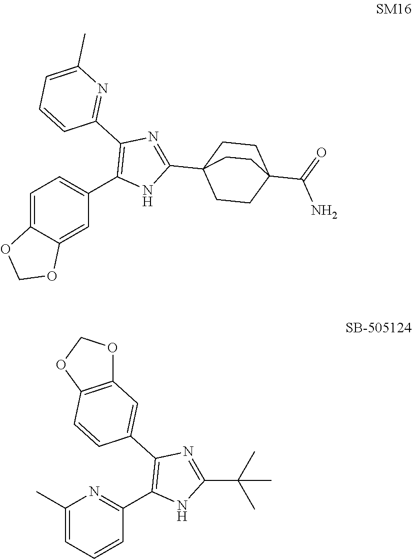 [SMILES: Cc1cccc(-c2nc(C34CCC(C(N)=O)(CC3)CC4)[nH]c2-c2ccc3c(c2)OCO3)n1 ; Cc1cccc(-c2[nH]c(C(C)(C)C)nc2-c2ccc3c(c2)OCO3)n1]

Additional examples of small molecule TGF-β antagonists include, without limitation, those that are described in, e.g., Callahan, J. F. et al., J. Med. Chem. 45:999-1001 (2002); Sawyer, J. S. et al., J. Med. Chem. 46:3953-3956 (2003); Gellibert, F. et al., J. Med. Chem. 47:4494-4506 (2004); Tojo, M. et al., Cancer Sci. 96:791-800 (2005); Valdimarsdottir, G. et al., APMIS 113:773-389 (2005); Petersen et al., Kidney International 73:705-715 (2008); Yingling, J. M. et al., Nature Rev. Drug Disc. 3:1011-1022 (2004); Byfield, S. D. et al., Mol. Pharmacol., 65:744-752 (2004); Dumont, N, et al., Cancer Cell 3:531-536 (2003); WO 2002/094833; WO 2004/026865; WO 2004/067530; WO 209/032667; WO 2004/013135; WO 2003/097639; WO 2007/048857; WO 2007/018818; WO 2006/018967; WO 2005/039570; WO 2000/031135; WO 1999/058128; U.S. Pat. Nos. 6,509,318; 6,090,383; 6,419,928; 7,223,766; 6,476,031; 6,419,928; 7,030,125; 6,943,191; US 2005/0245520; US 2004/0147574; US 2007/0066632; US 2003/0028905; US 2005/0032835; US 2008/0108656; US 2004/015781; US 2004/0204431; US 2006/0003929; US 2007/0155722; US 2004/0138188; and US 2009/0036382, the disclosures of each which are incorporated by reference as they pertain to TGF-β antagonists.

### Bone-Targeting Moieties

### Collagen-Binding Domains

A variety of collagen-binding domains can be used in conjunction with the compositions and methods described herein. For instance, a variety of peptides with collagen-binding activity have been described in U.S. Pat. No. 8,450,272, the disclosure of which is incorporated herein by reference in its entirety. Exemplary collagen-binding peptides described therein are summarized below.

(SEQ ID NO: 125)

Pro Val Tyr Pro Ile Gly Thr Glu Lys Glu Pro Asn
Asn Ser Lys Glu Thr Ala Ser Gly Pro Ile Val Pro
Gly Ile Pro Val Ser Gly Thr Ile Glu Asn Thr Ser
Asp Gln Asp Tyr Phe Tyr Phe Asp Val Ile Thr Pro
Gly Glu Val Lys Ile Asp Ile Asn Lys Leu Gly Tyr
Gly Gly Ala Thr Trp Val Val Tyr Asp Glu Asn Asn
Asn Ala Val Ser Tyr Ala Thr Asp Asp Gly Gln Asn
Leu Ser Gly Lys Phe Lys Ala Asp Lys Pro Gly Arg
Tyr Tyr Ile His Leu Tyr Met Phe Asn Gly Ser Tyr
Met Pro Tyr Arg Ile Asn Ile Glu Gly Ser Val Gly
Arg

(SEQ ID NO: 126)

Glu Ile Lys Asp Leu Ser Glu Asn Lys Leu Pro Val
Ile Tyr Met His Val Pro Lys Ser Gly Ala Leu Asn
Gln Lys Val Val Phe Tyr Gly Lys Gly Thr Tyr Asp
Pro Asp Gly Ser Ile Ala Gly Tyr Gln Trp Asp Phe
Gly Asp Gly Ser Asp Phe Ser Ser Glu Gln Asn Pro
Ser His Val Tyr Thr Lys Lys Gly Glu Tyr Thr Val
Thr Leu Arg Val Met Asp Ser Ser Gly Gln Met Ser
Glu Lys Thr Met Lys Ile Lys Ile Thr Asp Pro Val
Tyr Pro Ile Gly Thr Glu Lys Glu Pro Asn Asn Ser
Lys Glu Thr Ala Ser Gly Pro Ile Val Pro Gly Ile
Pro Val Ser Gly Thr Ile Glu Asn Thr Ser Asp Gln
Asp Tyr Phe Tyr Phe Asp Val Ile Thr Pro Gly Glu
Val Lys Ile Asp Ile Asn Lys Leu Gly Tyr Gly Gly
Ala Thr Trp Val Val Tyr Asp Glu Asn Asn Asn Ala
Val Ser Tyr Ala Thr Asp Asp Gly Gln Asn Leu Ser
Gly Lys Phe Lys Ala Asp Lys Pro Gly Arg Tyr Tyr
Ile His Leu Tyr Met Phe Asn Gly Ser Tyr Met Pro
Tyr Arg Ile Asn Ile Glu Gly Ser Val Gly Arg

Collagen-binding peptides useful in conjunction with the conjugates and methods described herein also include those having at least 85% sequence identity (e.g., at least 85%, 90%, 95%, 97%, 99%, or greater) to one of the foregoing sequences and/or having one or more conservative amino acid substitutions with respect to one of these sequences.

Additionally, collagen-binding peptides derived from human glycoprotein VI (GPVI) have been described, for instance, in U.S. Pat. No. 8,084,577, the disclosure of which is incorporated herein by reference in its entirety. Collagen-binding domains of GPVI can be incorporated into conjugates described herein, for instance, using the synthetic chemistry or protein expression methodologies described below. The sequence of the collagen-binding domain of GPVI is described below:

(SEQ ID NO: 127)

Gln Ser Gly Pro Leu Pro Lys Pro Ser Leu Gln Ala
Leu Pro Ser Ser Leu Val Pro Leu Glu Lys Pro Val
Thr Leu Arg Cys Gln Gly Pro Pro Gly Val Asp Leu
Tyr Arg Leu Glu Lys Leu Ser Ser Ser Arg Tyr Gln
Asp Gln Ala Val Leu Phe Ile Pro Ala Met Lys Arg
Ser Leu Ala Gly Arg Tyr Arg Cys Ser Tyr Gln Asn
Gly Ser Leu Trp Ser Leu Pro Ser Asp Gln Leu Glu
Leu Val Ala Thr Gly Val Phe Ala Lys Pro Ser Leu
Ser Ala Gln Pro Gly Pro Ala Val Ser Ser Gly Gly
Asp Val Thr Leu Gln Cys Gln Thr Arg Tyr Gly Phe
Asp Gln Phe Ala Leu Tyr Lys Glu Gly Asp Pro Ala
Pro Tyr Lys Asn Pro Glu Arg Trp Tyr Arg Ala Ser
Phe Pro Ile Ile Thr Val Thr Ala Ala His Ser Gly
Thr Tyr Arg Cys Tyr Ser Phe Ser Ser Arg Asp Pro
Tyr Leu Trp Ser Ala Pro Ser Asp Pro Leu Glu Leu
Val Val Thr

Collagen-binding peptides useful in conjunction with the conjugates and methods described herein also include those having at least 85% sequence identity (e.g., at least 85%, 90%, 95%, 97%, 99%, or greater) to the foregoing GPVI-derived sequence and/or having one or more conservative amino acid substitutions with respect to this sequence.

Additionally, collagen-binding peptides derived from human fibronectin can be incorporated into the conjugates described herein (e.g., peptides of about 340 residues corresponding to the amino acid sequence between and including Ala260 and Trp599 of human fibronectin) have been described in detail in WO 2000/049159, the disclosure of which is incorporated herein by reference in its entirety.

Collagen-binding peptides useful in conjunction with the conjugates and methods described herein also include those having at least 85% sequence identity (e.g., at least 85%, 90%, 95%, 97%, 99%, or greater) to the foregoing fibronectin-derived sequence and/or having one or more conservative amino acid substitutions with respect to this sequence.

Collagen-binding peptides derived from bone sialoprotein can be incorporated into the conjugates described herein. Such peptide have been described in detail in WO 2005/082941, the disclosure of which is incorporated herein by reference in its entirety. Exemplary sequences derived from the N-terminal domain of bone sialoprotein that bind collagen are summarized below:

(SEQ ID NO: 128)

NGVFKYRPRYFLYKHAYFYPPLKRFPVQ

(SEQ ID NO: 129)

NGVFKYRPRYFLYK

(SEQ ID NO: 130)

HAYFYPPLKRFPVQ

Collagen-binding peptides useful in conjunction with the conjugates and methods described herein also include those having at least 50% sequence identity (e.g., at least 50%, 55%, 60%, 65%, 70%, 75%, 80%, 85%, 90%, 95%, 97%, 99%, or greater) to one of the foregoing sequences and/or having one or more conservative amino acid substitutions with respect to these sequences.

### Hydroxyapatite-Binding Domains

A variety of Hydroxyapatite-binding domains that can be incorporated into conjugates described herein have been identified, for instance, using phage display techniques. Such peptides are described, for example, in U.S. Pat. No. 8,022,040, the disclosure of which is incorporated herein by reference in its entirety. Exemplary hydroxyapatite-binding domains described therein are summarized in Table 12, below.

TABLE 12
Exemplary hydroxyapatite-binding peptides

| SEQ ID NO. | Amino acid sequence |
|---|---|
| 131 | RPHTITN |
| 132 | QSSYNPI |
| 133 | QTHARHQ |
| 134 | ETRTQLL |
| 135 | HHQRSPA |
| 136 | LQKSPSL |
| 137 | PPKDSRG |
| 138 | SAKKVFS |
| 139 | SQHSTQD |
| 140 | TIHSKPA |
| 141 | TKDWLPS |
| 142 | ANPPLSL |
| 143 | AKQTVPV |
| 144 | ATFSPPL |
| 145 | DQYWGLR |
| 146 | EPNHTRF |
| 147 | HMLAQTF |
| 148 | IGYPVLP |
| 149 | KLSAWSF |
| 150 | MYPLPAP |
| 151 | FTLPTIR |
| 152 | SMAAKSS |
| 153 | SMYDTHS |
| 154 | STLASMR |
| 155 | TLMTTPP |
| 156 | WLPPRTQ |
| 157 | RTPLQPLEDFRP |
| 158 | NTTTDIPSPSQF |
| 159 | TLDKYTRLLSRY |
| 160 | YPIMSHTCCHGV |
| 161 | YEPAAAE |
| 162 | ANPYHRH |
| 163 | ASGPTNV |
| 164 | QNYLLPK |
| 165 | GTQTPQP |
| 166 | HSTGPTR |
| 167 | LSKNPLL |
| 168 | LSKNPLL |
| 169 | KLHASLA |
| 170 | PLTQPSH |
| 171 | PHNPGKL |
| 172 | PTTMTRW |
| 173 | VHLTHGQ |
| 174 | TLAPTFR |
| 175 | VHPRPSL |
| 176 | TLLRTQV |
| 177 | SSPPRVY |
| 178 | SSVPGRP |
| 179 | LPFQPPI |
| 180 | IQHQAKT |
| 181 | LPRDLHATPQQI |
| 182 | LTPTMFNMHGVL |
| 183 | SIPKMIPTESLL |
| 184 | SFQSMSLMTLVV |
| 185 | TQTWPQSSSHGL |
| 186 | YELQMP |
| 187 | AMSQTMTAAIEK |
| 188 | GSAGLKYPLYKS |
| 189 | INFQFLKPSTTR |
| 190 | RHTLPLH |
| 191 | NFAMNLR |
| 192 | NFAMNLR |
| 193 | NPQMQRS |
| 194 | NPQMQRS |
| 195 | NPQMQRS |
| 196 | NYPTLKS |
| 197 | NYPTLKS |
| 198 | QNPRQIY |
| 199 | QNPRQIY |
| 200 | QNPRQIY |
| 201 | QNPRQIY |
| 202 | ETYARPL |
| 203 | ETVCASS |
| 204 | KPMQFVH |
| 205 | KPMQFVH |
| 206 | PAKQKAH |
| 207 | PTTWGHL |
| 208 | PTTWGHL |
| 209 | SASGTPS |
| 210 | SSYEYHA |
| 211 | SSYEYHA |
| 212 | STQAHPW |
| 213 | TVLGTFP |
| 214 | WYPNHLA |
| 215 | TTYNSPP |
| 216 | MTSQTLR |
| 217 | WPANKLSTKSMY |
| 218 | WPANKLSTKSMY |
| 219 | NPYHPTIPQSVH |
| 220 | DKLHRLA |
| 221 | QPGLWPS |
| 222 | ESLKSIS |
| 223 | GSCPPKK |
| 224 | GSLFKAL |
| 225 | HQWDHKY |
| 226 | LSAPMEY |
| 227 | MKVHERS |
| 228 | FVNLLGQ |
| 229 | PIDAFFD |
| 230 | PPNMARA |
| 231 | PTNKPHT |
| 232 | SPNNTRE |
| 233 | SPEMKPR |
| 234 | SSSMAKM |
| 235 | TDHPPKA |
| 236 | TLAFQTA |
| 237 | APLSLSL |
| 238 | HYPTVNF |
| 239 | QHNFRGASSSAP |
| 240 | HQFPXSNLVWKP |
| 241 | LSLRASAATDFQ |
| 242 | MQFTPAPSPSDH |
| 243 | SVFLPTRHSPDL |
| 244 | SVSVGMKPSPRP |
| 245 | SVSVGMKPSPRP |
| 246 | SVSVGMKPSPRP |
| 247 | SVSVGMKPSPRP |
| 248 | SVSVGMNAESA |
| 249 | RHTLPLH |
| 250 | NPQMQRS |
| 251 | NYPTLKS |
| 252 | NYPTLKS |
| 253 | DMRQQRS |
| 254 | QNPRQIY |
| 255 | QNPRQIY |
| 256 | QNPRQIY |
| 257 | QNPRQIY |
| 258 | QNPRQIY |
| 259 | QNPRQIY |
| 260 | QNPRQIY |
| 261 | QNPRQIY |
| 262 | QNPRQIY |
| 263 | QTHSSLW |
| 264 | ETYQQPL |
| 265 | ETYARPL |
| 266 | GTSRLFS |
| 267 | LTQTLQY |
| 268 | KAFDKHG |
| 269 | RPMQFVH |
| 270 | KPMQFVH |
| 271 | KPMQFIH |
| 272 | PAKQKAH |
| 273 | SASGTPS |
| 274 | SSHHHRH |
| 275 | SSYEYHA |
| 276 | TGPTSLS |
| 277 | LRAFPSLPHTVT |
| 278 | NPRSQAT |
| 279 | HRLGHMS |
| 280 | LLPLKFK |
| 281 | LPSIHNL |
| 282 | KATITGM |
| 283 | PDIPLSR |
| 284 | PSMKHWR |
| 285 | SAKGRAD |
| 286 | SRTGAHH |
| 287 | SKTSSTS |
| 288 | SPNNPRE |
| 289 | TLQRMGQ |
| 290 | TMTNMAK |
| 291 | TTLSPRT |
| 292 | TTKNFNK |
| 293 | YPKALRN |
| 294 | VVKSNGE |
| 295 | ITGAY |
| 296 | LPLTPLP |
| 297 | HSMPHMGTYLLT |
| 298 | MQFTPAPSPSDH |
| 299 | MPQTLVLPRSLL |
| 300 | SSTQVQHTLLQT |
| 301 | SWPLYSRDSGLG |
| 302 | SVSVGTEAESXA |
| 303 | SVSVGMKPSPRP |
| 304 | SVSVGMKPSPRP |
| 305 | SVSVGMKPSPRP |
| 306 | SVSVGMNAESYG |
| 307 | THPVVFEDERLF |
| 308 | TLPSPLALLTVH |
| 309 | WPTYLNPSSLKA |
| 310 | ASHNPKL |
| 311 | PAKQKAH |
| 312 | PAKQKAH |
| 313 | SASGTPS |
| 314 | TRFYDSL |
| 315 | QNPRQIY |
| 316 | QNPRQIY |
| 317 | QNPRQIY |
| 318 | QNPRQIY |
| 319 | TGPTSLS |
| 320 | TGPTSLS |
| 321 | NPQMQRS |
| 322 | NPQMQRS |
| 323 | NPQMQRS |
| 324 | NPQMQRS |
| 325 | NPQMQRS |
| 326 | KPMQFVH |
| 327 | SSYEYHA |
| 328 | STQAHPW |
| 329 | GTSRLFS |
| 330 | NYPTLKS |
| 331 | NYPTLKS |
| 332 | NYPTLKS |
| 333 | NYPTLKS |
| 334 | NYPTLKS |
| 335 | NYPTLKS |
| 336 | NYPTLKS |
| 337 | HAPVQPN |
| 338 | NPYHPTIPQSVH |
| 339 | NPYHPTIPQSVH |
| 340 | NPYHPTIPQSVH |
| 341 | NPYHPTIPQSVH |
| 342 | HQFISPEPFLIS |
| 343 | SPNFSWLPLGTTSPNFS |
| 344 | WLPLGTT |
| 345 | SVSVGMKPSPRP |
| 346 | SVSVGMKPSPRP |
| 347 | TPLTSPSLVRPQ |
| 348 | TPLSYLKGLVTV |
| 349 | NPMIMNQ |
| 350 | NPMIMNQ |
| 351 | NITQLGS |
| 352 | HTLLSTT |
| 353 | HTLLSTT |
| 354 | HTLLSTT |
| 355 | LGPGKAF |
| 356 | LGPGKAF |
| 357 | LGPGKAF |
| 358 | LGPGKAF |
| 359 | KTSSWAN |
| 360 | KMNHMPN |
| 361 | SLLTPWL |
| 362 | TLGLPML |
| 363 | TGLAKT |
| 364 | IRLIS |
| 365 | LGPGKAF |
| 366 | LGPGKAF |
| 367 | LGPGKAF |
| 368 | DLNYFTLSSKRE |
| 369 | DLNYFTLSSKRE |
| 370 | TMGFTAPRFPHY |
| 371 | TMGFTAPRFPHY |
| 372 | TMGFTAPRFPHY |
| 373 | TMGFTAPRFPHY |
| 374 | HTLLSTT |
| 375 | HTLLSTT |
| 376 | LASTTHV |
| 377 | LGPGKAF |
| 378 | LGPGKAF |
| 379 | LGPGKAF |
| 380 | SLLTPWL |
| 381 | NERQMEL |
| 382 | NKPLSTL |
| 383 | HTLLSTT |
| 384 | LKPFSGA |
| 385 | LGPGKAF |
| 386 | LGPGKAF |
| 387 | LGPGKAF |
| 388 | LGPGKAF |
| 389 | STSAKHW |
| 390 | TMGFTAPRFPHY |
| 391 | TMGFTAPRFPHY |
| 392 | TMGFTAPRFPHY |
| 393 | TMGFTAPRFPHY |
| 394 | TMGFTAPRFPHY |
| 395 | TMGFTAPRFPHY |
| 396 | TMGFTAPRFPHY |
| 397 | CNYPTLKSC |

Hydroxyapatite-binding peptides useful in conjunction with the conjugates and methods described herein also include those having at least 50% sequence identity (e.g., at least 50%, 55%, 60%, 65%, 70%, 75%, 80%, 85%, 90%, 95%, 97%, 99%, or greater) to one of the foregoing sequences and/or having one or more conservative amino acid substitutions with respect to these sequences.

### Polyanionic Peptides

Exemplary targeting moieties that can be used to localize a TGF-β antagonist, such as a TGF-β receptor fusion protein described herein, to osseous tissue include polyanionic peptides, such as those that contain one or more amino acids bearing a side-chain substituent selected from the group consisting of carboxylate, sulfonate, phosphonate, and phosphate. For instance, hydroxyapatite-binding targeting moieties include those that feature a plurality of consecutive or discontinuous aspartate or glutamate residues. Polyanionic peptides can bind hydroxyapatite by virtue, for instance, of electrostatic interactions between negatively charged substituents within the peptide, such as one or more carboxylate, sulfonate, phosphonate, or phosphate substituents, among others, to positively charged calcium ions present within hydroxyapatite.

In some embodiments, the polyanionic peptide contains (e.g., consists of) one or more glutamate residues (e.g., 1-25 glutamate residues, or more, such as 1, 2, 3, 4, 5, 6, 7, 8, 9, 10, 11, 12, 13, 14, 15, 16, 17, 18, 19, 20, 21, 22, 23, 24, or 25, or more, glutamate residues). In some embodiments, the polyanionic peptide contains (e.g., consists of) from 3 to 20 glutamate residues (e.g., 3, 4, 5, 6, 7, 8, 9, 10, 11, 12, 13, 14, 15, 16, 17, 18, 19, or 20 glutamate residues). In some embodiments, the polyanionic peptide contains (e.g., consists of) from 5 to 15 glutamate residues (e.g., 5, 6, 7, 8, 9, 10, 11, 12, 13, 14, or 15 glutamate residues). In some embodiments, the polyanionic peptide contains (e.g., consists of) from 8 to 12 glutamate residues (e.g., 8, 9, 10, 11, or 12 glutamate residues). In some embodiments, the polyanionic peptide contains (e.g., consists of) 5 glutamate residues. In some embodiments, the polyanionic peptide contains (e.g., consists of) 6 glutamate residues. In some embodiments, the polyanionic peptide contains (e.g., consists of) 7 glutamate residues. In some embodiments, the polyanionic peptide contains (e.g., consists of) 8 glutamate residues. In some embodiments, the polyanionic peptide contains (e.g., consists of) 9 glutamate residues. In some embodiments, the polyanionic peptide contains (e.g., consists of) 10 glutamate residues. In some embodiments, the polyanionic peptide contains (e.g., consists of) 11 glutamate residues. In some embodiments, the polyanionic peptide contains (e.g., consists of) 12 glutamate residues. In some embodiments, the polyanionic peptide contains (e.g., consists of) 13 glutamate residues. In some embodiments, the polyanionic peptide contains (e.g., consists of) 14 glutamate residues. In some embodiments, the polyanionic peptide contains (e.g., consists of) 15 glutamate residues.

The polyanionic peptide may be a peptide of the formula Eₙ, wherein E designates a glutamate residue and n is an integer from 1 to 25. For instance, the polyanionic peptide may be of the formula E₁, E₂, E₃, E₄, E₅, E₆, E₇, E₈, E₉, E₁₀, E₁₁, E₁₂, E₁₃, E₁₄, E₁₅, E₁₆, E₁₇, E₁₈, E₁₉, E₂₀, E₂₁, E₂₂, E₂₃, E₂₄, or E₂₅. In some embodiments, the peptide is a peptide of the formula XₙEₘXₒEₚ, wherein E designates a glutamate residue, each X independently designates any naturally-occurring amino acid, m represents an integer from 1 to 25, and n and o each independently represent integers from 0 to 5, and p represents an integer from 1 to 10.

In some embodiments, the glutamate residues are consecutive. In some embodiments, the glutamate residues are discontinuous.

In some embodiments, the polyanionic peptide contains (e.g., consists of) one or more aspartate residues (e.g., 1-25 aspartate residues, or more, such as 1, 2, 3, 4, 5, 6, 7, 8, 9, 10, 11, 12, 13, 14, 15, 16, 17, 18, 19, 20, 21, 22, 23, 24, or 25, or more, aspartate residues). In some embodiments, the polyanionic peptide contains (e.g., consists of) from 3 to 20 aspartate residues (e.g., 3, 4, 5, 6, 7, 8, 9, 10, 11, 12, 13, 14, 15, 16, 17, 18, 19, or 20 aspartate residues). In some embodiments, the polyanionic peptide contains (e.g., consists of) from 5 to 15 aspartate residues (e.g., 5, 6, 7, 8, 9, 10, 11, 12, 13, 14, or 15 aspartate residues). In some embodiments, the polyanionic peptide contains (e.g., consists of) from 8 to 12 aspartate residues (e.g., 8, 9, 10, 11, or 12 aspartate residues). In some embodiments, the polyanionic peptide contains (e.g., consists of) 5 aspartate residues. In some embodiments, the polyanionic peptide contains (e.g., consists of) 6 aspartate residues. In some embodiments, the polyanionic peptide contains (e.g., consists of) 7 aspartate residues. In some embodiments, the polyanionic peptide contains (e.g., consists of) 8 aspartate residues. In some embodiments, the polyanionic peptide contains (e.g., consists of) 9 aspartate residues. In some embodiments, the polyanionic peptide contains (e.g., consists of) 10 aspartate residues. In some embodiments, the polyanionic peptide contains (e.g., consists of) 11 aspartate residues. In some embodiments, the polyanionic peptide contains (e.g., consists of) 12 aspartate residues. In some embodiments, the polyanionic peptide contains (e.g., consists of) 13 aspartate residues. In some embodiments, the polyanionic peptide contains (e.g., consists of) 14 aspartate residues. In some embodiments, the polyanionic peptide contains (e.g., consists of) 15 aspartate residues.

The polyanionic peptide may be a peptide of the formula Dₙ, wherein D designates an aspartate residue and n is an integer from 1 to 25. For instance, the polyanionic peptide may be of the formula D₁, D₂, D₃, D₄, D₅, D₆, D₇, D₈, D₉, D₁₀, D₁₁, D₁₂, D₁₃, D₁₄, D₁₅, D₁₆, D₁₇, D₁₈, D₁₉, D₂₀, D₂₁, D₂₂, D₂₃, D₂₄, or D₂₅. In some embodiments, the peptide is a peptide of the formula XₙDₘXₒDₚ, wherein D designates an aspartate residue, each X independently designates any naturally-occurring amino acid, m represents an integer from 1 to 25, and n and o each independently represent integers from 0 to 5, and p represents an integer from 1 to 10.

In some embodiments, the aspartate residues are consecutive. In some embodiments, the aspartate residues are discontinuous.

In some embodiments, the ratio of amino acids bearing a side-chain that is negatively-charged at physiological pH to the total quantity of amino acids in the polyanionic peptide is from about 0.5 to about 2.0.

### Bisphosphonates

Targeting moieties that may be used in conjunction with the compositions and methods described herein include bisphosphonates. Bisphosphonates are pyrophosphate analogues in which the oxygen bridge has been replaced by a carbon with various side chains (P—C—P). Like pyrophosphate, bisphosphonates bind with high affinity to the bone mineral, hydroxyapatite, due, at least in part, to the strong electrostatic interaction between the anionic phosphonate substituents within these compounds and positively-charged calcium ions within the hydroxyapatite matrix. Bisphosphonates, thus, can be used as targeting moieties to localize a therapeutic agent, such as a TGF-β antagonist described herein, to bone tissue. Exemplary bisphosphonates useful in conjunction with the compositions and methods described herein include compounds represented by Formula (I), below,

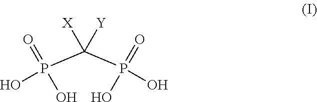

and pharmaceutically acceptable salts thereof, wherein X and Y are each independently hydrogen, halogen, hydroxy, optionally substituted alkoxy, optionally substituted aryloxy, optionally substituted heteroaryloxy, mercapto, optionally substituted alkylthio, optionally substituted arylthio, optionally substituted heteroarylthio, amino, optionally substituted alkylamino, optionally substituted arylamino, optionally substituted heteroarylamino, optionally substituted alkyl, optionally substituted alkenyl, optionally substituted alkynyl, optionally substituted aryl, optionally substituted heteroaryl, optionally substituted cycloalkyl, optionally substituted heterocycloalkyl, or the like.

For instance, particular bisphosphonates that may be used as targeting moieties in the conjugates described herein include those set forth in Table 13, below.

TABLE 13
Exemplary bisphosphonate targeting moieties

| Bisphosphonate | X | Y |
|---|---|---|
| Etidronate | 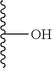 [SMILES: *O] | 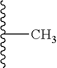 |
| Clodronate | 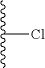 [SMILES: *Cl] | 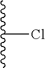 [SMILES: *Cl] |
| Tiludronate | 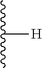 [SMILES: *[H]] | 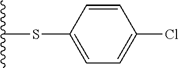 [SMILES: *Sc1ccc(Cl)cc1] |
| Pamidronate | 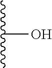 [SMILES: *O] | 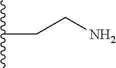 [SMILES: *CCN] |
| Neridronate | 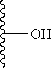 [SMILES: *O] | 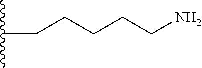 [SMILES: *CCCCCN] |
| Olpadronate | 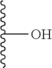 [SMILES: *O] | 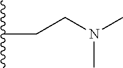 [SMILES: *CCN(C)C] |
| Alendronate | 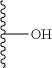 [SMILES: *O] | 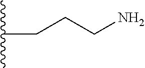 [SMILES: *CCCN] |
| Ibandronate | 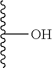 [SMILES: *O] | 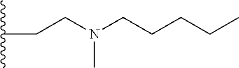 [SMILES: *CCN(C)CCCCC] |
| Risedronate | 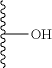 [SMILES: *O] | 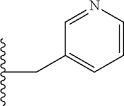 [SMILES: *Cc1cccnc1] |
| Zoledronate | 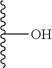 [SMILES: *O] | 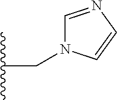 [SMILES: *Cn1ccnc1] |

When used herein in the context of a conjugate, terms for bisphosphonates, such as etidronate, clodronate, tiludronate, pamidronate, neridronate, olpadronate, alendronate, ibandronate, risedronate, and zoledronate, set forth in Table 13, above, refer to a form of the bisphosphonate that is covalently bound to the rest of the conjugate. For instance, a bisphosphonate may be conjugated to a TGF-β antagonist described herein, such as a fusion protein containing one or more domains of TGF-β receptor II each joined to one or more domains of TGF-β receptor III, by modifying one or more substituents of the bisphosphonate to render the molecule compatible with conjugation methods known in the art or described herein. Particularly, to prepare a bisphosphonate for conjugation to a TGF-β antagonist described herein, such as by way of a linker, a moiety on the bisphosphonate may be converted to a nucleophile, electrophile, or other reactive species, thereby rendering the bisphosphonate suitable for reaction with a linker or directly with a TGF-β antagonist. Exemplary methods for converting bisphosphonate compounds into reactive substrates suitable for conjugation are known in the art and are described, for example, in Uludag et al., Biotechnol. Prog. 16:258-267 (2000), the disclosure of which is incorporated herein by reference in its entirety.

### Monoclonal Antibodies

Exemplary TGF-β receptor fusion proteins may be bound to the N-terminal of an Fc domain of an immunoglobulin, either directly or via a hinge linker. Alternatively, exemplary TGF-β receptor fusion proteins may be bound to the C-terminal of an Fc domain of an immunoglobulin, either directly or via a hinge linker. A targeting moiety may be bound to the N-terminal of the Fc domain of the immunoglobulin either directly or via a targeting linker. Similarly, a targeting moiety may be bound to the C-terminal of the Fc domain of the immunoglobulin. Finally, the targeting moiety may be bound either directly or via a targeting linker to the C-terminal of the exemplary TGF-β receptor fusion proteins.

### Fc Domain of an Immunoglobulin

The Fc domain of the immunoglobulin may comprises the immunoglobulin CH2 and CH3 domain and, optionally, at least a part of the hinge region. The Fc domain may be an IgG, IgM, IgD or IgE immunoglobulin domain or a modified immunoglobulin domain derived, therefrom. The IgG immunoglobulin domain may be selected from IgG1, IgG2, IgG3, or IgG4 domains or from modified domains such as are described in U.S. Pat. No. 5,925,734. The immunoglobulin domain may exhibit effector functions, particularly effector functions selected from ADCC and/or CDC. In some embodiments, however, modified immunoglobulin domains having modified, e.g. at least partially deleted, effector functions may be used.

### Signal Peptides

Conjugates composed of proteinogenic amino acids and that may be used in conjunction with the compositions and methods described herein may contain a signal peptide, such as an N-terminal peptide capable of directing excretion of the conjugate from a mammalian cell. Exemplary signal peptides include the albumin signal peptide, MKWVTFLLLLFISGSAFSAAA (SEQ ID NO: 4) or alpha-lactalbumin peptide, MMSFVSLLLVGILFHATQ (SEQ ID NO: 42). Specific signal peptides, such as those described herein, can improve manufacturing of the TGF-β antagonists of the invention, and can be useful for in vivo therapeutic administration of nucleic acids encoding the TGF-β antagonists of the invention.

Exemplary conjugates that contain the albumin signal peptide include those that have the amino acid sequence of SEQ ID NO: 5, as well as those that have at least 70% sequence identity (e.g., at least 75%, 80%, 85%, 90%, 95%, 96%, 97%, 98%, 99%, or more, sequence identity thereto). The protein designated by SEQ ID NO: 5 contains a TGF-β receptor fusion protein composed of an N-terminal human TGF-β receptor II ectodomain, a central rat TGF-β receptor III endoglin domain, and a C-terminal TGF-β receptor II ectodomain. This TGF-β receptor fusion protein is bound at its C-terminus to a decaaspartate (D₁₀) hydroxyapatite-binding polyanionic peptide by way of a glycine- and serine-containing peptidic linker, and is bound at its N-terminus to the albumin signal peptide of SEQ ID NO: 4.

Exemplary TGF-β antagonist conjugate with
signal peptide

SEQ ID NO: 5

MKWVTFLLLLFISGSAFSAAANGAVKFPQLCKFCDVRFSTCDNQKSCMSN
CSITSICEKPQEVCVAVWRKNDENITLETVCHDPKLPYHDFILEDAASPK
CIMKEKKKPGETFFMCSCSSDECNDNIIFSEEYNTSNPDGLGPVESSPGH
GLDTAAAGPEPSTRCELSPINASHPVQALMESFTVLSGCASHGTTGLPRE
VHVLNLRSTDQGPGQRQREVTLHLNPIASVHTHHKPIVFLLNSPQPLVWR
LKTERLAAGVPRLFLVSEGSVVQFPSGNFSLTAETEERNFPQENEHLLRW
AQKEYGAVTSFTELKIARNIYIKVGEDQVFPPTCNIGKNFLSLNYLAEYL
QPKAAEGCVLPSQPHEKEVHIIELITPSSNPYSAFQVDIIVDIRPAQEDP
EVVKNLVLILKSKKSVNWVIKSFDVKGNLKVIAPNSIGFGKESERSMTMT
KLVRDDIPSTQENLMKWALDAGYRPVTSYTMAPVANRFHLRLENNEEMRD
EEVHTIPPELRILLDPDKLPQLCKFCDVRFSTCDNQKSCMSNCSITSICE
KPQEVCVAVWRKNDENITLETVCHDPKLPYHDFILEDAASPKCIMKEKKK
PGETFFMCSCSSDECNDNIIFSEEYNTSNPDGGGGSGGGGSGGGGSGDDD
DDDDDDD,

### Peptide Synthesis Techniques

Systems and processes for performing solid phase peptide synthesis of conjugates described herein include those that are known in the art and have been described, for instance, in U.S. Pat. Nos. 9,169,287; 9,388,212; 9,206,222; 6,028,172; and 5,233,044, among others, the disclosures of each of which are incorporated herein by reference as they pertain to protocols and techniques for the synthesis of peptides on solid support. Solid phase peptide synthesis is a known process in which amino acid residues are added to peptides that have been immobilized on a solid support, such as a polymeric resin (e.g., a hydrophilic resin, such as a polyethylene-glycol-containing resin, or hydrophobic resin, such as a polystyrene-based resin).

Peptides, such as those containing protecting groups at amino, hydroxy, thiol, and carboxy substituents, among others, may be bound to a solid support such that the peptide is effectively immobilized on the solid support. For example, the peptides may be bound to the solid support via their C termini, thereby immobilizing the peptides for subsequent reaction in at a resin-liquid interface.

The process of adding amino acid residues to immobilized peptides can include exposing a deprotection reagent to the immobilized peptides to remove at least a portion of the protection groups from at least a portion of the immobilized peptides. The deprotection reagent exposure step can be configured, e.g., such that side-chain protection groups are preserved, while N-termini protection groups are removed. For instance, an exemplary amino protecting may contain fluorenylmethyloxycarbonyl (Fmoc). A deprotection reagent containing piperidine (e.g., a piperidine solution in an appropriate organic solvent, such as dimethyl formamide (DMF)) may be exposed to the immobilized peptides such that the Fmoc protecting groups are removed from at least a portion of the immobilized peptides. Other protecting groups suitable for the protection of amino substituents include, for instance, the tert-butyloxycarbonyl (Boc) moiety. A deprotection reagent comprising a strong acid, such as trifluoroacetic acid (TFA) may be exposed to immobilized peptides containing a Boc-protected amino substituent so as to remove the Boc protecting group by an ionization process. In this way, peptides can be protected and deprotected at specific sites, such as at one or more side-chains or at the N- or C-terminus of an immobilized peptide so as to append chemical functionality regioselectively at one or more of these positions. This can be used, for instance, to derivatize a side-chain of an immobilized peptide, or to synthesize a peptide, e.g., from the C-terminus to the N-terminus.

The process of adding amino acid residues to immobilized peptides can include, for instance, exposing protected, activated amino acids to the immobilized peptides such that at least a portion of the activated amino acids are covalently bonded to the immobilized peptides to form newly-bonded amino acid residues. For example, the peptides may be exposed to activated amino acids that react with the deprotected N-termini of the peptides so as to elongate the peptide chain by one amino acid. Amino acids can be activated for reaction with the deprotected peptides by reaction of the amino acid with an agent that enhances the electrophilicity of the carbonyl carbon of the amino acid. For example, phosphonium and uronium salts can, in the presence of a tertiary base (e.g., diisopropylethylamine (DIPEA) and triethylamine (TEA), among others), convert protected amino acids into activated species (for example, BOP, PyBOP, HBTU, and TBTU all generate HOBt esters). Other reagents can be used to help prevent racemization that may be induced in the presence of a base. These reagents include carbodiimides (for example, DCC or WSCDI) with an added auxiliary nucleophile (for example, 1-hydroxy-benzotriazole (HOBt), 1-hydroxy-azabenzotriazole (HOAt), or HOSu) or derivatives thereof. Another reagent that can be utilized to prevent racemization is TBTU. The mixed anhydride method, using isobutyl chloroformate, with or without an added auxiliary nucleophile, can also be used, as well as the azide method, due to the low racemization associated with this reagent. These types of compounds can also increase the rate of carbodiimide-mediated couplings, as well as prevent dehydration of Asn and Gln residues. Typical additional reagents include also bases such as N,N-diisopropylethylamine (DIPEA), triethylamine (TEA) or N-methylmorpholine (NMM). These reagents are described in detail, for instance, in U.S. Pat. No. 8,546,350, the disclosure of which is incorporated herein in its entirety.

Cyclic peptides can be synthesized using solid-phase peptide synthesis techniques. For instance, a side-chain substituent, such as an amino, carboxy, hydroxy, or thiol moiety can be covalently bound to a resin, leaving the N-terminus and C-terminus of the amino acid exposed in solution. The N- or C-terminus can be chemically protected, for instance, while reactions are carried out that elongate the peptide chain. The termini of the peptide can then be selectively deprotected and coupled to one another while the peptide is immobilized by way of the side-chain linkage to the resin. Techniques and reagents for the synthesis of head-to-tail cyclic peptides are known in the art and are described, for instance, in U.S. Pat. Nos. 9,388,212 and 7,589,170, the disclosures of which are incorporated herein by reference in their entirety.

### Linkers for Fusion Protein and Conjugate Preparation

### Synthetic Linkers

A variety of linkers can be used to covalently couple reactive residues within a TGF-β antagonist, such as a TGF-β receptor or a domain, fragment, or variant thereof, to another TGF-β receptor or a domain, fragment, or variant thereof in the production of a TGF-β receptor fusion protein, to the Fc domain of an immunoglobulin, or to a bone-targeting moiety, such as a polyanionic peptide that binds hydroxyapatite, in the formation of a therapeutic conjugate as described herein. Exemplary linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for chemical coupling). Examples of linkers useful for the synthesis of conjugates described herein include those that contain electrophiles, such as Michael acceptors (e.g., maleimides), activated esters, electron-deficient carbonyl compounds, and aldehydes, among others, suitable for reaction with nucleophilic substituents present within antibodies, antigen-binding fragments, and ligands, such as amine and thiol moieties. For instance, linkers suitable for the synthesis of therapeutic conjugates include, without limitation, alkyl, cycloalkyl, and heterocycloalkyl linkers, such as open-chain ethyl, propyl, butyl, hexyl, heptyl, octyl, nonyl, or decyl chains, cyclohexyl groups, cyclopentyl groups, cyclobutyl groups, cyclopropyl groups, piperidinyl groups, morpholino groups, or others containing two reactive moieties (e.g., halogen atoms, aldehyde groups, ester groups, acyl chloride groups, acyl anhydride groups, tosyl groups, mesyl groups, or brosyl groups, among others, that can be displaced by reactive nucleophilic atoms present within a TGF-β antagonist peptide and/or bone-targeting moiety), aryl or heteroaryl linkers, such as benzyl, napthyl, or pyridyl groups containing two halomethyl groups that can be displaced by reactive nucleophilic atoms present within a TGF-β antagonist peptide and/or bone-targeting moiety. Exemplary linkers include succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, for instance, Liu et al., 18:690-697, 1979, the disclosure of which is incorporated herein by reference as it pertains to linkers for chemical conjugation. Additional linkers include the non-cleavable maleimidocaproyl linkers, which are described by Doronina et al., Bioconjugate Chem. 17:14-24, 2006, the disclosure of which is incorporated herein by reference as it pertains to linkers for chemical conjugation.

Additional linkers through which one component of a conjugate may be bound to another as described herein include linkers that are covalently bound to one component of the conjugate (e.g., a TGF-β receptor or domain, fragment, or variant thereof) on one end of the linker and, on the other end of the linker, contain a chemical moiety formed from a coupling reaction between a reactive substituent present on the linker and a reactive substituent present within the other component of the conjugate (e.g., another TGF-β receptor or domain, fragment, or variant thereof, or a hydroxyapatite-binding moiety, such as a polyanionic peptide). Exemplary reactive substituents that may be present within a component of the conjugate include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. Linkers useful in conjunction with the conjugates described herein include, without limitation, linkers containing chemical moieties formed by coupling reactions as depicted in Table 14 below. Curved lines designate points of attachment to each component of the conjugate.

TABLE 14
Exemplary chemical moieties formed by coupling
reactions in the formation of TGF-β antagonist conjugates

| Exemplary | |
|---|---|
| Coupling Reactions | Chemical Moiety Formed by Coupling Reactions |
| [3 + 2] Cycloaddition | 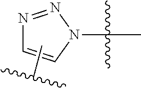 |
| [3 + 2] Cycloaddition | 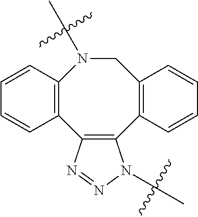 [SMILES: *N1Cc2ccccc2-c2c(nnn2*)-c2ccccc21] |
| [3 + 2] Cycloaddition, Esterification | 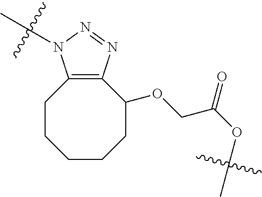 [SMILES: *OC(=O)COC1CCCCCc2c1nnn2*] |
| [3 + 2] Cycloaddition, Esterification | 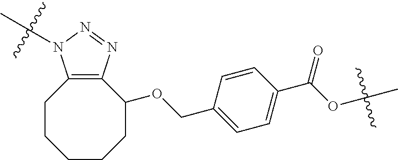 [SMILES: *OC(=O)c1ccc(COC2CCCCCc3c2nnn3*)cc1] |
| [3 + 2] Cycloaddition, Esterification | 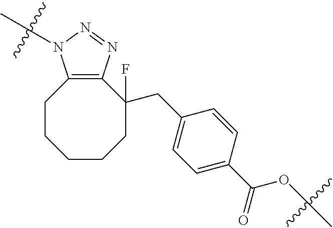 [SMILES: *OC(=O)c1ccc(CC2(F)CCCCCc3c2nnn3*)cc1] |
| [3 + 2] Cycloaddition, Esterification | 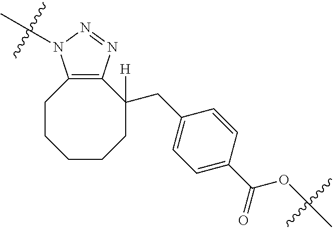 [SMILES: *OC(=O)c1ccc(CC2CCCCCc3c2nnn3*)cc1] |
| [3 + 2] Cycloaddition, Esterification | 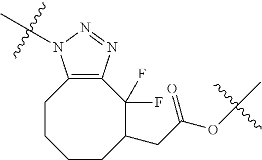 [SMILES: *OC(=O)CC1CCCCc2c(nnn2*)C1(F)F] |
| [3 + 2] Cycloaddition, Esterification | 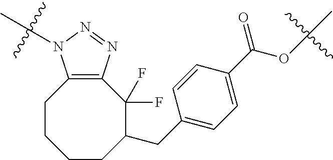 [SMILES: *OC(=O)c1ccc(CC2CCCCc3c(nnn3*)C2(F)F)cc1] |
| [3 + 2] Cycloaddition, Esterification | 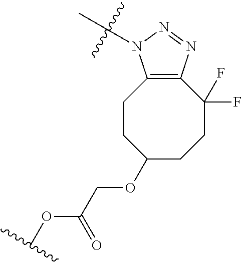 [SMILES: *OC(=O)COC1CCc2c(nnn2*)C(F)(F)CC1] |
| [3 + 2] Cycloaddition, Esterification | 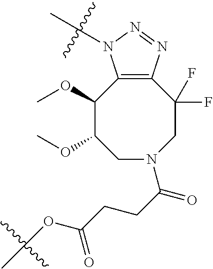 [SMILES: *OC(=O)CCC(=O)N1C[C@H](OC)[C@@H](OC)c2c(nnn2*)C(F)(F)C1] |
| [3 + 2] Cycloaddition, Esterification | 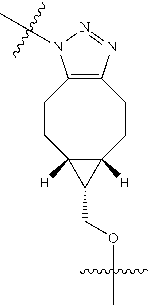 [SMILES: *OC[C@@H]1[C@@H]2CCc3c(nnn3*)CC[C@@H]21] |
| [3 + 2] Cycloaddition, Esterification | 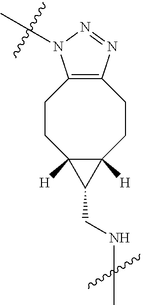 [SMILES: *NC[C@@H]1[C@@H]2CCc3c(nnn3*)CC[C@@H]21] |
| [3 + 2] Cycloaddition, Esterification | 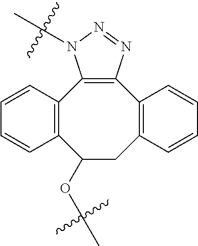 [SMILES: *OC1Cc2ccccc2-c2nnn(*)c2-c2ccccc21] |
| [3 + 2] Cycloaddition, Etherification | 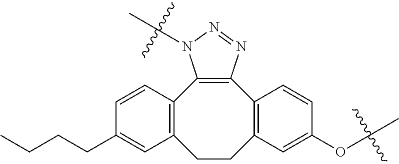 [SMILES: *Oc1ccc2c(c1)CCc1cc(CCCC)ccc1-c1c-2nnn1*] |
| [3 + 2] Cycloaddition | 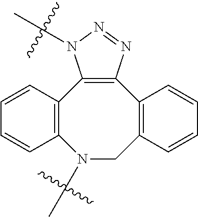 [SMILES: *N1Cc2ccccc2-c2nnn(*)c2-c2ccccc21] |
| Michael addition | 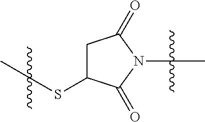 [SMILES: *SC1CC(=O)N(*)C1=O] |
| Michael addition | 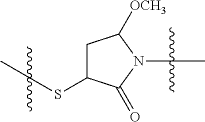 [SMILES: *SC1CC(OC)N(*)C1=O] |
| Imine condensation, Amidation | 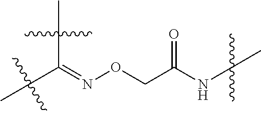 [SMILES: *NC(=O)CON=C(*)*] |
| Imine condensation | 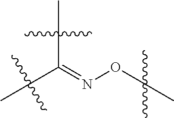 [SMILES: *ON=C(*)*] |
| Disulfide formation | 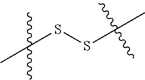 [SMILES: *SS*] |
| Thiol alkylation | 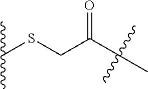 [SMILES: *SCC(*)=O] |
| Condensation, Michael addition | 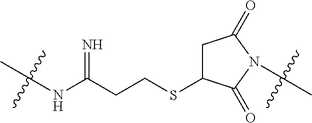 [SMILES: *NC(=N)CCSC1CC(=O)N(*)C1=O] |

### Peptidic Linkers

In addition to the synthetic linkers described above, the binding of one component of a TGF-β receptor fusion protein to another, or one component of a therapeutic conjugate to another (e.g., a TGF-β receptor or TGF-β receptor fusion protein to a hydroxyapatite-binding moiety) can be effectuated by way of a peptide linker, also referred to as a peptidic linker. Most typically, the peptide linker contains 50 or fewer amino acids, e.g., 45, 40, 35, 30, 25, 20, 15, 10, 9, 8, 7, 6, 5, 3, 4, 2, or 1 amino acid(s). In certain instances, the sequence of the peptide linker is a non-TGF-β type II or type III receptor amino acid sequence. In other instances, the sequence of the peptide linker is additional TGF-β type II or type III receptor amino acid sequence, e.g., the 2, 3, 4, 5, 6, 7, 8, 9, 10, 11, 12, 13, 14, 15, 16, 17, 18, 19, 20, 25, to 50 or fewer amino acids flanking the carboxy an/or amino terminal ends of the binding domains. TGF-β receptor fusion proteins and therapeutic conjugates composed of proteinogenic amino acids in which one or more components are joined by a peptide linker can be prepared, for instance, by expressing a nucleic acid encoding the linker in combination with the components of the fusion protein or conjugate. Exemplary peptide linkers include those that contain one or more glycine residues. Such linkers may be sterically flexible due to the ability of glycine to access a variety of torsional angles.

For instance, peptide linkers useful in conjunction with the compositions and methods described herein include one or more glycines, e.g., 1, 2, 3, 4, 5, 6, 7, 8, 9, 10, 11, 12, 15, 18, or more glycines. For example, the linker may comprise (GGG)n, where n=1, 2, 3, 4, 5, 6, 7, etc., such as GGG (SEQ ID NO: 6), and optional adaptor amino acids. Additional examples of peptidic linkers include those that also contain one or more polar amino acids, such as serine or threonine. For instance, linkers useful in conjunction with the compositions and methods described herein include glycine-serine linker, which contain a repeating amino acid sequence of the formula the sequence of (GGGS)n, where n=1, 2, 3, 4, 5, etc. (SEQ ID NO: 60), or the sequence of (GGGGS)n, where n=1, 2, 3, 4, 5, etc. (SEQ ID NO: 61), such as the peptide GGGGS (SEQ ID NO: 7) or GGGGSGGGGSGGGGSG (SEQ ID NO: 8), as well as those that contain one or more cationic or anionic residues, such as a lysine, arginine, aspartate, or glutamate residue.

Additional peptide linkers useful in conjunction with the compositions and methods described herein include amino acid sequences listed in SEQ ID NO: 36, SEQ ID NO: 37, SEQ ID NO: 38, SEQ ID NO: 39, SEQ ID NO: 40, SEQ ID NO: 41, SEQ ID NO: 402, SEQ ID NO: 53, SEQ ID NO: 54, SEQ ID NO: 55, SEQ ID NO: 56, SEQ ID NO: 57, SEQ ID NO: 58, and SEQ ID NO: 59.

### Methods for the Expression of Conjugates in Host Cells

In addition to synthetic chemistry techniques such as those described above, TGF-β antagonists and conjugates described herein (e.g., protein conjugates wherein the TGF-β antagonist is bound to a bone-targeting moiety by one or more peptide bonds) can be expressed in host cells, for instance, by delivering to the host cell a nucleic acid encoding the conjugate protein. The sections that follow describe a variety of established techniques that can be used for the purposes of delivering nucleic acids encoding therapeutic TGF-β antagonists and conjugates described herein to a host cell for the purposes of expressing the antagonist and conjugate protein.

### Transfection Techniques

Techniques that can be used to introduce a polynucleotide, such as nucleic acid encoding a TGF-β antagonist peptide describe herein, into a cell (e.g., a mammalian cell, such as a human cell) are well known in the art. For instance, electroporation can be used to permeabilize mammalian cells (e.g., human cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., Nucleic Acids Research 15:1311 (1987), the disclosure of which is incorporated herein by reference. A similar technique, Nucleofection™, utilizes an applied electric field in order to stimulate the uptake of exogenous polynucleotides into the nucleus of a eukaryotic cell. Nucleofection™ and protocols useful for performing this technique are described in detail, e.g., in Distler et al., Experimental Dermatology 14:315 (2005), as well as in US 2010/0317114, the disclosures of each of which are incorporated herein by reference.

Additional techniques useful for the transfection of cells of interest include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human cell. Squeeze-poration is described in detail, e.g., in Sharei et al., Journal of Visualized Experiments 81:e50980 (2013), the disclosure of which is incorporated herein by reference.

Lipofection represents another technique useful for transfection of cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for instance, by direct fusion of the liposome with the cell membrane or by endocytosis of the complex. Lipofection is described in detail, for instance, in U.S. Pat. No. 7,442,386, the disclosure of which is incorporated herein by reference. Similar techniques that exploit ionic interactions with the cell membrane to provoke the uptake of foreign nucleic acids include contacting a cell with a cationic polymer-nucleic acid complex. Exemplary cationic molecules that associate with polynucleotides so as to impart a positive charge favorable for interaction with the cell membrane include activated dendrimers (described, e.g., in Dennig, Topics in Current Chemistry 228:227 (2003), the disclosure of which is incorporated herein by reference) and diethylaminoethyl (DEAE)-dextran, the use of which as a transfection agent is described in detail, for instance, in Gulick et al., Current Protocols in Molecular Biology 40:1:9.2:9.2.1 (1997), the disclosure of which is incorporated herein by reference. Magnetic beads are another tool that can be used to transfect cells in a mild and efficient manner, as this methodology utilizes an applied magnetic field in order to direct the uptake of nucleic acids. This technology is described in detail, for instance, in US 2010/0227406, the disclosure of which is incorporated herein by reference.

Another useful tool for inducing the uptake of exogenous nucleic acids by cells is laserfection, a technique that involves exposing a cell to electromagnetic radiation of a particular wavelength in order to gently permeabilize the cells and allow polynucleotides to penetrate the cell membrane. This technique is described in detail, e.g., in Rhodes et al., Methods in Cell Biology 82:309 (2007), the disclosure of which is incorporated herein by reference.

Microvesicles represent another potential vehicle that can be used to modify the genome of a cell according to the methods described herein. For instance, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence. The use of such vesicles, also referred to as Gesicles, for the genetic modification of eukaryotic cells is described in detail, e.g., in Quinn et al., Genetic Modification of Target Cells by Direct Delivery of Active Protein [abstract]. In: Methylation changes in early embryonic genes in cancer [abstract], in: Proceedings of the 18th Annual Meeting of the American Society of Gene and Cell Therapy; 2015 May 13, Abstract No. 122.

### Incorporation of Genes by Gene Editing Techniques

In addition to the above, a variety of tools have been developed that can be used for the incorporation of exogenous genes, e.g., exogenous genes encoding a TGF-β antagonist peptide or conjugate described herein, into cells, such as a human cell. One such method that can be used for incorporating polynucleotides encoding a TGF-β antagonist or conjugate described herein into cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene encoding a TGF-β antagonist peptide or conjugate can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene encoding the TGF-β antagonist peptide or conjugate to the DNA of the mammalian cell genome completes the incorporation process. In some cases, the transposon may be a retrotransposon, such that the gene encoding the TGF-β antagonist peptide or conjugate is first transcribed to an RNA product and then reverse-transcribed to DNA before incorporation in the mammalian cell genome. Exemplary transposon systems include the piggybac transposon (described in detail in, e.g., WO 2010/085699) and the sleeping beauty transposon (described in detail in, e.g., US 2005/0112764), the disclosures of each of which are incorporated herein by reference as they pertain to transposons for use in gene delivery to a cell of interest, such as a mammalian cell (e.g., a human cell).

Another tool for the integration of genes encoding TGF-β antagonist peptides or conjugates described herein into the genome of a cell, such as a human cell, is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and an associated Cas9 nuclease. This ensemble of DNA and protein directs site specific DNA cleavage of a sequence of interest by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a particular sequence and localize the Cas9 nuclease to this site. In this manner, highly site-specific cas9-mediated DNA cleavage can be engendered in a foreign polynucleotide because the interaction that brings cas9 within close proximity of the DNA molecule of interest is governed by RNA:DNA hybridization. As a result, one can theoretically design a CRISPR/Cas system to cleave any DNA molecule of interest. This technique has been exploited in order to edit eukaryotic genomes (Hwang et al., Nature Biotechnology 31:227 (2013)) and can be used as an efficient means of site-specifically editing cell genomes in order to cleave DNA prior to the incorporation of a gene encoding a gene. The use of CRISPR/Cas to modulate gene expression has been described in, for instance, U.S. Pat. No. 8,697,359, the disclosure of which is incorporated herein by reference as it pertains to the use of the CRISPR/Cas system for genome editing. Alternative methods for site-specifically cleaving genomic DNA prior to the incorporation of a gene of interest in a cell include the use of zinc finger nucleases (ZFNs) and transcription activator-like effector nucleases (TALENs). Unlike the CRISPR/Cas system, these enzymes do not contain a guiding polynucleotide to localize to a specific sequence. Sequence specificity is instead controlled by DNA binding domains within these enzymes. The use of ZFNs and TALENs in genome editing applications is described, e.g., in Urnov et al., Nature Reviews Genetics 11:636 (2010); and in Joung et al., Nature Reviews Molecular Cell Biology 14:49 (2013), the disclosure of each of which are incorporated herein by reference as they pertain to compositions and methods for genome editing.

Additional genome editing techniques that can be used to incorporate polynucleotides encoding a TGF-β antagonist or conjugate described herein into the genome of a cell of interest, such as a mammalian cell, include the use of ARCUS™ meganucleases that can be rationally designed so as to site-specifically cleave genomic DNA. The use of these enzymes for the incorporation of genes encoding a TGF-β antagonist peptide or conjugate described herein into the genome of a mammalian cell (e.g., a human cell) is advantageous in view of the defined structure-activity relationships that have been established for such enzymes. Single chain meganucleases can be modified at certain amino acid positions in order to create nucleases that selectively cleave DNA at desired locations, enabling the site-specific incorporation of a gene of interest into the nuclear DNA of a cell, such as a mammalian cell (e.g., a human cell). These single-chain nucleases have been described extensively in, for example, U.S. Pat. Nos. 8,021,867 and 8,445,251, the disclosures of each of which are incorporated herein by reference as they pertain to compositions and methods for genome editing.

### Viral Vectors for Nucleic Acid Delivery

Viral genomes provide a rich source of vectors that can be used for the efficient delivery of exogenous genes encoding TGF-β antagonist peptides and conjugates described herein into the genome of a cell (e.g., a mammalian cell, such as a human cell). Viral genomes are particularly useful vectors for gene delivery because the polynucleotides contained within such genomes are typically incorporated into the genome of a cell by generalized or specialized transduction. These processes occur as part of the natural viral replication cycle, and do not require added proteins or reagents in order to induce gene integration. Examples of viral vectors include AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding TGF-β antagonist peptides described herein to a mammalian cell (e.g., a human cell) include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the disclosure of which is incorporated herein by reference as it pertains to viral vectors for use in gene delivery.

### Methods of Therapeutic Treatment

The present invention is based, in part, on the discovery that muscle weakness in diseases associated with elevated TGF-β activity and/or elevated bone turnover can be restored and/or improved through the use of TGF-β antagonists. In the case of osteogenesis imperfecta, active TGF-β is elevated as a consequence of defective collagen and/or excessive release of TGF-β as a result of increased osteoclast activity. The compositions and methods described herein are based, in part, on the finding that bone-derived TGF-β binds to TGF-β receptors on the surface of adjacent muscle, promoting internal signaling via phosphorylation of SMAD2/3 and inducing transcription of a variety of mRNAs associated with cell function. In muscle, elevated TGF-β induces transcription of the Nox4 gene, which encodes NADPH oxidase 4. This enzyme oxidizes the RyR1 calcium channel 1 to yield reactive oxygen species. Oxidation of RyR1 leads to loss of binding by the negative regulator calstabin1, and the ensuing opening of RyR1 causes Ca²⁺ to leak from the sarcoplasma reticulum, thereby depleting Ca²⁺ stores needed for normal muscle contraction and resulting in decreased muscle strength.

The compositions and methods described herein can be used to restore and/or improve muscle function in a patient, such as a human patient suffering from a disease associated with elevated TGF-β signaling, such as elevated bone turnover (e.g., osteogenesis imperfecta, among others described herein), and a muscle disorder, such as muscular dystrophy. For instance, using the compositions and methods described herein, a TGF-β antagonist, such as a TGF-β antagonist conjugated to a bone-targeting moiety, may be administered to a patient suffering from a disease associated with elevated TGF-β signaling, such as elevated bone turnover (e.g., a human patient suffering from osteogenesis imperfecta), so as to restore and/or improve muscle function in the patient.

Additionally, the compositions and methods described herein may be used to determine the propensity of a patient (e.g., a human patient suffering from elevated TGF-β signaling, osteogenesis imperfecta, or other conditions associated with elevated bone turnover) to respond to TGF-β antagonist therapy. Using a method for assessing muscle function (e.g., muscle mass, muscle strength, or muscle quality) described herein or known in the art, a physician may determine that the patient exhibits a level of muscle function that is less than that of a muscle function reference level, such as the level of muscle function of a healthy patient (e.g., a healthy patient of the same gender, age, and/or body mass, among other characteristics, as the patient) or the level of muscle function exhibited by the patient as assessed before the patient was diagnosed as having the disease. A finding that the patient exhibits, for instance, a level of muscle function that is less than that of the muscle function reference level may indicate that the patient is likely to respond to treatment with a TGF-β antagonist, such as a TGF-β antagonist described herein. Since TGF-β antagonism can restore and/or improve muscle function in patients suffering from osteogenesis imperfecta and other disorders associated with elevated bone turnover, patients that exhibit reduced muscle function relative to a muscle function reference level (e.g., the level of muscle function of a healthy patient, such as a healthy patient of the same gender, age, and/or body mass, among other characteristics, as the patient, or the level of muscle function exhibited by the patient as assessed before the patient was diagnosed as having the disease) are particularly likely to benefit from treatment with a TGF-β antagonist or conjugate thereof, such as a TGF-β antagonist or conjugated described herein.

### Routes of Administration

The TGF-β antagonists or conjugates described herein can be administered to a mammalian subject (e.g., a human) suffering from a disease associated with elevated TGF-β activity, e.g., heightened bone turnover, and/or muscle wasting, in order, for example, to improve the condition of the patient, e.g. to improve and/or restore muscle function, by attenuating TGF-β signaling, including at the site of bone tissue. The compositions described herein (e.g., compositions containing a TGF-β antagonist or conjugate thereof of the invention) can be administered to a subject, e.g., via any of the routes of administration described herein, such as subcutaneously, intradermally, intramuscularly, intraperitoneally, intravenously, or orally, or by nasal or by epidural administration. Conjugates described herein can be formulated with excipients, biologically acceptable carriers, and may be optionally conjugated to, admixed with, or co-administered separately (e.g., sequentially) with additional therapeutic agents. The sections that follow describe exemplary conditions that can be treated using the conjugates and pharmaceutical compositions described herein.

### Skeletal Disorders

Diseases and conditions that can be treated using the conjugates described herein include skeletal disorders, such as osteogenesis imperfecta (OI) (for instance, Type I osteogenesis imperfecta, Type II osteogenesis imperfecta, Type III osteogenesis imperfecta, Type IV osteogenesis imperfecta, Type V osteogenesis imperfecta, Type VI osteogenesis imperfecta, Type VII osteogenesis imperfecta, Type VIII osteogenesis imperfecta, Type XI osteogenesis imperfecta, Type X osteogenesis imperfecta, or Type XI osteogenesis imperfecta). These conditions are described, e.g., in Forlino, Nat. Rev. Endo. 7:540 (2011), the disclosure of which is incorporated herein by reference. Osteogenesis imperfecta encompasses a group of congenital bone disorders characterized by deficiencies in one or more proteins involved in bone matrix deposition or homeostasis. Though phenotypes vary among OI types, common symptoms include incomplete ossification of bones and teeth, reduced bone mass, brittle bones, and pathologic fractures. Type-I collagen is one of the most abundant connective tissue proteins in both calcified and non-calcified tissues. Accurate synthesis, post-translational modification, and secretion of type-I collagen are necessary for proper tissue development, maintenance, and repair. Most mutations identified in individuals with osteogenesis imperfecta result in reduced synthesis of type-I collagen, or incorrect synthesis and/or processing of type-I collagen.

In addition to mutations to the type-I collagen gene, other mutations in genes that participate in the intracellular trafficking and processing of collagens have been identified in individuals suffering from osteogenesis imperfecta. These genes include molecular chaperones, such as FK506 binding protein 10 (FKBPIO) and heat shock protein 47 (HSP47) (Alanay et al., 2010; Christiansen et al., 2010; Kelley et al., 2011). Additional mutations have been identified in intermolecular collagen cross-linking genes, such as procollagen-lysine, 2-oxoglutarate 5-dioxygenase 2 (PLOD2), and in members of the collagen prolyl hydroxylase family of genes, including leucine proline-enriched proteoglycan (leprecan) (LEPRE1), peptidylprolyl isomerase B (cyclophilin B) (CYPB), and cartilage associated protein (CRTAP) (Morello et al., 2006; Cabral et al., 2007; Baldridge et al., 2008; van Dijk et al., 2009; Choi et al., 2009; Barnes et al., 2010; Pyott et al., 2011). Mutations aside, proteins such as TGF-β and its corresponding receptors are involved in the onset and propagation of osteogenesis imperfecta (Gebken et al., 2000).

TGF-β expression may be regulated by molecules that bind type-I and type-II collagen. In some instances, TGF-β expression is regulated by a small leucine rich proteoglycan (SLRP) and/or by decorin. In a certain embodiment, decorin does not bind type-I or type-II collagen in which the 3-hydroxyproline site is absent at position 986 of the type-I and/or type-II collagen molecules.

The vertebrate skeleton is comprised of bone, which is a living, calcified tissue that provides structure, support, protection, and a source of minerals for regulating ion transport. Bone is a specialized connective tissue that is comprised of both cellular and acellular components. The acellular extracellular matrix (ECM) contains both collagenous and non-collagenous proteins, both of which participate in the calcification process. A correctly secreted and aligned ECM is critical for proper bone formation. Pathology results when any of the ECM proteins are absent, malformed or misaligned, as is evidenced in osteogenesis imperfecta.

Under normal homeostatic conditions, osteoblasts and osteoclasts work in unison to maintain bone integrity. Pathology results when bone deposition and bone resorption become uncoupled. For example, osteopetrosis is a bone disease characterized by overly dense, hard bone that is a result of unresorptive osteoclasts, while osteoporosis is a bone disorder characterized by brittle, porous bones which can result from increased osteoclast activity. Osteogenesis imperfecta, in particular, can arise as a result of elevated TGF-β expression, which causes an increase in osteoclast-mediated bone resorption. The conjugates described herein can be used to suppress bone resorption by attenuating TGF-β signaling, for instance specifically at the site of pathological bone tissue. The conjugates described herein provide the advantageous pharmacological property of being able to inhibit TGF-β selectively at the site of osseous tissue, thereby restoring bone turnover homeostasis (e.g., in patients suffering from osteogenesis imperfecta) while preserving the effects of TGF-β signaling on healthy tissues.

Several methods can be used to measure and characterize the structure, density, and quality of bone, including histology and histomorphometry, atomic force microscopy, confocal Raman microscopy, nanoindentation, three-point bending test, X-ray imaging, and micro computed tomography (μ-CT). Using these exemplary techniques, for instance, one of skill in the art can monitor the progression of treatment and the effectiveness of therapy. For instance, an improvement in bone integrity, a slowing of bone resorption, and a restoration of homeostasis of bone turnover among patients suffering from osteogenesis imperfecta (e.g., as determined by one or more of the above methods, or other methods known in the art) can be indicators of effective therapeutic treatment.

Additional patients in which muscle function may be improved and/or restored using the compositions or methods described herein or diseases and conditions that can be treated with the conjugates described herein include, for instance, renal osteodystrophy, hyperparathyroid induced bone disease, diabetic bone disease, osteoarthritis, steroid induced bone disease, disuse osteoporosis, and Cerebral Palsy, McCune-Albright Syndrome, Gaucher Disease, Hyperoxaluria, Paget Disease of bone, and Juvenile Paget Disease, metastatic bone cancer (e.g., wherein the metastasis is a secondary metastasis to breast cancer or prostate cancer), osteoporosis, fibrous dysplasia, Calmurati-Engleman Disease, Marfan's Syndrome, osteoglophonic dysplasia, autosomal dominant osteopetrosis, osteoporosis, osteoporosis-pseudoglioma syndrome, juvenile, gerodermia osteodysplastica, Duchenne muscular dystrophy, osteosarcoma, osteogenesis imperfecta congenita, microcephaly, cataracts, pseudohypoparathyroidism, Cleidocranial Dysplasia, Dyskeratosis Congenita, Exudative Vitreoretinopathy 1, Schimmelpenning-Feuerstein-Mims Syndrome, Prader-Willi Syndrome, Achondrogenesis, Antley-Bixler Syndrome, Aspartylglucosaminuria, Celiac Disease, Cerebrooculofacioskeletal Syndrome 1, Lysinuric Protein Intolerance, neuropathy, dyskeratosis congenita, Ehlers-Danlos Syndrome, epiphyseal dysplasia, hyaline fibromatosis syndrome, Perrault Syndrome 1, hemochromatosis, homocystinuria (e.g., due to cystathionine beta-synthase deficiency), hypophosphatemic rickets with hypercalciuria, desbuquois dysplasia, multiple pterygium syndrome, lethal congenital contracture syndrome 1, mitochondrial DNA depletion Ssndrome 6 (hepatocerebral Type), Niemann-Pick Disease, osteopetrosis, porphyria, Rothmund-Thomson Syndrome, Wilson Disease, Dent Disease 1, occipital horn syndrome, hyperglycerolemia, hypophosphatemic rickets, Lowe Oculocerebrorenal Syndrome, renal tubulopathy, diabetes mellitus, cerebellar ataxia, vitamin D hydroxylation-deficient rickets, Warburg micro syndrome 1, Stuve-Wiedemann Syndrome, Blue Rubber Bleb Nevus syndrome, Singleton-Merten Syndrome, microcephalic osteodysplastic primordial dwarfism, dysosteosclerosis, Hallermann-Streiff Syndrome, Bruck Syndrome 1, multiple pterygium syndrome (e.g., X-Linked), spondylometaphyseal dysplasia with dentinogenesis imperfecta, Hall-Riggs Mental Retardation Syndrome, infantile multisystem neurologic disease with osseous fragility, acrocephalopolysyndactyly Type III, acroosteolysis, ACTH-independent macronodular adrenal hyperplasia, amino aciduria with mental deficiency, arthropathy, bone fragility (e.g., with craniosynostosis, ocular proptosis, hydrocephalus, and distinctive facial features), brittle cornea syndrome, cerebrotendinous xanthomatosis, Cri-Du-Chat Syndrome, dysplasia epiphysealis hemimelica, autosomal dominant Ehlers-Danlos Syndrome, familial osteodysplasia, Flynn-Aird Syndrome, gerodermia osteodysplastica, glycogen storage disease Ia, Hutchinson-Gilford Progeria Syndrome, Infantile Systemic Hyalinosis, hypertrichotic osteochondrodysplasia, hyperzincemia with functional zinc depletion, hypophosphatasia, autosomal dominant hypophosphatemic rickets, X-linked recessive hypophosphatemic rickets, Lichtenstein Syndrome, macroepiphyseal dysplasia (e.g., with osteoporosis wrinkled skin, and aged appearance), Menkes Disease, Mental Retardation (e.g., X-Linked, Snyder-Robinson type), Jansen type metaphyseal chondrodysplasia, microspherophakia-metaphyseal dysplasia, morquio syndrome a, Morquio Syndrome B, ossified ear cartilages (e.g., with mental deficiency, muscle wasting, and osteocraniostenosis), osteoporosis and oculocutaneous hypopigmentation syndrome, osteoporosis-pseudoglioma syndrome, juvenile osteoporosis, osteosclerosis with ichthyosis and fractures, ovarian dysgenesis 1, ovarian dysgenesis 2, ovarian dysgenesis 3, ovarian dysgenesis 4, pituitary adenoma, polycystic lipomembranous osteodysplasia with sclerosing leukoencephalopathy, Prader-Willi Habitus, osteopenia, Okamoto type premature aging syndrome, Prieto X-linked mental retardation syndrome, pycnodysostosis, Pyle Disease, Reifenstein Syndrome, autosomal dominant distal renal tubular acidosis, Type 1 Schwartz-Jampel Syndrome, Type 2 Schwartz-Jampel Syndrome, Type 3 Schwartz-Jampel Syndrome, Type 4 Schwartz-Jampel Syndrome, X-linked spondyloepiphyseal dysplasia tarda, and Torg-Winchester Syndrome.

### Muscular Disorders

In addition to treating skeletal disorders, the compositions and methods described herein can be used to treat muscle diseases, such as muscular dystrophies, including Duchenne muscular dystrophy (DMD). DMD represents the most common inherited neuromuscular disease, and is characterized by a lack of dystrophin, muscle wasting, fibrosis, and elevated TGF-β signaling (Acuña et al., Human Molecular Genetics 23:1237-1249 (2014), the disclosure of which is incorporated herein by reference). Particularly, TGF-β signal transduction has been implicated in DMD pathology, and is known to stimulate fibrosis, promote myonecrosis, and inhibit muscle regeneration (Kemaladewi et al., Molecular Therapy—Nucleic Acids 3:e156 (2014) and Taniguti et al., Muscle & Nerve 43:82-87 (2011), the disclosure of which is incorporated herein by reference). By localizing to bone tissue and inhibiting the activity of TGF-β in the proximity of skeletal muscle, the conjugates and pharmaceutical compositions described herein can suppress fibrotic and myonecrotic activity, thereby improving muscle function in patients suffering from muscular dystrophies, such as DMD. As in the case of the treatment of skeletal disorders, the conjugates described herein provide the beneficial property of being able to inhibit TGF-β selectively at the site of skeletal-muscular interface, thereby improving muscle function (e.g., in patients suffering from a muscular dystrophy, such as DMD) while preserving the effects of TGF-β signaling on healthy tissues.

In addition to treating DMD, the compositions and methods described herein can be used to treat various other muscular dystrophies, such as inherited muscular dystrophies associated with a laminin-α2 deficiency. TGF-β inhibition has shown beneficial effects in the treatment of a mouse model of laminin-α2-deficient congenital muscular dystrophy. Particularly, it was found that chronic treatment of a mouse model with the TGF-β inhibitor, Losartan, significantly increased the lifespan of the mouse, decreased the percentage of fibrotic areas in the muscle, reduced collagen deposits, and significantly improved both the hindlimb and forelimb muscle strength of the mutant mice (see, e.g., Elbaz et al., Ann. Neurol. 71:699-708 (2012), the disclosure of which is incorporated herein by reference).

Further, the compositions and methods described herein can be used to treat muscular dystrophy caused by mutations in caveolin-3. This form of muscular dystrophy is amenable to treatment with agents that reduce TGF-β signaling, as it has been shown that caveolin-3-deficient mice treated with a TGF-β receptor type I kinase inhibitor exhibited weight gain and a reduction in hindlimb muscle atrophy (see, e.g., Ohsawa et al., Lab. Invest. 92:1100-1114 (2012), the disclosure of which is incorporated herein by reference).

The compositions and methods described herein can additionally be used to treat acquired muscle diseases, such as sarcopenia. Sarcopenia is described as the loss of muscle function (e.g., muscle mass) that is characterized by impaired regeneration and increased frailty in older populations. Recent studies have suggested that TGF-β signaling plays a significant role in the progression of this condition. It was recently shown that genetically normal, yet aged, sarcopenic muscle had reduced fibrosis and improved muscle function after injury when treated with Losartan (see, e.g., Burks et al., Sci. Transl. Med. 82:82ra37 (2011), the disclosure of which is incorporated herein by reference). Losartan also prevented the loss of muscle fibers in the exaggerated response to immobilization atrophy observed in sarcopenic muscle (Burks et al., 2011). Immobilization atrophy in aged muscle was found to be due to the loss of muscle fibers themselves, rather than to a reduction in fiber diameter. This loss of muscle fibers, the reduction in fibrosis, and the enhanced muscle regeneration with Losartan treatment were attributed to the blockade of both the canonical and non-canonical TGF-β signaling pathways. Thus, sarcopenia, and the fibrosis associated with this condition, can be treated with TGF-β antagonists. As with the muscular dystrophies described above, the conjugates described herein provide the beneficial property of being able to inhibit TGF-β selectively at the site of skeletal-muscular interface, thereby improving muscle function (e.g., in patients suffering from an acquired muscle disease, such as sarcopenia) while preserving the effects of TGF-β signaling on healthy tissues.

### Methods of Assaying Muscle Function

The compositions (e.g., compositions containing a TGF-β antagonist or conjugate thereof) and methods described herein can be used to treat a mammalian subject (e.g., a human) suffering from a disease associated with elevated TGF-β signaling in order to improve muscle function in the subject. For instance, treatment of a patient suffering from a muscular dystrophy, such as DMD, may improve muscle function in the subject. This improvement in muscle function may be assessed, for instance, by any methodology known in the art for measuring muscle strength, muscle quality, muscle mass, and/or the general functional status of the subject. A variety of quantitative or qualitative approaches may be used to assess muscle function (e.g., manual muscle testing, dynamometry, isokinetics, cable tensiometry, muscle mechanography, imaging techniques, functional status assessments, or biochemical assays), examples of which are further described below. Using one or more such approaches to assess muscle function, for instance, one of skill in the art can identify subjects who exhibit reduced muscle function relative to a muscle function reference level (e.g., the level of muscle function of a healthy patient, such as a healthy patient of the same gender, age, and/or body mass, among other characteristics, as the patient) and therefore may benefit from treatment with the compositions described herein. Further, one or more of the methods described herein may be used to monitor changes (e.g., improvements or lack of improvement) in muscle function over time, e.g., to evaluate therapeutic efficacy. Given the range of accepted methodologies available for assessing muscle function, it will be appreciated by one skilled in the art that the particular methodologies used to assess muscle function in a subject may vary based on the skills or judgement of the practitioner carrying out the assessment. In some instances, one or more particular methodologies may be selected based on considerations of a subject's abilities or limitations, as deemed appropriate by a skilled artisan. Methods for assessing muscle function are described, for example, in Waning et al., Nature Medicine 21:1262-1275 (2015), the disclosure of which is incorporated herein by reference as it pertains to methods of assessing muscle function. Exemplary approaches for assessing muscle function are described in further detail, below.

In some instances, muscle function may be assessed by manual muscle testing (MMT). MMT is a procedure for the evaluation of the function of individual muscles and muscle groups based on the effective performance of a movement in relation to the forces of gravity and manual resistance. Various test positions and procedures for MMT and examples of common grading scales may be used with MMT (e.g., Medical Research Council, Daniels and Worthingham, or Kendall and McCreary grading scale). The particular grading system selected or additional devices (e.g., dynamometer) used during MMT may vary depending on the practitioner and/or the subject. See, for example, Hislop et al. (2013). Daniels and Worthingham's Muscle Testing: Techniques of Manual Examination and Performance Testing. Elsevier Health Sciences., the disclosure of which is incorporated herein by reference.

In some instances, muscle function may be assessed by dynamometry. Dynamometry includes methods of strength testing that use strength measuring devices (e.g., hand-grip, hand-held, fixed, and isokinetic dynamometers). For example, is some instances, a hand-held dynamometer (HHD) instrument is used to measure muscle function, e.g., during the aforementioned MMT. In some instances, a grip strength test may be used to assess muscle strength (e.g., upper extremity muscle force) using a hand-grip dynamometer. Further, a dynamometer can be used to measure the isometric muscle strength in the shoulder abductors, hip flexors, ankle dorsal flexor, and grip strength bilaterally, for instance. See, for example, Payton, C., & Bartlett, R. (Eds.). (2007). Biomechanical evaluation of movement in sport and exercise: the British Association of Sport and Exercise Sciences guide. Routledge, the disclosure of which is incorporated herein by reference.

In some instances, muscle function may be assessed by muscle mechanography. Muscle mechanography is a method that can quantitatively assess muscle function based on the performance of movements by the subject such as heel raises, chair rises, single two-legged countermovement jumps, serial one- or two-legged jumps (hopping), or sway on a ground reaction force plate. Muscle mechanography directly measures the applied force vector and calculates measures of muscle force, velocity, power, jump height, and balance or sway (i.e., the change of the center of gravity during a balance test).

In some instances, muscle function is assessed based on measurements of muscle cross-sectional area, volume, density, or mass using any known or otherwise effective technique that provides muscle area, volume or mass, such as DEXA, or using visual or imaging techniques (e.g., magnetic resonance imaging (MRI) or computed tomography (CT) scans). For example, in some instances, peripheral quantitative computer tomography (pQCT) may be used to measure the cross-sectional area or density of a muscle.

In some instances, muscle function is assessed based on clinical assays that assess the impact of elevated TGF-β on muscles on a biochemical level by testing a muscle biopsy. For example, TGF-β elevation can be confirmed via demonstration that the downstream signaling molecules SMAD2 and SMAD3 are activated. This can be measured by immunoblot analysis showing an increased amount of phosphorylated SMAD2 or SMAD3 is present relative to total SMAD2 or SMAD3 in muscle lysates. To assess involvement of NADPH oxidase 4, Nox4 mRNA can be measured using standard RT-PCR in muscle derived from individuals with bone disorders and can be compared to muscle from healthy individuals. Immunoblots of muscle lysates may also be performed to demonstrate oxidation and nitrosylation of RyR1, two downstream consequences of NADPH oxidase 4. Finally, co-immunoprecipation of RyR1 and its associated regulatory protein, Calstabin can be performed. Demonstration that calstabin binding to RyR1 is reduced in muscles from individuals with bone disorders relative to healthy individuals can be used as a surrogate to monitor calcium leak in muscles and associated muscle weakness.

Other non-limiting examples of methods to assess muscle function include the following: self-selected or usual walking gait speed (e.g., where gait speed is the distance traveled divided by the ambulation time); maximum walking gait speed; step length (e.g., wherein step length is the perpendicular distance between the heel of one foot-strike to the heel of the next foot-strike of the opposite foot); step time (e.g., wherein step time is the time elapsed from floor contact of one foot to floor contact of the next foot); stride length (e.g., wherein stride length is the perpendicular distance between the heel of one foot-strike to the heel of the next foot-strike of the same foot); stride time; base width (e.g., wherein base width is the perpendicular distance from the heel of one foot-strike to the line of progression between two foot-strikes of the opposite foot); step width; stride width; gait cycling time; stance time; swing time; double support phase (e.g., wherein double support phase is the phase of the gait cycle when both feet are in contact with the ground); gait parameters measured on an inclined plane, declined plane, or throughout progressively increased velocity on a treadmill; intraindividual variability for gait measures; chair rise test (e.g., wherein the amount of time to complete 5 chair rises is measured); Katz Index of Independence in Activities of Daily Living; Palliative Performance Scale; Tinetti Gait and Balance Scale; Star Excursion Balance Test; tandem standing and tandem walking to measure balance; single-leg dynamic postural sway on a force plate; single-leg stance time on a hard surface; single-leg stance time on a balance pad; manual muscle testing; isokinetic or isometric measurements of muscle strength; one-repetition maximum strength; maximum rate of force development; electromyography (EMG) of muscle; median activation frequency determined by EMG of soleus and gastrocnemius medialis during plantar-flexion; mean amplitude voltage determined by EMG of muscle; EMG of muscle before or after activity; EMG during stance perturbation, training or jumping; nerve stimulation twitch response (e.g., of the soleus and gastrocnemius muscle); reflex activity during flexion; Hoffman's reflex (H-reflex) or other mechanical stretch reflex; H-reflex measured during 2 tasks such as plantar flexion and stance perturbation; Berg Balance Scale; Short Physical Performance Battery; mechanography; jump height; twenty-meter sprint performance; ten yard sprint performance; forty yard sprint performance; countermovement jump power; bounce-drop jump power; and vertical impact force before jumping.

Muscle function can be based on one or more muscles or muscle groups in a subject, e.g., muscles associated with fingers, hands, arms, torso, abdominals, shoulders, back, neck, legs, knees, ankle, foot, or toes. For example, in some instances the muscle function may be tested for one or more muscles selected from one or more of the following muscles: pectoralis major, pectoralis minor, serratus anterior, flexor halluces brevis, flexor digitorum brevis, flexor hallucis longus, flexor digitorum longus, extensor digitorum longus and brevis, fibularis tertius, extensor hallucis longus and brevis, tibialis anterior, tibialis posterior, fibularis longus and brevis, triceps brachii and anconeus, latissimus dorsi, teres major, infraspinatus and teres minor, rhomboid and levator scapulae, middle trapezius, lower trapezius, soleus, adductor pollicis, abductor pollicis brevis, opponens pollicis, flexor pollicis longus, flexor pollicis brevis, extensor pollicis longus, extensor pollicis brevis, abductor pollicis longus, abductor digiti minimi, opponens digiti minimi, flexor digiti minimi, lumbricals and interossei, palmaris longus, extensor digitorum, flexor digitorum superficialis, flexor digitorum profundus, flexor carpi radialis, flexor carpi ulnaris, extensor carpi radialis longus, extensor carpi radialis brevis, extensor capri ulnaris, pronator teres, pronator quadratus, supinator and biceps, brachioradialis, coracobrachialis, biceps brachii, brachialis, supraspinatus and middle deltoid, anterior deltoid, posterior deltoid, upper trapezius, supraspinatus, and gastrocnemius.

In some instances, the muscle function assessment may assess certain bodily movements or other functional manifestations of muscle function, e.g., shoulder shrug, shoulder abduction, elbow flexion or supinated arm, elbow flexion of neutral arm, elbow extension, radial wrist extension, wrist flexion, thumb extension, fifth digit abduction, hip flexion, knee extension, big toe extension, knee flexion, ankle plantar flexion, posture, gripping, jumping, hopping (one feet or two feet), standing up, or sitting down.

Assessments of muscle function can be performed at any point before, during, or after treatment. In some instances, a muscle function assessment performed prior to treatment may be used for prognostic, diagnostic, or predictive purposes. For example, an individual who displays muscle weakness based on the assessments described herein may be identified as one who may benefit from treatment. Muscle function may also be assessed during or after treatment to monitor changes in muscle function. In some instances, assessments of muscle function at multiple time points before or during treatment. For example, an improvement in muscle function in a subject overtime following administration of the compositions described herein may be an indicator that the treatment is effective or that the subject is responsive to treatment. In contrast, a lack of change or a decrease in muscle function over time following administration of the compositions described herein may be an indicator of lack of therapeutic efficacy

The results of the muscle function assessment can be used to identify subjects with muscle weakness (e.g., subjects in need of treatment). For example, in instances of quantitative determinations of muscle function, a measurement of muscle function that is lower than a reference value (e.g., muscle function that is lower by about 1%, about 2%, about 5%, about 10%, about 15%, about 20%, about 25%, about 30%, about 35%, about 40%, about 45%, about 50%, about 55%, about 60%, about 65%, about 70%, about 80%, about 85%, about 90%, about 95%, about 100%, or more than 100% relative to a reference value) may indicate that the individual is experiencing muscle weakness. In some instances, a measurement of muscle function that is lower than a reference value (e.g., a value that is lower by about 2×, 3×, 4×, 5×, 6×, 7×, 8×, 9×, 10×, 15×, 20×, 25×, 30×, 35×, 40×, 45×, 50× or more than 50× relative to a reference value) may indicate that the individual is experiencing muscle weakness. The reference value may be, for instance, a measure of muscle function from one or more control subjects (e.g., a healthy individual or healthy population), a pre-assigned reference value, or a measure of muscle function measured at one or more previous time points in an individual.

In instances of qualitative assessments that involve (e.g., functional status assessments or MMT), a determination of muscle weakness may be made based on well-known grading scales accepted in the art. In some instances, a lack of an ability to perform a certain movement or physical task may be indicative of muscle weakness.

The results of the muscle function assessment may also be used to monitor whether treatment is effective in improving muscle function in an individual. For example, in instances of quantitative determinations of muscle function, a measurement of muscle function that is higher than a reference value (e.g., muscle function that is higher by about 1%, about 2%, about 5%, about 10%, about 15%, about 20%, about 25%, about 30%, about 35%, about 40%, about 45%, about 50%, about 55%, about 60%, about 65%, about 70%, about 80%, about 85%, about 90%, about 95%, about 100%, or more than 100%) indicates that the individual is responsive to treatment. In some instances, a measurement of muscle function that is higher than a reference value (e.g., a value that is lower by about 2×, 3×, 4×, 5×, 6×, 7×, 8×, 9×, 10×, 15×, 20×, 25×, 30×, 35×, 40×, 45×, 50× or more than 50×) indicates that the individual is responsive to treatment. In instances of qualitative assessments (e.g., functional status assessments or MMT), a determination of improvements, or lack thereof, in muscle function overtime may be made based on well-known grading scales accepted in the art. In some instances, an ability to perform a certain movement or physical task that could not be performed previously may be indicative of improvements in muscle function.

Assessing Propensity to Benefit from TGF-β Antagonist Therapy

The compositions and methods described herein may be used to determine the propensity of a patient (e.g., a human patient conditions associated with elevated TGF-β) signalling to respond to TGF-β antagonist therapy. Using a method for assessing muscle function (e.g., muscle mass, muscle strength, or muscle quality) described above or known in the art, a physician may determine that the patient exhibits a level of muscle function that is less than that of a muscle function reference level, such as the level of muscle function of a healthy patient (e.g., a healthy patient of the same gender, age, and/or body mass, among other characteristics, as the patient). A finding that the patient exhibits, for instance, a level of muscle function that is less than that of the muscle function reference level may indicate that the patient is likely to respond to treatment with a TGF-β antagonist, such as a TGF-β antagonist described herein.

For example, a physician of skill in the art may assess a patient's likelihood to benefit from TGF-β antagonist therapy by determining a level of muscle function exhibited by the patient, such as a level of muscle mass, muscle strength, or muscle quality exhibited by the patient, and comparing the level of muscle function exhibited by the patient to a muscle function reference level. A finding that the patient exhibits a level of muscle function that is less than the muscle function reference level (e.g., by 5%, 10%, 15%, 20%, 25%, 30%, 35%, 40%, 45%, 50%, 55%, 60%, 65%, 70%, 75%, 80%, 85%, 90%, 95%, or more) indicates that the patient is likely to benefit from TGF-β antagonist therapy.

Upon determining that the patient is likely to benefit from treatment with a TGF-β antagonist, such as a TGF-β antagonist described herein, the patient may be administered a TGF-β antagonist accordingly. The TGF-β antagonist may be, for instance, conjugated to a bone-targeting moiety, thereby reducing TGF-β signalling in the proximity of the skeletal-muscular interface. In this way, for instance, TGF-β signalling in healthy tissues may be preserved. The TGF-β antagonist or conjugate thereof may be administered to the patient, for instance, by one or more of the routes of administration described herein, such as subcutaneously, intradermally, intramuscularly, intraperitoneally, intravenously, or orally, or by nasal or by epidural administration. The TGF-β antagonist or conjugate thereof may, for instance, be formulated with one or more excipients and/or biologically acceptable carriers, and may be optionally conjugated to, admixed with, or co-administered separately (e.g., sequentially) with one or more additional therapeutic agents

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1

### Expression, Surface Plasmon Resonance (SPR), and Neutralization Assay of RER-Fc-D10 TGF-β Trap (PCT-0015; SEQ ID NO: 14)

### Expression Vector

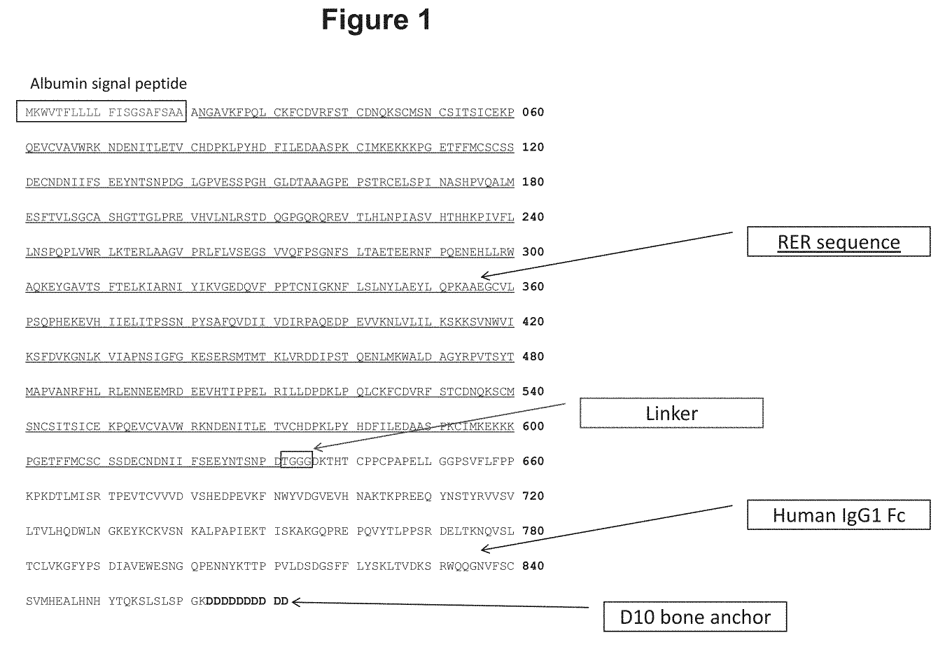

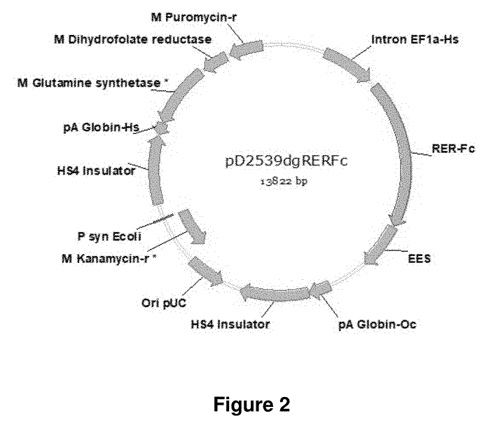

The coding region of the TGF-β receptor fusion protein RER-Fc-D10 TGF-β Trap (PCT-0015) (FIG. 1) was synthesized by Atum Bio and subcloned into a eukaryotic expression vector for transfection into CHO suspension cells using standard Molecular Biological techniques. Briefly, the synthesized fragment was excised from the parental vector by restriction enzyme digest with Sapl. The appropriate sized fragment was gel purified on a 0.8% Agarose, 0.5× TAE gel and ligated into eukaryotic expression vector pD2539dg (FIG. 2). After transformation, bacterial clones positive for insert were confirmed by Sanger sequencing. Construct, pD2539dg was used for transfections to generate stable pools given the behavior of the EF-1a promoter in long term stable culture. The correct cDNA clone was grown at large scale and purified for transfection using a commercially available kit (Zymogen).

### Transient Transfection of RER-Fc-D10 TGF-β Trap (PCT-0015) Expression in CHO Suspension Cells

CHO suspension cells were maintained in serum free medium and routinely passed at cell densities between 3×10⁵ to 3×10⁶/ml. For transfection, CHO cells were harvested and suspended at 1×10⁶ cells/ml and one milliliter was plated into each well of a 6 well dish. Transfections were carried out using Lipofectamine® 2000 following manufacturer's instructions. Post transfection, cell culture supernatants were analyzed for RER fusion protein expression by immunoblot. Supernatant samples were taken at 24, 48 and 72 hr. The transient transfected pools were subsequently placed under selection using puromycin at 10 μg/ml and a cell density of 3×10⁵ cells/ml. After one week of static culture, the selected pools were placed on a shaker platform and cultured until cell densities reached 1×10⁶ cells/ml and viability of >90% for 3 passages. At this stage the cells are deemed to have recovered after phase I and the expression of RER fusion protein was determined.

Stable pools of RER fusion protein (Pool 2 and Pool 3) were tested for protein expression post selection with puromycin. Cells were seeded at 3×10⁵ cells/ml in serum free medium without puromycin and grown with agitation at 125 rpm at 37° C. and 8% CO₂. Cell viability was determined every other day using Trypan blue exclusion to delineate viable from non-viable cells.

### Purification of RER-Fc-D10 TGF-β Trap (PCT-0015) Using Protein A Sepharose

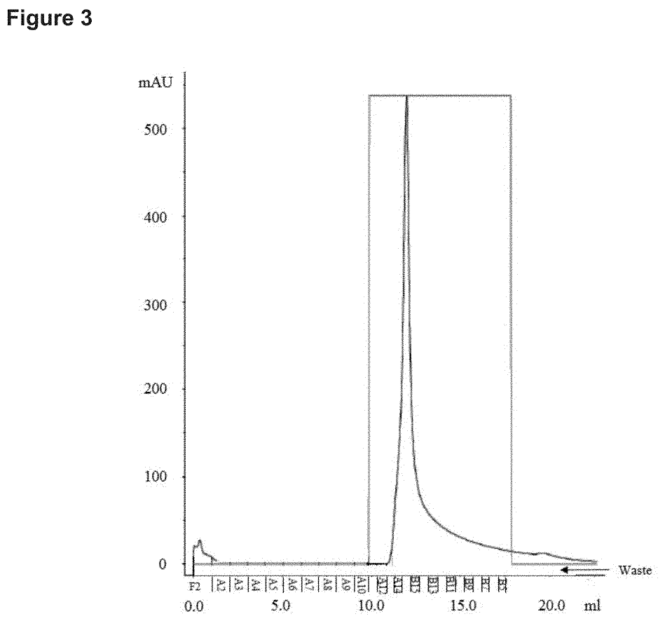

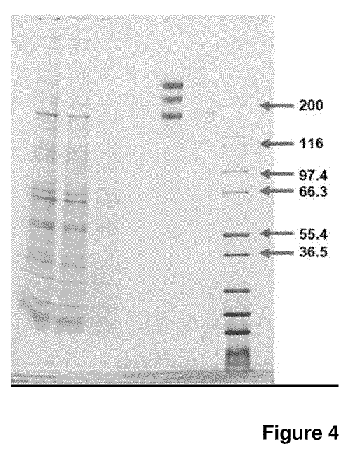

After 10 days of culture the supernatant was clarified by centrifugation and subsequently dialyzed against 1× PBS prior to purification using Protein A Sepharose (FIG. 3). Briefly, the column was equilibrated with at least 5 column volumes of 1× PBS. The dialyzed sample was applied by to the column at a flow rate of 2 ml/min. The column was washed with 5 column volumes of PBS until the absorbance reached a steady baseline or no material remained in the effluent. The RER fusion protein was eluted with 0.25M Glycine, pH 2.5, into tubes pre-filled with 1M Tris, pH 9.0. The fractions were analyzed by SDS-PAGE followed by Coomassie staining and Immunoblot. A sample Coomassie stained gel is shown in FIG. 4.

### SPR and TGF-β Neutralization Testing of PCT-0015 (SEQ ID NO: 14)

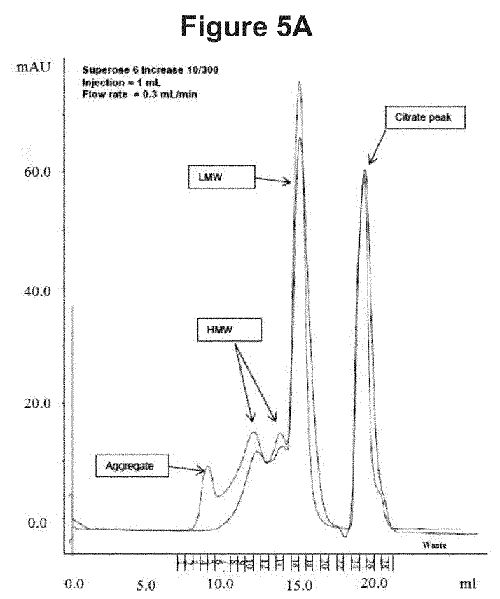

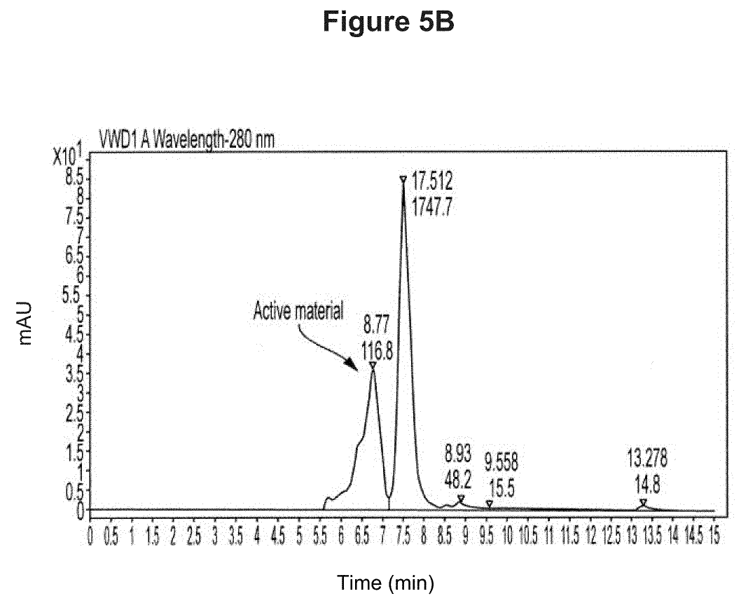

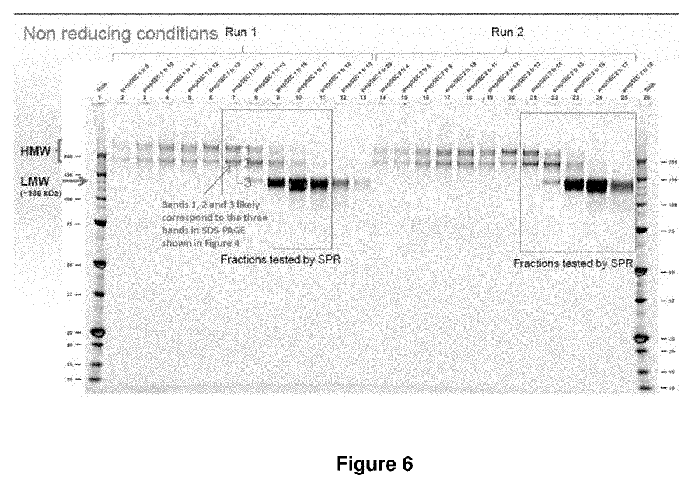

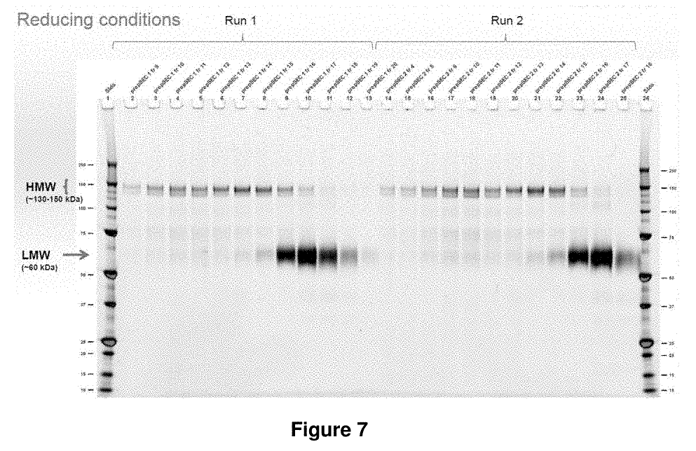

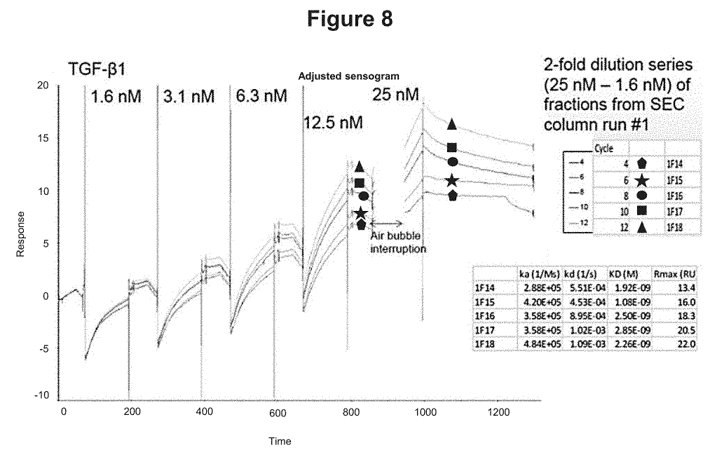

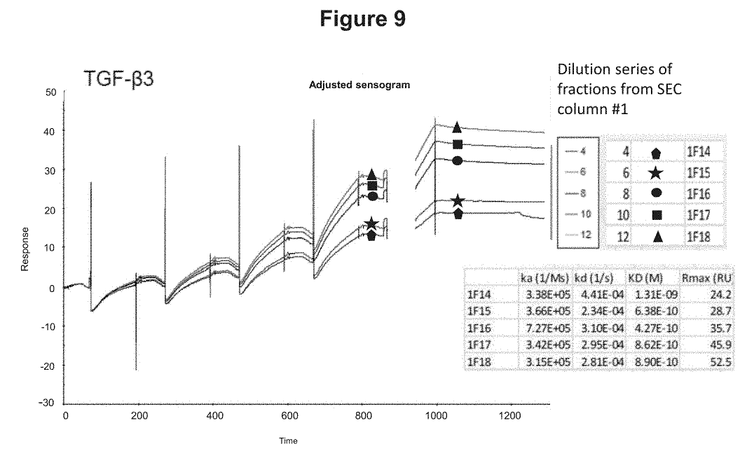

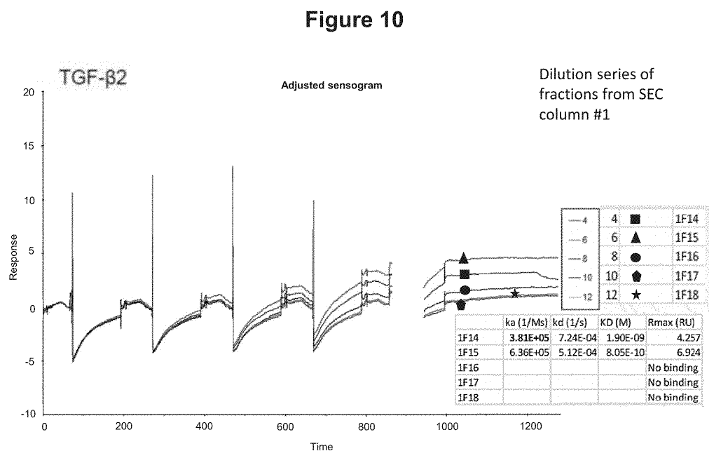

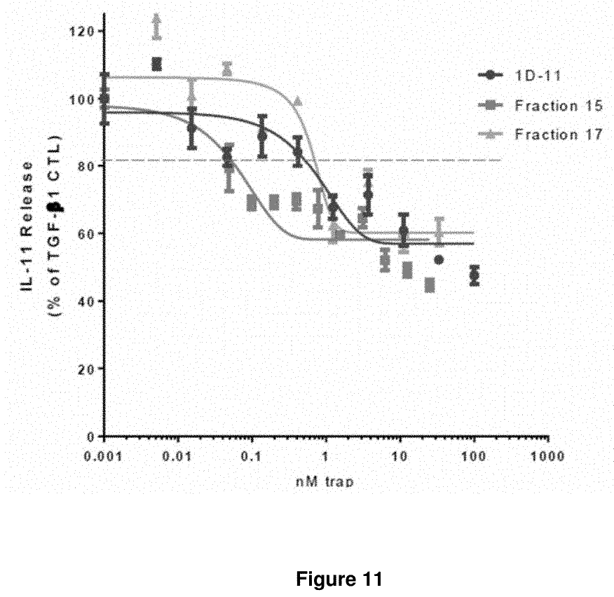

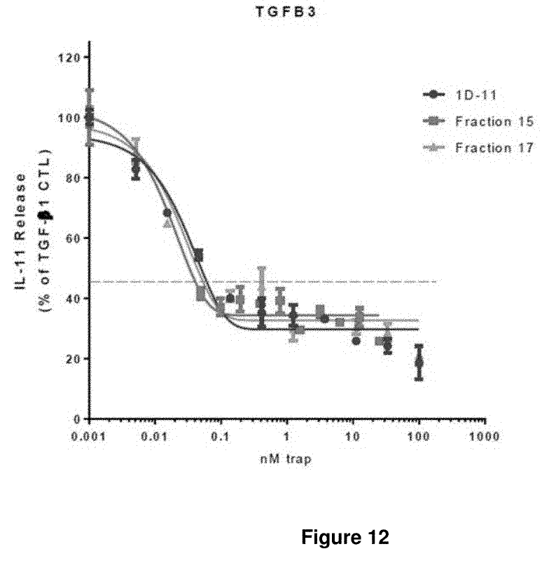

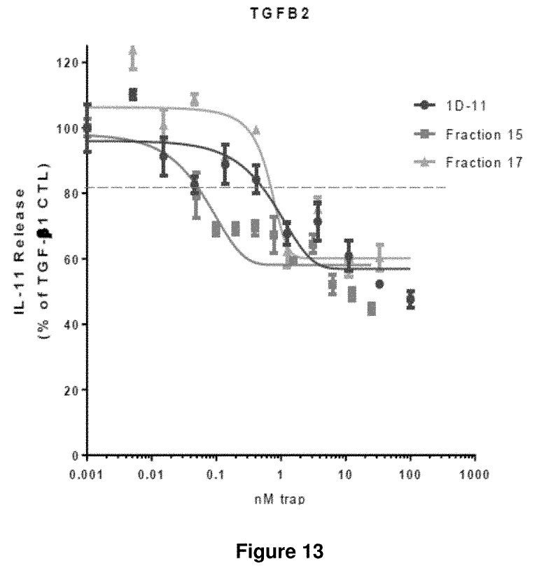

Material purified on Protein A Sepharose shows three major bands migrating with apparent MW around 200 kDa (FIG. 4). This material was further purified by Size Exclusion Chromatography (SEC) on a Superose 6 column allowing some separation of LMW protein from HMW protein populations (FIG. 5A). SEC-HPLC analysis of PCT-0015 is shown in FIG. 5B. The high molecular weight (HMW) (bands 1 & 2, ≥240 kDa) and low molecular weight (LMW) (band 3, ˜130 kDa) forms in a non-reducing gel (FIG. 6) collapse down to ˜130 kDa and ˜60 kDa, respectively, in a reducing gel (FIG. 7). The predominant protein eluting from the SEC column and in the collected fractions is the LMW band. However, the ˜130 kDa size is lower than expected, based on the assumed theoretical MW of PCT-0015 of 190.9 kDa dimer (2×95,452), and hence may be truncated protein. The HMW bands could be the full length dimeric protein and higher order oligomeric forms. Surface plasmon resonance (SPR) analysis of binding of PCT-0015 SEC fractions to TGF-βs are shown in FIGS. 8-10 and the SPR measurements are show in Table 15. TGF-β neutralization for selected SEC fractions of PCT-0015 are shown in FIGS. 11-13.

In summary, PCT-0015 HMW (fractions 14 and15) and LMW (fractions 16,17, and 18) fractions show good binding to TGF-β1 and TGF-β3. HMW shows low binding to TGF-β2, and LMW shows little or no binding. By contrast, the monomeric RER domain showed good binding to TGF-β2. Taken together, the results indicate that dimeric Fc-fused PCT-0015 has partially lost TGF-β2 binding activity. The apparent binding affinities are in the low to sub-nM range.

TGF-β neutralization evaluated using IL-11 release assay indicates that PCT-0015 fractions 15 (HMW) and 17 (LMW) neutralizes TGF-β1 and -β3 with potencies in sub-nano molar range. These fractions also showed TGF-β2 neutralization activity, but EC50s could not be determined as the neutralization window was too small.

TABLE 15
SPR analysis of SEC fractions: measurements of binding to TGF-βs

| | | | |
|---|---|---|---|
| | | | Rmax |
| ka (1/Ms) | kd (1/s) | KD (M) | (RU) |

TGF-β1

| | | | | |
|---|---|---|---|---|
| 1F14 | 2.88E+05 | 5.51E−04 | 1.92E−09 | 13.4 |
| 1F15 | 4.20E+05 | 4.53E−04 | 1.08E−09 | 16.0 |
| 1f16 | 3.58E+05 | 8.95E−04 | 2.50E−09 | 18.3 |
| 1F17 | 3.58E+05 | 1.02E−04 | 2.85E−09 | 20.5 |
| 1F18 | 4.84E+05 | 1.09E−04 | 2.26E−09 | 22.0 |

TGF-β3

| | | | | |
|---|---|---|---|---|
| 1F14 | 3.38E+05 | 4.41E−04 | 1.31E−09 | 24.2 |
| 1F15 | 3.66E+05 | 2.34E−04 | 6.38E−09 | 28.7 |
| 1f16 | 7.27E+05 | 3.10E−04 | 4.27E−09 | 35.7 |
| 1F17 | 3.42E+05 | 2.95E−04 | 8.62E−09 | 45.9 |
| 1F18 | 3.15E+05 | 2.81E−04 | 8.90E−09 | 52.5 |

TGF-β2

| | | | | |
|---|---|---|---|---|
| 1F14 | 3.81E+05 | 7.24E−04 | 1.90E−09 | 4.257 |
| 1F15 | 6.36E+05 | 5.12E−04 | 8.05E−09 | 6.924 |
| 1f16 | | | | |
| 1F17 | | | | |
| 1F18 | | | | |

### Example 2

### Purification, SPR, and TGF-β Neutralization Assay of PCT-0016NT (SEQ ID NO: 33)

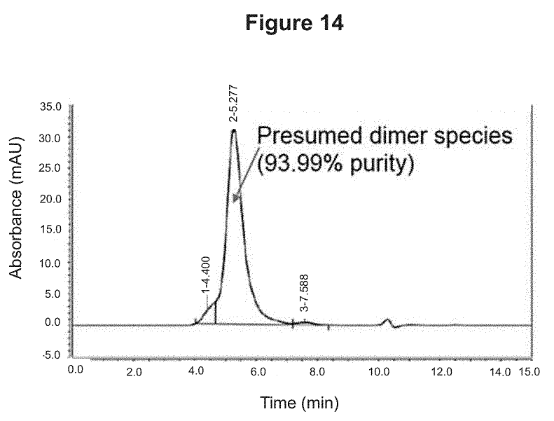

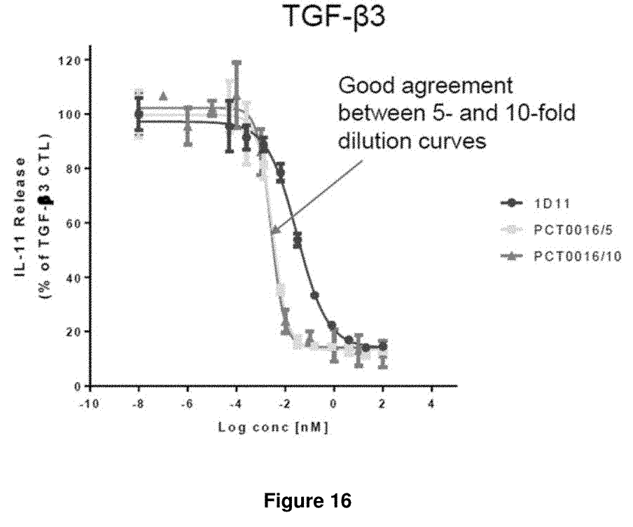

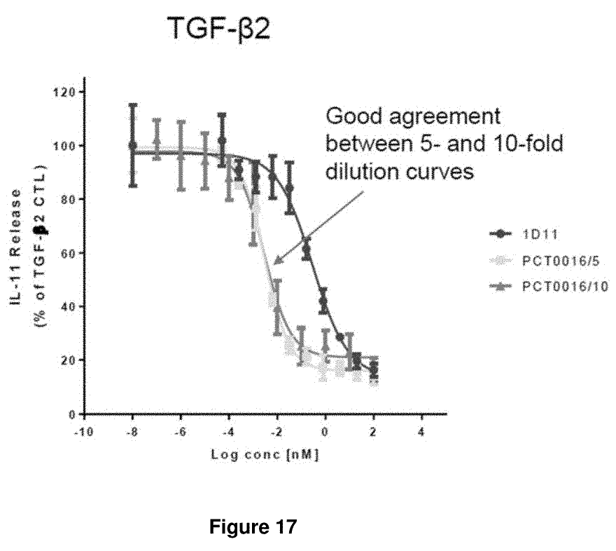

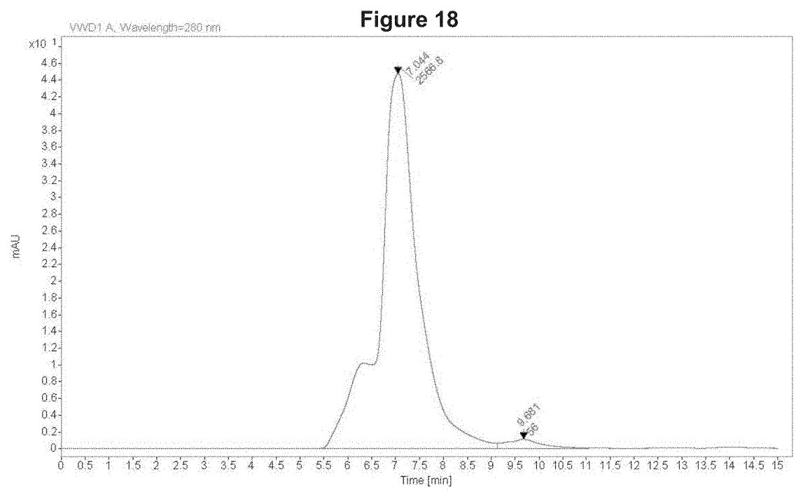

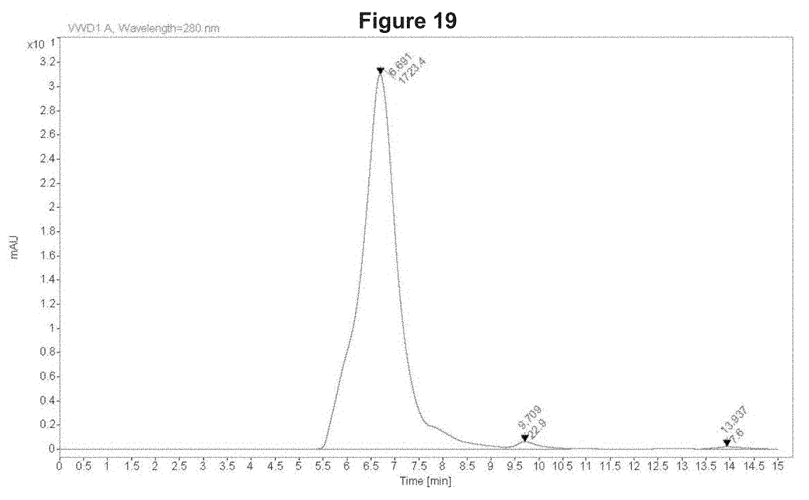

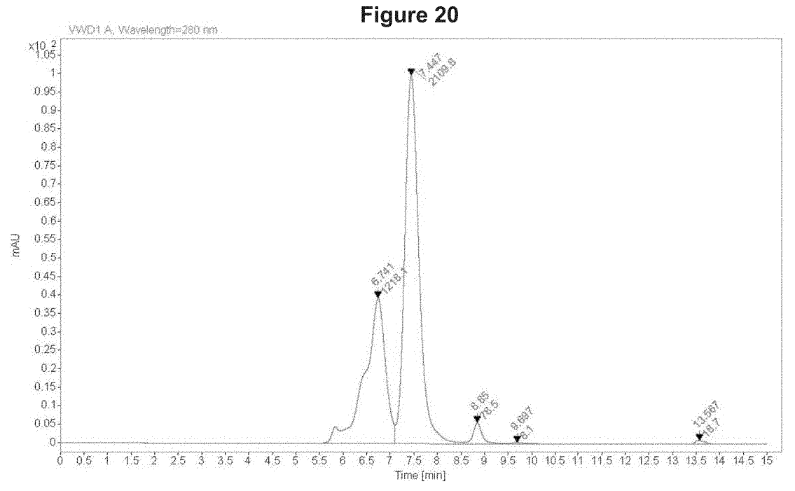

Purification, SPR, and TGF-β neutralization testing of PCT-0016NT (SEQ ID NO: 33) are shown in FIGS. 14-17. Binding affinities for purified peak fractions in SPR assays are shown in Table 16. SPR binding indicates that PCT-0016NT binds tightly to TGF-βisoforms, with a very slow off-rate. The amount of PCT-0016NT bound to TGF-β relative to 1D11, indicates that a proportion of PCT-0016NT protein may be inactive. 1D11 (PCT-001) is a mouse monoclonal anti-TGF-β antibody developed by Genzyme that is not bone-targeted. In summary, SPR binding indicates that PCT-0016NT binds tightly to TGF-β isoforms, with a very slow off-rate.

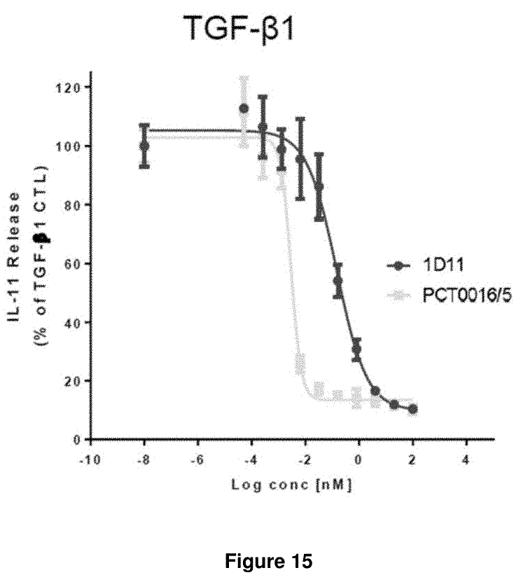

A549 IL-11 neutralization assay indicates high potency for PCT-0016NT, with EC50s in the low pM range. PCT-0016NT is ˜10-60 fold more potent for TGF-β3 and TGF-β1, and ˜100-fold more potent for TGF-β2, compared to 1D11. (FIGS. 15-17).

TABLE 16
KD determination of purified peak fraction in SPR
assay for PCT-0016NT (SEQ ID NO: 33)

| | | | | | |
|---|---|---|---|---|---|
| Cycle | Sample | ka (1/Ms) | kd (1/s) | KD (M) | Rmax (RU) |

TGF-B1

| | | | | |
|---|---|---|---|---|
| 4 PCT0016 | 5.21E+05 | 2.58E−05 | 4.96E−11 | 21 |
| 6 PCT0016 | 5.59E+05 | 1.75E−05 | 3.13E−11 | 21 |
| 8 PCT0016 | 5.80E+05 | 2.45E−05 | 4.23E−11 | 20 |
| Average: | 5.53E+05 | 2.26E−05 | 4.10E−11 | 21 |
| 10 1D11 | 4.16E+05 | 4.23E−05 | 1.02E−10 | 102 |
| 12 1D11 | 4.79E+05 | 4.18E−05 | 8.73E−11 | 92 |
| 14 1D11 | 5.15E+05 | 3.78E−05 | 7.33E−11 | 87 |
| Average: | 4.70E+05 | 4.06E−05 | 8.73E−11 | 94 |

TGF-B2

| | | | | |
|---|---|---|---|---|
| 4 PCT0016 | 4.14E+05 | 1.59E−05 | 3.85E−11 | 15 |
| 6 PCT0016 | 6.24E+05 | 1.08E−05 | 1.72E−11 | 13 |
| 8 PCT0016 | 6.10E+05 | 1.71E−05 | 2.80E−11 | 13 |
| Average: | 5.49E+05 | 1.46E−05 | 2.79E−11 | 14 |
| 10 1D11 | 6.21E+05 | 5.17E−05 | 8.32E−11 | 49 |
| 12 1D11 | 6.75E+05 | 5.54E−05 | 8.20E−11 | 47 |
| 14 1D11 | 6.90E+05 | 4.98E−05 | 7.23E−11 | 46 |
| Average: | 6.62E+05 | 5.23E−05 | 7.92E−11 | 48 |

TGF-B3

| | | | | |
|---|---|---|---|---|
| 4 PCT0016 | 4.10E+05 | 2.93E−05 | 7.14E−11 | 57 |
| 6 PCT0016 | 4.20E+05 | 2.64E−05 | 6.27E−11 | 58 |
| 8 PCT0016 | 4.37E+05 | 2.99E−05 | 6.85E−11 | 56 |
| Average: | 4.22E+05 | 2.85E−05 | 6.75E−11 | 57 |
| 10 1D11 | 2.68E+05 | 2.90E−06 | 1.08E−11 | 296 |
| 12 1D11 | 3.16E+05 | 5.06E−06 | 1.60E−11 | 263 |
| 14 1D11 | 3.46E+05 | 2.75E−06 | 7.93E−12 | 243 |
| Average: | 3.10E+05 | 3.57E−06 | 1.16E−11 | 267 |

### Example 3

### A549 Cells IL-11 Release Assay for TGF-β Neutralization

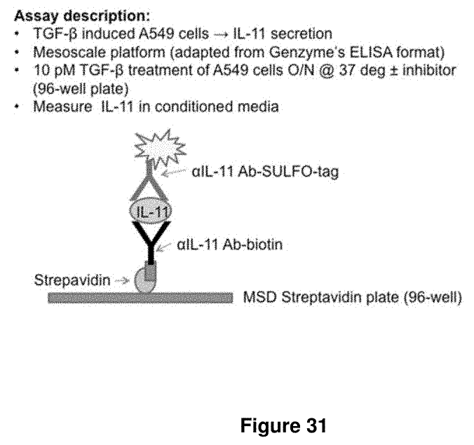

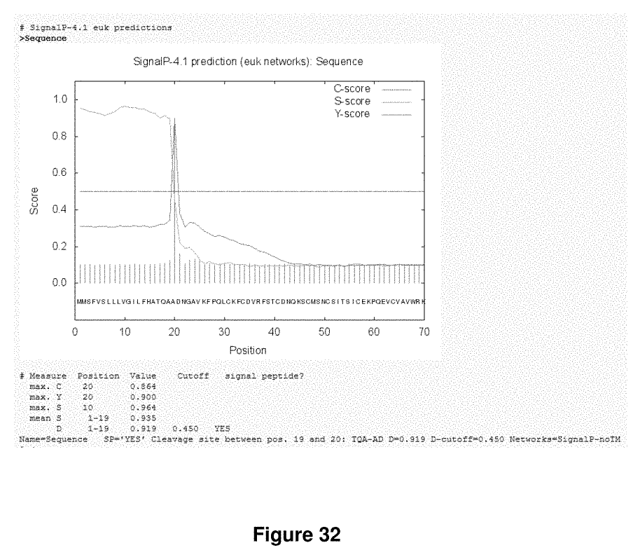

A549 lung cancer cells were seeded on 96-well plates (5×106 cells/well). The following day, 10 pM TGF-β in complete media was incubated with a dilution series of antagonist (range 0.005 to 100 nM) for 30 min at RT. The cells were then treated with 10 pM TGF-β±antagonist and incubated for 18 h at 37□C. Aliquots of conditioned media were added to MSD Streptavidin Gold plates (Meso Scale Diagnostics, Gaithersburg, Md.) coated with 2 μg/mL biotinylated mouse anti-human IL-11 antibody (MAB618, R&D Systems, Minneapolis, Minn.) and incubated 18 h at RT. The plates were washed with PBS containing 0.02% Tween 20, then treated with 2 μg/mL SULFO-tagged goat anti-human IL-11 antibody (AF-218-NA, R&D Systems) for 1 h at RT. After a final wash, plates were read in a MESO QuickPlex SQ 120 machine (Meso Scale Diagnostics). IL-11 readouts were normalized to cell number/well (CyQUANT, Thermo Fisher Sci) and expressed as percent IL-11 release compared to control cells treated with TGF-β alone. Percent of IL-11 released is used as a measurement for TGF-β neutralization by the TGF-β antagonist. FIG. 31 describes the steps for assaying TGF-β induced IL-11 release and an example of an MSD Streptavidin plate.

### Example 4

### Proposed Signal Peptide Cleavage Site

Using an alpha-lactalbumin signal peptide promotes cleavage of the signal peptide with a high probability (0.919) at the position indicated below, which is essential for generating the mature form of the TGF-β receptor fusion proteins. This leaves two additional amino acids on the mature N-terminus before the asparagine residues of the mature TGF-β receptor fusion proteins.

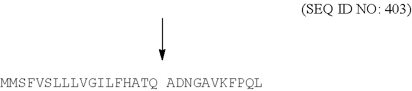

Specific signal peptides, such as those described herein, can improve manufacturing of the TGF-β receptor fusion proteins of the invention, and can be useful for in vivo therapeutic administration of nucleic acids encoding the TGF-β receptor fusion proteins of the invention.

### Example 5

### PCT-0015 (SEQ ID NO: 14) and PCT-0016NT (SEQ ID NO: 33)

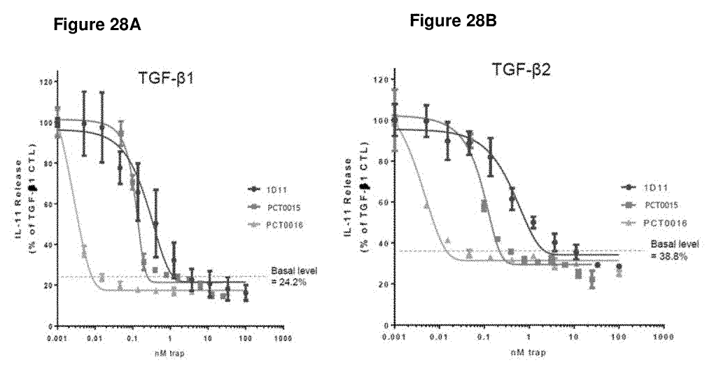

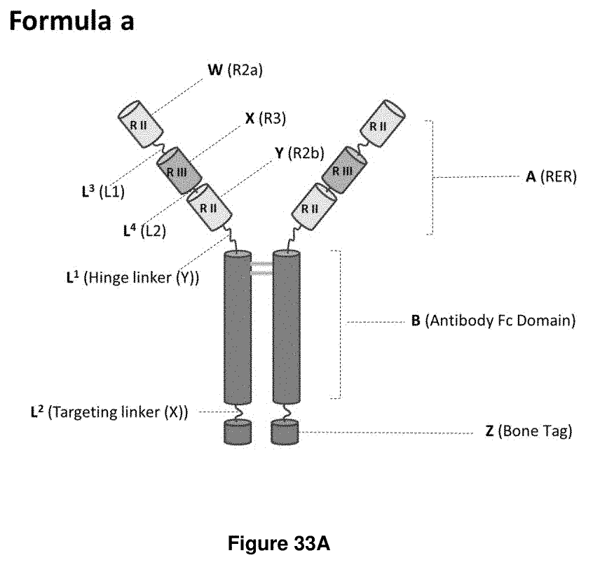

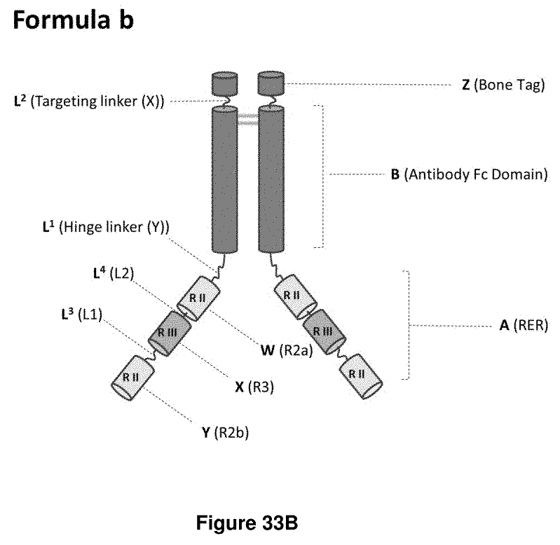

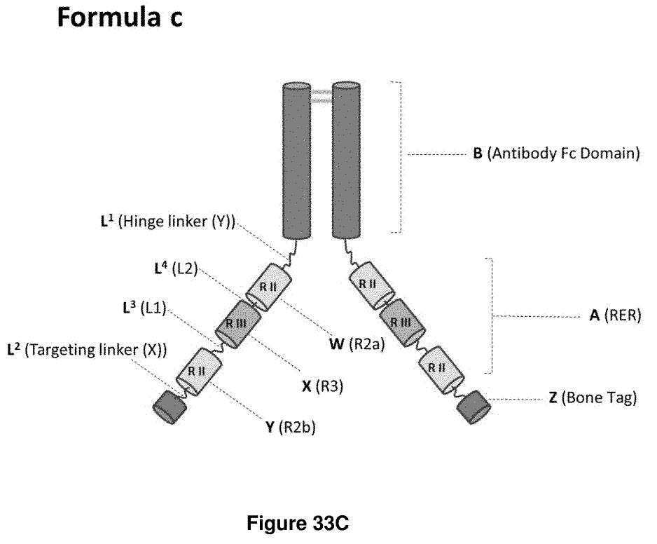

There are a variety of linkers that may be inserted between the RII ectodomains and RIII endoglin domain of the trimeric TGF-β fusion proteins (designated as L³ in FIGS. 33A-C), and between the RER and the Fc domain of an immunoglobulin (designated as a “hinge linker” or L¹ in FIGS. 33A-C). The shortest of these hinge linkers may be TGGG (SEQ ID NO: 36), a threonine-glycine linker (U.S. Pat. No. 9,809,637 B2). PCT-0015 (SEQ ID NO: 14) is an exemplary TGF-β fusion protein (illustrated by “Formula a” in FIG. 33A) conjugate that have this short hinge linker between the C-terminal of the RER and the N-terminal of the immunoglobulin Fc domain, and have a bone-targeting moiety (D10) bound directly to the C-terminal of the immunoglobulin Fc domain. PCT-0015 maintains nanomolar potency for all three isoforms of TGF-β. This may be compared to another TGF-β fusion protein conjugate PCT-0016NT, in which the N-terminus of the RER is bound via a glycine-serine rich hinge linker (GGGGSGGGGSGGGGSG) (SEQ ID NO: 8) found in many different constructs in the literature, to the immunoglobulin Fc domain. PCT-0016NT is illustrated by “Formula b” in FIG. 33B and “Formula c” in FIG. 33C, except that this construct does not have the bone-targeting moiety. PCT-0016NT construct demonstrates picomolar activity. FIGS. 28B and 28C illustrate the relative potency of these two constructs as assayed by TGF-β1 and TGF-β2 neutralization assays.

### Example 6

### PCT-0020 (SEQ ID NO: 18)

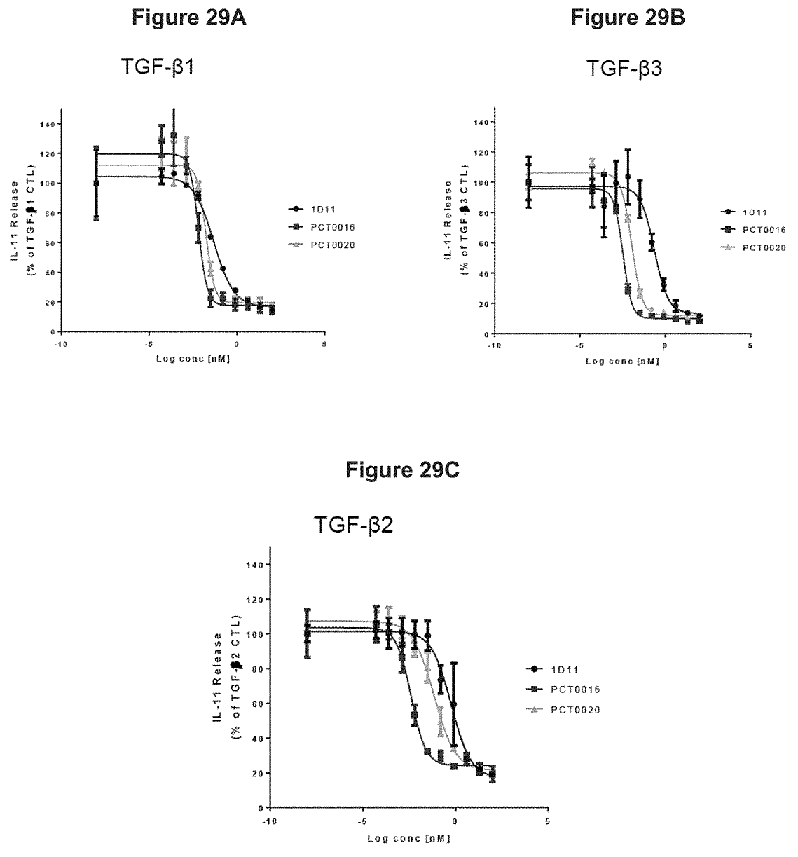

Another exemplary TGF-β fusion protein conjugate (illustrated by “Formula a” in FIG. 33A) may have the hinge linker sequence of LLLVIFQVTGISLLPPLGGGGS (SEQ ID NO: 37), which includes a C-terminal RER RII ectodomain extension. The RII extension is made possible through an extension of the TGF-β RII coding sequence. This hinge linker introduces a kink in the protein, which may provide a constraint preventing the RER from interacting with the Fc hinge region. The resulting construct is PCT-0020. FIGS. 29A-C illustrate relative TGF-β isoform neutralization of PCT-0020 compared to PCT-0016NT and 1D11 antibody. PCT-0020 is well-produced with high purity and expected molecular weight.

PCT-0020 is more potent than 1D11 antibody, but is less potent than PCT-0016NT by ˜2-3 fold for TGF-β1 and TGF-β3 and ˜17-fold for TGF-β2 (Table 17). PCT-0020 is a potent compound from the option 1 series (illustrated by “Formula a”) but it does not equal to the potency of PCT-0016NT, especially for TGF-β2.

TABLE 17
Comparison of PCT-0020 (SEQ ID NO: 18) potency with PCT-0016NT
(SEQ ID NO: 33) and 1D11
Summary EC50 [nM]

| | 1D11 | PCT0016 | PCT0020 | Fold 0020/0016 |
|---|---|---|---|---|
| TGFb1 | 0.0478 | 0.0065 | 0.0171 | 2.6 |
| TGFb3 | 0.2249 | 0.0032 | 0.0105 | 3.3 |
| TGFb2 | 0.5905 | 0.0041 | 0.0710 | 17.4 |

### Example 7

### PCT-0021 (SEQ ID NO: 20) and PCT-0022 (SEQ ID NO: 22)

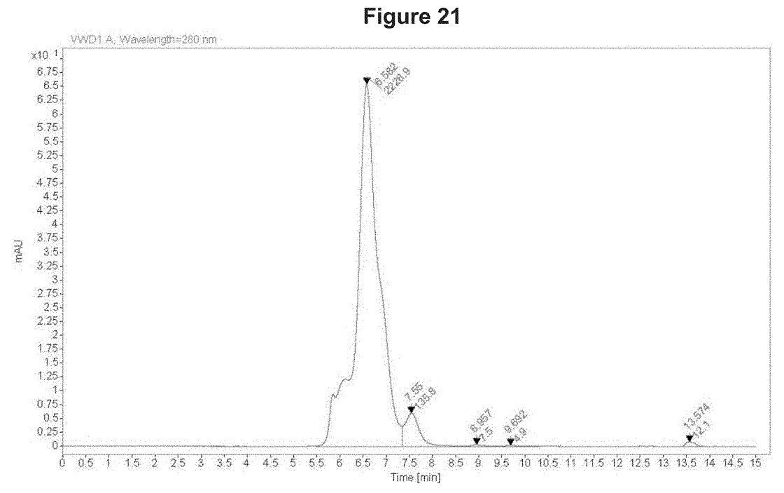

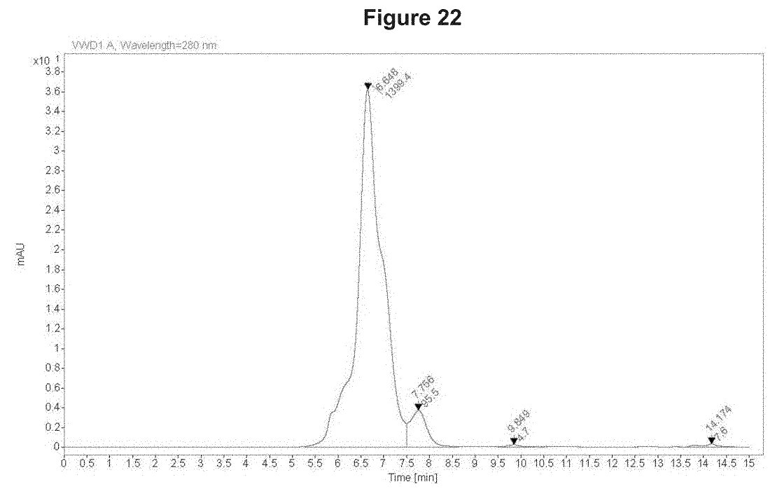

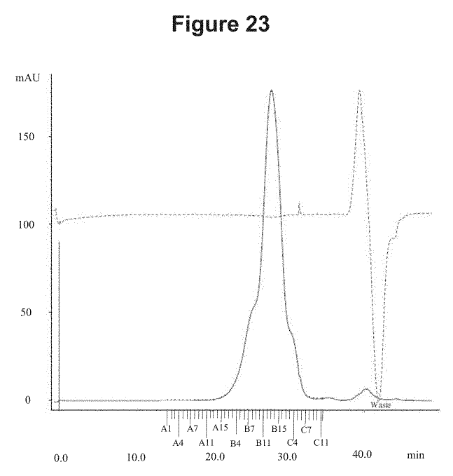

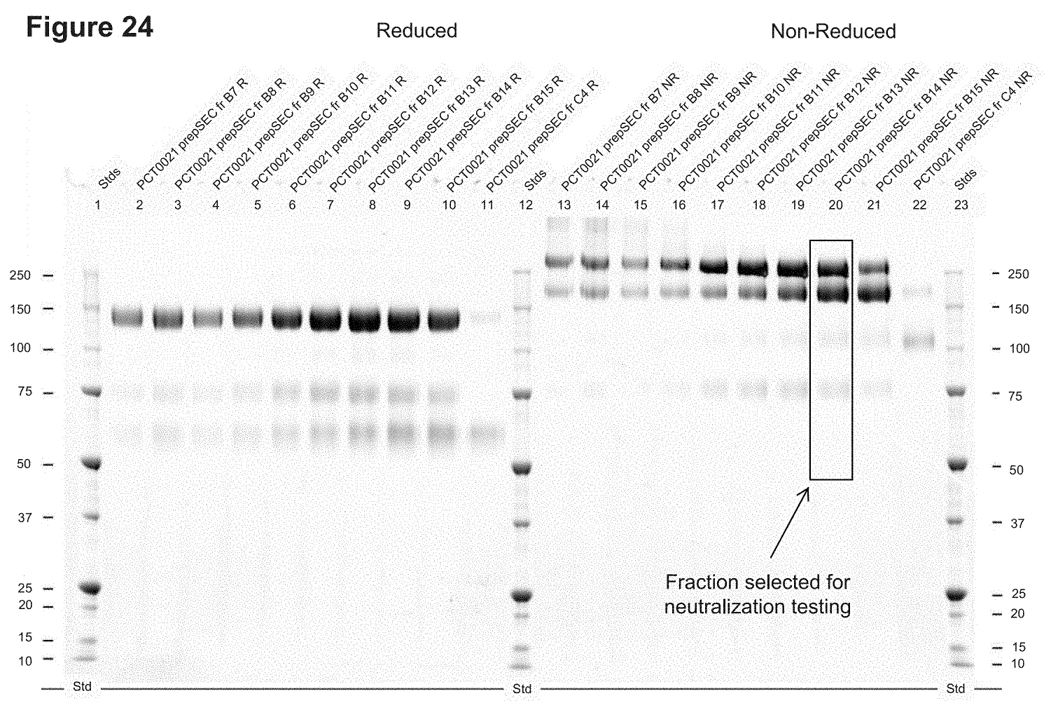

The linker between the RII ectodomain and RIII endoglin domain (designated as L³ in FIGS. 33A-C) and the hinge linker (designated as L¹ in FIGS. 33A-C) of the TGF-β fusion proteins affect structural integrity and potency, and can be further modified to reduce immunogenicity. Compared to PCT-0020 above, PCT-0021 has a longer natural hinge linker sequence to reduce immunogenicity and improve potency. Thus, PCT-0021 is identical to PCT-0020, except for the hinge linker in PCT-0021 includes a longer extension of the coding sequence of TGF-β RII and does not include the artificial GGGGS sequence (SEQ ID NO: 7) of PCT-0020 hinge linker. The linker between RER and the Fc domain of the PCT-0021 construct consists entirely of native TGF-β RII sequence. SEC and purification data as well as SDS-PAGE analysis for PCT-0021 are shown in FIGS. 22-24.

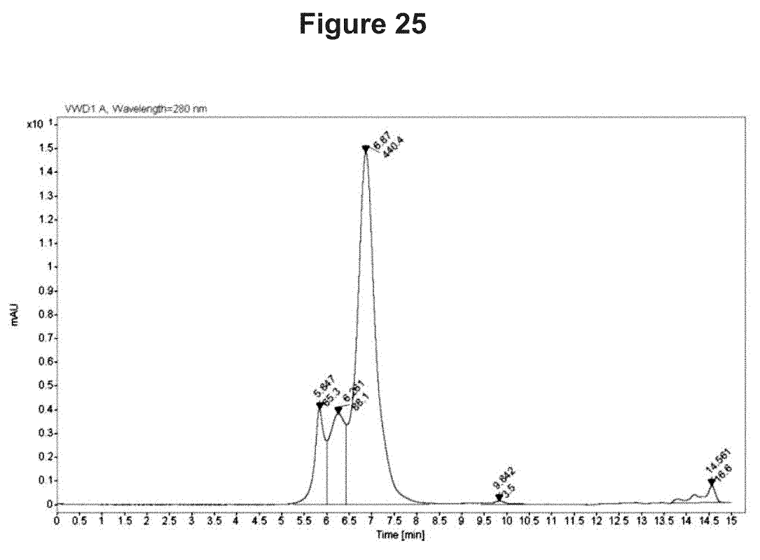

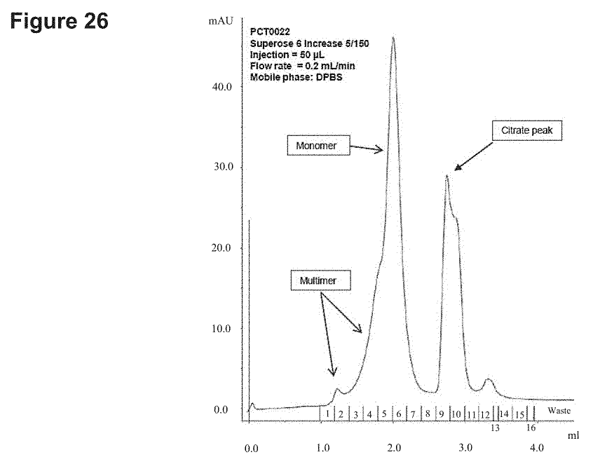

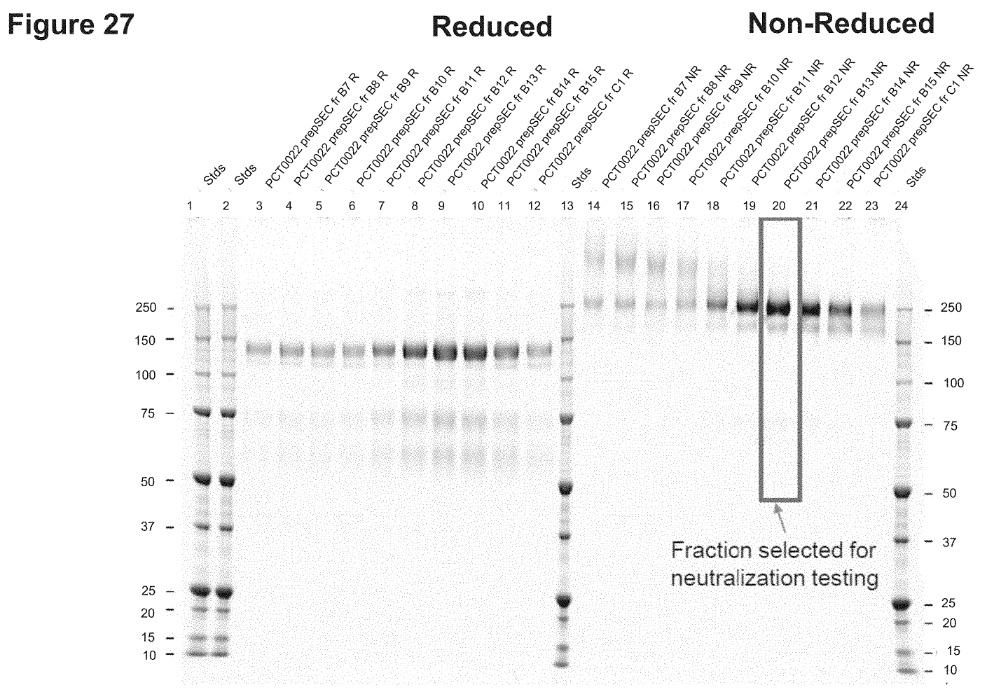

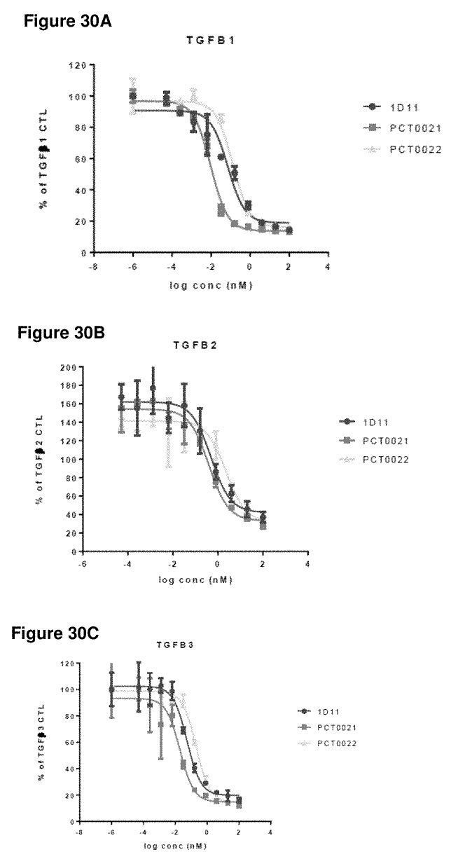

PCT-0022 includes a human RII sequence as the L³ linker between the RII ectodomain and RIII endoglin domain. SEC data for PCT-0022 are shown in FIGS. 25 and 26, and SDS-PAGE analysis is shown in FIG. 27. FIGS. 30A-C show comparative neutralization of the three TGF-β isoforms for selected SEC fractions of PCT-0021 and PCT-0022. Table 18 shows IC50 for neutralization of the three isoforms of TGF-β by PCT-0021 and PCT-0022 as compared to 1D11.

In summary, PCT-0021 is more potent than 1D11 for TGF-β1 and TGF-β3, whereas PCT-0022 is modestly less potent than 1D11. The SEC fractions contain more than one species, particularly for PCT-0021 FrB14, and this may alter the neutralization efficiency.

TABLE 18
IC50 for neutralization of the three isoforms of TGF-β as
compared to 1D11

| Compound | TGF-β1 | TGF-β3 | TGF-β2 |
|---|---|---|---|
| 1D11 | 0.0722 | 0.0547 | 0.492 |
| PCT0021 FrB14 | 0.0096 | 0.0198 | 0.396 |
| PCT0022 FrB13 | 0.1289 | 0.2047 | 2.273 |

### Example 8

### PCT-0025 (SEQ ID NO: 28) and PCT-0026 (SEQ ID NO: 30)

PCT-0025 and PCT-0026 are fully humanized and increase the length of both linker L¹ (hinge linker) and L³ (shown in “Formula a” in FIG. 33A). PCT-0025 uses LLLVIFQVTGISLLPPLGVAISVIII (SEQ ID NO: 38) as the L¹ and L³ linkers. This linker sequence contains a TGF-β receptor II sequence extension. The PCT-0026 uses an artificial L³ sequence (GLGPVESSPGHGLDTAA) (SEQ ID NO: 40) and a hybrid L¹ sequence (LLLVIFQVTGISLLPPLGGGGS) (SEQ ID NO: 37). PCT-0025 and PCT-0026 (of “Formula a”) are considered the lead therapeutic compounds because of their superior characteristics and performance.

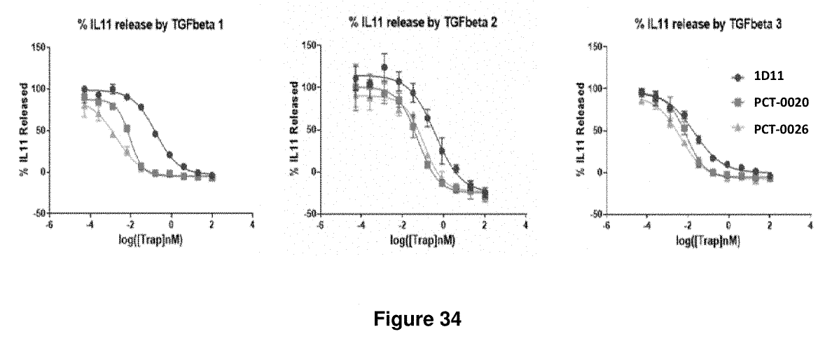

FIG. 34 shows comparative neutralization of the three TGF-β isoforms (TGF-β1, TGF-β2, and TGF-β3) by PCT-0026 as compared to PCT-0020 and 1D11 antibody. Table 19 shows EC50 values for neutralization of the three isoforms of TGF-β by PCT-0026 as compared to PCT-0020 and 1D11. PCT-0026 is more potent than PCT-0020 and 1D11 for TGF-β1 and TGF-β3, whereas PCT-0020 is more potent than PCT-0026 and 1D11 for TGF-β2.

TABLE 19
EC50 for neutralization of the three isoforms of TGF-β by PCT-0026 as
compared to PCT-0020 and 1D11
EC50 Values (picomol/L)

| | 1D11 | PCT-0020 | PCT-0026 |
|---|---|---|---|
| TGF-β1 | 163.9 | 8.8 | 1.9 |
| TGF-β2 | 381.9 | 55.5 | 105.4 |
| TGF-β3 | 19.0 | 8.0 | 4.2 |

### Example 9

### Distribution of PCT-0026 (SEQ ID NO: 30) to Bone

PCT-0026 (SEQ ID NO: 30) was radioactively labeled with zirconium-89 via standard methods. At the start of the study, nude mice (3 per group) were injected intraperitoneally (i.p.) with radiolabeled PCT-0026 (SEQ ID NO: 30) at a concentration of 10 mg/kg. Mice were anesthetized and whole-body images were taken using PET imaging to visualize radioactive distribution at 1 h, 4 h, 24 h, 48 h and 7 days post injection. 7 days post injection, animals were euthanized, femurs were isolated and counted on gamma scintillation counter. Serum was isolated from duplicate mice at 15 min, 1 h, 2 h, 4 h, 24 h, and 48 h post-injection and counted on a gamma scintillation counter. The data is expressed as the percentage of counts relative to total injected counts.

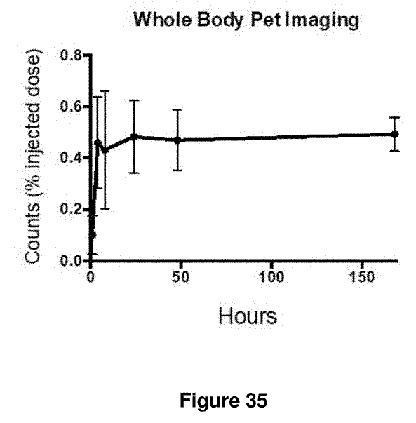

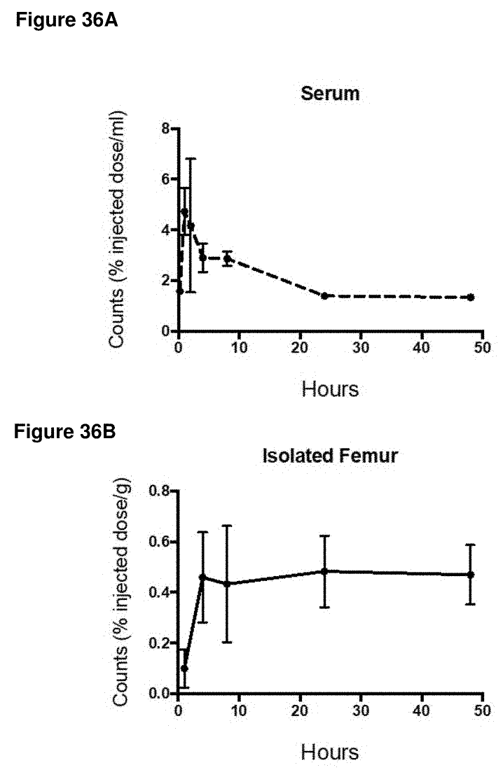

Positron emission tomography (PET) imaging revealed accumulation of radiolabel within 48 hours post-injection (FIG. 35) that was maintained across the 7-day study length. Gamma counts of isolated femurs demonstrated that 0.98%±0.34% of total injected protein was retained per gram of isolated bone at 7 days post injection. Parallel analysis of serum levels revealed maximum serum exposure was obtained between 1 and 2 hours post i.p. injection (FIG. 36A). In contrast, maximal accumulation of counts were observed in long bones within 4 hours post injection (FIG. 36B). The results suggest a rapid clearance of PCT-0026 (SEQ ID NO: 30) from the bloodstream with a half-life of about 15 hours, and a femur accumulation corresponding to 0.5% of the total injected dose. The amount of material targeted to bone however appears to be stable for at least 50 hours, suggesting that repeated injections can achieve a bone exposure sufficient to induce the expected therapeutic response.

### Example 10

### Administration of a TGF-β Antagonist for the Treatment of Diseases Associated with Elevated TGF-β Activity

Using the compositions and methods described herein, a physician of skill in the art can administer to a patient (e.g., a human patient) a conjugate containing a TGF-β receptor fusion protein, such as a TGF-β receptor fusion protein having the amino acid sequence of SEQ ID NO: 9, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, and SEQ ID NO: 35, or a TGF-β receptor fusion protein having at least 70% sequence identity (e.g., at least 75%, 80%, 85%, 90%, 95%, 97%, 98%, 99%, or more, sequence identity) thereto. The TGF-β receptor fusion protein can be bound to a bone-targeting hydroxyapatite-binding domain, such as a polyanionic peptide of the formula Dn or En, in which D and E designate aspartate and glutamate, respectively, and each n designates an integer from 1 to 25 (e.g., 1, 2, 3, 4, 5, 6, 7, 8, 9, 10, 11, 12, 13, 14, 15, 16, 17, 18, 19, 20, 21, 22, 23, 24, or 25). For example, TGF-β receptor fusion protein can be bound to a bone-targeting hydroxyapatite-binding domain of the formula D10. For example, the conjugate may be a protein having the amino acid sequence of SEQ ID NO: 5, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, and SEQ ID NO: 34, or at least 70% sequence identity (e.g., at least 75%, 80%, 85%, 90%, 95%, 97%, 98%, 99%, or more, sequence identity) thereto. The patient may be one that is suffering from a disease associated with elevated TGF-β activity, muscle weakness, and/or elevated bone turnover relative to a healthy individual not suffering from the disease, such as osteogenesis imperfecta.

For instance, a physician of skill in the art may assess a patient suffering from osteogenesis imperfecta by first evaluating muscle function in the patient using one or more methodologies, such as manual muscle testing, dynamometry, or muscle mechanography, or imaging techniques to assess muscle-cross sectional area, volume, density, or muscle mass (e.g., MRI or CT scans). A finding that the individual has reduced muscle function relative to a muscle function reference level, such as the level of muscle function in a healthy subject (e.g., a healthy subject of the same gender, age, and/or body mass) can indicate that the patient may be particularly well suited for treatment with a TGF-β antagonist capable of improving muscle function. A physician of skill in the art may additionally assess the patient's muscle function over time so as to monitor the progression of the disease during the course of treatment. The physician may administer to the patient a therapeutically effective amount (e.g., an amount sufficient to attenuate TGF-β signaling and/or to reduce bone turnover) of a composition containing a TGF-β antagonist or conjugate thereof. The TGF-β antagonist may be any antagonist described herein, such as, for instance, a TGF-β receptor fusion protein, such as a protein having at least 70% sequence identity (e.g., at least 75%, 80%, 85%, 90%, 95%, 96%, 97%, 98%, 99%, or 100% sequence identity) to SEQ ID NO: 9, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, and SEQ ID NO: 35. Optionally, the TGF-β antagonist construct may be conjugated to a polyanionic peptide, such as a deca-aspartate peptide, and may have, for instance, at least 70% sequence identity (e.g., at least 75%, 80%, 85%, 90%, 95%, 96%, 97%, 98%, 99%, or 100% sequence identity) to SEQ ID NO: 5, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, and SEQ ID NO: 34.

In some other instances, a physician of skill in the art may assess a patient suffering from a bone disease, such as osteogenesis imperfecta, by first evaluating the concentration of one or more biomarkers of bone turnover, such as serum and bone alkaline phosphatase, serum osteocalcin (sOC), serum type I collagen C-telopeptide breakdown products (sCTX), urinary free-deoxypyridinoline (ufDPD), and urinary cross-linked N-telopeptides of type I collagen (uNTX). A finding that one or more of these biomarkers is elevated may signal an elevated bone turnover rate, indicating that the patient may be particularly well suited for treatment with a TGF-β antagonist capable of localizing to bone tissue. A physician of skill in the art may additionally assess the patients frequency of, and propensity for, bone fracture so as to monitor the progression of the disease during the course of treatment.

In some instances, the physician may administer to the patient a therapeutically effective amount (e.g., an amount sufficient to attenuate TGF-β signaling) of a composition containing a TGF-β antagonist, optionally bound to a bone-targeting moiety. The bone-targeting moiety may be any bone-targeting moiety described herein, such as, for instance, a collagen-binding domain or a hydroxyapatite-binding domain as described herein, e.g., a hydroxyapatite-binding domain containing a deca-poly(Asp) sequence motif.

The physician may administer to the patient a therapeutically effective amount (e.g., an amount sufficient to attenuate TGF-β signaling and/or to reduce bone turnover) of a conjugate containing a TGF-β receptor fusion protein, including those bound to a bone-targeting moiety, at a dosing schedule determined by the patients age, weight, gender, and/or severity of the disease. For example, the conjugate may be administered to the subject in one or more doses (e.g., 1, 2, 3, 4, 5, 6, 7, 8, 9, 10, 15, 20, 25, 30, or more) per a specified time interval, such as one or more doses per day, per week, per month, or per year. The patient may be evaluated between doses so as to monitor the effectiveness of the therapy and to increase or reduce the dosage based on the patients response. For example, a reduction in the incidence of bone fractures, an improved ability of the patient to walk, and/or a reduction in the concentration of one or more biomarkers of bone turnover in a sample isolated from the patient may indicate that the therapy is effectively treating the condition.

The therapy may be administered to the patient by a variety of routes of administration, for instance, as determined by a physician of skill in the art. For example, the therapy may be administered to the patient in one or more repeat doses subcutaneously, intradermally, intramuscularly, intraperitoneally, intravenously, or orally, or by nasal or by epidural administration.

Prior to the conclusion of therapy, the physician may prescribe progressively lower doses of the conjugate to the patient so as to gradually reduce the concentration of the therapy. The therapy may involve only a single dosing of the therapeutic conjugate. Alternatively, the therapy may continue, for instance, for a period of days, weeks, months, or years prior to completion.

### Example 11

### Administration of a TGF-β Antagonist Conjugated to a Bone-Targeting Hydroxyapatite-Binding Polyanionic Peptide for the Treatment of Osteogenesis Imperfecta

Using the compositions and methods described herein, a physician of skill in the art can administer to a patient (e.g., a human patient) a conjugate containing a TGF-β receptor fusion protein, such as a TGF-β receptor fusion protein having the amino acid sequence of SEQ ID NO: 9, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, and SEQ ID NO: 35, or a TGF-β receptor fusion protein having at least 70% sequence identity (e.g., at least 75%, 80%, 85%, 90%, 95%, 97%, 98%, 99%, or more, sequence identity) thereto. The TGF-β receptor fusion protein can be bound to a bone-targeting hydroxyapatite-binding domain, such as a polyanionic peptide of the formula Dn or En, in which D and E designate aspartate and glutamate, respectively, and each n designates an integer from 1 to 25 (e.g., 1, 2, 3, 4, 5, 6, 7, 8, 9, 10, 11, 12, 13, 14, 15, 16, 17, 18, 19, 20, 21, 22, 23, 24, or 25). For example, TGF-β receptor fusion protein can be bound to a bone-targeting hydroxyapatite-binding domain of the formula D10. For example, the conjugate may be a protein having the amino acid sequence of SEQ ID NO: 5, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, and SEQ ID NO: 34, or at least 70% sequence identity (e.g., at least 75%, 80%, 85%, 90%, 95%, 97%, 98%, 99%, or more, sequence identity) thereto. The patient may be one that is suffering from a disease associated with elevated TGF-β activity and/or elevated bone turnover relative to a healthy individual not suffering from the disease, such as osteogenesis imperfecta.

For instance, a physician of skill in the art may assess a patient suffering from osteogenesis imperfecta by first evaluating the concentration of one or more biomarkers of bone turnover, such as serum and bone alkaline phosphatase, serum osteocalcin (sOC), serum type I collagen C-telopeptide breakdown products (sCTX), urinary free-deoxypyridinoline (ufDPD), and urinary cross-linked N-telopeptides of type I collagen (uNTX). A finding that one or more of these biomarkers is elevated may signal an elevated bone turnover rate, indicating that the patient may be particularly well suited for treatment with a TGF-β antagonist capable of localizing to bone tissue. A physician of skill in the art may additionally assess the patients frequency of, and propensity for, bone fracture so as to monitor the progression of the disease during the course of treatment.

The physician may administer to the patient a therapeutically effective amount (e.g., an amount sufficient to attenuate TGF-β signaling and/or to reduce bone turnover) of a conjugate containing a TGF-β receptor fusion protein bound to a bone-targeting moiety at a dosing schedule determined by the patient's age, weight, gender, and/or severity of the disease. For example, the conjugate may be administered to the subject in one or more doses (e.g., 1, 2, 3, 4, 5, 6, 7, 8, 9, 10, 15, 20, 25, 30, or more) per a specified time interval, such as one or more doses per day, per week, per month, or per year. The patient may be evaluated between doses so as to monitor the effectiveness of the therapy and to increase or reduce the dosage based on the patients response. For example, a reduction in the incidence of bone fractures, an improved ability of the patient to walk, and/or a reduction in the concentration of one or more biomarkers of bone turnover in a sample isolated from the patient may indicate that the therapy is effectively treating the condition.

The therapy may be administered to the patient by a variety of routes of administration, for instance, as determined by a physician of skill in the art. For example, the therapy may be administered to the patient in one or more repeat doses subcutaneously, intradermally, intramuscularly, intraperitoneally, intravenously, or orally, or by nasal or by epidural administration.

Prior to the conclusion of therapy, the physician may prescribe progressively lower doses of the conjugate to the patient so as to gradually reduce the concentration of the therapy. The therapy may involve only a single dosing of the therapeutic conjugate. Alternatively, the therapy may continue, for instance, for a period of days, weeks, months, or years prior to completion.

### Example 12

### Study of Muscle Function in a Mouse Model of Osteogenesis Imperfecta

A study is performed to monitor the Nox4 dependent oxidation pathway of RyR1 in muscle from a mouse model of OI with associated muscle weakness. OI models include, but are not limited to, the Brtl+/−, oim−/−, Crtap−/− and Jrt+/− mouse models.

Muscle function may be tested in vivo by testing the forearm grip of OI mice compared to normal mice. Alternatively, muscle weakness may be assessed by measuring the ex vivo specific force of the extensor digitorum longus muscle. TGF-β elevation can be confirmed via demonstration that its downstream signaling molecules, SMAD2 and SMAD3 are activated. This may be measured by immunoblot analysis showing an increased amount of phosphorylated SMAD2 or SMAD3 is present relative to total SMAD2 or SMAD3 in muscle lysates. To assess involvement of NADPH oxidase 4, Nox4 mRNA may be measured using standard RT-PCR to confirm increased expression in muscle derived from OI mice relative to muscle from normal mice. Immunoblots of muscle lysates may also be performed to demonstrate oxidation and nitrosylation of RyR1, two downstream consequences of NADPH oxidase 4. Finally, co-immunoprecipation of RyR1 and its associated regulatory protein, calstabin may be performed. Demonstration that calstabin binding to RyR1 is reduced in muscles from OI mice relative to normal mice can be used as a surrogate to monitor calcium leak in muscles and associated muscle weakness. Final demonstration of TGF-β involvement in this mechanism can be demonstrating by showing that these parameters are reversed in OI mice treated with a TGF-β antagonist.

Methods

Grip strength. Forelimb grip strength can be assessed by allowing each mouse to grab a wire mess attached to a force transducer (Bioseb, Bitrolles, France) that records the peak force as the mouse is pulled by the tail horizontally away from the mesh (Bellinger, A. M. et al. Hypernitrosylated ryanodine receptor calcium release channels are leaky in dystrophic muscle. Nat. Med. 15, 325-330, 2009; Bonetto, A., Andersson, D. C. & Waning, D. L. Bonekey Rep. 4, 732; 2015). In this context, a reduction in grip strength relative to a normal mouse is indicative of reduced muscle function. Similarly, in the context of a human, reduced grip strength in a patient relative to a healthy individual is indicative of reduced muscle function.

Contractility. Ex vivo contractility of the extensor digitorum longus (EDL) muscles can be determined as described (Andersson, D. C. et al. Cell Metab. 14, 196-207, 2011; Bonetto, A., Andersson, D. C. & Waning, D. L. Bonekey Rep. 4, 732; 2015). EDL can be dissected from the hind limbs and stainless-steel hooks, tied to the tendons of the muscles using 4-0 silk sutures and the muscles mounted between a force transducer (Aurora Scientific, Aurora, ON, Canada) and an adjustable hook. The muscles are immersed in a stimulation chamber containing O2/CO2 (95/5%) bubbled Tyrode solution (121 mM NaCl, 5.0 mM KCl, 1.8 mM CaCl2, 0.5 mM MgCl2, 0.4 mM NaH2PO4, 24 mM NaHCO3, 0.1 mM EDTA, 5.5 mM glucose). The muscle is stimulated to contract using a supramaximal stimulus between two platinum electrodes. Data can be collected via Dynamic Muscle Control/Data Acquisition (DMC) and Dynamic Muscle Control Data Analysis (DMA) programs (Aurora Scientific). At the start of each experiment the muscle length can be adjusted to yield the maximum force. The force-frequency relationships can be determined by triggering contraction using incremental stimulation frequencies (0.5-ms pulses at 1-150 Hz for 350 ms at supramaximal voltage). Between stimulations the muscle is allowed to rest for 3 min. At the end of the force measurement, the length (L0) and weight of the muscle are measured and the muscle are snap frozen in liquid N2. To quantify the specific force, the absolute force is normalized to the muscle size, specifically the cross-sectional area, calculated as the muscle weight divided by the length using a muscle density constant of 1.056 kg/m3 (Yamada, T. et al. Arthritis Rheum. 60, 3280-3289; 2009). In this context, a reduction in ex vivo contractility relative to a normal mouse is indicative of reduced muscle function. In the context of a human, measures of muscle function in the terms of muscle force can be determined using, for example, muscle mechanography (e.g., hopping on a force plate) and muscle size, density, volume, or cross-sectional area can be measured using visual or imaging techniques (e.g., magnetic resonance imaging (MRI) or computed tomography (CT) scans). In humans, a decrease in muscle force or muscle size, density, volume, or cross-sectional area in a patient relative to a healthy individual is indicative of reduced muscle function.

Measurement of protein oxidation and ROS production. To determine channel oxidation the carbonyl groups on the protein side chains can be derivatized to 2,4-dinitrophenylhydrazone (DNP-hydrazone) by reaction with 2,4-dinitrophenylhydrazine (DNPH) (Oxyblot, Millipore, Darmstadt, Germany). The DNP signal on RyR1 can be detected by immunoblotting with an antibody specific to DNP (Millipore, Darmstadt, Germany). Protein carbonyl concentration in tissue lysates can be determined using the OxiSelect Protein Carbonyl ELISA Kit (Cell BioLabs, Inc., San Diego, Calif.). For example, 0.5 mg of EDL lysate can be added to a 96-well protein-binding plate, which is incubated overnight at 4° C. After washing the plate three times with PBS, the protein carbonyl groups are derivatized with DNPH for 45 min at room temperature (in the dark). Plates are developed with a DNP-specific antibody followed by a HRP-conjugated secondary antibody. Protein carbonyl concentration is determined by comparison with a standard curve of oxidized BSA. ROS production is determined in C2C12 myotubes using the OxiSelect in vitro ROS/RNS Assay kit (Cell BioLabs, Inc.). ROS production is measured using 0.25 mg of cell lysate according to the manufacturer's recommendations. For H2O2-treated cells, cells are incubated with 1 mM H2O2 for 30 min before lysis. The investigators are blinded to treatment of subjects. In this context, an increase in protein oxidation and ROS production as determined used these methods is indicative of increased expression of NADPH oxidase 4, which can be associated with reduced muscle function. Increased protein oxidation and ROS production in a mouse model of OI relative to a normal mouse is indicative of reduced muscle function. In the context of humans, similar methods can be used to assess protein oxidation and ROS production in a muscle biopsy, where an increase in protein oxidation and ROS production relative to a healthy individual is indicative of reduced muscle function.

RyR1 immunoprecipitation and immunoblotting. RyR1 oxidation and nitrosylation and calstabin1 binding can be determined as previously described (Andersson et. al. Cell Metab. 14, 196-207 (2011)). Extensor digitorum longus (EDL) muscles can be isotonically lysed in 0.5 ml of a buffer containing 50 mM Tris-HCl (pH 7.4), 150 mM NaCl, 20 mM NaF, 1.0 mM Na3VO4, and protease inhibitors. C2C12 cells were lysed in NP-40 lysis buffer containing 50 mM Tris-HCl (pH 8.0) 150 mM NaCl, 1.0% NP-40 and protease inhibitors. An anti-RyR antibody (e.g., 4 μg anti-RyR1 antibody 5029, a custom antibody against the last nine amino acids (CRKQYEDQLS (SEQ ID NO: 404); a cysteine is added at the N terminus) of the rabbit skeletal muscle RyR1) can be used to immunoprecipitate RyR1 from 250 μg of tissue homogenate (Andersson et. al. Cell Metab. 14, 196-207 (2011)). Samples can be incubated with antibody in 0.75 ml of a modified RIPA buffer (50 mM Tris-HCl pH 7.4, 0.9% NaCl, 5.0 mM NaF, 1.0 mM Na3VO4, 1% Triton-X100 and protease inhibitors) for 1 h at 4° C. The immune complexes are incubated with protein A-sepharose beads (Sigma) overnight at 4° C. and the beads are washed twice with modified RIPA buffer. Proteins are separated on 4-12% Bis-Tris gels (Life Technologies) and transferred to nitrocellulose for 1 h at 100 V (Bio-Rad, Hercules, Calif.). After incubation with blocking solution to prevent nonspecific antibody binding, immunoblots are developed with anti-RyR (Affinity Bioreagents, cat. MA3-916, Golden, Colo.; 1:2,000) and anti-Cys-NO antibody (Sigma, cat. N0409, St. Louis, Mo.; 1:2,000) or an anti-calstabin antibody (Santa Cruz Biotechnology, cat. sc-6173, Santa Cruz, Calif.; 1:2,500). Immunoblots are developed and quantified using the Odyssey Infrared Imaging System (LICOR Biosystems, Lincoln, Nebr.) and infrared-labeled secondary antibodies. Detection of pSMAD3, SMAD3, Nox4 (Abcam, Cambridge, UK; 1:1,000 each), GAPDH and tubulin (Sigma; 1:500 each) from mouse muscle, human biopsies, and C2C12 cells can be via lysis in NP-40 buffer and detection and quantification of immobilized proteins can be performed using the Odyssey Infrared Imaging System or GE ImageQuantLAS4000Imaging System (GEHealthcare Bio-sciences, Pittsburgh, Pa.). Increased RyR1 oxidation, nitrosylation, and/or calstabin1 binding in a mouse model of OI relative to a normal mouse is indicative of reduced muscle function. In the context of humans, similar methods can be used to assess RyR1 oxidation, nitrosylation, and/or calstabin1 binding in a muscle biopsy, where an increase in RyR1 oxidation, nitrosylation, and/or calstabin1 binding relative to a healthy individual is indicative of reduced muscle function.

### Example 13

### Administration of a Conjugate Containing a TGF-β Antagonist and a Bone-Targeting Moiety for the Treatment of a Patient Having DMD

Using the compositions and methods described herein, a physician of skill in the art can administer to a patient (e.g., a human patient) suffering from a muscular dystrophy (e.g., DMD) a conjugate containing a bone-targeting moiety and a TGF-β receptor fusion protein, such as a TGF-β receptor fusion protein having the amino acid sequence of SEQ ID NO: 9, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, and SEQ ID NO: 35, or a TGF-β receptor fusion protein having at least 70% sequence identity (e.g., at least 75%, 80%, 85%, 90%, 95%, 97%, 98%, 99%, or more, sequence identity) thereto. The TGF-β receptor fusion protein can be bound to a bone-targeting hydroxyapatite-binding domain, such as a polyanionic peptide of the formula Dn or En, in which D and E designate aspartate and glutamate, respectively, and each n designates an integer from 1 to 25 (e.g., 1, 2, 3, 4, 5, 6, 7, 8, 9, 10, 11, 12, 13, 14, 15, 16, 17, 18, 19, 20, 21, 22, 23, 24, or 25). For example, TGF-β receptor fusion protein can be bound to a bone-targeting hydroxyapatite-binding domain of the formula D10. For example, the conjugate may be a protein having the amino acid sequence of SEQ ID NO: 5, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, and SEQ ID NO: 34, or at least 70% sequence identity (e.g., at least 75%, 80%, 85%, 90%, 95%, 97%, 98%, 99%, or more, sequence identity) thereto. The physician may assess the patient by first evaluating muscle function in the patient using one or more methodologies, such as manual muscle testing, dynamometry, or muscle mechanography, or imaging techniques to assess muscle-cross sectional area, volume, density, or muscle mass (e.g., MRI or CT scans). A finding that the individual has reduced muscle function relative to a control indicates that the patient may be particularly well suited for treatment with a conjugate described herein.

The physician may administer to the patient a therapeutically effective amount (e.g., an amount sufficient to attenuate TGF-β signaling and/or to reduce bone turnover) of a conjugate described herein according to a dosing schedule determined, for instance, by the patient's age, weight, gender, and/or severity of the disease. For example, the conjugate may be administered to the subject in one or more doses (e.g., 1, 2, 3, 4, 5, 6, 7, 8, 9, 10, 15, 20, 25, 30, or more) per a specified time interval, such as one or more doses per day, per week, per month, or per year. The patient may be evaluated between doses so as to monitor the effectiveness of the therapy and to increase or reduce the dosage based on the patient's response. For example, an improvement in one or more, or all, of muscle mass, muscle strength, or muscle quality throughout the course of treatment may indicate that the therapy is effectively treating the muscular dystrophy.

The conjugate may be administered to the patient by a variety of routes of administration, for instance, as determined by a physician of skill in the art. For example, the conjugate may be administered to the patient in one or more repeat doses subcutaneously, intradermally, intramuscularly, intraperitoneally, intravenously, or orally, or by nasal or by epidural administration. Conjugates described herein can be formulated with excipients, biologically acceptable carriers, and may be optionally conjugated to, admixed with, or co-administered separately (e.g., sequentially) with additional therapeutic agents.

Prior to the conclusion of therapy, the physician may prescribe progressively lower doses of the conjugate to the patient so as to gradually reduce the concentration of the therapy. In some instances, the therapy may involve only a single dosing of the therapeutic conjugate.

Alternatively, the therapy may continue, for instance, for a period of days, weeks, months, or years prior to completion.

### Example 14

### TGF-β and Muscle Strength in Mice with Osteogenesis Imperfecta (OI)

Osteogenesis imperfecta (OI) is a disease attributable to any of a large number of possible mutations of type I collagen. The homozygous murine model (OIM) recapitulates many of the features of human OI including, the skeletal phenotype of severe osteogenesis imperfecta in humans (OI type III). The OIM mice experience spontaneous fractures, reduced bone mineral density, gross changes in skeletal structure, and increased osteoclast activity (Chipman S D. Proc Natl Acad Sci USA. 1993; 90:1701-5). Additionally, OIM mice also have impaired mobility due to reduced muscle strength (Veilleux L N et al. Bone. 2015; 79:52-7; Gentry B A et al. Matrix Biol. 2010; 29(7):638-44).

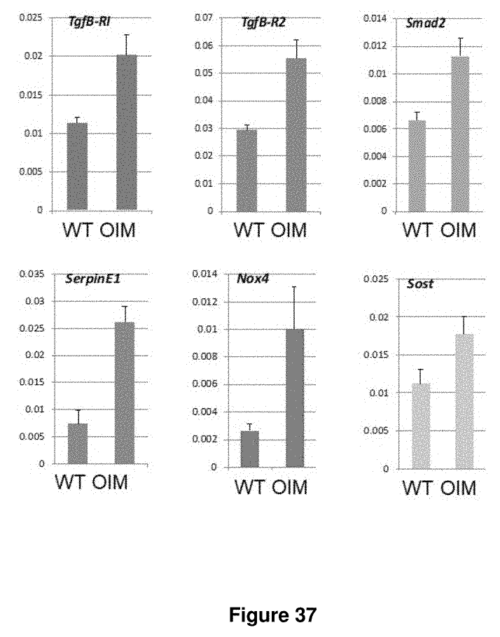

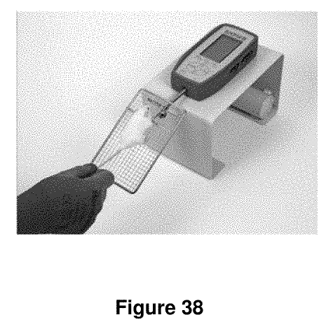

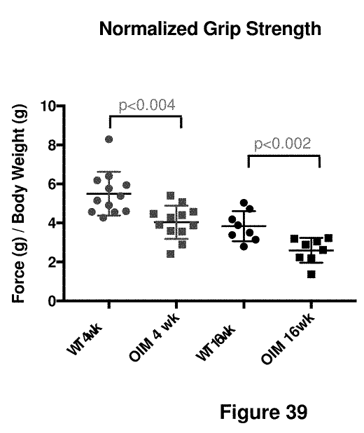

RT-PCR of representative TGF-β inducible genes confirms that TGF-β is elevated in OIM bones relative to WT bones (FIG. 37). To assess muscle strength, a forelimb grip test was performed using a commercial automatic grip strength meter. Specifically, mice were allowed to grip a wire screen as the experimenter pulled each mouse horizontally by the tail. The pulling force was increased steadily by the tail until the mouse lost its grip and the peak force was measured by the meter (FIG. 38). This noninvasive test is widely used to evaluate forelimb strength and to assess the effects of the disease (Bonetto A et al. Bonekey Rep. 2015; 4:732). Grip strength in OIM mice was reduced relative to wild-type (WT) mice, and decreased as the animals aged (FIG. 39). A study was performed to assess the ability of TGF-β antagonists with or without a bone-targeting moiety D10 (10 aspartate repeat) to improve muscle functions in OIM mice.

### Example 15

### Immunostaining Detection of Bone-Targeted TGF-β Antibody in Skeleton of Mice Treated with Bone-Targeted TGF-β Antagonists

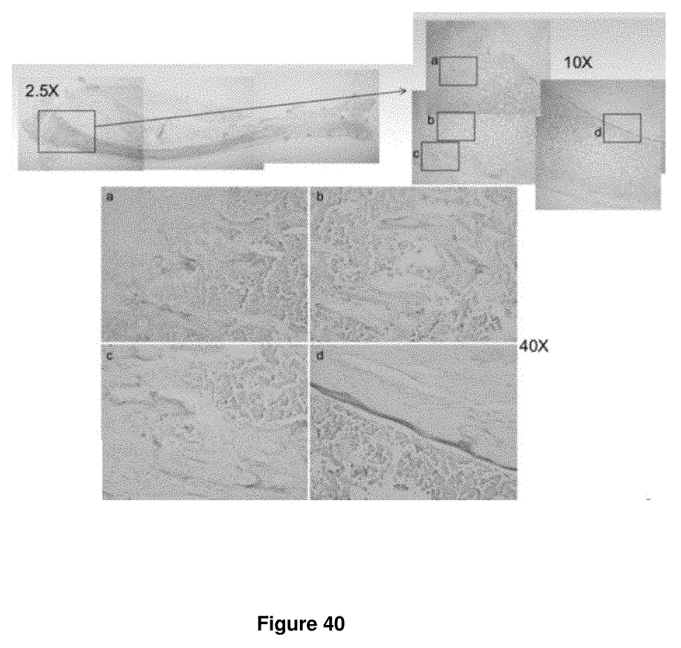

To demonstrate that bone-targeted TGF-β antibody is localized in the skeleton, mice treated with a TGF-β neutralizing antibody (Fresolimumab, GC1008) (U.S. Pat. No. 9,598,486) containing the bone-targeting moiety D10 (10 aspartate repeat) designated as PCT-0011 at a dose of 5 mg/kg one time weekly, from 12 weeks to 16 weeks of age were euthanized following treatment. Tibia and mandible were isolated and decalcified at 4 oC for 2 weeks in 8% EDTA and 1% formaldehyde. These were embedded in paraffin, sectioned, and processed for IHC using the rabbit anti-human-IgG antibody from Abcam. The DAKO envision-HRP kit was used for the detection. A representative picture of tibia in a 16-week-old mouse taken at 2.5, 10 and 40+ magnification under a light microscope is shown (FIG. 40). Detection is visualized by brown staining at surface of trabecular (panels a, b, and c) and cortical bone (panel d). Staining was absent in the bones of PBS-treated mice. The immunostaining results confirmed skeletal localization of bone-targeted TGF-β antagonist in skeleton of treated mice.

### Example 16

### Mobility Assessments of Mice with Osteogenesis Imperfecta (OIM) Treated with Non-Targeted and Bone-Targeted TGF-β Antagonists

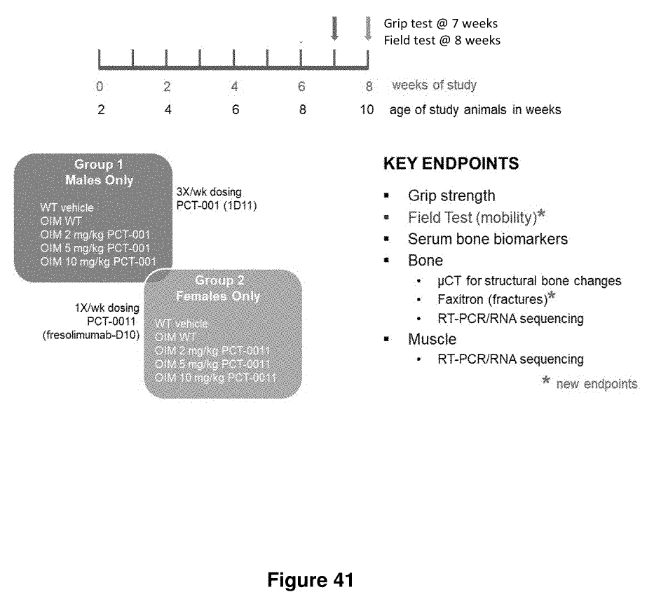

A detailed study was performed to assess the ability of TGF-β antagonists with or without a bone-targeting moiety D10 (10 aspartate repeat) to improve muscle functions in OIM mice. OIM mice (2-week-old) were treated with PBS vehicle, or 2, 5 or 10 mg/kg per injection of a neutralizing antibody against TGF-β without the bone-targeting moiety D10 (anti-TGF-β Antibody 1D11) designated as PCT-001 or the TGF-β neutralizing antibody containing the bone-targeting moiety D10 designated as PCT-0011. The study design is shown on FIG. 41. Key endpoints included mobility (identified by an open field test measuring distance covered, speed, and vertical movement) and forelimb grip strength.

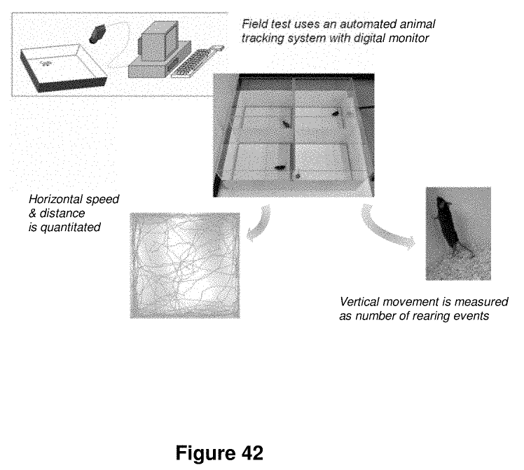

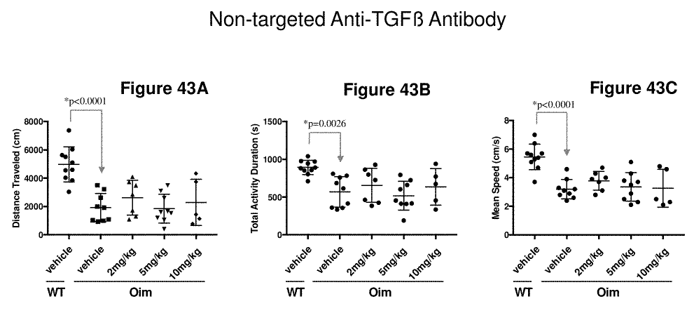

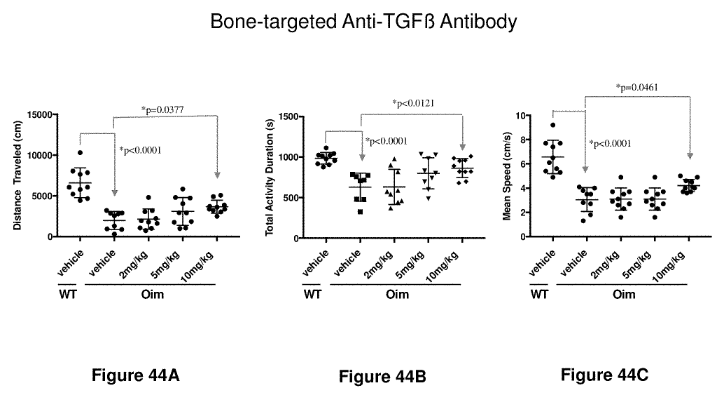

The open field test used to measure overall mobility/general locomotor activity (FIG. 42) is based on the tendency of mice to explore new environments and monitors the overall distance, speed and average time spent moving during a defined period (20 minutes). The open field apparatus contains an arena with walls to prevent escape. The apparatus used in this study monitored movement with a video camera linked to a computer with a software to quantitate the specified movements. The open field test assessment (FIG. 42) was performed on mice after 8 weeks of treatment. Significant decreases (p<0.05) in the distance traveled, the total duration of activity and mean speed per 20-minute assessment (n=6-10 mice per group) was observed for control vehicle treated OIM mice relative to wild-type mice (FIGS. 43 and 44). There were no observable effects upon treatment with non-targeted TGF-β antibody PCT-001 (FIG. 43). In contrast, a dose-dependent increase in each of the assessed parameters were observed after treatment with bone-targeted TGF-β antibody PCT-0011 with statistical significance (p<0.05) achieved at the highest dose tested (10 mg/kg) (FIG. 44). These results demonstrate that treatment with a bone-targeted TGF-β antibody led to improvement in overall mobility, an indirect measurement of muscle function.

### Example 17

### Forelimb Grip Strength Assessment of Mice Osteogenesis Imperfecta (OIM) Treated with Non-Targeted and Bone-Targeted TGF-β Antagonists

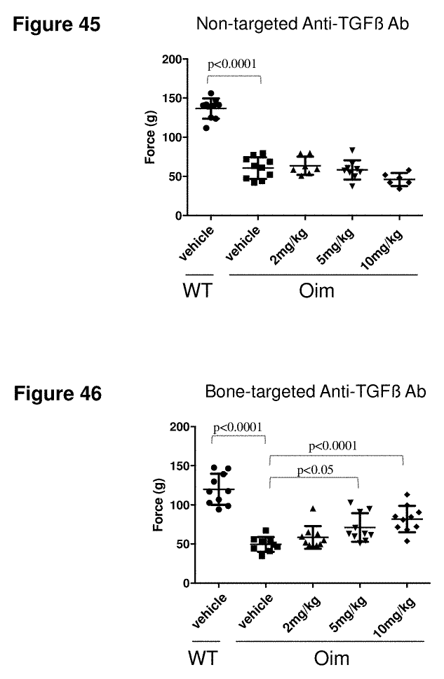

In the same experiment described in Example 16, a forelimb grip test was performed using a commercial automatic grip strength meter (FIG. 38). The results presented here represent five (5) replicate measurements per mouse. OIM mice (2-week-old) were treated with PBS vehicle, or 2, 5 or 10 mg/kg per injection of non-targeted TGF-β antibody PCT-001 or bone-targeted TGF-β antibody PCT-0011. Significant decreases (p<0.05) in the forearm grip strength was however observed for OIM mice relative to wild-type mice (FIGS. 45 and 46). There were no observable effects upon treatment with non-targeted TGF-β antagonist PCT-001 (FIG. 45). In contrast, a dose-dependent increase in grip strength was observed after treatment with bone-targeted TGF-β antibody PCT-0011 with statistical significance achieved at the two highest doses tested (5 and 10 mg/kg) (FIG. 46). These results demonstrate that treatment with a bone-targeted TGF-β antibody but not with a non-targeted antibody led to improvement in forelimb muscle strength, which is a measurement of muscle function.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

Sequences Listing
(Full length human TGF-β receptor II)
SEQ ID NO: 1
MGRGLLRGLWPLHIVLWTRIASTIPPHVQKSVNNDMIVTDNNGAVKFPQLCKFCDVRFSTCDNQKSC
MSNCSITSICEKPQEVCVAVWRKNDENITLETVCHDPKLPYHDFILEDAASPKCIMKEKKKPGETFFM
CSCSSDECNDNIIFSEEYNTSNPDLLLVIFQVTGISLLPPLGVAISVIIIFYCYRVNRQQKLSSTWETGKT
RKLMEFSEHCAIILEDDRSDISSTCANNINHNTELLPIELDTLVGKGRFAEVYKAKLKQNTSEQFETVAV
KIFPYEEYASWKTEKDIFSDINLKHENILQFLTAEERKTELGKQYWLITAFHAKGNLQEYLTRHVISWED
LRKLGSSLARGIAHLHSDHTPCGRPKMPIVHRDLKSSNILVKNDLTCCLCDFGLSLRLDPTLSVDDLAN
SGQVGTARYMAPEVLESRMNLENVESFKQTDVYSMALVLWEMTSRCNAVGEVKDYEPPFGSKVRE
HPCVESMKDNVLRDRGRPEIPSFWLNHQGIQMVCETLTECWDHDPEARLTAQCVAERFSELEHLDR
LSGRSCSEEKIPEDGSLNTTK
(Full length rat TGF-β receptor III)
SEQ ID NO: 2
MAVTSHHMIPVMVVLMSACLATAGPEPSTRCELSPINASHPVQALMESFTVLSGCASRGTTGLPREV
HVLNLRSTDQGPGQRQREVTLHLNPIASVHTHHKPIVFLLNSPQPLVWHLKTERLAAGVPRLFLVSEG
SVVQFPSGNFSLTAETEERNFPQENEHLLRWAQKEYGAVTSFTELKIARNIYIKVGEDQVFPPTCNIG
KNFLSLNYLAEYLQPKAAEGCVLPSQPHEKEVHIIELITPSSNPYSAFQVDIIVDIRPAQEDPEVVKNLVL
ILKCKKSVNWVIKSFDVKGNLKVIAPNSIGFGKESERSMTMTKLVRDDIPSTQENLMKWALDNGYRPV
TSYTMAPVANRFHLRLENNEEMRDEEVHTIPPELRILLDPDHPPALDNPLFPGEGSPNGGLPFPFPDI
PRRGWKEGEDRIPRPKQPIVPSVQLLPDHREPEEVQGGVDIALSVKCDHEKMVVAVDKDSFQTNGY
SGMELTLLDPSCKAKMNGTHFVLESPLNGCGTRHRRSTPDGVVYYNSIVVQAPSPGDSSGWPDGY
EDLESGDNGFPGDGDEGETAPLSRAGVVVFNCSLRQLRNPSGFQGQLDGNATFNMELYNTDLFLVP
SPGVFSVAENEHVYVEVSVTKADQDLGFAIQTCFLSPYSNPDRMSDYTIIENICPKDDSVKFYSSKRV
HFPIPHAEVDKKRFSFLFKSVFNTSLLFLHCELTLCSRKKGSLKLPRCVTPDDACTSLDATMIWTMMQ
NKKTFTKPLAVVLQVDYKENVPSTKDSSPIPPPPPQIFHGLDTLTVMGIAFAAFVIGALLTGALWYIYSH
TGETARRQQVPTSPPASENSSAAHSIGSTQSTPCSSSSTA
(Full length human TGF-β receptor III)
SEQ ID NO: 3
MTSHYVIAIFALMSSCLATAGPEPGALCELSPVSASHPVQALMESFTVLSGCASRGTTGLPQEVHVLN
LRTAGQGPGQLQREVTLHLNPISSVHIHHKSVVFLLNSPHPLVWHLKTERLATGVSRLFLVSEGSVVQ
FSSANFSLTAETEERNFPHGNEHLLNWARKEYGAVTSFTELKIARNIYIKVGEDQVFPPKCNIGKNFLS
LNYLAEYLQPKAAEGCVMSSQPQNEEVHIIELITPNSNPYSAFQVDITIDIRPSQEDLEVVKNLILILKCK
KSVNWVIKSFDVKGSLKIIAPNSIGFGKESERSMTMTKSIRDDIPSTQGNLVKWALDNGYSPITSYTMA
PVANRFHLRLENNEEMGDEEVHTIPPELRILLDPGALPALQNPPIRGGEGQNGGLPFPFPDISRRVWN
EEGEDGLPRPKDPVIPSIQLFPGLREPEEVQGSVDIALSVKCDNEKMIVAVEKDSFQASGYSGMDVTL
LDPTCKAKMNGTHFVLESPLNGCGTRPRWSALDGVVYYNSIVIQVPALGDSSGWPDGYEDLESGDN
GFPGDMDEGDASLFTRPEIVVFNCSLQQVRNPSSFQEQPHGNITFNMELYNTDLFLVPSQGVFSVPE
NGHVYVEVSVTKAEQELGFAIQTCFISPYSNPDRMSHYTIIENICPKDESVKFYSPKRVHFPIPQADMD
KKRFSFVFKPVFNTSLLFLQCELTLCTKMEKHPQKLPKCVPPDEACTSLDASIIWAMMQNKKTFTKPL
AVIHHEAESKEKGPSMKEPNPISPPIFHGLDTLTVMGIAFAAFVIGALLTGALWYIYSHTGETAGRQQV
PTSPPASENSSAAHSIGSTQSTPCSSSSTA
(Albumin signal peptide)
SEQ ID NO: 4
MKWVTFLLLLFISGSAFSAAA
(Exemplary TGF-β antagonist conjugate)
SEQ ID NO: 5
MKWVTFLLLLFISGSAFSAAANGAVKFPQLCKFCDVRFSTCDNQKSCMSNCSITSICEKPQEVCVAV
WRKNDENITLETVCHDPKLPYHDFILEDAASPKCIMKEKKKPGETFFMCSCSSDECNDNIIFSEEYNTS
NPDGLGPVESSPGHGLDTAAAGPEPSTRCELSPINASHPVQALMESFTVLSGCASHGTTGLPREVHV
LNLRSTDQGPGQRQREVTLHLNPIASVHTHHKPIVFLLNSPQPLVWRLKTERLAAGVPRLFLVSEGSV
VQFPSGNFSLTAETEERNFPQENEHLLRWAQKEYGAVTSFTELKIARNIYIKVGEDQVFPPTCNIGKN
FLSLNYLAEYLQPKAAEGCVLPSQPHEKEVHIIELITPSSNPYSAFQVDIIVDIRPAQEDPEVVKNLVLIL
KSKKSVNWVIKSFDVKGNLKVIAPNSIGFGKESERSMTMTKLVRDDIPSTQENLMKWALDAGYRPVT
SYTMAPVANRFHLRLENNEEMRDEEVHTIPPELRILLDPDKLPQLCKFCDVRFSTCDNQKSCMSNCSI
TSICEKPQEVCVAVWRKNDENITLETVCHDPKLPYHDFILEDAASPKCIMKEKKKPGETFFMCSCSSD
ECNDNIIFSEEYNTSNPDGGGGSGGGGSGGGGSGDDDDDDDDDD
(Polyglycine linker)
SEQ ID NO: 6
GGG
(G4S linker)
SEQ ID NO: 7
GGGGS
(linker)
SEQ ID NO: 8
GGGGSGGGGSGGGGSG
(Exemplary TGF-β receptor fusion protein of the
structure R₂ₐ-R₃-R_{2b} or RII ectodomain-RIII
endoglin domain-RII ectodomain (RER))
SEQ ID NO: 9
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGPEPSTRCELSPINASHP
VQALMESFTVLSGCASHGTTGLPREVHVLNLRSTDQGPGQRQREVTLHLNPIASVHTHHKPIVFLLNS
PQPLVWRLKTERLAAGVPRLFLVSEGSVVQFPSGNFSLTAETEERNFPQENEHLLRWAQKEYGAVT
SFTELKIARNIYIKVGEDQVFPPTCNIGKNFLSLNYLAEYLQPKAAEGCVLPSQPHEKEVHIIELITPSSN
PYSAFQVDIIVDIRPAQEDPEVVKNLVLILKSKKSVNWVIKSFDVKGNLKVIAPNSIGFGKESERSMTMT
KLVRDDIPSTQENLMKWALDAGYRPVTSYTMAPVANRFHLRLENNEEMRDEEVHTIPPELRILLDPDP
QLCKFCDVRFSTCDNQKSCMSNCSITSICEKPQEVCVAVWRKNDENITLETVCHDPKLPYHDFILEDA
ASPKCIMKEKKKPGETFFMCSCSSDECNDNIIFSEEYNTSNPD
(Human R₂ ectodomain - residues 42-159 of human R₂)
SEQ ID NO: 10
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPD
(Human R₂ ectodomain - residues 48-159 of human R₂)
SEQ ID NO: 11
PQLCKFCDVRFSTCDNQKSCMSNCSITSICEKPQEVCVAVWRKNDENITLETVCHDPKLPYHDFILED
AASPKCIMKEKKKPGETFFMCSCSSDECNDNIIFSEEYNTSNPD
(Rat R₃ endoglin domain residues 24-383 containing R58H,
H116R, C278S, and N337A mutations)
SEQ ID NO: 12
GPEPSTRCELSPINASHPVQALMESFTVLSGCASHGTTGLPREVHVLNLRSTDQGPGQRQREVTLHL
NPIASVHTHHKPIVFLLNSPQPLVWRLKTERLAAGVPRLFLVSEGSVVQFPSGNFSLTAETEERNFPQ
ENEHLLRWAQKEYGAVTSFTELKIARNIYIKVGEDQVFPPTCNIGKNFLSLNYLAEYLQPKAAEGCVLP
SQPHEKEVHIIELITPSSNPYSAFQVDIIVDIRPAQEDPEVVKNLVLILKSKKSVNWVIKSFDVKGNLKVIA
PNSIGFGKESERSMTMTKLVRDDIPSTQENLMKWALDAGYRPVTSYTMAPVANRFHLRLENNEEMR
DEEVHTIPPELRILLDPD
(Human R₃ endoglin domain residues 21-380 containing
the C275S mutation)
SEQ ID NO: 13
GPEPGALCELSPVSASHPVQALMESFTVLSGCASRGTTGLPQEVHVLNLRTAGQGPGQLQREVTLH
LNPISSVHIHHKSVVFLLNSPHPLVWHLKTERLATGVSRLFLVSEGSVVQFSSANFSLTAETEERNFPH
GNEHLLNWARKEYGAVTSFTELKIARNIYIKVGEDQVFPPKCNIGKNFLSLNYLAEYLQPKAAEGCVM
SSQPQNEEVHIIELITPNSNPYSAFQVDITIDIRPSQEDLEVVKNLILILKSKKSVNWVIKSFDVKGSLKIIA
PNSIGFGKESERSMTMTKSIRDDIPSTQGNLVKWALDNGYSPITSYTMAPVANRFHLRLENNEEMGD
EEVHTIPPELRILLDPG
(PCT-0015 without SP, with bone-targeting moiety)
SEQ ID NO: 14
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGLGPVESSPGHGLDTAA
AGPEPSTRCELSPINASHPVQALMESFTVLSGCASHGTTGLPREVHVLNLRSTDQGPGQRQREVTL
HLNPIASVHTHHKPIVFLLNSPQPLVWRLKTERLAAGVPRLFLVSEGSVVQFPSGNFSLTAETEERNF
PQENEHLLRWAQKEYGAVTSFTELKIARNIYIKVGEDQVFPPTCNIGKNFLSLNYLAEYLQPKAAEGCV
LPSQPHEKEVHIIELITPSSNPYSAFQVDIIVDIRPAQEDPEVVKNLVLILKSKKSVNWVIKSFDVKGNLK
VIAPNSIGFGKESERSMTMTKLVRDDIPSTQENLMKWALDAGYRPVTSYTMAPVANRFHLRLENNEE
MRDEEVHTIPPELRILLDPDKLPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYNTSNPDTGG
GDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGKDDDDDDDDDD
(PCT-0015 without SP, without bone-targeting moiety)
SEQ ID NO: 15
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGLGPVESSPGHGLDTAA
AGPEPSTRCELSPINASHPVQALMESFTVLSGCASHGTTGLPREVHVLNLRSTDQGPGQRQREVTL
HLNPIASVHTHHKPIVFLLNSPQPLVWRLKTERLAAGVPRLFLVSEGSVVQFPSGNFSLTAETEERNF
PQENEHLLRWAQKEYGAVTSFTELKIARNIYIKVGEDQVFPPTCNIGKNFLSLNYLAEYLQPKAAEGCV
LPSQPHEKEVHIIELITPSSNPYSAFQVDIIVDIRPAQEDPEVVKNLVLILKSKKSVNWVIKSFDVKGNLK
VIAPNSIGFGKESERSMTMTKLVRDDIPSTQENLMKWALDAGYRPVTSYTMAPVANRFHLRLENNEE
MRDEEVHTIPPELRILLDPDKLPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYNTSNPDTGG
GDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
(PCT-0019, without SP, with bone-targeting moiety)
SEQ ID NO: 16
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGLGPVESSPGHGLDTAA
AGPEPSTRCELSPINASHPVQALMESFTVLSGCASHGTTGLPREVHVLNLRSTDQGPGQRQREVTL
HLNPIASVHTHHKPIVFLLNSPQPLVWRLKTERLAAGVPRLFLVSEGSVVQFPSGNFSLTAETEERNF
PQENEHLLRWAQKEYGAVTSFTELKIARNIYIKVGEDQVFPPTCNIGKNFLSLNYLAEYLQPKAAEGCV
LPSQPHEKEVHIIELITPSSNPYSAFQVDIIVDIRPAQEDPEVVKNLVLILKSKKSVNWVIKSFDVKGNLK
VIAPNSIGFGKESERSMTMTKLVRDDIPSTQENLMKWALDAGYRPVTSYTMAPVANRFHLRLENNEE
MRDEEVHTIPPELRILLDPDKLPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYNTSNPDGGG
GSGGGGSGGGGSGDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGKDDDDDDDDDD
(PCT-0019, without SP, without bone-targeting moiety)
SEQ ID NO: 17
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGLGPVESSPGHGLDTAA
AGPEPSTRCELSPINASHPVQALMESFTVLSGCASHGTTGLPREVHVLNLRSTDQGPGQRQREVTL
HLNPIASVHTHHKPIVFLLNSPQPLVWRLKTERLAAGVPRLFLVSEGSVVQFPSGNFSLTAETEERNF
PQENEHLLRWAQKEYGAVTSFTELKIARNIYIKVGEDQVFPPTCNIGKNFLSLNYLAEYLQPKAAEGCV
LPSQPHEKEVHIIELITPSSNPYSAFQVDIIVDIRPAQEDPEVVKNLVLILKSKKSVNWVIKSFDVKGNLK
VIAPNSIGFGKESERSMTMTKLVRDDIPSTQENLMKWALDAGYRPVTSYTMAPVANRFHLRLENNEE
MRDEEVHTIPPELRILLDPDKLPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYNTSNPDGGG
GSGGGGSGGGGSGDKTHTCPPCPAPELLGGPSVFLFPPKPKDTLMISRTPEVTCVVVDVSHEDPEV
KFNWYVDGVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK
(PCT-0020, without SP, with bone-targeting moiety)
SEQ ID NO: 18
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGLGPVESSPGHGLDTAA
AGPEPSTRCELSPINASHPVQALMESFTVLSGCASHGTTGLPREVHVLNLRSTDQGPGQRQREVTL
HLNPIASVHTHHKPIVFLLNSPQPLVWRLKTERLAAGVPRLFLVSEGSVVQFPSGNFSLTAETEERNF
PQENEHLLRWAQKEYGAVTSFTELKIARNIYIKVGEDQVFPPTCNIGKNFLSLNYLAEYLQPKAAEGCV
LPSQPHEKEVHIIELITPSSNPYSAFQVDIIVDIRPAQEDPEVVKNLVLILKSKKSVNWVIKSFDVKGNLK
VIAPNSIGFGKESERSMTMTKLVRDDIPSTQENLMKWALDAGYRPVTSYTMAPVANRFHLRLENNEE
MRDEEVHTIPPELRILLDPDKLPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYNTSNPDLLLV
IFQVTGISLLPPLGGGGSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGKDDDDDDDDDD
(PCT-0020, without SP, without bone targeting moiety)
SEQ ID NO: 19
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGLGPVESSPGHGLDTAA
AGPEPSTRCELSPINASHPVQALMESFTVLSGCASHGTTGLPREVHVLNLRSTDQGPGQRQREVTL
HLNPIASVHTHHKPIVFLLNSPQPLVWRLKTERLAAGVPRLFLVSEGSVVQFPSGNFSLTAETEERNF
PQENEHLLRWAQKEYGAVTSFTELKIARNIYIKVGEDQVFPPTCNIGKNFLSLNYLAEYLQPKAAEGCV
LPSQPHEKEVHIIELITPSSNPYSAFQVDIIVDIRPAQEDPEVVKNLVLILKSKKSVNWVIKSFDVKGNLK
VIAPNSIGFGKESERSMTMTKLVRDDIPSTQENLMKWALDAGYRPVTSYTMAPVANRFHLRLENNEE
MRDEEVHTIPPELRILLDPDKLPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYNTSNPDLLLV
IFQVTGISLLPPLGGGGSDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
(PCT-0021, without SP, with bone targeting moiety)
SEQ ID NO: 20
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGLGPVESSPGHGLDTAA
AGPEPSTRCELSPINASHPVQALMESFTVLSGCASHGTTGLPREVHVLNLRSTDQGPGQRQREVTL
HLNPIASVHTHHKPIVFLLNSPQPLVWRLKTERLAAGVPRLFLVSEGSVVQFPSGNFSLTAETEERNF
PQENEHLLRWAQKEYGAVTSFTELKIARNIYIKVGEDQVFPPTCNIGKNFLSLNYLAEYLQPKAAEGCV
LPSQPHEKEVHIIELITPSSNPYSAFQVDIIVDIRPAQEDPEVVKNLVLILKSKKSVNWVIKSFDVKGNLK
VIAPNSIGFGKESERSMTMTKLVRDDIPSTQENLMKWALDAGYRPVTSYTMAPVANRFHLRLENNEE
MRDEEVHTIPPELRILLDPDKLPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYNTSNPDLLLV
IFQVTGISLLPPLGVAISVIIIDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKDDDDDDDDDD
(PCT-0021, without SP, without bone-targeting moiety)
SEQ ID NO: 21
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGLGPVESSPGHGLDTAA
AGPEPSTRCELSPINASHPVQALMESFTVLSGCASHGTTGLPREVHVLNLRSTDQGPGQRQREVTL
HLNPIASVHTHHKPIVFLLNSPQPLVWRLKTERLAAGVPRLFLVSEGSVVQFPSGNFSLTAETEERNF
PQENEHLLRWAQKEYGAVTSFTELKIARNIYIKVGEDQVFPPTCNIGKNFLSLNYLAEYLQPKAAEGCV
LPSQPHEKEVHIIELITPSSNPYSAFQVDIIVDIRPAQEDPEVVKNLVLILKSKKSVNWVIKSFDVKGNLK
VIAPNSIGFGKESERSMTMTKLVRDDIPSTQENLMKWALDAGYRPVTSYTMAPVANRFHLRLENNEE
MRDEEVHTIPPELRILLDPDKLPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYNTSNPDLLLV
IFQVTGISLLPPLGVAISVIIIDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK
(PCT-0022, without SP, with bone-targeting moiety)
SEQ ID NO: 22
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDLLLVIFQVTGISLLPPLAG
PEPSTRCELSPINASHPVQALMESFTVLSGCASHGTTGLPREVHVLNLRSTDQGPGQRQREVTLHLN
PIASVHTHHKPIVFLLNSPQPLVWRLKTERLAAGVPRLFLVSEGSVVQFPSGNFSLTAETEERNFPQE
NEHLLRWAQKEYGAVTSFTELKIARNIYIKVGEDQVFPPTCNIGKNFLSLNYLAEYLQPKAAEGCVLPS
QPHEKEVHIIELITPSSNPYSAFQVDIIVDIRPAQEDPEVVKNLVLILKSKKSVNWVIKSFDVKGNLKVIAP
NSIGFGKESERSMTMTKLVRDDIPSTQENLMKWALDAGYRPVTSYTMAPVANRFHLRLENNEEMRD
EEVHTIPPELRILLDPDKLPQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNIIFSEEYNTSNPDLLLVIFQ
VTGISLLPPLGVAISVIIIDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGKDDDDDDDDDD
(PCT-0022, without SP, without bone-targeting moiety)
SEQ ID NO: 23
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDLLLVIFQVTGISLLPPLAG
PEPSTRCELSPINASHPVQALMESFTVLSGCASHGTTGLPREVHVLNLRSTDQGPGQRQREVTLHLN
PIASVHTHHKPIVFLLNSPQPLVWRLKTERLAAGVPRLFLVSEGSVVQFPSGNFSLTAETEERNFPQE
NEHLLRWAQKEYGAVTSFTELKIARNIYIKVGEDQVFPPTCNIGKNFLSLNYLAEYLQPKAAEGCVLPS
QPHEKEVHIIELITPSSNPYSAFQVDIIVDIRPAQEDPEVVKNLVLILKSKKSVNWVIKSFDVKGNLKVIAP
NSIGFGKESERSMTMTKLVRDDIPSTQENLMKWALDAGYRPVTSYTMAPVANRFHLRLENNEEMRD
EEVHTIPPELRILLDPDKLPQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNIIFSEEYNTSNPDLLLVIFQ
VTGISLLPPLGVAISVIIIDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK
(PCT-0023, without SP, with bone-targeting moiety)
SEQ ID NO: 24
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDLLLVIFQVTGISLLPPL
AGPEPGALCELSPVSASHPVQALMESFTVLSGCASRGTTGLPQEVHVLNLRTAGQGPGQLQREVTL
HLNPISSVHIHHKSVVFLLNSPHPLVWHLKTERLATGVSRLFLVSEGSVVQFSSANFSLTAETEERNFP
HGNEHLLNWARKEYGAVTSFTELKIARNIYIKVGEDQVFPPKCNIGKNFLSLNYLAEYLQPKAAEGCV
MSSQPQNEEVHIIELITPNSNPYSAFQVDITIDIRPSQEDLEVVKNLILILKSKKSVNWVIKSFDVKGSLKII
APNSIGFGKESERSMTMTKSIRDDIPSTQGNLVKWALDNGYSPITSYTMAPVANRFHLRLENNEEMG
DEEVHTIPPELRILLDPGALPQLCKFCDVRFSTCDNQKSCMSNCSITSICEKPQEVCVAVWRKNDENI
TLETVCHDPKLPYHDFILEDAASPKCIMKEKKKPGETFFMCSCSSDECNDNIIFSEEYNTSNPDLLLVIF
QVTGISLLPPLGVAISVIIIDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGKDDDDDDDDDD
(PCT-0023, without SP, without bone-targeting moiety)
SEQ ID NO: 25
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDLLLVIFQVTGISLLPPL
AGPEPGALCELSPVSASHPVQALMESFTVLSGCASRGTTGLPQEVHVLNLRTAGQGPGQLQREVTL
HLNPISSVHIHHKSVVFLLNSPHPLVWHLKTERLATGVSRLFLVSEGSVVQFSSANFSLTAETEERNFP
HGNEHLLNWARKEYGAVTSFTELKIARNIYIKVGEDQVFPPKCNIGKNFLSLNYLAEYLQPKAAEGCV
MSSQPQNEEVHIIELITPNSNPYSAFQVDITIDIRPSQEDLEVVKNLILILKSKKSVNWVIKSFDVKGSLKII
APNSIGFGKESERSMTMTKSIRDDIPSTQGNLVKWALDNGYSPITSYTMAPVANRFHLRLENNEEMG
DEEVHTIPPELRILLDPGALPQLCKFCDVRFSTCDNQKSCMSNCSITSICEKPQEVCVAVWRKNDENI
TLETVCHDPKLPYHDFILEDAASPKCIMKEKKKPGETFFMCSCSSDECNDNIIFSEEYNTSNPDLLLVIF
QVTGISLLPPLGVAISVIIIDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
(PCT-0024, without SP, with bone-targeting moiety)
SEQ ID NO: 26
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDGLGPVESSPGHGLDT
AAAGPEPGALCELSPVSASHPVQALMESFTVLSGCASRGTTGLPQEVHVLNLRTAGQGPGQLQREV
TLHLNPISSVHIHHKSVVFLLNSPHPLVWHLKTERLATGVSRLFLVSEGSVVQFSSANFSLTAETEERN
FPHGNEHLLNWARKEYGAVTSFTELKIARNIYIKVGEDQVFPPKCNIGKNFLSLNYLAEYLQPKAAEGC
VMSSQPQNEEVHIIELITPNSNPYSAFQVDITIDIRPSQEDLEVVKNLILILKSKKSVNWVIKSFDVKGSL
KIIAPNSIGFGKESERSMTMTKSIRDDIPSTQGNLVKWALDNGYSPITSYTMAPVANRFHLRLENNEEM
GDEEVHTIPPELRILLDPGALPQLCKFCDVRFSTCDNQKSCMSNCSITSICEKPQEVCVAVWRKNDEN
ITLETVCHDPKLPYHDFILEDAASPKCIMKEKKKPGETFFMCSCSSDECNDNIIFSEEYNTSNPDLLLVI
FQVTGISLLPPLGVAISVIIIDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGKDDDDDDDDDD
(PCT-0024, without SP, without bone-targeting moiety)
SEQ ID NO: 27
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDGLGPVESSPGHGLDT
AAAGPEPGALCELSPVSASHPVQALMESFTVLSGCASRGTTGLPQEVHVLNLRTAGQGPGQLQREV
TLHLNPISSVHIHHKSVVFLLNSPHPLVWHLKTERLATGVSRLFLVSEGSVVQFSSANFSLTAETEERN
FPHGNEHLLNWARKEYGAVTSFTELKIARNIYIKVGEDQVFPPKCNIGKNFLSLNYLAEYLQPKAAEGC
VMSSQPQNEEVHIIELITPNSNPYSAFQVDITIDIRPSQEDLEVVKNLILILKSKKSVNWVIKSFDVKGSL
KIIAPNSIGFGKESERSMTMTKSIRDDIPSTQGNLVKWALDNGYSPITSYTMAPVANRFHLRLENNEEM
GDEEVHTIPPELRILLDPGALPQLCKFCDVRFSTCDNQKSCMSNCSITSICEKPQEVCVAVWRKNDEN
ITLETVCHDPKLPYHDFILEDAASPKCIMKEKKKPGETFFMCSCSSDECNDNIIFSEEYNTSNPDLLLVI
FQVTGISLLPPLGVAISVIIIDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
(PCT-0025, without SP, with bone-targeting moiety)
SEQ ID NO: 28
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDLLLVIFQVTGISLLPPL
GVAISVIIIAGPEPGALCELSPVSASHPVQALMESFTVLSGCASRGTTGLPQEVHVLNLRTAGQGPGQ
LQREVTLHLNPISSVHIHHKSVVFLLNSPHPLVWHLKTERLATGVSRLFLVSEGSVVQFSSANFSLTAE
TEERNFPHGNEHLLNWARKEYGAVTSFTELKIARNIYIKVGEDQVFPPKCNIGKNFLSLNYLAEYLQPK
AAEGCVMSSQPQNEEVHIIELITPNSNPYSAFQVDITIDIRPSQEDLEVVKNLILILKSKKSVNWVIKSFD
VKGSLKIIAPNSIGFGKESERSMTMTKSIRDDIPSTQGNLVKWALDNGYSPITSYTMAPVANRFHLRLE
NNEEMGDEEVHTIPPELRILLDPGALPQLCKFCDVRFSTCDNQKSCMSNCSITSICEKPQEVCVAVW
RKNDENITLETVCHDPKLPYHDFILEDAASPKCIMKEKKKPGETFFMCSCSSDECNDNIIFSEEYNTSN
PDLLLVIFQVTGISLLPPLGVAISVIIIDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGKDDDDDDDDDD
(PCT-0025, without SP, without bone-targeting moiety)
SEQ ID NO: 29
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDLLLVIFQVTGISLLPPL
GVAISVIIIAGPEPGALCELSPVSASHPVQALMESFTVLSGCASRGTTGLPQEVHVLNLRTAGQGPGQ
LQREVTLHLNPISSVHIHHKSVVFLLNSPHPLVWHLKTERLATGVSRLFLVSEGSVVQFSSANFSLTAE
TEERNFPHGNEHLLNWARKEYGAVTSFTELKIARNIYIKVGEDQVFPPKCNIGKNFLSLNYLAEYLQPK
AAEGCVMSSQPQNEEVHIIELITPNSNPYSAFQVDITIDIRPSQEDLEVVKNLILILKSKKSVNWVIKSFD
VKGSLKIIAPNSIGFGKESERSMTMTKSIRDDIPSTQGNLVKWALDNGYSPITSYTMAPVANRFHLRLE
NNEEMGDEEVHTIPPELRILLDPGALPQLCKFCDVRFSTCDNQKSCMSNCSITSICEKPQEVCVAVW
RKNDENITLETVCHDPKLPYHDFILEDAASPKCIMKEKKKPGETFFMCSCSSDECNDNIIFSEEYNTSN
PDLLLVIFQVTGISLLPPLGVAISVIIIDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
(PCT-0026, without SP, with bone-targeting moiety)
SEQ ID NO: 30
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDGLGPVESSPGHGLDT
AAAGPEPGALCELSPVSASHPVQALMESFTVLSGCASRGTTGLPQEVHVLNLRTAGQGPGQLQREV
TLHLNPISSVHIHHKSVVFLLNSPHPLVWHLKTERLATGVSRLFLVSEGSVVQFSSANFSLTAETEERN
FPHGNEHLLNWARKEYGAVTSFTELKIARNIYIKVGEDQVFPPKCNIGKNFLSLNYLAEYLQPKAAEGC
VMSSQPQNEEVHIIELITPNSNPYSAFQVDITIDIRPSQEDLEVVKNLILILKSKKSVNWVIKSFDVKGSL
KIIAPNSIGFGKESERSMTMTKSIRDDIPSTQGNLVKWALDNGYSPITSYTMAPVANRFHLRLENNEEM
GDEEVHTIPPELRILLDPGALPQLCKFCDVRFSTCDNQKSCMSNCSITSICEKPQEVCVAVWRKNDEN
ITLETVCHDPKLPYHDFILEDAASPKCIMKEKKKPGETFFMCSCSSDECNDNIIFSEEYNTSNPDLLLVI
FQVTGISLLPPLGGGGSDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGKDDDDDDDDDD
(PCT-0026, without SP, without bone-targeting moiety)
SEQ ID NO: 31
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDGLGPVESSPGHGLDT
AAAGPEPGALCELSPVSASHPVQALMESFTVLSGCASRGTTGLPQEVHVLNLRTAGQGPGQLQREV
TLHLNPISSVHIHHKSVVFLLNSPHPLVWHLKTERLATGVSRLFLVSEGSVVQFSSANFSLTAETEERN
FPHGNEHLLNWARKEYGAVTSFTELKIARNIYIKVGEDQVFPPKCNIGKNFLSLNYLAEYLQPKAAEGC
VMSSQPQNEEVHIIELITPNSNPYSAFQVDITIDIRPSQEDLEVVKNLILILKSKKSVNWVIKSFDVKGSL
KIIAPNSIGFGKESERSMTMTKSIRDDIPSTQGNLVKWALDNGYSPITSYTMAPVANRFHLRLENNEEM
GDEEVHTIPPELRILLDPGALPQLCKFCDVRFSTCDNQKSCMSNCSITSICEKPQEVCVAVWRKNDEN
ITLETVCHDPKLPYHDFILEDAASPKCIMKEKKKPGETFFMCSCSSDECNDNIIFSEEYNTSNPDLLLVI
FQVTGISLLPPLGGGGSDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQPENNYKTTPPVLDSDGSFF
LYSKLTVDKSRWQQGNVFSCSVMHEALHNHYTQKSLSLSPGK
(PCT-0017, without SP, with bone-targeting moiety)
SEQ ID NO: 32
DDDDDDDDDDGGGGSGGGGSGGGGSGGGGSGDKTHTCPPCPAPELLGGPSVFLFPPKPKDTLMI
SRTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPGKGGGG
SGGGGSGGGGSGNGAVKFPQLCKFCDVRFSTCDNQKSCMSNCSITSICEKPQEVCVAVWRKNDEN
ITLETVCHDPKLPYHDFILEDAASPKCIMKEKKKPGETFFMCSCSSDECNDNIIFSEEYNTSNPDGLGP
VESSPGHGLDTAAAGPEPSTRCELSPINASHPVQALMESFTVLSGCASHGTTGLPREVHVLNLRSTD
QGPGQRQREVTLHLNPIASVHTHHKPIVFLLNSPQPLVWRLKTERLAAGVPRLFLVSEGSVVQFPSG
NFSLTAETEERNFPQENEHLLRWAQKEYGAVTSFTELKIARNIYIKVGEDQVFPPTCNIGKNFLSLNYL
AEYLQPKAAEGCVLPSQPHEKEVHIIELITPSSNPYSAFQVDIIVDIRPAQEDPEVVKNLVLILKSKKSVN
WVIKSFDVKGNLKVIAPNSIGFGKESERSMTMTKLVRDDIPSTQENLMKWALDAGYRPVTSYTMAPV
ANRFHLRLENNEEMRDEEVHTIPPELRILLDPDKLPQLCKFCDVRFSTCDNQKSCMSNCSITSICEKP
QEVCVAVWRKNDENITLETVCHDPKLPYHDFILEDAASPKCIMKEKKKPGETFFMCSCSSDECNDNII
FSEEYNTSNPD
(PCT-0016, without SP, no bone-targeting moiety)
SEQ ID NO: 33
DKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGNGAVKFPQLCKFCDVRFSTCD
NQKSCMSNCSITSICEKPQEVCVAVWRKNDENITLETVCHDPKLPYHDFILEDAASPKCIMKEKKKPG
ETFFMCSCSSDECNDNIIFSEEYNTSNPDGLGPVESSPGHGLDTAAAGPEPSTRCELSPINASHPVQ
ALMESFTVLSGCASHGTTGLPREVHVLNLRSTDQGPGQRQREVTLHLNPIASVHTHHKPIVFLLNSPQ
PLVWRLKTERLAAGVPRLFLVSEGSVVQFPSGNFSLTAETEERNFPQENEHLLRWAQKEYGAVTSFT
ELKIARNIYIKVGEDQVFPPTCNIGKNFLSLNYLAEYLQPKAAEGCVLPSQPHEKEVHIIELITPSSNPYS
AFQVDIIVDIRPAQEDPEVVKNLVLILKSKKSVNWVIKSFDVKGNLKVIAPNSIGFGKESERSMTMTKLV
RDDIPSTQENLMKWALDAGYRPVTSYTMAPVANRFHLRLENNEEMRDEEVHTIPPELRILLDPDKLPQ
LCKFCDVRFSTCDNQKSCMSNCSITSICEKPQEVCVAVWRKNDENITLETVCHDPKLPYHDFILEDAA
SPKCIMKEKKKPGETFFMCSCSSDECNDNIIFSEEYNTSNPD
(PCT-0018; without SP, with bone-targeting moiety)
SEQ ID NO: 34
DKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGNGAVKFPQLCKFCDVRFSTCD
NQKSCMSNCSITSICEKPQEVCVAVWRKNDENITLETVCHDPKLPYHDFILEDAASPKCIMKEKKKPG
ETFFMCSCSSDECNDNIIFSEEYNTSNPDGLGPVESSPGHGLDTAAAGPEPSTRCELSPINASHPVQ
ALMESFTVLSGCASHGTTGLPREVHVLNLRSTDQGPGQRQREVTLHLNPIASVHTHHKPIVFLLNSPQ
PLVWRLKTERLAAGVPRLFLVSEGSVVQFPSGNFSLTAETEERNFPQENEHLLRWAQKEYGAVTSFT
ELKIARNIYIKVGEDQVFPPTCNIGKNFLSLNYLAEYLQPKAAEGCVLPSQPHEKEVHIIELITPSSNPYS
AFQVDIIVDIRPAQEDPEVVKNLVLILKSKKSVNWVIKSFDVKGNLKVIAPNSIGFGKESERSMTMTKLV
RDDIPSTQENLMKWALDAGYRPVTSYTMAPVANRFHLRLENNEEMRDEEVHTIPPELRILLDPDKLPQ
LCKFCDVRFSTCDNQKSCMSNCSITSICEKPQEVCVAVWRKNDENITLETVCHDPKLPYHDFILEDAA
SPKCIMKEKKKPGETFFMCSCSSDECNDNIIFSEEYNTSNPDGGGGSGGGGSGGGGSGGGGSGDD
DDDDDDDD
(PCT-0018, without SP, without bone-targeting moiety)
SEQ ID NO: 35
DKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQG
NVFSCSVMHEALHNHYTQKSLSLSPGKGGGGSGGGGSGGGGSGNGAVKFPQLCKFCDVRFSTCD
NQKSCMSNCSITSICEKPQEVCVAVWRKNDENITLETVCHDPKLPYHDFILEDAASPKCIMKEKKKPG
ETFFMCSCSSDECNDNIIFSEEYNTSNPDGLGPVESSPGHGLDTAAAGPEPSTRCELSPINASHPVQ
ALMESFTVLSGCASHGTTGLPREVHVLNLRSTDQGPGQRQREVTLHLNPIASVHTHHKPIVFLLNSPQ
PLVWRLKTERLAAGVPRLFLVSEGSVVQFPSGNFSLTAETEERNFPQENEHLLRWAQKEYGAVTSFT
ELKIARNIYIKVGEDQVFPPTCNIGKNFLSLNYLAEYLQPKAAEGCVLPSQPHEKEVHIIELITPSSNPYS
AFQVDIIVDIRPAQEDPEVVKNLVLILKSKKSVNWVIKSFDVKGNLKVIAPNSIGFGKESERSMTMTKLV
RDDIPSTQENLMKWALDAGYRPVTSYTMAPVANRFHLRLENNEEMRDEEVHTIPPELRILLDPDKLPQ
LCKFCDVRFSTCDNQKSCMSNCSITSICEKPQEVCVAVWRKNDENITLETVCHDPKLPYHDFILEDAA
SPKCIMKEKKKPGETFFMCSCSSDECNDNIIFSEEYNTSNPD
(Linker)
SEQ ID NO: 36
TGGG
(Linker)
SEQ ID NO: 37
LLLVIFQVTGISLLPPLGGGGS
(Linker)
SEQ ID NO: 38
LLLVIFQVTGISLLPPLGVAISVIII
(Linker)
SEQ ID NO: 39
LLLVIFQVTGISLLPPL
(Linker)
SEQ ID NO: 40
GLGPVESSPGHGLDTAA
(Linker)
SEQ ID NO: 41
GGGGSGGGGSGGGGSGGGGSG
(Alpha-lactalbumin used as signal peptide
alternative to albumin)
SEQ ID NO: 42
MMSFVSLLLVGILFHATQ
(Human R₂ₐ for PCT-0015, PCT-0015NT, PCT-0019,
PCT-0019NT, PCT-0021, PCT-0021NT, PCT-0022, PCT-0022NT, PCT-0016NT,
PCT-0017, PCT-0018, PCT-0018)
SEQ ID NO: 43
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPD
(Human R₃ for PCT-0023, PCT-0023NT, PCT-0024, PCT-0024NT,
PCT-0025, PCT-0025NT, PCT-0026, PCT-0023)
SEQ ID NO: 44
AGPEPGALCELSPVSASHPVQALMESFTVLSGCASRGTTGLPQEVHVLNLRTAGQGPGQLQREVTL
HLNPISSVHIHHKSVVFLLNSPHPLVWHLKTERLATGVSRLFLVSEGSVVQFSSANFSLTAETEERNFP
HGNEHLLNWARKEYGAVTSFTELKIARNIYIKVGEDQVFPPKCNIGKNFLSLNYLAEYLQPKAAEGCV
MSSQPQNEEVHIIELITPNSNPYSAFQVDITIDIRPSQEDLEVVKNLILILKSKKSVNWVIKSFDVKGSLKII
APNSIGFGKESERSMTMTKSIRDDIPSTQGNLVKWALDNGYSPITSYTMAPVANRFHLRLENNEEMG
DEEVHTIPPELRILLDPGAL
(Human R_{2b} for PCT-0015 to PCT-0026)
SEQ ID NO: 45
PQLCKFCDVRFSTCDNQKSCMSNCSITSICEKPQEVCVAVWRKNDENITLETVCHDPKLPYHDFILED
AASPKCIMKEKKKPGETFFMCSCSSDECNDNIIFSEEYNTSNPD
(D10 bone-targeting moiety)
SEQ ID NO: 46
DDDDDDDDDD
(Fc domain of immunoglobulin)
SEQ ID NO: 47
DKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKSRWQQG
NVFSCSVMHEALHNHYTQKSLSLSPGK
(RER of PCT-0020)
SEQ ID NO: 48
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDGLGPVESSPGHGLDTAA
AGPEPSTRCELSPINASHPVQALMESFTVLSGCASHGTTGLPREVHVLNLRSTDQGPGQRQREVTL
HLNPIASVHTHHKPIVFLLNSPQPLVWRLKTERLAAGVPRLFLVSEGSVVQFPSGNFSLTAETEERNF
PQENEHLLRWAQKEYGAVTSFTELKIARNIYIKVGEDQVFPPTCNIGKNFLSLNYLAEYLQPKAAEGCV
LPSQPHEKEVHIIELITPSSNPYSAFQVDIIVDIRPAQEDPEVVKNLVLILKSKKSVNWVIKSFDVKGNLK
VIAPNSIGFGKESERSMTMTKLVRDDIPSTQENLMKWALDAGYRPVTSYTMAPVANRFHLRLENNEE
MRDEEVHTIPPELRILLDPDKLPQLCKFCDVRFSTCDNQKSCMSNCSITSICEKPQEVCVAVWRKNDE
NITLETVCHDPKLPYHDFILEDAASPKCIMKEKKKPGETFFMCSCSSDECNDNIIFSEEYNTSNPD
(RER of PCT-0022)
SEQ ID NO: 49
NGAVKFPQLCKFCDVRFSTCDNQKSCMSNCSITSICEKPQEVCVAVWRKNDENITLETVCHDPKLPY
HDFILEDAASPKCIMKEKKKPGETFFMCSCSSDECNDNIIFSEEYNTSNPDLLLVIFQVTGISLLPPLAG
PEPSTRCELSPINASHPVQALMESFTVLSGCASHGTTGLPREVHVLNLRSTDQGPGQRQREVTLHLN
PIASVHTHHKPIVFLLNSPQPLVWRLKTERLAAGVPRLFLVSEGSVVQFPSGNFSLTAETEERNFPQE
NEHLLRWAQKEYGAVTSFTELKIARNIYIKVGEDQVFPPTCNIGKNFLSLNYLAEYLQPKAAEGCVLPS
QPHEKEVHIIELITPSSNPYSAFQVDIIVDIRPAQEDPEVVKNLVLILKSKKSVNWVIKSFDVKGNLKVIAP
NSIGFGKESERSMTMTKLVRDDIPSTQENLMKWALDAGYRPVTSYTMAPVANRFHLRLENNEEMRD
EEVHTIPPELRILLDPDKLPQLCKFCDVRFSTCDNQKSCMSNCSITSICEKPQEVCVAVWRKNDENITL
ETVCHDPKLPYHDFILEDAASPKCIMKEKKKPGETFFMCSCSSDECNDNIIFSEEYNTSNPD
(RER of PCT-0023)
SEQ ID NO: 50
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDLLLVIFQVTGISLLPPL
AGPEPGALCELSPVSASHPVQALMESFTVLSGCASRGTTGLPQEVHVLNLRTAGQGPGQLQREVTL
HLNPISSVHIHHKSVVFLLNSPHPLVWHLKTERLATGVSRLFLVSEGSVVQFSSANFSLTAETEERNFP
HGNEHLLNWARKEYGAVTSFTELKIARNIYIKVGEDQVFPPKCNIGKNFLSLNYLAEYLQPKAAEGCV
MSSQPQNEEVHIIELITPNSNPYSAFQVDITIDIRPSQEDLEVVKNLILILKSKKSVNWVIKSFDVKGSLKII
APNSIGFGKESERSMTMTKSIRDDIPSTQGNLVKWALDNGYSPITSYTMAPVANRFHLRLENNEEMG
DEEVHTIPPELRILLDPGALPQLCKFCDVRFSTCDNQKSCMSNCSITSICEKPQEVCVAVWRKNDENI
TLETVCHDPKLPYHDFILEDAASPKCIMKEKKKPGETFFMCSCSSDECNDNIIFSEEYNTSNPD
(RER of PCT-0024 and PCT-0026)
SEQ ID NO: 51
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDGLGPVESSPGHGLDT
AAAGPEPGALCELSPVSASHPVQALMESFTVLSGCASRGTTGLPQEVHVLNLRTAGQGPGQLQREV
TLHLNPISSVHIHHKSVVFLLNSPHPLVWHLKTERLATGVSRLFLVSEGSVVQFSSANFSLTAETEERN
FPHGNEHLLNWARKEYGAVTSFTELKIARNIYIKVGEDQVFPPKCNIGKNFLSLNYLAEYLQPKAAEGC
VMSSQPQNEEVHIIELITPNSNPYSAFQVDITIDIRPSQEDLEVVKNLILILKSKKSVNWVIKSFDVKGSL
KIIAPNSIGFGKESERSMTMTKSIRDDIPSTQGNLVKWALDNGYSPITSYTMAPVANRFHLRLENNEEM
GDEEVHTIPPELRILLDPGALPQLCKFCDVRFSTCDNQKSCMSNCSITSICEKPQEVCVAVWRKNDEN
ITLETVCHDPKLPYHDFILEDAASPKCIMKEKKKPGETFFMCSCSSDECNDNIIFSEEYNTSNPD
(RER of PCT-0025)
SEQ ID NO: 52
ADNGAVKFPQLCKFCDVRFSTCDNQKSCMSNCSITSICEKPQEVCVAVWRKNDENITLETVCHDPKL
PYHDFILEDAASPKCIMKEKKKPGETFFMCSCSSDECNDNIIFSEEYNTSNPDLLLVIFQVTGISLLPPL
GVAISVIIIAGPEPGALCELSPVSASHPVQALMESFTVLSGCASRGTTGLPQEVHVLNLRTAGQGPGQ
LQREVTLHLNPISSVHIHHKSVVFLLNSPHPLVWHLKTERLATGVSRLFLVSEGSVVQFSSANFSLTAE
TEERNFPHGNEHLLNWARKEYGAVTSFTELKIARNIYIKVGEDQVFPPKCNIGKNFLSLNYLAEYLQPK
AAEGCVMSSQPQNEEVHIIELITPNSNPYSAFQVDITIDIRPSQEDLEVVKNLILILKSKKSVNWVIKSFD
VKGSLKIIAPNSIGFGKESERSMTMTKSIRDDIPSTQGNLVKWALDNGYSPITSYTMAPVANRFHLRLE
NNEEMGDEEVHTIPPELRILLDPGALPQLCKFCDVRFSTCDNQKSCMSNCSITSICEKPQEVCVAVW
RKNDENITLETVCHDPKLPYHDFILEDAASPKCIMKEKKKPGETFFMCSCSSDECNDNIIFSEEYNTSN
PD
(Linker)
SEQ ID NO: 402
GGGGSGGGGS
(Linker)
SEQ ID NO: 53
TGGGDGGGGS
(Linker)
SEQ ID NO: 54
GGGGSLLLVIFQVTGISLLPPLGVAISVIII
(Linker)
SEQ ID NO: 55
GGGGSLLLVIFQVTGISLLPPL
(Linker)
SEQ ID NO: 56
GGGGSGGGGSLLLVIFQVTGISLLPPL
(Linker)
SEQ ID NO: 57
GGGGSGLGPVESSPGHGLDTAA
(Linker)
SEQ ID NO: 58
GGGGSGGGGSGLGPVESSPGHGLDTAA
(Linker)
SEQ ID NO: 59
KL
(Linker)
SEQ ID NO: 60
(GGGS)n, wherein n = 1, 2, 3, 4, or 5
(Linker)
SEQ ID NO: 61
(GGGGS)n, wherein n = 1, 2, 3, 4, or 5
(Heavy chain of PCT-0011 with D10 bone-targeting moiety)
SEQ ID NO: 62
QVQLVQSGAEVKKPGSSVKVSCKASGYTFSSNVISWVRQAPGQGLEWMGGVIPIVDIANYAQRFKG
RVTITADESTSTTYMELSSLRSEDTAVYYCASTLGLVLDAMDYWGQGTLVTVSSASTKGPSVFPLAP
CSRSTSESTAALGCLVKDYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNGKEYKCKVSNKGLPSSIE
KTISKAKGQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGKDDDDDDDDDD
(Light chain of PCT-0011)
SEQ ID NO: 63
ETVLTQSPGTLSLSPGERATLSCRASQSLGSSYLAWYQQKPGQAPRLLIYGASSRAPGIPDRFSGSG
SGTDFTLTISRLEPEDFAVYYCQQYADSPITFGQGTRLEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGL
SSPVTKSFNRGEC
(Nucleic acid sequence of PCT-0023 construct)
SEQ ID NO: 398
TTTAAGCTTGCCGCCACCATGATGTCCTTTGTCTCTCTGCTCCTGGTTGGCATCCTATTCCATGC
CACCCAGGCCGACAACGGTGCAGTCAAGTTTCCACAACTGTGTAAATTTTGTGATGTGAGATTTT
CCACCTGTGACAACCAGAAATCCTGCATGAGCAACTGCAGCATCACCTCCATCTGTGAGAAGCC
ACAGGAAGTCTGTGTGGCTGTATGGAGAAAGAATGACGAGAACATAACACTAGAGACAGTTTGC
CATGACCCCAAGCTCCCCTACCATGACTTTATTCTGGAAGATGCTGCTTCTCCAAAGTGCATTAT
GAAGGAAAAAAAAAAGCCTGGAGAGACTTTCTTCATGTGTTCCTGTAGCTCTGATGAGTGCAATG
ACAACATCATCTTCTCAGAAGAATATAACACCAGCAATCCTGACTTGTTGCTAGTCATATTTCAAG
TGACAGGCATCAGCCTCCTGCCACCACTGGCAGGTCCAGAGCCTGGTGCACTGTGTGAACTGTC
ACCTGTCAGTGCCTCCCATCCTGTCCAGGCCTTGATGGAGAGCTTCACTGTTTTGTCAGGCTGT
GCCAGCAGAGGCACAACTGGGCTGCCACAGGAGGTGCATGTCCTGAATCTCCGCACTGCAGGC
CAGGGGCCTGGCCAGCTACAGAGAGAGGTCACACTTCACCTGAATCCCATCTCCTCAGTCCACA
TCCACCACAAGTCTGTTGTGTTCCTGCTCAACTCCCCACACCCCCTGGTGTGGCATCTGAAGACA
GAGAGACTTGCCACTGGGGTCTCCAGACTGTTTTTGGTGTCTGAGGGTTCTGTGGTCCAGTTTTC
ATCAGCAAACTTCTCCTTGACAGCAGAAACAGAAGAAAGGAACTTCCCCCATGGAAATGAACATC
TGTTAAATTGGGCCCGAAAAGAGTATGGAGCAGTTACTTCATTCACCGAACTCAAGATAGCAAGA
AACATTTATATTAAAGTGGGGGAAGATCAAGTGTTCCCTCCAAAGTGCAACATAGGGAAGAATTT
TCTCTCACTCAATTACCTTGCTGAGTACCTTCAACCCAAAGCAGCAGAAGGGTGTGTGATGTCCA
GCCAGCCCCAGAATGAGGAAGTACACATCATCGAGCTAATCACCCCCAACTCTAACCCCTACAG
TGCTTTCCAGGTGGATATAACAATTGATATAAGACCTTCTCAAGAGGATCTTGAAGTGGTCAAAAA
TCTCATCCTGATCTTGAAGTCTAAAAAGTCTGTCAACTGGGTGATCAAATCTTTTGATGTTAAGGG
AAGCCTGAAAATTATTGCTCCTAACAGTATTGGCTTTGGAAAAGAGAGTGAAAGATCTATGACAAT
GACCAAATCAATAAGAGATGACATTCCTTCAACCCAAGGGAATCTGGTGAAGTGGGCATTTGGACA
ATGGCTATAGTCCAATAACTTCATACACAATGGCTCCTGTGGCTAATAGATTTCATCTTCGGCTTG
AAAATAATGAGGAGATGGGAGATGAGGAAGTCCACACTATTCCTCCTGAGCTACGGATCCTGCT
GGACCCTGGTGCCCTGCCGCAACTTTGCAAGTTCTGCGACGTGCGATTCTCTACGTGCGATAAT
CAAAAGTCCTGTATGTCAAACTGCAGTATTACTTCTATTTGTGAGAAGCCTCAGGAGGTTTGTGTC
GCGGTCTGGCGGAAAAACGACGAAAATATCACATTGGAAACGGTCTGCCACGACCCCAAACTTC
CCTATCATGATTTCATACTTGAGGATGCAGCTTCACCTAAGTGTATTATGAAAGAGAAGAAGAAAC
CAGGCGAAACGTTCTTTATGTGCAGTTGCTCCTCCGATGAATGCAATGATAACATCATTTTCTCC
GAGGAGTACAATACTTCAAATCCAGACCTCCTTCTCGTCATTTTTCAAGTTACAGGTATTTCACTG
CTCCCCCCTCTCGGCGTTGCGATATCAGTTATCATCATCGACAAAACTCACACATGCCCACCGTG
CCCAGCACCTGAACTCCTGGGGGGACCGTCAGTCTTCCTCTTCCCCCCAAAACCCAAGGACACC
CTCATGATCTCCCGGACCCCTGAGGTCACATGCGTGGTGGTGGACGTGAGCCACGAAGACCCT
GAGGTCAAGTTCAACTGGTACGTGGACGGCGTGGAGGTGCATAATGCCAAGACAAAGCCGCGG
GAGGAGCAGTACAACAGCACGTACCGTGTGGTCAGCGTCCTCACCGTCCTGCACCAGGACTGG
CTGAATGGCAAGGAGTACAAGTGCAAGGTCTCCAACAAAGCCCTCCCAGCCCCCATCGAGAAAA
CCATCTCCAAAGCCAAAGGGCAGCCCCGAGAACCACAGGTGTACACCCTGCCCCCATCCCGGG
ATGAGCTGACCAAGAACCAGGTCAGCCTGACCTGCCTGGTCAAAGGCTTCTATCCCAGCGACAT
CGCCGTGGAGTGGGAGAGCAATGGGCAGCCGGAGAACAACTACAAGACCACGCCTCCCGTGCT
GGACTCCGACGGCTCCTTCTTCCTCTATTCCAAGCTCACCGTGGACAAGAGCAGGTGGCAGCAG
GGGAACGTCTTCTCATGCTCCGTGATGCATGAGGCTCTGCACAACCACTACACGCAGAAGAGCC
TCTCCCTGTCTCCCGGGAAAGACGACGATGATGATGACGATGACGACGATTGATGACTCGAGTTT
(Nucleic acid sequence of PCT-0024 construct)
SEQ ID NO: 399
TTTAAGCTTGCCGCCACCATGATGTCCTTTGTCTCTCTGCTCCTGGTTGGCATCCTATTCCATGC
CACCCAGGCCGACAACGGTGCAGTCAAGTTTCCACAACTGTGTAAATTTTGTGATGTGAGATTTT
CCACCTGTGACAACCAGAAATCCTGCATGAGCAACTGCAGCATCACCTCCATCTGTGAGAAGCC
ACAGGAAGTCTGTGTGGCTGTATGGAGAAAGAATGACGAGAACATAACACTAGAGACAGTTTGC
CATGACCCCAAGCTCCCCTACCATGACTTTATTCTGGAAGATGCTGCTTCTCCAAAGTGCATTAT
GAAGGAAAAAAAAAAGCCTGGAGAGACTTTCTTCATGTGTTCCTGTAGCTCTGATGAGTGCAATG
ACAACATCATCTTCTCAGAAGAATATAACACCAGCAATCCTGACGGTCTTGGCCCCGTCGAGAGT
AGCCCTGGGCATGGCCTTGATACCGCAGCGGCAGGTCCAGAGCCTGGTGCACTGTGTGAACTG
TCACCTGTCAGTGCCTCCCATCCTGTCCAGGCCTTGATGGAGAGCTTCACTGTTTTGTCAGGCTG
TGCCAGCAGAGGCACAACTGGGCTGCCACAGGAGGTGCATGTCCTGAATCTCCGCACTGCAGG
CCAGGGGCCTGGCCAGCTACAGAGAGAGGTCACACTTCACCTGAATCCCATCTCCTCAGTCCAC
ATCCACCACAAGTCTGTTGTGTTCCTGCTCAACTCCCCACACCCCCTGGTGTGGCATCTGAAGAC
AGAGAGACTTGCCACTGGGGTCTCCAGACTGTTTTTGGTGTCTGAGGGTTCTGTGGTCCAGTTTT
CATCAGCAAACTTCTCCTTGACAGCAGAAACAGAAGAAAGGAACTTCCCCCATGGAAATGAACAT
CTGTTAAATTGGGCCCGAAAAGAGTATGGAGCAGTTACTTCATTCACCGAACTCAAGATAGCAAG
AAACATTTATATTAAAGTGGGGGAAGATCAAGTGTTCCCTCCAAAGTGCAACATAGGGAAGAATT
TTCTCTCACTCAATTACCTTGCTGAGTACCTTCAACCCAAAGCAGCAGAAGGGTGTGTGATGTCC
AGCCAGCCCCAGAATGAGGAAGTACACATCATCGAGCTAATCACCCCCAACTCTAACCCCTACA
GTGCTTTCCAGGTGGATATAACAATTGATATAAGACCTTCTCAAGAGGATCTTGAAGTGGTCAAA
AATCTCATCCTGATCTTGAAGTCTAAAAAGTCTGTCAACTGGGTGATCAAATCTTTTGATGTTAAG
GGAAGCCTGAAAATTATTGCTCCTAACAGTATTGGCTTTGGAAAAGAGAGTGAAAGATCTATGAC
AATGACCAAATCAATAAGAGATGACATTCCTTCAACCCAAGGGAATCTGGTGAAGTGGGCTTTGG
ACAATGGCTATAGTCCAATAACTTCATACACAATGGCTCCTGTGGCTAATAGATTTCATCTTCGGC
TTGAAAATAATGAGGAGATGGGAGATGAGGAAGTCCACACTATTCCTCCTGAGCTACGGATCCT
GCTGGACCCTGGTGCCCTGCCGCAACTTTGCAAGTTCTGCGACGTGCGATTCTCTACGTGCGAT
AATCAAAAGTCCTGTATGTCAAACTGCAGTATTACTTCTATTTGTGAGAAGCCTCAGGAGGTTTGT
GTCGCGGTCTGGCGGAAAAACGACGAAAATATCACATTGGAAACGGTCTGCCACGACCCCAAAC
TTCCCTATCATGATTTCATACTTGAGGATGCAGCTTCACCTAAGTGTATTATGAAAGAGAAGAAGA
AACCAGGCGAAACGTTCTTTATGTGCAGTTGCTCCTCCGATGAATGCAATGATAACATCATTTTCT
CCGAGGAGTACAATACTTCAAATCCAGACCTCCTTCTCGTCATTTTTCAAGTTACAGGTATTTCAC
TGCTCCCCCCTCTCGGCGTTGCGATATCAGTTATCATCATCGACAAAACTCACACATGCCCACCG
TGCCCAGCACCTGAACTCCTGGGGGGACCGTCAGTCTTCCTCTTCCCCCCAAAACCCAAGGACA
CCCTCATGATCTCCCGGACCCCTGAGGTCACATGCGTGGTGGTGGACGTGAGCCACGAAGACC
CTGAGGTCAAGTTCAACTGGTACGTGGACGGCGTGGAGGTGCATAATGCCAAGACAAAGCCGC
GGGAGGAGCAGTACAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCAGCCCCCATCGAGAA
AACCATCTCCAAAGCCAAAGGGCAGCCCCGAGAACCACAGGTGTACACCCTGCCCCCATCCCG
GGATGAGCTGACCAAGAACCAGGTCAGCCTGACCTGCCTGGTCAAAGGCTTCTATCCCAGCGAC
ATCGCCGTGGAGTGGGAGAGCAATGGGCAGCCGGAGAACAACTACAAGACCACGCCTCCCGTG
CTGGACTCCGACGGCTCCTTCTTCCTCTATTCCAAGCTCACCGTGGACAAGAGCAGGTGGCAGC
AGGGGAACGTCTTCTCATGCTCCGTGATGCATGAGGCTCTGCACAACCACTACACGCAGAAGAG
CCTCTCCCTGTCTCCCGGGAAAGACGACGATGATGATGACGATGACGACGATTGATGACTCGAG
TTT
(Nucleic acid sequence of PCT-0025 construct)
SEQ ID NO: 400
TTTAAGCTTGCCGCCACCATGATGTCCTTTGTCTCTCTGCTCCTGGTTGGCATCCTATTCCATGC
CACCCAGGCCGACAACGGTGCAGTCAAGTTTCCACAACTGTGTAAATTTTGTGATGTGAGATTTT
CCACCTGTGACAACCAGAAATCCTGCATGAGCAACTGCAGCATCACCTCCATCTGTGAGAAGCC
ACAGGAAGTCTGTGTGGCTGTATGGAGAAAGAATGACGAGAACATAACACTAGAGACAGTTTGC
CATGACCCCAAGCTCCCCTACCATGACTTTATTCTGGAAGATGCTGCTTCTCCAAAGTGCATTAT
GAAGGAAAAAAAAAAGCCTGGAGAGACTTTCTTCATGTGTTCCTGTAGCTCTGATGAGTGCAATG
ACAACATCATCTTCTCAGAAGAATATAACACCAGCAATCCTGACTTGTTGCTAGTCATATTTCAAG
TGACAGGCATCAGCCTCCTGCCACCACTGGGAGTTGCCATATCTGTCATCATCATCGCAGGTCC
AGAGCCTGGTGCACTGTGTGAACTGTCACCTGTCAGTGCCTCCCATCCTGTCCAGGCCTTGATG
GAGAGCTTCACTGTTTTGTCAGGCTGTGCCAGCAGAGGCACAACTGGGCTGCCACAGGAGGTG
CATGTCCTGAATCTCCGCACTGCAGGCCAGGGGCCTGGCCAGCTACAGAGAGAGGTCACACTT
CACCTGAATCCCATCTCCTCAGTCCACATCCACCACAAGTCTGTTGTGTTCCTGCTCAACTCCCC
ACACCCCCTGGTGTGGCATCTGAAGACAGAGAGACTTGCCACTGGGGTCTCCAGACTGTTTTTG
GTGTCTGAGGGTTCTGTGGTCCAGTTTTCATCAGCAAACTTCTCCTTGACAGCAGAAACAGAAGA
AAGGAACTTCCCCCATGGAAATGAACATCTGTTAAATTGGGCCCGAAAAGAGTATGGAGCAGTTA
CTTCATTCACCGAACTCAAGATAGCAAGAAACATTTATATTAAAGTGGGGGAAGATCAAGTGTTC
CCTCCAAAGTGCAACATAGGGAAGAATTTTCTCTCACTCAATTACCTTGCTGAGTACCTTCAACCC
AAAGCAGCAGAAGGGTGTGTGATGTCCAGCCAGCCCCAGAATGAGGAAGTACACATCATCGAGC
TAATCACCCCCAACTCTAACCCCTACAGTGCTTTCCAGGTGGATATAACAATTGATATAAGACCTT
CTCAAGAGGATCTTGAAGTGGTCAAAAATCTCATCCTGATCTTGAAGTCTAAAAAGTCTGTCAACT
GGGTGATCAAATCTTTTGATGTTAAGGGAAGCCTGAAAATTATTGCTCCTAACAGTATTGGCTTTG
GAAAAGAGAGTGAAAGATCTATGACAATGACCAAATCAATAAGAGATGACATTCCTTCAACCCAA
GGGAATCTGGTGAAGTGGGCTTTGGACAATGGCTATAGTCCAATAACTTCATACACAATGGCTCC
TGTGGCTAATAGATTTCATCTTCGGCTTGAAAATAATGAGGAGATGGGAGATGAGGAAGTCCACA
CTATTCCTCCTGAGCTACGGATCCTGCTGGACCCTGGTGCCCTGCCGCAACTTTGCAAGTTCTG
CGACGTGCGATTCTCTACGTGCGATAATCAAAAGTCCTGTATGTCAAACTGCAGTATTACTTCTAT
TTGTGAGAAGCCTCAGGAGGTTTGTGTCGCGGTCTGGCGGAAAAACGACGAAAATATCACATTG
GAAACGGTCTGCCACGACCCCAAACTTCCCTATCATGATTTCATACTTGAGGATGCAGCTTCACC
TAAGTGTATTATGAAAGAGAAGAAGAAACCAGGCGAAACGTTCTTTATGTGCAGTTGCTCCTCCG
ATGAATGCAATGATAACATCATTTTCTCCGAGGAGTACAATACTTCAAATCCAGACCTCCTTCTCG
TCATTTTTCAAGTTACAGGTATTTCACTGCTCCCCCCTCTCGGCGTTGCGATATCAGTTATCATCA
TCGACAAAACTCACACATGCCCACCGTGCCCAGCACCTGAACTCCTGGGGGGACCGTCAGTCTT
CCTCTTCCCCCCAAAACCCAAGGACACCCTCATGATCTCCCGGACCCCTGAGGTCACATGCGTG
GTGGTGGACGTGAGCCACGAAGACCCTGAGGTCAAGTTCAACTGGTACGTGGACGGCGTGGAG
GTGCATAATGCCAAGACAAAGCCGCGGGAGGAGCAGTACAACAGCACGTACCGTGTGGTCAGC
GTCCTCACCGTCCTGCACCAGGACTGGCTGAATGGCAAGGAGTACAAGTGCAAGGTCTCCAACA
AAGCCCTCCCAGCCCCCATCGAGAAAACCATCTCCAAAGCCAAAGGGCAGCCCCGAGAACCAC
AGGTGTACACCCTGCCCCCATCCCGGGATGAGCTGACCAAGAACCAGGTCAGCCTGACCTGCC
TGGTCAAAGGCTTCTATCCCAGCGACATCGCCGTGGAGTGGGAGAGCAATGGGCAGCCGGAGA
ACAACTACAAGACCACGCCTCCCGTGCTGGACTCCGACGGCTCCTTCTTCCTCTATTCCAAGCTC
ACCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCCGGGAAAGACGACGATGATGATG
ACGATGACGACGATTGATGACTCGAGTTT
(Nucleic acid sequence of PCT-0026 construct)
SEQ ID NO: 401
TTTAAGCTTGCCGCCACCATGATGTCCTTTGTCTCTCTGCTCCTGGTTGGCATCCTATTCCATGC
CACCCAGGCCGACAACGGTGCAGTCAAGTTTCCACAACTGTGTAAATTTTGTGATGTGAGATTTT
CCACCTGTGACAACCAGAAATCCTGCATGAGCAACTGCAGCATCACCTCCATCTGTGAGAAGCC
ACAGGAAGTCTGTGTGGCTGTATGGAGAAAGAATGACGAGAACATAACACTAGAGACAGTTTGC
CATGACCCCAAGCTCCCCTACCATGACTTTATTCTGGAAGATGCTGCTTCTCCAAAGTGCATTAT
GAAGGAAAAAAAAAAGCCTGGAGAGACTTTCTTCATGTGTTCCTGTAGCTCTGATGAGTGCAATG
ACAACATCATCTTCTCAGAAGAATATAACACCAGCAATCCTGACGGTCTTGGCCCCGTCGAGAGT
AGCCCTGGGCATGGCCTTGATACCGCAGCGGCAGGTCCAGAGCCTGGTGCACTGTGTGAACTG
TCACCTGTCAGTGCCTCCCATCCTGTCCAGGCCTTGATGGAGAGCTTCACTGTTTTGTCAGGCTG
TGCCAGCAGAGGCACAACTGGGCTGCCACAGGAGGTGCATGTCCTGAATCTCCGCACTGCAGG
CCAGGGGCCTGGCCAGCTACAGAGAGAGGTCACACTTCACCTGAATCCCATCTCCTCAGTCCAC
ATCCACCACAAGTCTGTTGTGTTCCTGCTCAACTCCCCACACCCCCTGGTGTGGCATCTGAAGAC
AGAGAGACTTGCCACTGGGGTCTCCAGACTGTTTTTGGTGTCTGAGGGTTCTGTGGTCCAGTTTT
CATCAGCAAACTTCTCCTTGACAGCAGAAACAGAAGAAAGGAACTTCCCCCATGGAAATGAACAT
CTGTTAAATTGGGCCCGAAAAGAGTATGGAGCAGTTACTTCATTCACCGAACTCAAGATAGCAAG
AAACATTTATATTAAAGTGGGGGAAGATCAAGTGTTCCCTCCAAAGTGCAACATAGGGAAGAATT
TTCTCTCACTCAATTACCTTGCTGAGTACCTTCAACCCAAAGCAGCAGAAGGGTGTGTGATGTCC
AGCCAGCCCCAGAATGAGGAAGTACACATCATCGAGCTAATCACCCCCAACTCTAACCCCTACA
GTGCTTTCCAGGTGGATATAACAATTGATATAAGACCTTCTCAAGAGGATCTTGAAGTGGTCAAA
AATCTCATCCTGATCTTGAAGTCTAAAAAGTCTGTCAACTGGGTGATCAAATCTTTTGATGTTAAG
GGAAGCCTGAAAATTATTGCTCCTAACAGTATTGGCTTTGGAAAAGAGAGTGAAAGATCTATGAC
AATGACCAAATCAATAAGAGATGACATTCCTTCAACCCAAGGGAATCTGGTGAAGTGGGCTTTGG
ACAATGGCTATAGTCCAATAACTTCATACACAATGGCTCCTGTGGCTAATAGATTTCATCTTCGGC
TTGAAAATAATGAGGAGATGGGAGATGAGGAAGTCCACACTATTCCTCCTGAGCTACGGATCCT
GCTGGACCCTGGTGCCCTGCCGCAACTTTGCAAGTTCTGCGACGTGCGATTCTCTACGTGCGAT
AATCAAAAGTCCTGTATGTCAAACTGCAGTATTACTTCTATTTGTGAGAAGCCTCAGGAGGTTTGT
GTCGCGGTCTGGCGGAAAAACGACGAAAATATCACATTGGAAACGGTCTGCCACGACCCCAAAC
TTCCCTATCATGATTTCATACTTGAGGATGCAGCTTCACCTAAGTGTATTATGAAAGAGAAGAAGA
AACCAGGCGAAACGTTCTTTATGTGCAGTTGCTCCTCCGATGAATGCAATGATAACATCATTTTCT
CCGAGGAGTACAATACTTCAAATCCAGACCTCCTTCTCGTCATTTTTCAAGTTACAGGTATTTCAC
TGCTCCCCCCTCTCGGCGGAGGCGGTTCTGACAAAACTCACACATGCCCACCGTGCCCAGCAC
CTGAACTCCTGGGGGGACCGTCAGTCTTCCTCTTCCCCCCAAAACCCAAGGACACCCTCATGAT
CTCCCGGACCCCTGAGGTCACATGCGTGGTGGTGGACGTGAGCCACGAAGACCCTGAGGTCAA
GTTCAACTGGTACGTGGACGGCGTGGAGGTGCATAATGCCAAGACAAAGCCGCGGGAGGAGCA
GTACAACAGCACGTACCGTGTGGTCAGCGTCCTCACCGTCCTGCACCAGGACTGGCTGAATGG
CAAGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGATGAGCTG
ACCAAGAACCAGGTCAGCCTGACCTGCCTGGTCAAAGGCTTCTATCCCAGCGACATCGCCGTGG
AGTGGGAGAGCAATGGGCAGCCGGAGAACAACTACAAGACCACGCCTCCCGTGCTGGACTCCG
ACGGCTCCTTCTTCCTCTATTCCAAGCTCACCGTGGACAAGAGCAGGTGGCAGCAGGGGAACGT
CTTCTCATGCTCCGTGATGCATGAGGCTCTGCACAACCACTACACGCAGAAGAGCCTCTCCCTG
TCTCCCGGGAAAGACGACGATGATGATGACGATGACGACGATTGATGACTCGAGTTT

1. A method of treating a human patient suffering from a bone disease associated with elevated TGF-β signaling, said method comprising administering to said patient a therapeutically effective amount of a composition comprising a fusion protein comprising a TGF-β receptor antagonist and a bone-targeting moiety.

2. The method of claim 1, wherein said disease is a disease associated with elevated bone turnover.

3. The method of claim 1, wherein said disease is selected from the group consisting of osteogenesis imperfecta, McCune-Albright syndrome, Gaucher disease, hyperoxaluria, Paget disease of bone, and juvenile Paget disease.

4. The method of claim 3, wherein said disease is osteogenesis imperfecta.

5. (canceled)

6. The method of claim 1, wherein said fusion protein has the amino acid sequence of SEQ ID NO: 28; or a variant of said amino acid sequence.

7. The method of claim 1, wherein said fusion protein has the amino acid sequence of SEQ ID NO: 30; or a variant of said amino acid sequence.

8. A composition comprising a homodimer of a compound of the formula:
(A-L¹-B-L²-Z), I(a).
(Z-L²-B-L¹-A), or I(b).
(B-L¹-A-L²-Z), I(c).
wherein each A is independently an RER heterotrimeric fusion polypeptide;
wherein each L¹ is independently a linker;
wherein each B is independently an Fc domain of an immunoglobulin or is absent;
wherein each L² is independently a linker or is absent;
wherein each Z is independently a bone-targeting moiety or is absent;
wherein each A, the RER heterotrimeric fusion polypeptide, independently comprises a polypeptide of the formula: W-L³-X-L⁴-Y, wherein
W is a TGF-β type II receptor ectodomain or a portion thereof;
L³ is a linker or is absent;
X is a TGF-β type III receptor endoglin domain or a portion thereof;
L⁴ is a linker or is absent;
Y is a TGF-β type II receptor ectodomain or a portion thereof, and
wherein at least one of B and Z is present.

9. (canceled)

10. The composition of claim 8, wherein B is present.

11-17. (canceled)

18. The composition of claim 8, wherein Z is present.

19. (canceled)

20. The composition of claim 18, wherein each Z independently comprises a polyanionic peptide, a bisphosphonate, or a peptide having the amino acid sequence of SEQ ID NO: 46 or a variant of said amino acid sequence.

21. The composition of claim 8, wherein the TGF-β type II receptor ectodomain, W, is at the N-terminus of the RER heterotrimeric fusion polypeptide and the TGF-β type II receptor ectodomain, Y, is at the C-terminus of the RER heterotrimeric fusion polypeptide.

22. The composition of claim 21, wherein:
a) the C-terminus of the TGF-β type II receptor ectodomain, Y, is covalently joined to the N-terminus of B, Fc domain of an immunoglobulin, via the linker L¹ as in formula I(a); or
b) the N-terminus of the TGF-β type II receptor ectodomain, W, is covalently joined to the C-terminus of B via the linker L¹ as in formula I(b) or I(c).

23. (canceled)

24. The composition of claim 22, wherein the amino acid sequence of the TGF-β type II receptor ectodomain, W, is different than the amino acid sequence of the TGF-β type II receptor ectodomain, Y.

25-37. (canceled)

38. The composition of claim 8, comprising the homodimer of a compound of the formula I(a). (A-L¹-B-L²-Z),
wherein A is an RER heterotrimeric fusion polypeptide;
wherein L¹ is a linker;
wherein B is an Fc domain of an immunoglobulin;
wherein L² is a linker that is absent;
wherein Z is a bone-targeting moiety;
wherein A, the RER heterotrimeric fusion polypeptide, comprises a polypeptide of the formula: W-L³-X-L⁴-Y, wherein
W is a TGF-β type II receptor ectodomain or a portion thereof;
L³ is a linker;
X is a TGF-β type III receptor endoglin domain or a portion thereof;
L⁴ is a linker that is absent; and
Y is a TGF-β type II receptor ectodomain or a portion thereof.

39-68. (canceled)

69. A method of treating a human patient suffering from a disease associated with elevated TGF-β signaling, said method comprising administering to said patient a therapeutically effective amount of the composition of claim 8.

70-74. (canceled)

75. The method of claim 69, wherein said disease is osteogenesis imperfecta.

76-110. (canceled)

111. The method of claim 69, wherein said homodimer comprises the amino acid sequence of:
a) SEQ ID NO: 29, or a variant of said amino acid sequence;
b) SEQ ID NO: 28, or a variant of said amino acid sequence;
c) SEQ ID NO: 31, or a variant of said amino acid sequence; or
d) SEQ ID NO: 30, or a variant of said amino acid sequence.

112-114. (canceled)

115. A method of improving muscle function in a human patient suffering from a disease associated with elevated TGF-β signaling, said method comprising administering to said patient a therapeutically effective amount of the composition of claim 8.

116-119. (canceled)

120. The method of claim 115, wherein said disease is osteogenesis imperfecta.

121-173. (canceled)

174. A method of treating a human patient suffering from a bone disease associated with elevated TGF-β signaling, said method comprising administering to said patient a therapeutically effective amount of a TGF-β antagonist comprising an antibody or antigen-binding fragment thereof that binds TGF-β, wherein said antibody or antigen-binding fragment thereof is conjugated to a targeting moiety that binds a protein or mineral present in human bone tissue.

175-185. (canceled)

186. A method of improving muscle function in a human patient suffering from a disease associated with elevated TGF-β signaling, said method comprising administering to said patient a therapeutically effective amount of a TGF-β antagonist comprising an antibody or antigen-binding fragment thereof that binds TGF-β, wherein said antibody or antigen-binding fragment thereof is conjugated to a targeting moiety that binds a protein or mineral present in human bone tissue.

187-238. (canceled)

239. The method of claim 174, wherein:
said antibody or antigen-binding fragment thereof comprises the following complementarity determining regions (CDRs):
a CDR-H1 having the amino acid sequence SNVIS (SEQ ID NO: 64);
a CDR-H2 having the amino acid sequence GVIPIVDIANYAQRFKG (SEQ ID NO: 65);
a CDR-H3 having the amino acid sequence TLGLVLDAMDY (SEQ ID NO: 66);
a CDR-L1 having the amino acid sequence RASQSLGSSYLA (SEQ ID NO: 67);
a CDR-L2 having the amino acid sequence GASSRAP (SEQ ID NO: 68); and
a CDR-L3 having the amino acid sequence QQYADSPIT (SEQ ID NO: 69); or
said antibody or antigen binding fragment thereof competitively inhibits the binding of TGF-β to an antibody or antigen binding fragment thereof that comprises the following CDRs:
a CDR-H1 having the amino acid sequence SNVIS (SEQ ID NO: 64);
a CDR-H2 having the amino acid sequence GVIPIVDIANYAQRFKG (SEQ ID NO: 65);
a CDR-H3 having the amino acid sequence TLGLVLDAMDY (SEQ ID NO: 66);
a CDR-L1 having the amino acid sequence RASQSLGSSYLA (SEQ ID NO: 67);
a CDR-L2 having the amino acid sequence GASSRAP (SEQ ID NO: 68); and
a CDR-L3 having the amino acid sequence QQYADSPIT (SEQ ID NO: 69).

240-262. (canceled)

263. A kit comprising the pharmaceutical formulation comprising the composition of claim 8, wherein the kit further comprises a package insert instructing a user of said kit to treat a human patient suffering from a disease associated with elevated TGF-β signaling and/or elevated turnover, fibrosis, or cancer by administering to said patient a therapeutically effective amount of said pharmaceutical formulation.

264. (canceled)

265. A cell comprising a nucleic acid encoding the composition of claim 8.

266. A method of manufacturing the composition of claim 8, said method comprising:
culturing a cell comprising a nucleic acid encoding the composition in a suitable growth medium; and
isolating the mature form of the polypeptide encoded by said nucleic acid.

267. The composition of claim 8, wherein:
W has an amino acid sequence that is at least 90% identical to the amino acid sequence of SEQ ID NO: 10;
X has an amino acid sequence that is at least 90% identical to the amino acid sequence of SEQ ID NO: 12; and
Y has an amino acid sequence that is at least 90% identical to the amino acid sequence of SEQ ID NO: 11.

268. The composition of claim 8, wherein the homodimer has the amino acid sequence of SEQ ID NO: 28 or 30.
